(19) United States
(12) Patent Application Publication
(10) Pub. No.: US 2005/0131017 A1
(43) Pub. Date: Jun. 16, 2005

(54) HIV protease inhibiting compounds

(76) Inventors: David A. DeGoey, Salem, WI (US); Charles A. Flentge, Salem, WI (US); William J. Flosi, Evanston, IL (US); David J. Grampovnik, Waukegan, IL (US); Dale J. Kempf, Libertyville, IL (US); Larry L. Klein, Lake Forest, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/733,946

(22) Filed: Dec. 11, 2003

(51) Int. Cl. A61K 31/4709; A61K 31/4439; A61K 31/427; C07D 43/02; C07D 417/02

(52) U.S. Cl. 514/312; 514/341; 546/272.7; 546/153; 514/365

(57) ABSTRACT

A compound of the formula 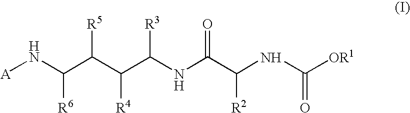 is disclosed as an HIV protease inhibitor. Methods and compositions for inhibiting an HIV infection are also disclosed.

### TECHNICAL FIELD

The present invention relates to novel compounds and a composition and a method for inhibiting human immunodeficiency virus (HIV) protease, a composition and method for inhibiting or treating an HIV infection, processes for making the compounds and synthetic intermediates employed in the processes.

### BACKGROUND OF THE INVENTION

The genome of the human immunodeficiency virus (HIV) encodes a protease that is responsible for the proteolytic processing of one or more polyprotein precursors such as the pol and gag gene products. HIV protease processes the gag precursor into core proteins and also processes the pol precursor into reverse transcriptase and protease.

The correct processing of the precursor polyproteins by HIV protease is necessary for the assembly of infectious virions. Therefore, inhibition of HIV protease provides a useful target for development of therapeutic agents for treatment of HIV infection.

In recent years, inhibitors of HIV protease have become an important class of therapeutic agents for inhibition and treatment of HIV infection in humans. HIV protease inhibitors are especially effective when administered in combination with other classes of HIV therapeutic agents, especially inhibitors of HIV reverse transcriptase, in “cocktails” of HIV therapeutic agents.

At the present time, the HIV protease inhibitors saquinavir, ritonavir, indinavir, nelfinavir, amprenavir, lopinavir/ritonavir, fosamprenavir, and atazanavir have been approved in the U.S. for treatment of HIV infection. There is a continuing need for improved HIV protease inhibitors that are very potent, that have reduced side-effects and that are effective against resistant strains of HIV.

### SUMMARY OF THE INVENTION

The present invention provides a compound of formula (I) 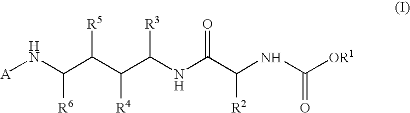
or a pharmaceutically acceptable salt form, stereoisomer, ester, salt of an ester, prodrug, salt of a prodrug, or combination thereof, wherein: 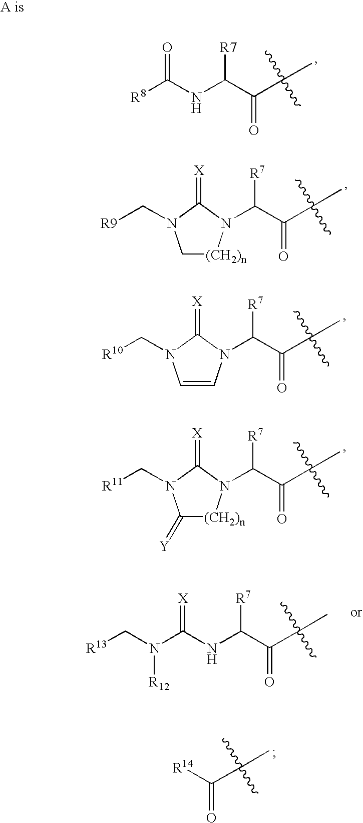
X is O, S or NH;
Y is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R¹²;
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroalkyl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁸ is —ORₐ or -alkylORₐ;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁴ is —ORₐ or -alkylORₐ;
R¹⁶ is hydrogen or R¹⁵;
R¹⁵ is 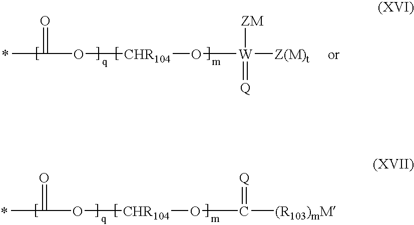
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H; alkyl, alkenyl or R₁₀₆; wherein 1 to 4 —CH₂ radicals of the alkyl or alkenyl is optionally replaced by a heteroatom group selected from O, S, S(O), SO₂ or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl)-alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

The present invention also provides the processes of making a compound of the present invention and intermediates employed in the processes.

The present invention further provides a pharmaceutical composition comprising a therapeutically effective amount of a compound or combination of compounds of the present invention, or a pharmaceutically acceptable salt form, stereoisomer, ester, salt of an ester, prodrug, salt of a prodrug, or combination thereof, and a pharmaceutically acceptable carrier.

The present invention yet further provides a pharmaceutical composition comprising a therapeutically effective amount of a compound or combination of compounds of the present invention, or a pharmaceutically acceptable salt form, stereoisomer, ester, salt of an ester, prodrug, salt of a prodrug, or combination thereof, and one, two, three, four, five or six agents selected from the group consisting of a second HIV protease inhibitor, a HIV reverse transcriptase inhibitor, an HIV entry/fusion inhibitor, an HIV integrase inhibitor and an HIV budding/maturation inhibitor, and a pharmaceutically acceptable carrier.

The present invention also provides a pharmaceutical composition comprising a therapeutically effective amount of a compound or combination of compounds of the present invention, or a pharmaceutically acceptable salt form, stereoisomer, ester, salt of an ester, prodrug, salt of a prodrug, or combination thereof, ritonavir, and a pharmaceutically acceptable carrier.

The present invention still further provides a method of inhibiting the replication of an HIV virus comprising contacting said virus with a therapeutically effective amount of a compound or combination of compounds of the present invention, or a pharmaceutically acceptable salt form, stereoisomer, ester, salt of an ester, prodrug, salt of a prodrug, or combination thereof, and a pharmaceutically acceptable carrier.

The present invention still further provides a method of inhibiting the replication of an HIV virus comprising contacting said virus with the pharmaceutical composition of the present invention.

The present invention further provides a method of inhibiting HIV protease comprising contacting said HIV protease with a therapeutically effective amount of a compound or combination of compounds of the present invention, or a pharmaceutically acceptable salt form, stereoisomer, ester, salt of an ester, prodrug, salt of a prodrug, or combination thereof, and a pharmaceutically acceptable carrier.

The present invention further provides a method of inhibiting HIV protease comprising contacting said HIV protease with the pharmaceutical composition of the present invention.

The present invention also provides a method of treating or preventing an HIV infection comprising administering to a patient in need of such treatment a therapeutically effective amount of a compound or combination of compounds of the present invention, or a pharmaceutically acceptable salt form, stereoisomer, ester, salt of an ester, prodrug, salt of a prodrug, or combination thereof, and a pharmaceutically acceptable carrier.

The present invention also provides a method of treating or preventing an HIV infection comprising administering to a patient in need of such treatment the pharmaceutical composition of the present invention.

### DETAILED DESCRIPTION OF THE INVENTION

As used in the present specification the following terms have the meanings indicated:

As used herein, the singular forms “a”, “an”, and “the” may include plural reference unless the context clearly dictates otherwise.

The term “activated carboxylic acid group” as used herein refers to acid halides such as acid chlorides and also refers to activated ester derivatives including, but not limited to, formic and acetic acid derived anhydrides, anhydrides derived from alkoxycarbonyl halides such as isobutyloxycarbonylchloride and the like, anhydrides derived from reaction of the carboxylic acid with N,N′-carbonyldiimidazole and the like, N-hydroxysuccinimide derived esters, N-hydroxyphthalimide derived esters, N-hydroxybenzotriazole derived esters, N-hydroxy-5-norbornene-2,3-dicarboximide derived esters, 2,4,5-trichlorophenol derived esters, p-nitrophenol derived esters, phenol derived esters, pentachlorophenol derived esters, 8-hydroxyquinoline derived esters and the like.

The term “alkanoyl” as used herein refers to an alkyl group attached to the parent molecular moiety through a carbonyl group. Representative examples of alkanoyl include, but not limited to, methylcarbonyl, ethylcarbonyl and tert-butylcarbonyl.

The term “alkyl,” as used herein, refers to a group derived from a straight or branched chain saturated hydrocarbon containing 1, 2, 3, 4, 5, 6, 7, 8, 9 or 10 carbon atoms. Representative examples of alkyl groups include, but not limited to, butyl, methyl, 1-methylpropyl, 2-methylbutyl, tert-butyl and isopropyl(1-methylethyl).

The term “alkylamino” as used herein refers to —N(H)R⁹⁰ wherein R⁹⁰ is alkyl.

The term “alkylaminocarbonyl” as used herein refers to an alkylamino group attached to the parent molecular moiety through a carbonyl group.

The term “alkenyl,” as used herein, refers to a straight or branched chain group of 2, 3, 4, 5, 6, 7, 8, 9 or 10 carbon atoms containing at least one carbon-carbon double bond. Representative examples of alkenyl groups include, but not limited to, allyl, propenyl and 3-methyl-2-butenyl.

The term “alkynyl,” as used herein, refers to a straight or branched chain hydrocarbon of 2, 3, 4, 5, 6, 7, 8, 9 or 10 carbon atoms containing at least one carbon-carbon triple bond. Representative examples of alkynyl groups include, but not limited to, ethynyl, 2-methyl-3-butynyl and 3-pentynyl.

The term “alkoxy,” as used herein, refers to an alkyl group attached to the parent molecular moiety through an oxygen atom. Representative examples of alkoxy groups include, but not limited to, tert-butoxy, methoxy and isopropoxy.

The term “alkoxyalkyl,” as used herein, refers to an alkyl group substituted by at least one alkoxy group.

The term “alkoxycarbonyl,” as used herein, refers to an alkoxy group attached to the parent molecular moiety through a carbonyl group. Representative examples of alkoxycarbonyl groups include, but not limited to, tert-butoxycarbonyl, ethoxycarbonyl and methoxycarbonyl.

The term “aryl” as used herein, refers to a phenyl group, or a bicyclic or tricyclic hydrocarbon fused ring systems wherein one or more of the rings is a phenyl group. Bicyclic fused ring systems have a phenyl group fused to a monocyclic cycloalkenyl group, as defined herein, a monocyclic cycloalkyl group, as defined herein, or another phenyl group. Tricyclic fused ring systems are exemplified by a bicyclic fused ring system fused to a monocyclic cycloalkenyl group, as defined herein, a monocyclic cycloalkyl group, as defined herein, or another phenyl group. Representative examples of aryl groups include, but not limited to, anthracenyl, azulenyl, fluorenyl, indanyl, indenyl, naphthyl, phenyl and tetrahydronaphthyl. The aryl groups of the present invention can be substituted or unsubstituted, and are connected to the parent molecular moiety through any substitutable carbon atom of the group.

The term “arylalkyl”, as used herein, refers to an aryl group, as defined herein, attached to the parent molecular moiety through an alkyl group.

The term “carbonyl” as used herein, refers to C(═O).

The term “cyano,” as used herein, refers to CN.

The term “cyanoalkyl,” as used herein, refers to a cyano group attached to the parent molecular moiety through an alkyl group.

The term “cycloalkenyl,” as used herein, refers to a non-aromatic, partially unsaturated, monocyclic, bicyclic or tricyclic hydrocarbon ring system, having three to fourteen carbon atoms and zero heteroatom. Representative examples of cycloalkenyl groups include, but not limited to, cyclopropyl, cyclobutyl, cyclopentyl, cyclohexenyl, octahydronaphthalenyl and norbornylenyl. The cycloalkenyl groups of the present invention can be unsubstituted or substituted, and are attached to the parent molecular moiety through any substitutable carbon atom of the group.

The term “cycloalkenylalkyl”, as used herein, refers to a cycloalkenyl group attached to the parent molecular moiety through an alkyl group.

The term “cycloalkyl,” as used herein, refers to a saturated monocyclic, bicyclic, or tricyclic hydrocarbon ring system having three to fourteen carbon atoms and zero heteroatom. Representative examples of cycloalkyl groups include, but not limited to, cyclopropyl, cyclobutyl, cyclopentyl, cyclohexyl, cycloheptyl, bicyclo[3.1.1]heptyl, 6,6-dimethylbcyclo[3.1.1]heptyl and adamanty like. The cycloalkyl groups of the present invention can be unsubstituted or substituted, and are connected to the parent molecula moiety through any substitutable carbon atom of the group.

The term “cycloalkylalkyl”, as used herein, refers to a cycloalkyl group attached to the parent molecular moiety through an alkyl group.

The term “dialkylamino” as used herein refers to —NR⁹⁰R⁹¹, wherein R⁹⁰ and R⁹¹ are alkyls.

The term “dialkylaminocarbonyl” as used herein refers to a dialkylamino group as defined herein, appended to the parent molecular moiety through a carbonyl group.

The terms “halo,” and “halogen” as used herein, refer to F, Cl, Br, and I.

The term “haloalkoxy,” as used herein, refers to a haloalkyl group attached to the parent molecular moiety through an oxygen atom.

The term “haloalkenyl” as used herein, refers to an alkenyl group substituted by one, two, three or four halogen atoms.

The term “haloalkyl” as used herein, refers to an alkyl group substituted by one, two, three, or four halogen atoms.

The term “heteroaryl” as used herein, refers to an aromatic five- or six-membered ring where at least one atom is selected from the group consisting of N, O, and S, and the remaining atoms are carbon. The term “heteroaryl” also includes bicyclic systems where a heteroaryl ring is fused to a phenyl group, a monocyclic cycloalkyl group, as defined herein, a heterocycle group, as defined herein, or an additional heteroaryl group. The term “heteroaryl” also includes tricyclic systems where a bicyclic system is fused to a phenyl group, a monocyclic cycloalkyl group, as defined herein, a heterocycle group, as defined herein, or an additional heteroaryl group. Representative examples of heteroaryl groups include, but not limited to, benzothienyl, benzoxazolyl, benzimidazolyl, benzoxadiazolyl, dibenzofuranyl, dihydrobenzothiazolyl, furanyl, imidazolyl, indazolyl, indolyl, isoindolyl, isoxazolyl, isoquinolinyl, isothiazolyl, oxadiazolyl, oxazolyl, thiazolyl, thienopyridinyl, thienyl, triazolyl, thiadiazolyl, tetrazolyl, pyridoimidazolyl, pyridyl, pyridazinyl, pyrimidinyl, pyrazinyl, pyrazolyl, pyrrolyl, quinolinyl, tetrahydroquinolinyl and triazinyl. The heteroaryl groups of the present invention can be substituted or unsubstituted, and are connected to the parent molecular moiety through any substitutable carbon or nitrogen atom in the groups. In addition, the nitrogen heteroatoms may or may not be quaternized or oxidized to the N-oxide. Also, the nitrogen containing rings may or may not be N-protected.

The term “heteroarylalkyl” as used herein, refers to an heteroaryl group as defined herein, appended to the parent molecular moiety through an alkyl group as defined herein.

The term “heterocycle” as used herein, refers to cyclic, non-aromatic, saturated or partially unsaturated, three, four, five-, six-, or seven-membered rings containing at least one atom selected from the group consisting of oxygen, nitrogen, and sulfur. The term “heterocycle” also includes bicyclic systems where a heterocycle ring is fused to a phenyl group, a monocyclic cycloalkenyl group, as defined herein, a monocyclic cycloalkyl group, as defined herein, or an additional monocyclic heterocycle group. The term “heterocycle” also includes tricyclic systems where a bicyclic system is fused to a phenyl group, a monocyclic cycloalkenyl group, as defined herein, a monocyclic cycloalkyl group, as defined herein, or an additional monocyclic heterocycle group. The heterocycle groups of the invention are substituted or unsubstituted, and are connected to the parent molecular moiety through any substitutable carbon or nitrogen atom in the groups. Representative examples of heterocycle groups include, but not limited to, benzoxazinyl, dihydroindolyl, dihydropyridinyl, 1,3-dioxanyl, 1,4-dioxanyl, 1,3-dioxolanyl, hexahydrofurofuranyl, isoindolinyl, morpholinyl, piperazinyl, pyrrolidinyl, tetrahydropyridinyl, piperidinyl, thiomorpholinyl and tetrahydropyranyl. The nitrogen heteroatoms may or may not be quaternized or oxidized to the N-oxide. In addition, the nitrogen containing heterocyclic rings may or may not be N-protected.

The term “heterocyclealkyl” as used herein, refers to an heterocycle group as defined herein, appended to the parent molecular moiety through an alkyl group as defined herein.

The term “hydroxy,” as used herein, refers to —OH.

The term “hydroxyalkyl” as used herein, refers to an alkyl group substituted by at least one hydroxy group.

The term “nitro,” as used herein, refers to —NO₂.

The term “nitroalkyl” as used herein, refers to an alkyl group substituted by at least one nitro group.

The term “oxo,” as used herein, refers to ═O.

It is understood that each of the terms as defined hereinabove: alkanoyl, alkenyl, alkoxy, alkoxyalkyl, alkoxycarbonyl, alkyl, alkylamino, alkylaminocarbonyl, alkynyl, aryl, arylalkyl, cyanoalkyl, cycloalkenyl, cycloalkenylalkyl, cycloalkyl, cycloalkylalkyl, dialkylamino, dialkylaminocarbonyl, haloalkoxy, haloalkenyl, haloalkyl, heteroaryl, heteroarylalkyl, heterocycle, heterocyclealkyl, hydroxyalkyl, nitroalkyl, may be unsubstituted or substituted.

In a first embodiment the present invention provides a compound of formula (I) 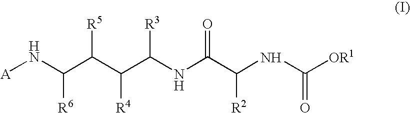
or a pharmaceutically acceptable salt form, stereoisomer, ester, salt of an ester, prodrug, salt of a prodrug, or combination thereof, wherein:
A is 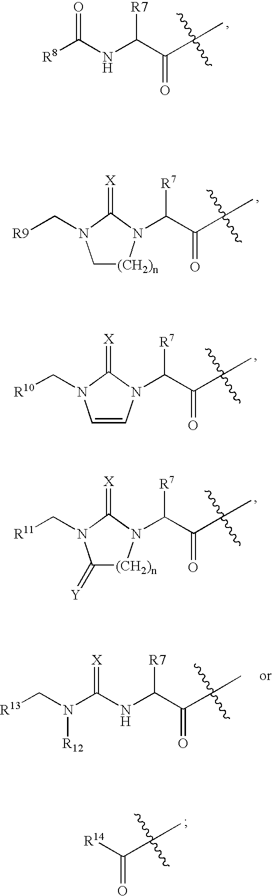
X is O, S or NH;
Y is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁸ is —ORₐ or -alkylORₐ;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁴ is —ORₐ or -alkylORₐ;
R¹⁶ is hydrogen or R¹⁵;
R¹⁵ is 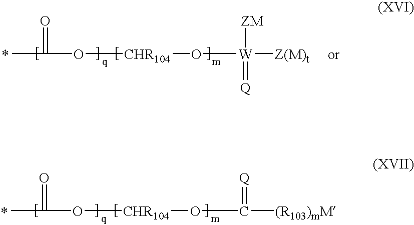
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆-SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

For example, the present invention provides a compound of formula (I) wherein R⁴ is H and R⁵ is OR¹⁶.

For example, the present invention provides a compound of formula (I) wherein R⁴ is OR¹⁶ and R⁵ is H.

For example, the present invention provides a compound of formula (I) wherein X is O and Y is O.

For example, the present invention provides a compound of formula (I) wherein X is O, Y is O, R⁴ is H and R⁵ is OR¹⁶.

For example, the present invention provides a compound of formula (I) wherein X is O, Y is O, R⁴ is OR¹⁶ and R⁵ is H.

For example, the present invention provides a compound of formula (I) wherein X is O and Y is O, R⁴ is H, R⁵ is OR¹⁶ and R² is alkyl.

For example, the present invention provides a compound of formula (I) wherein X is O and Y is O, R⁴ is OR¹⁶, R⁵ is H and R² is alkyl.

For example, the the present invention provides a compound of formula (I) wherein X is O, Y is O, R⁴ is H, R⁵ is OR¹⁶, R² is alkyl, and R³ is arylalkyl.

For example, the the present invention provides a compound of formula (I) wherein X is O, Y is O, R⁴ is OR¹⁶, R⁵ is H, R² is alkyl, and R³ is arylalkyl.

For example, the the present invention provides a compound of formula (I) wherein X is O, Y is O, R⁴ is H, R⁵ is OR¹⁶, R² is alkyl, and R³ is arylalkyl substituted with R^{1a}.

For example, the the present invention provides a compound of formula (I) wherein X is O, Y is O, R⁴ is OR¹⁶, R⁵ is H, R² is alkyl, and R³ is arylalkyl substituted with Rₐ.

For example, the the present invention provides a compound of formula (I) wherein X is O, Y is O, R⁴ is H, R⁵ is OR¹⁶, R² is alkyl, R³ is arylalkyl substituted with R^{3a} and R^{3a} is aryl or heteroaryl.

For example, the the present invention provides a compound of formula (I) wherein X is O and Y is O, R⁴ is OR¹⁶, R⁵ is H, R² is alkyl, R³ is arylalkyl substituted with R^{3a} and R^{3a} is aryl or heteroaryl.

For example, the present invention provides a compound of formula (I) wherein X is O, Y is O, R⁴ is H, R⁵ is OR¹⁶, R² is C1, C2, C3, C4 or C5 alkyl, R³ is phenylmethyl substituted with R^{3a}, and R^{3a} is aryl or heteroaryl.

For example, the present invention provides a compound of formula (I) wherein X is O and Y is O, R⁴ is OR¹⁶, R⁵ is H, R² is C1, C2, C3, C4 or C5 alkyl, R³ is phenylmethyl substituted with R³, and R^{3a} is aryl or heteroaryl.

For example, the present invention provides a compound of formula (I) wherein X is O and Y is O, R⁴ is H, R⁵ is OR¹⁶, R² is C1, C2, C3, C4 or C5 alkyl, R³ is phenylmethyl substituted with R^{3a}, and R^{3a} is pyridyl.

For example, the present invention provides a compound of formula (I) wherein X is O and Y is O, R⁴ is OR¹⁶, R⁵ is H, R² is C1, C2, C3, C4 or C5 alkyl, R³ is phenylmethyl substituted with R^{3a}, and R^{3a} is pyridyl.

For example, the present invention provides a compound of formula (I) wherein X is O and Y is O, R⁴ is —H, R⁵ is OR¹⁶, R² is 1-methylpropyl, tert-butyl or isopropyl, R³ is phenylmethyl substituted with R³³, and R^{3a} is 2-pyridyl.

For example, the present invention provides a compound of formula (I) wherein X is O and Y is O, R⁴ is OR¹⁶, R⁵ is H, R² is 1-methylpropyl, tert-butyl or isopropyl, R³ is phenylmethyl substituted with R^{3a}, and R^{3a} is 2-pyridyl.

Exemplary compounds of the present invention of formula (I) include, but not limited to, the following:
methyl 7-benzyl 1,10-ditert-butyl-6-hydroxy-2,9,12-trioxo-4-[4-(2-pyridinyl)benzyl]-13-oxa-3,8,11-triazatetradec-1-ylcarbamate;
methyl 4-benzyl-1,10-ditert-butyl-5-hydroxy-2,9,12-trioxo-7-[4-(2-pyridinyl)benzyl]-13-oxa-3,8,1-triazatetradec-1-ylcarbamate;
methyl 1-{[(1-benzyl-3-hydroxy-4-{[3-methyl-2-(2-oxo-3-{[2-(2-pyridinyl)-1,3-thiazol-4-yl]methyl}-1-imidazolidinyl)pentanoyl]amino}-5-phenylpentyl)amino]carbonyl}-2,2-dimethylpropylcarbamate;
methyl 1-({[1-benzyl-3-hydroxy-4-({3-methyl-2-[2-oxo-3-(4-quinolinylmethyl)-1-imidazolidinyl]pentanoyl}amino)-5-phenylpentyl]amino}carbonyl)-2,2-dimethylpropylcarbamate;
methyl 1-({[1-benzyl-3-hydroxy-4-({3-methyl-2-[2-oxo-3-(4-quinolinylmethyl)-1-imidazolidinyl]pentanoyl}amino)-5-phenylpentyl]amino}carbonyl)-2-methylbutylcarbamate;
methyl 1-{[(1-benzyl-3-hydroxy-4-{[2-(3-{[2-(methoxymethyl)-1,3-thiazol-4-yl]methyl}-2-oxo-1-imidazolidinyl)-3,3-dimethylbutanoyl]amino}-5-phenylpentyl)amino]carbonyl}-2,2-dimethylpropylcarbamate;
methyl 1-[({1-benzyl-3-hydroxy-4-[(3-methyl-2-{3-[(6-methyl-2-pyridinyl)methyl]-2-oxo-1-imidazolidinyl}pentanoyl)amino]-5-phenylpentyl}amino)carbonyl]-2,2-dimethylpropylcarbamate;
methyl 1-[({1-benzyl-2-hydroxy-4-[(3-methyl-2-{3-[(6-methyl-2-pyridinyl)methyl]-2-oxo-1-imidazolidinyl}pentanoyl)amino]-5-phenylpentyl}amino)carbonyl]-2,2-dimethylpropylcarbamate;
methyl 1-[({1-benzyl-2-hydroxy-4-[(3-methyl-2-{3-[(6-methyl-2-pyridinyl)methyl]-2-oxo-1-imidazolidinyl}pentanoyl)amino]-5-phenylpentyl}amino)carbonyl]-2-methylbutylcarbamate;
methyl 1-[({1-benzyl-4-[(3,3-dimethyl-2-{3-[(6-methyl-2-pyridinyl)methyl]-2-oxo-1-imidazolidinyl}butanoyl)amino]-2-hydroxy-5-phenylpentyl}amino)carbonyl]-2,2-dimethylpropylcarbamate;
methyl 1-[({1-benzyl-2-hydroxy-4-[(3-methyl-2-{3-[(2-methyl-1,3-thiazol-5-yl)methyl]-2-oxo-1-imidazolidinyl}pentanoyl)amino]-5-phenylpentyl}amino)carbonyl]-2,2-dimethylpropylcarbamate;
methyl 1-{[(1-benzyl-2-hydroxy-4-{[3-methyl-2-(2-oxo-3-{[2-(3-pyridinyl)-1,3-thiazol-4-yl]methyl}-1-imidazolidinyl)pentanoyl]amino}-5-phenylpentyl)amino]carbonyl}-2,2-dimethylpropylcarbamate;
methyl 1-[({1-benzyl-2-hydroxy-4-[(3-methyl-2-{3-[(6-methyl-3-pyridinyl)methyl]-2-oxo-1-imidazolidinyl}pentanoyl)amino]-5-phenylpentyl}amino)carbonyl]-2,2-dimethylpropylcarbamate;
methyl 1-[({1-benzyl-4-[(3,3-dimethyl-2-{3-[(2-methyl-1,3-thiazol-4-yl)methyl]-2-oxo-1-imidazolidinyl}butanoyl)amino]-2-hydroxy-5-phenylpentyl}amino)carbonyl]-2,2-dimethylpropylcarbamate;
methyl 1-[({1-benzyl-2-hydroxy-4-[(3-methyl-2-{3-[(2-methyl-3-pyridinyl)methyl]-2-oxo-1-imidazolidinyl}pentanoyl)amino]-5-phenylpentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-{[(1-benzyl-2-hydroxy-4-{[2-(3-{[6-(1-hydroxy-1-methylethyl)-2-pyridinyl]methyl}-2-oxo-1-imidazolidinyl)-3,3-dimethylbutanoyl]amino}-5-phenylpentyl)amino]carbonyl}-2,2-dimethylpropylcarbamate;
methyl 1-[({3-hydroxy-4-[(3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-{[(1-benzyl-4-{[3,3-dimethyl-2-(2-oxo-3-{[2-(3-pyridinyl)-1,3-thiazol-4-yl]methyl}-1-imidazolidinyl)butanoyl]amino-}2-hydroxy-5-phenylpentyl)amino]carbonyl}-2,2-dimethylpropylcarbamate;
methyl 1-({[1-benzyl-4-({3,3-dimethyl-2-[2-oxo-3-(3-pyridinylmethyl)-1-imidazolidinyl]butanoyl}amino)-2-hydroxy-5-phenylpentyl]amino}carbonyl)-2,2-dimethylpropylcarbamate;
methyl 1-[({3-hydroxy-4-[(3-methyl-2-{3-[(2-methyl-3-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({1-benzyl-4-[(3,3-dimethyl-2-{3-[(6-methyl-2-pyridinyl)methyl]-2-oxo-1-imidazolidinyl}butanoyl)amino]-3-hydroxy-5-phenylpentyl}amino)carbonyl]-2,2-dimethylpropylcarbamate;
methyl 1-[({4[(3,3-dimethyl-2-{3-[(6-methyl-2-pyridinyl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl 1-[({1-benzyl-4-[(3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentyl}amino)carbonyl]-2,2-dimethylpropylcarbamate;
methyl 7-benzyl-1,10-ditert-butyl-5-hydroxy-2,9,12-trioxo-4-[4-(2-pyridinyl)benzyl]-13-oxa-3,8,1-triazatetradec-1-ylcarbamate;
1:1 mixture of (3R,3aS,6aR)-hexahydrofuro[2,3-b]furan-3-yl 1-benzyl-3-hydroxy-4-({2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl 1-benzyl-3-hydroxy-4-({2-[(methoxycarbonyl)amino]-3,3-dimethylbutanoyl}amino)-5-[4-(2-pyridinyl)phenyl]pentylcarbamate;
1:1 mixture of (3R,3aS,6aR)-hexahydrofuro[2,3-b]furan-3-yl 1-benzyl-2-hydroxy-4-({2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl 1-benzyl-2-hydroxy-4-({2-[(methoxycarbonyl)amino]-3,3-dimethylbutanoyl}amino)-5-[4-(2-pyridinyl)phenyl]pentylcarbamate;
methyl 1-[({2-hydroxy-4-[(3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 4-benzyl-10-tert-butyl-5-hydroxy-1-[1-methyl-1-(methylsulfanyl)ethyl]-2,9,12-trioxo-7-[4-(2-pyridinyl)benzyl]-13-oxa-3,8,11-triazatetradec-1-ylcarbamate;
methyl 4-benzyl-10-tert-butyl-5-hydroxy-1-[1-methyl-1-(methylsulfonyl)ethyl]-2,9,12-trioxo-7-[4-(2-pyridinyl)benzyl]-13-oxa-3,8,11-triazatetradec-1-ylcarbamate;
methyl 4-benzyl-10-tert-butyl-6-hydroxy-1-[1-methyl-1-(methylsulfanyl)ethyl]-2,9,12-trioxo-7-[4-(2-pyridinyl)benzyl]-13-oxa-3,8,11-triazatetradec-1-ylcarbamate;
methyl 4-benzyl-10-tert-butyl-6-hydroxy-1-[1-methyl-1-(methylsulfonyl)ethyl]-2,9,12-trioxo-7-[4-(2-pyridinyl)benzyl]-13-oxa-3,8,11-triazatetradec-1-ylcarbamate;
methyl 1-[({4-{[(2S)-3,3-dimethyl-2-(2-oxo-3-{[2-(3-pyridinyl)-1,3-thiazol-4-yl]methyl}-1-imidazolidinyl)butanoyl]amino}-3-hydroxy-5-phenyl-1-[4-(2-pyridinyl)benzyl]pentyl}amino)carbonyl]-2,2-dimethylpropylcarbamate;
methyl 1-[({4-{[3,3-dimethyl-2-(2-oxo-3-{[2-(3-pyridinyl)-1,3-thiazol-4-yl]methyl}-1-imidazolidinyl)butanoyl]amino}-2-hydroxy-5-phenyl-1-[4-(2-pyridinyl)benzyl]pentyl}amino)carbonyl]-2,2-dimethylpropylcarbamate;
methyl 1-[({3-hydroxy-4-[(3-methyl-2-{3-[(6-methyl-2-pyridinyl)methyl]-2,4-dioxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({2-hydroxy-4-[(3-methyl-2-{3-[(6-methyl-2-pyridinyl)methyl]-2,4-dioxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({4-{[(2,6-dimethylphenoxy)acetyl]amino}-3-hydroxy-5-phenyl-1-[4-(2-pyridinyl)benzyl]pentyl}amino)carbonyl]-2,2-dimethylpropylcarbamate;
methyl 1-[({4-{[(2,6-dimethylphenoxy)acetyl]amino}-2-hydroxy-5-phenyl-1-[4-(2-pyridinyl)benzyl]pentyl}amino)carbonyl]-2,2-dimethylpropylcarbamate;
methyl 1-[({3-hydroxy-4-({(2S)-2-[3-(imidazo[1,5-a]pyridin-3-ylmethyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({2-hydroxy-4-({2-[3-(imidazo[1,5-a]pyridin-3-ylmethyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({4-({3,3-dimethyl-2-[2-oxo-3-(4-quinolinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({(2S)-3,3-dimethyl-2-[2-oxo-3-(4-quinolinylmethyl)-1-imidazolidinyl]butanoyl}amino)-2-hydroxy-5-phenyl-1-[4-(2-pyridinyl)benzyl]pentyl}amino)carbonyl]-2,2-dimethylpropylcarbamate;
methyl 1-[({2-hydroxy-4-{[2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl 1-[({3-hydroxy-4-{[2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2,4-dioxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
1:1 mixture of (3R,3aS,6aR)-hexahydrofuro[2,3-b]furan-3-yl 1-benzyl-2-hydroxy-4-({2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl 1-benzyl-2-hydroxy-4-({2-[(methoxycarbonyl)amino]-3,3-dimethylbutanoyl}amino)-5-[4-(2-pyridinyl)phenyl]pentylcarbamate;
methyl 1-[({4-{[3,3-dimethyl-2-(2-oxo-3-{[2-(3-pyridinyl)-1,3-thiazol-4-yl]methyl}-1-imidazolidinyl)butanoyl]amino}-3-hydroxy-5-phenyl-1-[4-(2-pyridinyl)benzyl]pentyl}amino)carbonyl]-2,2-dimethylpropylcarbamate;
methyl 1-[({4-{[(2,6-dimethylphenoxy)acetyl]amino}-3-hydroxy-5-phenyl-1-[4-(2-pyridinyl)benzyl]pentyl}amino)carbonyl]-2,2-dimethylpropylcarbamate;
methyl 1-[({3-hydroxy-4-{[2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2,4-dioxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2,4-dioxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[2-oxo-3-(4-quinolinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[(phenoxyacetyl)amino]butanoyl}amino)-3-hydroxy-5-phenyl-1-[4-(2-pyridinyl)benzyl]pentyl}amino)carbonyl]-2,2-dimethylpropylcarbamate;
methyl 7-benzyl-1,10-ditert-butyl-6-hydroxy-2,9,12-trioxo-4-[4-(2-pyridinyl)benzyl]-14-oxa-3,8,1-triazapentadec-1-ylcarbamate;
methyl 1-[({3-hydroxy-4-[(2-{3-[(2-isopropyl-1,3-thiazol-4-yl)methyl]-2,4-dioxo-1-imidazolidinyl}-3-methylpentanoyl)amino]-5-phenyl-1-[4-(2-pyridinyl)benzyl]pentyl}amino)carbonyl]-2,2-dimethylpropylcarbamate;
methyl 1-[({4-{[2-(2,4-dioxo-3-{[2-(3-pyridinyl)-1,3-thiazol-4-yl]methyl}-1-imidazolidinyl)-3-methylpentanoyl]amino}-3-hydroxy-5-phenyl-1-[4-(2-pyridinyl)benzyl]pentyl}amino)carbonyl]-2,2-dimethylpropylcarbamate;
methyl 1-[({4-{[3,3-dimethyl-2-({[(6-methyl-3-pyridinyl)oxy]acetyl}amino)butanoyl]amino}-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(1-methyl-1H-benzimidazol-2-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(2-methyl-1,3-thiazol-4-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
3-pyridinylmethyl 4-benzyl-1,10-ditert-butyl-5-hydroxy-2,9,12-trioxo-7-[4-(2-pyridinyl)benzyl]-13-oxa-3,8,1-triazatetradec-1-ylcarbamate;
benzyl 4-benzyl-1,10-ditert-butyl-5-hydroxy-2,9,12-trioxo-7-[4-(2-pyridinyl)benzyl]-13-oxa-3,8,1-triazatetradec-1-ylcarbamate;
methyl 7-benzyl-1,10-ditert-butyl-6-hydroxy-13-methyl-2,9,12-trioxo-14-phenyl-4-[4-(2-pyridinyl)benzyl]-3,8,11,13-tetraazatetradec-1-ylcarbamate;
methyl 7-benzyl-1,10-ditert-butyl-6-hydroxy-13-methyl-2,9,12-trioxo-14-phenyl-4-[4-(2-pyridinyl)benzyl]-3,8,11,13-tetraazatetradec-1-ylcarbamate;
methyl 1-[({4-({3,3-dimethyl-2-[3-(2-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[3-(3-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(2-methyl-1,3-thiazol-4-yl)methyl]-2-oxo-1-imidazolidinyl}butanoyl)amino]-2-hydroxy-5-phenyl-1-[4-(3-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-1-[4-(6-methyl-2-pyridinyl)benzyl]-5-phenylpentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-5-phenyl-1-[4-(3-pyridinyl)benzyl]pentyl}amino)carbonyl]-2,2-dimethylpropylcarbamate;
methyl 1-[({4-{[2-(3-benzyl-2-oxo-1-imidazolidinyl)-3,3-dimethylbutanoyl]amino}-2-hydroxy-5-phenyl-1-[4-(3-pyridinyl)benzyl]pentyl}amino)carbonyl]-2,2-dimethylpropylcarbamate;
methyl 1-[({3-hydroxy-4-({2-[3-(3-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({4-{[2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-5-phenyl-1-[4-(4-pyridinyl)benzyl]pentyl}amino)carbonyl]-2,2-dimethylpropylcarbamate;
methyl 1-[({4-[((2S)-3,3-dimethyl-2-{3-[(6-methyl-2-pyridinyl)methyl]-2-oxo-1-imidazolidinyl}butanoyl)amino]-2-hydroxy-1-[4-(5-methyl-2-pyridinyl)benzyl]-5-phenylpentyl}amino)carbonyl]-2,2-dimethylpropylcarbamate;
methyl 1-[({3-hydroxy-4-({2-[3-(2-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({4-({3,3-dimethyl-2-[3-(2-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[3-(3-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({3-hydroxy-4-({2-[3-(2-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({3-hydroxy-4-({2-[3-(3-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-1-[4-(4-methyl-2-pyridinyl)benzyl]-5-phenylpentyl}amino)carbonyl]-2,2-dimethylpropylcarbamate;
methyl 1-[({4-{[2-(3-benzyl-2-oxo-1-imidazolidinyl)-3,3-dimethylbutanoyl]amino}-2-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(2-methyl-3-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-3-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[2-oxo-3-(3-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[2-oxo-3-(4-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[2-oxo-3-(2-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 7-benzyl-1,10-ditert-butyl-5-hydroxy-4-[4-(6-methyl-3-pyridinyl)benzyl]-2,9,12-trioxo-13-oxa-3,8,11-triazatetradec-1-ylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-1-[4-(6-methyl-3-pyridinyl)benzyl]-5-phenylpentyl}amino)carbonyl]-2,2-dimethylpropylcarbamate;
methyl 1-[({4-[(3,3-dimethyl-2-{3-[(2-methyl-1,3-thiazol-4-yl)methyl]-2-oxo-1-imidazolidinyl}butanoyl)amino]-2-hydroxy-1-[4-(5-methyl-2-pyridinyl)benzyl]-5-phenylpentyl}amino)carbonyl]-2,2-dimethylpropylcarbamate;
methyl 7-benzyl-1,10-ditert-butyl-5-hydroxy-4-[4-(5-methyl-2-pyridinyl)benzyl]-2,9,12-trioxo-13-oxa-3,8,11-triazatetradec-1-ylcarbamate;
methyl 1-[({4-[(3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2-methylbutylcarbamate;
methyl 4-benzyl-1,10-ditert-butyl-5-hydroxy-7-[4-(5-methyl-2-pyridinyl)benzyl]-2,9,12-trioxo-13-oxa-3,8,11-triazatetradec-1-ylcarbamate;
methyl 1-[({4-{[2-(3-benzyl-2-oxo-1-imidazolidinyl)-3,3-dimethylbutanoyl]amino}-3-hydroxy-1-[4-(5-methyl-2-pyridinyl)benzyl]-5-phenylpentyl}amino)carbonyl]-2,2-dimethylpropylcarbamate;
methyl 1-[({1-benzyl-4-[(3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentyl}amino)carbonyl]-2,2-dimethylpropylcarbamate;
methyl 4-benzyl-10-tert-butyl-5-hydroxy-1-[1-methyl-1-((R)-methylsulfinyl)ethyl]-2,9,12-trioxo-7-[4-(2-pyridinyl)benzyl]-13-oxa-3,8,11-triazatetradec-1-ylcarbamate;
methyl 1-[({2-hydroxy-4-[(3-methyl-2-{3-[(1-methyl-1H-benzimidazol-2-yl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({2-hydroxy-4-[(2-{3-[(2-isopropyl-1,3-thiazol-4-yl)methyl]-2-oxo-1-imidazolidinyl}-3-methylpentanoyl)amino]-5-phenyl-1-[4-(2-pyridinyl)benzyl]pentyl}amino)carbonyl]-2,2-dimethylpropylcarbamate;
methyl 1-[({3-hydroxy-4-[(3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(1-methyl-1H-benzimidazol-2-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(1-methyl-1H-benzimidazol-2-yl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl 1-[({3-hydroxy-4-[(2-{3-[(2-isopropyl-1,3-thiazol-4-yl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl 1-[({2-hydroxy-4-[(2-{3-[(2-isopropyl-1,3-thiazol-4-yl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl 7-benzyl-1,10-ditert-butyl-5-hydroxy-2,9,12-trioxo-4-[4-(3-pyridinyl)benzyl]-13-oxa-3,8,11-triazatetradec-1-ylcarbamate;
methyl 7-benzyl-1,10-ditert-butyl-5-hydroxy-2,9,12-trioxo-4-[4-(4-pyridinyl)benzyl]-13-oxa-3,8,11-triazatetradec-1-ylcarbamate;
methyl 1-[({4-[(3,3-dimethyl-2-{3-[(2-methyl-1,3-thiazol-4-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(2-methyl-1,3-thiazol-4-yl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl 1-[({4-[((2S)-3,3-dimethyl-2-{3-[(6-methyl-2-pyridinyl)methyl]-2-oxo-1-imidazolidinyl butanoyl)amino]-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 7-benzyl-10-sec-butyl-1-tert-butyl-6-hydroxy-13-methyl-14-(2-methyl-1,3-thiazol-4-yl)-2,9,12-trioxo-4-[4-(2-pyridinyl)benzyl]-3,8,11,13-tetraazatetradec-1-ylcarbamate;
methyl 7-benzyl-10-sec-butyl-1-tert-butyl-5-hydroxy-13-methyl-14-(2-methyl-1,3-thiazol-4-yl)-2,9,12-trioxo-4-[4-(2-pyridinyl)benzyl]-3,8,11,13-tetraazatetradec-1-ylcarbamate;
methyl 1-[({4-{[2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate;
1,2,5,6-tetradeoxy-2,5-bis({2-[(methoxycarbonyl)amino]-3,3-dimethylbutanoyl}amino)-1,6-bis[4-(2-pyridinyl)phenyl]-D-iditol;
methyl 1-[({4-[(3,3-dimethyl-2-{3-[(6-methyl-2-pyridinyl)methyl]-2-oxo-1-imidazolidinyl}butanoyl)amino]-3-hydroxy-1-[4-(6-methoxy-2-pyridinyl)benzyl]-5-phenylpentyl}amino)carbonyl]-2,2-dimethylpropylcarbamate;
methyl 1-[({4-{[2-(3-benzyl-2-oxo-1-imidazolidinyl)-3,3-dimethylbutanoyl]amino}-3-hydroxy-1-[4-(6-methoxy-2-pyridinyl)benzyl]-5-phenylpentyl}amino)carbonyl]-2,2-dimethylpropylcarbamate;
methyl 1-[({4-[(2-{3-[(6-tert-butyl-2-pyridinyl)methyl]-2-oxo-1-imidazolidinyl}-3,3-dimethylbutanoyl)amino]-3-hydroxy-5-phenyl-1-[4-(2-pyridinyl)benzyl]pentyl}amino)carbonyl]-2,2-dimethylpropylcarbamate;
methyl 1,10-ditert-butyl-5-hydroxy-2,9,12-trioxo-4,7-bis[4-(2-pyridinyl)benzyl]-13-oxa-3,8,11-triazatetradec-1-ylcarbamate;
methyl 1-[({3-hydroxy-4-[(2-{3-[(6-isopropyl-2-pyridinyl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl 1-[({4-[(2-{3-[(6-tert-butyl-2-pyridinyl)methyl]-2-oxo-1-imidazolidinyl}-3,3-dimethylbutanoyl)amino]-3-hydroxy-5-phenyl-1-[4-(2-pyridinyl)benzyl]pentyl}amino)carbonyl]-2,2-dimethylpropylcarbamate;
methyl 1-[({-4-{[2-(3-benzyl-2-oxo-1-imidazolidinyl)-3,3-dimethylbutanoyl]amino}-2-hydroxy-1-[4-(6-methoxy-2-pyridinyl)benzyl]-5-phenylpentyl}amino)carbonyl]-2,2-dimethylpropylcarbamate;
methyl 7-benzyl-1,10-ditert-butyl-5-hydroxy-4-[4-(6-methoxy-2-pyridinyl)benzyl]-2,9,12-trioxo-13-oxa-3,8,11-triazatetradec-1-ylcarbamate;
methyl 4-benzyl-1,10-ditert-butyl-5-hydroxy-7-[4-(6-methoxy-2-pyridinyl)benzyl]-2,9,12-trioxo-13-oxa-3,8,11-triazatetradec-1-ylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-1-[4-(6-methoxy-2-pyridinyl)benzyl]-5-phenylpentyl}amino)carbonyl]-2,2-dimethylpropylcarbamate;
methyl 1-[({4-({2-[3-(2-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl 1-[({4-({2-[3-(4-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl 1-[({4-({2-[3-(3-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-3-hydroxy-1-[4-(5-methyl-2-pyridinyl)benzyl]-5-phenylpentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-3-hydroxy-1-[4-(5-methyl-2-pyridinyl)benzyl]-5-phenylpentyl}amino)carbonyl]-2,2-dimethylpropylcarbamate;
methyl 1-[({3-hydroxy-4-[(2-{3-[(6-isopropyl-2-pyridinyl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl 1-[({1-benzyl-4-[(3,3-dimethyl-2-{3-[(6-methyl-2-pyridinyl)methyl]-2-oxo-1-imidazolidinyl}butanoyl)amino]-3-hydroxy-5-[4-(2-pyridinyl)phenyl]pentyl}amino)carbonyl]-2,2-dimethylpropylcarbamate;
methyl 4-benzyl-1,10-disec-butyl-5-hydroxy-2,9,12-trioxo-7-[4-(2-pyridinyl)benzyl]-13-oxa-3,8,11-triazatetradec-1-ylcarbamate; and
methyl 1-({[1-benzyl-2-hydroxy-4-({3-methyl-2-[2-oxo-3-(4-quinolinylmethyl)-1-imidazolidinyl]pentanoyl}amino)-5-phenylpentyl]amino}carbonyl)-2,2-dimethylpropylcarbamate; or a pharmaceutical acceptable salt form, ester, salt of an ester, prodrug, salt of a prodrug, or combination thereof.

In a second embodiment, the present invention provides a compound of formula (II) 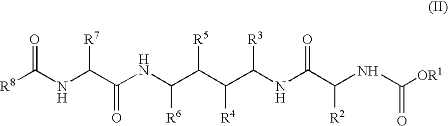
or a pharmaceutically acceptable salt form, stereoisomer, ester, salt of an ester, prodrug, salt of a prodrug, or combination thereof, wherein:
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from tte group consisting of halo, —ORₐ, —SRₐ, —SORₐ, —SO₂Rₐ, —NRₐR_{b}, —NR_{b}C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁸ is —ORₐ or -alkylORₐ;
R¹⁶ is hydrogen or R¹⁵;
R¹⁵ is 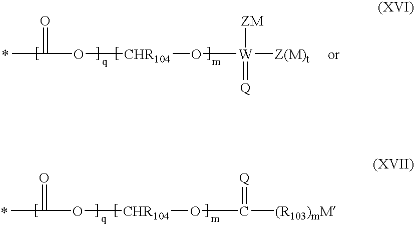
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c}; and
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂.

For example, the present invention provides a compound of formula (II) wherein R⁴ is H and R⁵ is OR¹⁶.

For example, the present invention provides a compound of formula (II) wherein R⁴ is OR¹⁶ and R⁵ is H.

For example, the present invention provides a compound of formula (II) wherein R⁴ is H, R⁵ is OR¹⁶ and R² is alkyl.

For example, the present invention provides a compound of formula (II) wherein R⁴ is OR¹⁶, R⁵ is H, and R² is alkyl.

For example, the present invention provides a compound of formula (II) wherein R⁴ is H, R⁵ is OR¹⁵, R² is alkyl and R³ is arylalkyl.

For example, the present invention provides a compound of formula (II) wherein R⁴ is OR¹⁶, R⁵ is H, R² is alkyl and R³ is arylalkyl.

For example, the present invention provides a compound of formula (II) wherein R⁴ is H, R⁵ is OR¹⁶, R² is alkyl and R³ is arylalkyl substituted with R^{3a}.

For example, the present invention provides a compound of formula (II) wherein R⁴ is OR¹⁶, R⁵ is H, R² is alkyl and R³ is arylalkyl substituted with R^{3a}.

For example, the present invention provides a compound of formula (II) wherein R⁴ is H, R⁵ is OR¹⁶, R² is alkyl, R³ is arylalkyl substituted with R^{3a}, and R^{3a} is aryl or heteroaryl.

For example, the present invention provides a compound of formula (II) wherein R⁴ is OR¹⁶, R⁵ is H, R² is alkyl, R³ is arylalkyl substituted with R³, and R^{3a} is aryl or heteroaryl.

For example, the present invention provides a compound of formula (II) wherein R⁴ is H, R⁵ is OR¹⁶, R² is alkyl, R³ is arylalkyl substituted with R^{3a}, R⁸ is —ORₐ or -alkylORₐ, R^{3a} is aryl or heteroaryl, and Rₐ is alkyl, aryl or heteroaryl.

For example, the present invention provides a compound of formula (II) wherein R⁴ is OR¹⁶, R⁵ is H, R² is alkyl, R³ is arylalkyl substituted with R^{3a}, It is —ORₐ or -alkylORₐ, R^{3a} is aryl or heteroaryl, and Rₐ is alkyl, aryl or heteroaryl.

For example, the present invention provides a compound of formula (II) wherein R⁴ is H, R⁵ is OR¹⁶, R² is alkyl, R³ is arylalkyl substituted with R^{3a}, R⁸ is —ORₐ or -alkylORₐ, R⁷ is alkyl, R^{3a} is aryl or heteroaryl, and Rₐ is alkyl, aryl or heteroaryl.

For example, the present invention provides a compound of formula (II) wherein R⁴ is OR¹⁶, R⁵ is H, R² is alkyl, R³ is arylalkyl substituted with R^{3a} and R⁸ is —ORₐ or -alkylORₐ, R⁷ is alkyl, R^{3a} is aryl or heteroaryl, and Rₐ is alkyl, aryl or heteroaryl.

For example, the present invention provides a compound of formula (II) wherein R⁴ is H, R⁵ is OR¹⁶, R² is alkyl, R³ is arylalkyl substituted with R^{3a}, R⁸ is —ORₐ or -alkylORₐ, R⁷ is alkyl, R⁶ is arylalkyl, R^{3a} is aryl or heteroaryl, and Rₐ is alkyl, aryl or heteroaryl.

For example, the present invention provides a compound of formula (II) wherein R⁴ is OR¹⁶, R⁵ is H, R² is alkyl, R³ is arylalkyl substituted with R^{3a}, It is —ORₐ or alkylORₐ, R⁷ is alkyl, R⁶ is arylalkyl, R^{3a} is aryl or heteroaryl, and Rₐ is alkyl, aryl or heteroaryl.

For example, the present invention provides a compound of formula (II) wherein R⁴ is H, R⁵ is OR¹⁶, R² is C1, C2, C3, C4 or C5 alkyl, R³ is arylalkyl substituted with R^{3a}, R⁸ is —ORₐ or -alkylORₐ, R⁷ is alkyl, R¹ is arylalkyl, R^{3a} is aryl or heteroaryl, and Rₐ is alkyl, aryl or heteroaryl.

For example, the present invention provides a compound of formula (II) wherein R⁴ is OR¹⁶, R⁵ is H, R² is C1, C2, C3, C4 or C5 alkyl, R³ is arylalkyl substituted with R^{1a}, R⁸ is —ORₐ or -alkylORₐ, R⁷ is alkyl, R⁶ is arylalkyl, R^{3a} is aryl or heteroaryl, and Rₐ is alkyl, aryl or heteroaryl.

For example, the present invention provides a compound of formula (II) wherein R⁴ is H, R⁵ is OR¹⁶, R² is C1, C2, C3, C4 or C5 alkyl, R³ is arylalkyl substituted with R^{3a}, R⁸ is ORₐ or -alkylORₐ, R⁷ is alkyl, R⁶ is arylalkyl, R^{3a} is aryl or heteroaryl, and Rₐ is methyl, phenyl or pyridyl.

For example, the present invention provides a compound of formula (II) wherein R⁴ is OR¹⁶, R⁵ is H, R² is C1, C2, C3, C4 or C5 alkyl, R³ is arylalkyl substituted with R^{3a}, R⁸ is —ORₐ or -alkylORₐ, R⁷ is alkyl, R⁶ is arylalkyl, R^{3a} is aryl or heteroaryl, and Rₐ is methyl, phenyl or pyridyl.

For example, the present invention provides a compound of formula (II) wherein R⁴ is H, R⁵ is OR¹⁶, R¹ is alkyl, R² is C1, C2, C3, C4 or C5 alkyl, R³ is phenylmethyl substituted with R^{3a}, R⁸ is —ORₐ or -alkylORₐ, R⁷ is C1, C2, C3, C4 or C5 alkyl, R⁶ is phenylmethyl, R^{3a} is pyridyl, and Rₐ is methyl, phenyl, pyridyl, or methyl substituted with one substituent selected from the group consisting of phenyl and pyridyl.

For example, the present invention provides a compound of formula (II) wherein R⁴ is OR¹⁶, R⁵ is H, R¹ is alkyl, R² is C1, C2, C3, C4 or C5 alkyl, R³ is phenylmethyl substituted with R^{3a}, R⁸ is —ORₐ or -alkylORₐ, R⁷ is C1, C2, C3, C4 or C5 alkyl, R⁶ is phenylmethyl, R^{3a} is pyridyl, and Rₐ is methyl, phenyl, pyridyl, or methyl substituted with one susbtituent selected from the group consisting of phenyl and pyridyl.

For example, the present invention provides a compound of formula (II) wherein R⁴ is H, R⁵ is OR¹⁶, R¹ is methyl, R² is 1-methylpropyl, tert-butyl or isopropyl, R¹ is phenylmethyl substituted with R^{3a}, R⁸ is —ORₐ or -alkylORₐ, R⁷ is 1-methylpropyl, tert-butyl or isopropyl, R⁶ is phenylmethyl, R^{3a} is pyridyl, and Rₐ is methyl, phenyl, pyridyl, or methyl substituted with one substituent selected from the group consisting of phenyl and pyridyl.

For example, the present invention provides a compound of formula (II) wherein R⁴ is OR¹⁶, R⁵ is H, R¹ is methyl, R² is 1-methylpropyl, tert-butyl or isopropyl, R³ is phenylmethyl substituted with R^{3a}, R⁸ is —ORₐ or -alkylORₐ, R⁷ is 1-methylpropyl, tert-butyl or isopropyl, R⁶ is phenylmethyl, R^{3a} is pyridyl, and Rₐ is methyl, phenyl, pyridyl, or methyl substituted with one susbtituent selected from the group consisting of phenyl and pyridyl.

Exemplary compounds of the present invention of formula (II) include, but not limited, to the following:
methyl(1S,4R,6S,7S,10S)-7-benzyl-1,10-ditert-butyl-6-hydroxy-2,9,12-trioxo-4-[4-(2-pyridinyl)benzyl]-13-oxa-3,8,11-triazatetradec-1-ylcarbamate;
methyl(1S,4S,5S,7S,10S)-4-benzyl-1,10-ditert-butyl-5-hydroxy-2,9,12-trioxo-7-[4-(2-pyridinyl)benzyl]-13-oxa-3,8,11-triazatetradec-1-ylcarbamate;
methyl(1S,4S,5S,7S,10S)-7-benzyl-1,10-ditert-butyl-5-hydroxy-2,9,12-trioxo-4-[4-(2-pyridinyl)benzyl]-13-oxa-3,8,11-triazatetradec-1-ylcarbamate;
methyl(1R,4S,5S,7S,10S)-4-benzyl-10-tert-butyl-5-hydroxy-1-[1-methyl-1-(methylsulfanyl)ethyl]-2,9,12-trioxo-7-[4-(2-pyridinyl)benzyl]-13-oxa-3,8,11-triazatetradec-1-ylcarbamate;
methyl(1R,4S,5S,7S,10S)-4-benzyl-10-tert-butyl-5-hydroxy-1-[1-methyl-1-(methylsulfonyl)ethyl]-2,9,12-trioxo-7-[4-(2-pyridinyl)benzyl]-13-oxa-3,8,11-triazatetradec-1-ylcarbamate;
methyl(1R,4S,6S,7S,10S)-4-benzyl-10-tert-butyl-6-hydroxy-1-[1-methyl-1-(methylsulfanyl)ethyl]-2,9,12-trioxo-7-[4-(2-pyridinyl)benzyl]-13-oxa-3,8,11-triazatetradec-1-ylcarbamate;
methyl(1R,4S,6S,7S,10S)-4-benzyl-10-tert-butyl-6-hydroxy-1-[i-methyl-1-(methylsulfonyl)ethyl]-2,9,12-trioxo-7-[4-(2-pyridinyl)benzyl]-13-oxa-3,8,111-triazatetradec-1-ylcarbamate;
methyl(1S)-1-[({(1S,3S,4S)-4-({(2S)-3,3-dimethyl-2-[(phenoxyacetyl)amino]butanoyl}amino)-3-hydroxy-5-phenyl-1-[4-(2-pyridinyl)benzyl]pentyl}amino)carbonyl]-2,2-dimethylpropylcarbamate;
methyl(1S,4S,6S,7S,10S)-7-benzyl-1,10-ditert-butyl-6-hydroxy-2,9,12-trioxo-4-[4-(2-pyridinyl)benzyl]-14-oxa-3,8,11-triazapentadec-1-ylcarbamate;
methyl(1S)-1-[({(1S,3S,4S)-4-{[(2S)-3,3-dimethyl-2-({[(6-methyl-3-pyridinyl)oxy]acetyl}amino)butanoyl]amino}-3-hydroxy-5-phenyl-1-[4-(2-pyridinyl)benzyl]pentyl}amino)carbonyl]-2,2-dimethylpropylcarbamate;
3-pyridinylmethyl(1S,4S,5S,7S,10S)-4-benzyl-1,10-ditert-butyl-5-hydroxy-2,9,12-trioxo-7-[4-(2-pyridinyl)benzyl]-13-oxa-3,8,11-triazatetradec-1-ylcarbamate;
benzyl(1S,4S,5S,7S,10S)-4-benzyl-1,10-ditert-butyl-5-hydroxy-2,9,12-trioxo-7-[4-(2-pyridinyl)benzyl]-13-oxa-3,8,11-triazatetradec-1-ylcarbamate;
methyl(1S,4S,5S,7S,10S)-7-benzyl-1,10-ditert-butyl-5-hydroxy-4-[4-(6-methyl-3-pyridinyl)benzyl]-2,9,12-trioxo-13-oxa-3,8,11-triazatetradec-1-ylcarbamate;
methyl(1S,4S,5S,7S,10S)-7-benzyl-1,10-ditert-butyl-5-hydroxy-4-[4-(5-methyl-2-pyridinyl)benzyl]-2,9,12-trioxo-13-oxa-3,8,11-triazatetradec-1-ylcarbamate;
methyl(1S,4S,5S,7S,10S)-4-benzyl-1,10-ditert-butyl-5-hydroxy-7-[4-(5-methyl-2-pyridinyl)benzyl]-2,9,12-trioxo-13-oxa-3,8,11-triazatetradec-1-ylcarbamate;
1:1 mixture of methyl(1R,4S,5S,7S,10S)-4-benzyl-10-tert-butyl-5-hydroxy-1-[1-methyl-1-((R)-methylsulfinyl)ethyl]-2,9,12-trioxo-7-[4-(2-pyridinyl)benzyl]-13-oxa-3,8,11-triazatetradec-1-ylcarbamate and methyl(1R,4S,5S,7S,10S)-4-benzyl-10-tert-butyl-5-hydroxy-1-[1-methyl-1-((S)-methylsulfinyl)ethyl]-2,9,12-trioxo-7-[4-(2-pyridinyl)benzyl]-13-oxa-3,8,11-triazatetradec-1-ylcarbamate;
methyl(1S,4S,5S,7S,10S)-7-benzyl-1,10-ditert-butyl-5-hydroxy-2,9,12-trioxo-4-[4-(3-pyridinyl)benzyl]-13-oxa-3,8,11-triazatetradec-1-ylcarbamate;
methyl(1S,4S,5S,7S,10S)-7-benzyl-1,10-ditert-butyl-5-hydroxy-2,9,12-trioxo-4-[4-(4-pyridinyl)benzyl]-13-oxa-3,8,11-triazatetradec-1-ylcarbamate;
1,2,5,6-tetradeoxy-2,5-bis({(2S)-2-[(methoxycarbonyl)amino]-3,3-dimethylbutanoyl}amino)-1,6-bis[4-(2-pyridinyl)phenyl]-D-iditol;
methyl(1S,4R,5R,7R,10S)-1,10-ditert-butyl-5-hydroxy-2,9,12-trioxo-4,7-bis[4-(2-pyridinyl)benzyl]-13-oxa-3,8,11-triazatetradec-1-ylcarbamate;
methyl(1S,4S,5S,7S,10S)-7-benzyl-1,10-ditert-butyl-5-hydroxy-4-[4-(6-methoxy-2-pyridinyl)benzyl]-2,9,12-trioxo-13-oxa-3,8,11-triazatetradec-1-ylcarbamate;
methyl(1S,4S,5S,7S,10S)-4-benzyl-1,10-ditert-butyl-5-hydroxy-7-[4-(6-methoxy-2-pyridinyl)benzyl]-2,9,12-trioxo-13-oxa-3,8,11-triazatetradec-1-ylcarbamate; and
methyl(1S,4S,5S,7S,10S)-4-benzyl-1,10-disec-butyl-5-hydroxy-2,9,12-trioxo-7-[4-(2-pyridinyl)benzyl]-13-oxa-3,8,11-triazatetradec-1-ylcarbamate; or a pharmaceutical acceptable salt form, stereoisomer, ester, salt of an ester, prodrug, salt of a prodrug, or combination thereof.

In a third embodiment, the present invention provides a compound of formula (III) 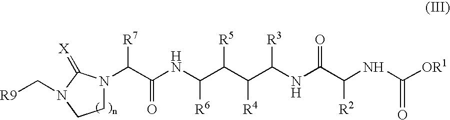
or a pharmaceutically acceptable salt form, stereoisomer, ester, salt of an ester, prodrug, salt of a prodrug, or combination thereof, wherein:
X is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylN-H₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁶ is hydrogen or R¹⁵;
R¹⁵ is 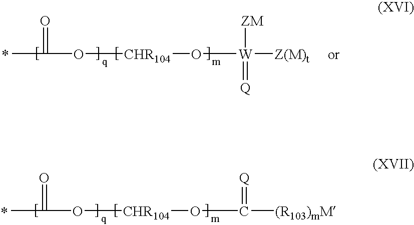
   wherein
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

For example, the present invention provides a compound of formula (III) wherein R⁴ is H and R⁵ is OR¹⁶.

For example, the present invention provides a compound of formula (III) wherein R⁴ is OR¹⁶ and R⁵ is H.

For example, the present invention provides a compound of formula (III) wherein R¹ is alkyl.

For example, the present invention provides a compound of formula (III) wherein R¹ is methyl.

For example, the present invention provides a compound of formula (III) wherein R² is alkyl.

For example, the present invention provides a compound of formula (III) wherein R² is tert-butyl.

For example, the present invention provides a compound of formula (III) wherein R³ is arylalkyl.

For example, the present invention provides a compound of formula (III) wherein R³ is phenylmethyl.

For example, the present invention provides a compound of formula (III) wherein R⁶ is arylalkyl.

For example, the present invention provides a compound of formula (III) wherein R⁶ is phenylmethyl.

For example, the present invention provides a compound of formula (III) wherein R⁷ is alkyl.

For example, the present invention provides a compound of formula (III) wherein R⁷ is tert-butyl.

For example, the present invention provides a compound of formula (III) wherein R⁹ is aryl.

For example, the present invention provides a compound of formula (III) wherein R⁹ is phenyl.

For example, the present invention provides a compound of formula (III) wherein R⁹ is heteroaryl.

For example, the present invention provides a compound of formula (III) wherein R⁹ is pyridyl.

For example, the present invention provides a compound of formula (III) wherein R⁴ is H, R⁵ is OR¹⁶, X is O and R² is alkyl.

For example, the present invention provides a compound of formula (III) wherein R⁴ is OR¹⁶, R⁵ is H, X is O and R² is alkyl.

For example, the present invention provides a compound of formula (III) wherein R⁴ is H, R⁵ is OR¹⁶, X is O and R¹ is alkyl.

For example, the present invention provides a compound of formula (III) wherein R⁴ is OR¹⁶, R⁵ is H, X is O and R¹ is alkyl.

For example, the present invention provides a compound of formula (III) wherein R⁴ is H, R⁵ is OR¹⁶, X is O and R³ is arylalkyl.

For example, the present invention provides a compound of formula (III) wherein R⁴ is OR¹⁶, R⁵ is H, X is O and R³ is arylalkyl.

For example, the present invention provides a compound of formula (III) wherein R⁴ is H, R⁵ is OR¹⁶, X is O and R⁶ is arylalkyl.

For example, the present invention provides a compound of formula (III) wherein R⁴ is OR¹⁶, R⁵ is H, X is O and R⁶ is arylalkyl.

For example, the present invention provides a compound of formula (III) wherein R⁴ is H, R⁵ is OR¹⁶, X is O and R⁷ is alkyl.

For example, the present invention provides a compound of formula (III) wherein R⁴ is OR¹⁶, R⁵ is H, X is O and R⁷ is alkyl.

For example, the present invention provides a compound of formula (III) wherein R⁴ is H, R⁵ is OR¹⁶, X is O and R⁹ is aryl.

For example, the present invention provides a compound of formula (III) wherein R⁴ is OR¹⁶, R⁵ is H, X is O and R⁹ is aryl.

For example, the present invention provides a compound of formula (III) wherein R⁴ is H, R⁵ is OR¹⁶, X is O and R⁹ is heteroaryl.

For example, the present invention provides a compound of formula (III) wherein R⁴ is OR¹⁶, R⁵ is H, X is O and R⁹ is heteroaryl.

For example, the present invention provides a compound of formula (III) wherein R⁴ is H, R⁵ is OR¹⁶, X is O, R² is alkyl and R³ is arylalkyl.

For example, the present invention provides a compound of formula (III) wherein R⁴ is OR¹⁶, R⁵ is H, X is O, R² is alkyl and R³ is arylalkyl.

For example, the present invention provides a compound of formula (III) wherein R⁴ is H, R⁵ is OR¹⁶, X is O, R² is alkyl and R³ is arylalkyl substituted with R^{3a}.

For example, the present invention provides a compound of formula (III) wherein R⁴ is OR¹⁶, R⁵ is H, X is O, R² is alkyl and R³ is arylalkyl substituted with R₃ₐ.

For example, the present invention provides a compound of formula (III) wherein R⁴ is H, R⁵ is OR¹⁶, X is O, R² is alkyl, R³ is arylalkyl substituted with R^{3a}, and R^{3a} is aryl or heteroaryl.

For example, the present invention provides a compound of formula (III) wherein R⁴ is OR¹⁶, R⁵ is H, X is O, R² is alkyl, R³ is arylalkyl substituted with R^{3a}, and R^{3a} is aryl or heteroaryl.

For example, the present invention provides a compound of formula (III) wherein R⁴ is H, R⁵ is OR¹⁶, X is O, R² is alkyl, R¹ is arylalkyl substituted with R^{3a}, R⁹ is aryl or heteroaryl, and R³ is aryl or heteroaryl.

For example, the present invention provides a compound of formula (III) wherein R⁴ is OR¹⁶, R⁵ is H, X is O, R² is alkyl, R³ is arylalkyl substituted with R^{3a}, R⁹ is aryl or heteroaryl, and R^{3a} is aryl or heteroaryl.

For example, the present invention provides a compound of formula (III) wherein R¹ is alkyl, R² is alkyl, R³ is arylalkyl, R⁴ is H, R⁵ is OR¹⁶, R⁶ is arylalkyl, R⁷ is alkyl and R⁹ is aryl.

For example, the present invention provides a compound of formula (III) wherein R¹ is methyl, R² is tert-butyl, R³ is phenylmethyl, R⁴ is H, R⁵ is OH, R⁶ is phenylmethyl, R⁷ is tert-butyl and R⁹ is phenyl.

For example, the present invention provides a compound of formula (III) wherein R¹ is alkyl, R² is alkyl, R³ is arylalkyl, R⁴ is H, R⁵ is OR¹⁶, R⁶ is arylalkyl, R⁷ is alkyl and R⁹ is heteroaryl.

For example, the present invention provides a compound of formula (III) wherein R¹ is methyl, R² is tert-butyl, R³ is phenylmethyl, R⁴ is H, R⁵ is OH, R⁶ is phenylmethyl, R⁷ is tert-butyl and R⁹ is pyridyl.

For example, the present invention provides a compound of formula (III) wherein R¹ is alkyl, R² is alkyl, R³ is arylalkyl, R⁴ is OR¹⁶, R⁵ is H, R⁶ is arylalkyl, R⁷ is alkyl and R⁹ is aryl.

For example, the present invention provides a compound of formula (III) wherein R¹ is methyl, R² is tert-butyl, R³ is phenylmethyl, R⁴ is OH, R⁵ is H, R⁶ is phenylmethyl, R⁷ is tert-butyl and R⁹ is phenyl.

For example, the present invention provides a compound of formula (III) wherein R¹ is alkyl, R² is alkyl, R³ is arylalkyl, R⁴ is OR¹⁶, R⁵ is H, R⁶ is arylalkyl, R⁷ is alkyl and R⁹ is heteroaryl.

For example, the present invention provides a compound of formula (III) wherein R¹ is methyl, R² is tert-butyl, R³ is phenylmethyl, R⁴ is OH, R⁵ is H, R⁶ is phenylmethyl, R⁷ is tert-butyl and R⁹ is pyridyl.

For example, the present invention provides a compound of formula (III) wherein R⁴ is H, R⁵ is OR¹⁶, X is O, R² is alkyl, R³ is arylalkyl substituted with R^{3a}, R⁹ is aryl or heteroaryl, R¹ is arylalkyl, and R^{3a} is aryl or heteroaryl.

For example, the present invention provides a compound of formula (III) wherein R⁴ is OR¹⁶, R⁵ is H, X is O, R² is alkyl, R³ is arylalkyl substituted with R^{3a}, R⁹ is aryl or heteroaryl, R¹ is arylalkyl, and R^{3a} is aryl or heteroaryl.

For example, the present invention provides a compound of formula (III) wherein R⁴ is H, R⁵ is OR¹⁶, X is O, R² is alkyl, R³ is arylalkyl substituted with R³, R⁹ is aryl or heteroaryl, R¹ is arylalkyl, R⁷ is alkyl, and R^{3a} is aryl or heteroaryl.

For example, the present invention provides a compound of formula (III) wherein R⁴ is OR¹⁶, R⁵ is H, X is O, R² is alkyl, R¹ is arylalkyl substituted with R^{3a}, R⁹ is aryl or heteroaryl, R¹ is arylalkyl, R⁷ is alkyl, and R^{3a} is aryl or heteroaryl.

For example, the present invention provides a compound of formula (III) wherein R⁴ is H, R⁵ is OR¹⁶, X is O, R² is alkyl, R³ is arylalkyl substituted with R^{3a}, R⁹ is aryl or heteroaryl, R¹ is arylalkyl and R⁷ is alkyl; wherein R^{3a} is heteroaryl.

For example, the present invention provides a compound of formula (III) wherein R⁴ is OR¹⁶, R⁵ is H, X is O, R² is alkyl, R¹ is arylalkyl substituted with R^{3a}, R⁹ is aryl or heteroaryl, R¹ is arylalkyl, R⁷ is alkyl and R^{1a} is heteroaryl.

For example, the present invention provides a compound of formula (III) wherein R⁴ is H, R⁵ is OR¹⁶, X is O, R² is C1, C2, C3, C4 or C5 alkyl, R³ is phenylmethyl substituted with R^{3a}, R⁹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R⁶ is phenylmethyl, R⁷ is C1, C2, C3, C4 or C5 alkyl, nd R^{3a} is heteroaryl.

For example, the present invention provides a compound of formula (III) wherein R⁴ is OR¹⁶, R⁵ is H, X is O, R² is C1, C2, C3, C4 or C5 alkyl, R³ is phenylmethyl substituted with R^{3a}, R⁹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R⁶ is phenylmethyl, R⁷ is C1, C2, C3, C4 or C5 alkyl, and R^{3a} is heteroaryl.

For example, the present invention provides a compound of formula (III) wherein R⁴ is H, R⁵ is OR¹⁶, X is O, R² is isopropyl, 1-methylpropyl or tert-butyl, R³ is phenylmethyl substituted with R^{3a}, R⁹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R⁶ is phenylmethyl, R⁷ is isopropyl, 1-methylpropyl or tert-butyl, and R^{3a} is pyridyl.

For example, the present invention provides a compound of formula (III) wherein R⁴ is OR¹⁶, R⁵ is H, X is O, R² is isopropyl, 1-methylpropyl or tert-butyl, R³ is phenylmethyl substituted with R^{3a}, R⁹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R⁶ is phenylmethyl, R⁷ is isopropyl, 1-methylpropyl or tert-butyl, and R^{3a} is pyridyl.

For example, the present invention provides a compound of formula (III) wherein R⁴ is H, R⁵ is OR¹⁶, X is O, R² is isopropyl, 1-methylpropyl or tert-butyl, R³ is phenylmethyl substituted with R^{3a}, R⁹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R⁶ is phenylmethyl and R⁷ is isopropyl, 1-methylpropyl or tert-butyl; wherein R^{3a} is 2-pyridyl.

For example, the present invention provides a compound of formula (III) wherein R⁴ is OR¹⁶, R⁵ is H, X is O, R² is isopropyl, 1-methylpropyl or tert-butyl, R³ is phenylmethyl substituted with R^{3a}, R⁹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R¹ is phenylmethyl substituted with R^{6a}, R⁷ is isopropyl, 1-methylpropyl or tert-butyl, and R^{3a} is 2-pyridyl.

Exemplary compounds of the present invention of formula (III) include, but not limited to, the following:
methyl(1S)-1-{[((1S,3S,4S)-1-benzyl-3-hydroxy-4-{[(2S)-3-methyl-2-(2-oxo-3-{[2-(2-pyridinyl)-1,3-thiazol-4-yl]methyl}-1-imidazolidinyl)pentanoyl]amino}-5-phenylpentyl)amino]carbonyl}-2,2-dimethylpropylcarbamate;
methyl(1S)-1-({[(1S,3S,4S)-1-benzyl-3-hydroxy-4-({(2S)-3-methyl-2-[2-oxo-3-(4-quinolinylmethyl)-1-imidazolidinyl]pentanoyl}amino)-5-phenylpentyl]amino}carbonyl)-2,2-dimethylpropylcarbamate;
methyl(1S)-1-({[(1S,3S,4S)-1-benzyl-3-hydroxy-4-({(2S)-3-methyl-2-[2-oxo-3-(4-quinolinylmethyl)-1-imidazolidinyl]pentanoyl}amino)-5-phenylpentyl]amino}carbonyl)-2-methylbutylcarbamate;
methyl(1S)-1-{[((1S,3S,4S)-1-benzyl-3-hydroxy-4-{[(2S)-2-(3-{[2-(methoxymethyl)-1,3-thiazol-4-yl]methyl}-2-oxo-1-imidazolidinyl)-3,3-dimethylbutanoyl]amino}-5-phenylpentyl)amino]carbonyl}-2,2-dimethylpropylcarbamate;
methyl(1S)-1-[({(1S,3S,4S)-1-benzyl-3-hydroxy-4-[((2S)-3-methyl-2-{3-[(6-methyl-2-pyridinyl)methyl]-2-oxo-1-imidazolidinyl}pentanoyl)amino]-5-phenylpentyl}amino)carbonyl]-2,2-dimethylpropylcarbamate;
methyl(1S)-1-[({(1S,2S,4S)-1-benzyl-2-hydroxy-4-[((2S)-3-methyl-2-{3-[(6-methyl-2-pyridinyl)methyl]-2-oxo-1-imidazolidinyl}pentanoyl)amino]-5-phenylpentyl}amino)carbonyl]-2,2-dimethylpropylcarbamate;
methyl(1S)-1-[({(1S,2S,4S)-1-benzyl-2-hydroxy-4-[((2S)-3-methyl-2-{3-[(6-methyl-2-pyridinyl)methyl]-2-oxo-1-imidazolidinyl}pentanoyl)amino]-5-phenylpentyl}amino)carbonyl]-2-methylbutylcarbamate;
methyl(1S)-1-[({(1S,2S,4S)-1-benzyl-4-[((2S)-3,3-dimethyl-2-{3-[(6-methyl-2-pyridinyl)methyl]-2-oxo-1-imidazolidinyl}butanoyl)amino]-2-hydroxy-5-phenylpentyl}amino)carbonyl]-2,2-dimethylpropylcarbamate;
methyl(1S)-1-[({(1S,2S,4S)-1-benzyl-2-hydroxy-4-[((2S)-3-methyl-2-{3-[(2-methyl-1,3-thiazol-5-yl)methyl]-2-oxo-1-imidazolidinyl}pentanoyl)amino]-5-phenylpentyl}amino)carbonyl]-2,2-dimethylpropylcarbamate;
methyl(1S)-1-{[((1S,2S,4S)-1-benzyl-2-hydroxy-4-{[(2S)-3-methyl-2-(2-oxo-3-{[2-(3-pyridinyl)-1,3-thiazol-4-yl]methyl}-1-imidazolidinyl)pentanoyl]amino}-5-phenylpentyl)amino]carbonyl}-2,2-dimethylpropylcarbamate;
methyl(1S)-1-[({(1S,2S,4S)-1-benzyl-2-hydroxy-4-[((2S)-3-methyl-2-1 {3-[(6-methyl-3-pyridinyl)methyl]-2-oxo-1-imidazolidinyl}pentanoyl)amino]-5-phenylpentyl}amino)carbonyl]-2,2-dimethylpropylcarbamate;
methyl(1S)-1-[({(1S,2S,4S)-1-benzyl-4-[((2S)-3,3-dimethyl-2-{3-[(2-methyl-1,3-thiazol-4-yl)methyl]-2-oxo-1-imidazolidinyl}butanoyl)amino]-2-hydroxy-5-phenylpentyl}amino)carbonyl]-2,2-dimethylpropylcarbamate;
methyl(1S)-1-[({(1S,2S,4S)-1-benzyl-2-hydroxy-4-[((2S)-3-methyl-2-{3-[(2-methyl-3-pyridinyl)methyl]-2-oxo-1-imidazolidinyl}pentanoyl)amino]-5-phenylpentyl}amino)carbonyl]-2,2-dimethylpropylcarbamate;
methyl(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-{[((1S,2S,4S)-1-benzyl-2-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenylpentyl)amino]carbonyl}-2,2-dimethylpropylcarbamate;
methyl(1S)-1-[({(1S,3S,4S)-3-hydroxy-4-[((2S)-3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-{[((1S,2S,4S)-1-benzyl-4-{[(2S)-3,3-dimethyl-2-(2-oxo-3-{[2-(3-pyridinyl)-1,3-thiazol-4-yl]methyl}-1-imidazolidinyl)butanoyl]amino}-2-hydroxy-5-phenylpentyl)amino]carbonyl}-2,2-dimethylpropylcarbamate;
methyl(1S)-1-({[(1S,2S,4S)-1-benzyl-4-({(2S)-3,3-dimethyl-2-[2-oxo-3-(3-pyridinylmethyl)-1-imidazolidinyl]butanoyl}amino)-2-hydroxy-5-phenylpentyl]amino}carbonyl)-2,2-dimethylpropylcarbamate;
methyl(1S)-1-[({(1S,3S,4S)-3-hydroxy-4-[((2S)-3-methyl-2-{3-[(2-methyl-3-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1S,3S,4S)-1-benzyl-4-[((2S)-3,3-dimethyl-2-{3-[(6-methyl-2-pyridinyl)methyl]-2-oxo-1-imidazolidinyl}butanoyl)amino]-3-hydroxy-5-phenylpentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-1-benzyl-4-[((2S)-3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentyl}amino)carbonyl]-2,2-dimethylpropylcarbamate;
methyl(1S)-1-[({(1S,2S,4S)-2-hydroxy-4-[((2S)-3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1R,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-{[(2S)-3,3-dimethyl-2-(2-oxo-3-{[2-(3-pyridinyl)-1,3-thiazol-4-yl]methyl}-1-imidazolidinyl)butanoyl]amino}-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-{[(2S)-3,3-dimethyl-2-(2-oxo-3-{[2-(3-pyridinyl)-1,3-thiazol-4-yl]methyl}-1-imidazolidinyl)butanoyl]amino}-2-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-3-hydroxy-4-({(2S)-2-[3-(imidazo[1,5-a]pyridin-3-ylmethyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1S,2S,4S)-2-hydroxy-4-({(2S)-2-[3-(imidazo[1,5-a]pyridin-3-ylmethyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[2-oxo-3-(4-quinolinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-({(2S)-3,3-dimethyl-2-[2-oxo-3-(4-quinolinylmethyl)-1-imidazolidinyl]butanoyl}amino)-2-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-2-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl(1S)-1-[({(1S,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl(1S)-1-[({(1R,3S,4S)-4-{[(2S)-3,3-dimethyl-2-(2-oxo-3-{[2-(3-pyridinyl)-1,3-thiazol-4-yl]methyl}-1-imidazolidinyl)butanoyl]amino}-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl(1S)-1-[({(1R,3S,4S)-4-({(2S)-3,3-dimethyl-2-[2-oxo-3-(4-quinolinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-4-[((2S)-3,3-dimethyl-2-{3-[(1-methyl-1H-benzimidazol-2-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-4-[((2S)-3,3-dimethyl-2-{3-[(2-methyl-1,3-thiazol-4-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[3-(2-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[3-(3-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(2-methyl-1,3-thiazol-4-yl)methyl]-2-oxo-1-imidazolidinyl}butanoyl)amino]-2-hydroxy-5-phenyl-1-[4-(3-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methyl-2-pyridinyl)benzyl]-5-phenylpentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-5-phenyl-1-[4-(3-pyridinyl)benzyl]pentyl}amino)carbonyl]-2,2-dimethylpropylcarbamate;
methyl(1S)-1-[({(1S,2S,4S)-4-{[(2S)-2-(3-benzyl-2-oxo-1-imidazolidinyl)-3,3-dimethylbutanoyl]amino}-2-hydroxy-5-phenyl-1-[4-(3-pyridinyl)benzyl]pentyl}amino)carbonyl]-2,2-dimethylpropylcarbamate;
methyl(1S)-1-[({(1S,3S,4S)-3-hydroxy-4-({(2S)-2-[3-(3-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1R,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-5-phenyl-1-[4-(4-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(5-methyl-2-pyridinyl)benzyl]-5-phenylpentyl}amino)carbonyl]-2,2-dimethylpropylcarbamate;
methyl(1S)-1-[({(1S,3S,4S)-3-hydroxy-4-({(2S)-2-[3-(2-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1R,3S,4S)-4-({(2S)-3,3-dimethyl-2-[3-(2-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-4-({(2S)-3,3-dimethyl-2-[3-(3-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-3-hydroxy-4-({(2S)-2-[3-(2-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1R,3S,4S)-3-hydroxy-4-({(2S)-2-[3-(3-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(4-methyl-2-pyridinyl)benzyl]-5-phenylpentyl}amino)carbonyl]-2,2-dimethylpropylcarbamate;
methyl(1S)-1-[({(1S,2S,4S)-4-{[(2S)-2-(3-benzyl-2-oxo-1-imidazolidinyl)-3,3-dimethylbutanoyl]amino}-2-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(2-methyl-3-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(6-methyl-3-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[2-oxo-3-(3-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[2-oxo-3-(4-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[2-oxo-3-(2-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methyl-3-pyridinyl)benzyl]-5-phenylpentyl}amino)carbonyl]-2,2-dimethylpropylcarbamate;
methyl(1S)-1-[({(1S,2S,4S)-4-[((2S)-3,3-dimethyl-2-{3-[(2-methyl-1,3-thiazol-4-yl)methyl]-2-oxo-1-imidazolidinyl}butanoyl)amino]-2-hydroxy-1-[4-(5-methyl-2-pyridinyl)benzyl]-5-phenylpentyl}amino)carbonyl]-2,2-dimethylpropylcarbamate;
methyl(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2-methylbutylcarbamate;
methyl(1S)-1-[({(1S,3S,4S)-4-{[(2S)-2-(3-benzyl-2-oxo-1-imidazolidinyl)-3,3-dimethylbutanoyl]amino}-3-hydroxy-1-[4-(5-methyl-2-pyridinyl)benzyl]-5-phenylpentyl}amino)carbonyl]-2,2-dimethylpropylcarbamate;
methyl(1S)-1-[({(1S,2S,4R)-1-benzyl-4-[((2S)-3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentyl}amino)carbonyl]-2,2-dimethylpropylcarbamate;
methyl(1S)-1-[({(1S,2S,4S)-2-hydroxy-4-[((2S)-3-methyl-2-{3-[(1-methyl-1H-benzimidazol-2-yl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1S,2S,4S)-2-hydroxy-4-[((2S)-2-{3-[(2-isopropyl-1,3-thiazol-4-yl)methyl]-2-oxo-1-imidazolidinyl}-3-methylpentanoyl)amino]-5-phenyl-1-[4-(2-pyridinyl)benzyl]pentyl}amino)carbonyl]-2,2-dimethylpropylcarbamate;
methyl(1S)-1-[({(1R,3S,4S)-3-hydroxy-4-[((2S)-3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(1-methyl-1H-benzimidazol-2-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(1-methyl-1H-benzimidazol-2-yl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-3-hydroxy-4-[((2S)-2-{3-[(2-isopropyl-1,3-thiazol-4-yl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl(1S)-1-[({(1S,2S,4S)-2-hydroxy-4-[((2S)-2-{3-[(2-isopropyl-1,3-thiazol-4-yl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(2-methyl-1,3-thiazol-4-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(2-methyl-1,3-thiazol-4-yl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl(1S)-1-[({(1S,3R,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-di-methylpropylcarbamate;
methyl(1S)-1-[({(1R,3R,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate;
pyridinyl)methyl]-2-oxo-1-imidazolidinyl}butanoyl)amino]-3-hydroxy-1-[4-(6-methoxy-2-pyridinyl)benzyl]-5-phenylpentyl}amino)carbonyl]-2,2-dimethylpropylcarbamate;
methyl(1S)-1-[({(1S,3S,4S)-4-{[(2S)-2-(3-benzyl-2-oxo-1-imidazolidinyl)-3,3-dimethylbutanoyl]amino}-3-hydroxy-1-[4-(6-methoxy-2-pyridinyl)benzyl]-5-phenylpentyl}amino)carbonyl]-2,2-dimethylpropylcarbamate;
methyl(1S)-1-[({(1S,3S,4S)-4-[((2S)-2-{3-[(6-tert-butyl-2-pyridinyl)methyl]-2-oxo-1-imidazolidinyl}-3,3-dimethylbutanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-3-hydroxy-4-[((2S)-2-{3-[(6-isopropyl-2-pyridinyl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl(1S)-1-[({(1R,3S,4S)-4-[((2S)-2-{3-[(6-tert-butyl-2-pyridinyl)methyl]-2-oxo-1-imidazolidinyl}-3,3-dimethylbutanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-{[(2S)-2-(3-benzyl-2-oxo-1-imidazolidinyl)-3,3-dimethylbutanoyl]amino}-2-hydroxy-1-[4-(6-methoxy-2-pyridinyl)benzyl]-5-phenylpentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methoxy-2-pyridinyl)benzyl]-5-phenylpentyl}amino)carbonyl]-2,2-dimethylpropylcarbamate;
methyl(1S)-1-[({(1S,3S,4S)-4-({(2S)-2-[3-(2-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-2-[3-(4-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-2-[3-(3-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-1-[4-(5-methyl-2-pyridinyl)benzyl]-5-phenylpentyl}amino)carbonyl]-2,2-dimethylpropylcarbamate;
methyl(1S)-1-[({(1R,3S,4S)-4-[((2S)-3,3-dimethyl-2-{3-[(6-methyl-2-pyridinyl)methyl]-2-oxo-1-imidazolidinyl}butanoyl)amino]-3-hydroxy-1-[4-(5-methyl-2-pyridinyl)benzyl]-5-phenylpentyl}amino)carbonyl]-2,2-dimethylpropylcarbamate;
methyl(1S)-1-[({(1S,3S,4S)-3-hydroxy-4-[((2S)-2-{3-[(6-isopropyl-2-pyridinyl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl(1S)-1-[({(1S,3S,4S)-1-benzyl-4-[((2S)-3,3-dimethyl-2-{3-[(6-methyl-2-pyridinyl)methyl]-2-oxo-1-imidazolidinyl}butanoyl)amino]-3-hydroxy-5-[4-(2-pyridinyl)phenyl]pentyl}amino)carbonyl]-2,2-dimethylpropylcarbamate; and
methyl(1S)-1-({[(1S,2S,4S)-1-benzyl-2-hydroxy-4-({(2S)-3-methyl-2-[2-oxo-3-(4-quinolinylmethyl)-1-imidazolidinyl]pentanoyl}amino)-5-phenylpentyl]amino}carbonyl)-2,2-dimethylpropylcarbamate; or a pharmaceutically acceptable salt form, ester, salt of an ester, prodrug, salt of a prodrug, or combination thereof.

In a fourth embodiment, the present invention provides a compound of formula (IV) 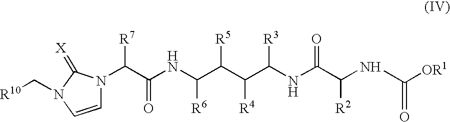
or a pharmaceutically acceptable salt form, stereoisomer, ester, salt of an ester, prodrug, salt of a prodrug, or combination thereof, wherein:
X is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁶ is hydrogen or R¹⁵;
R¹⁵ is 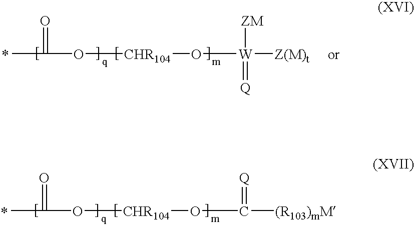
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each P, and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c}; and
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂.

For example, the present invention provides a compound of formula (IV) wherein X is O, R⁴ is H and R⁵ is OR¹⁶.

For example, the present invention provides a compound of formula (IV) wherein X is O, R⁴ is OR¹⁶ and R⁵ is H.

For example, the present invention provides a compound of formula (IV) wherein X is O, R⁴ is H, R⁵ is OR¹⁶ and R² is alkyl.

For example, the present invention provides a compound of formula (IV) wherein X is O, R⁴ is OR¹⁶, R⁵ is H and R² is alkyl.

For example, the present invention provides a compound of formula (IV) wherein X is O, R⁴ is H, R⁵ is OR¹⁶, R² is alkyl and R³ is arylalkyl.

For example, the present invention provides a compound of formula (IV) wherein X is O, R⁴ is OR¹⁶ and R⁵ is H, R² is alkyl and R³ is arylalkyl.

For example, the present invention provides a compound of formula (IV) wherein X is O, R⁴ is H, R⁵ is OR¹⁶, R² is alkyl, R³ is arylalkyl substituted with R^{3a}, and R^{3a} is aryl or heteroaryl.

For example, the present invention provides a compound of formula (IV) wherein X is O, R⁴ is OR¹⁶ and R⁵ is H, R² is alkyl, R³ is arylalkyl substituted with R^{3a}, and R^{3a} is aryl or heteroaryl.

For example, the present invention provides a compound of formula (IV) wherein X is O, R⁴ is H, R⁵ is OR¹⁶, R² is alkyl, R³ is arylalkyl substituted with R^{3a}, R⁶ is arylalkyl and R^{3a} is aryl or heteroaryl.

For example, the present invention provides a compound of formula (IV) wherein X is O, R⁴ is OR¹⁶ and R⁵ is H, R¹ is alkyl, R³ is arylalkyl substituted with R^{3a}, R⁶ is arylalkyl and R^{3a} is aryl or heteroaryl.

For example, the present invention provides a compound of formula (IV) wherein X is O, R⁴ is H, R⁵ is OR¹⁶, R² is alkyl, R³ is arylalkyl substituted with R^{3a}, R⁶ is arylalkyl and R^{3a} is heteroaryl.

For example, the present invention provides a compound of formula (IV) wherein X is O, R⁴ is OR¹⁶ and R⁵ is H, R² is alkyl, R³ is arylalkyl substituted with R^{3a}, R⁶ is arylalkyl, and R^{3a} is heteroaryl.

For example, the present invention provides a compound of formula (IV) wherein X is O, R⁴ is H, R⁵ is OR¹⁶, R² is alkyl, R³ is arylalkyl substituted with R^{1a}, R⁶ is arylalkyl, R⁷ is alkyl and R^{3a} is heteroaryl.

For example, the present invention provides a compound of formula (I) wherein X is O, R⁴ is OR¹⁶, R⁵ is H, R² is alkyl, R³ is arylalkyl substituted with R^{3a}, R⁶ is arylalkyl, R⁷ is alkyl and R^{3a} is heteroaryl.

For example, the present invention provides a compound of formula (IV) wherein X is O, R⁴ is H, R⁵ is OR¹⁶, R² is alkyl, R³ is arylalkyl substituted with R^{3a}, R⁶ is arylalkyl, R⁷ is alkyl, R¹⁰ is aryl or heteroaryl and R^{3a} is heteroaryl.

For example, the present invention provides a compound of formula (IV) wherein X is O, R⁴ is OR¹⁶, R⁵ is H, R³ is alkyl, R³ is arylalkyl substituted with R^{3a}, R⁶ is arylalkyl, R⁷ is alkyl, R¹⁰ is aryl or heteroaryl and R³, is heteroaryl.

In a fifth embodiment the present invention provides a compound of formula (V) 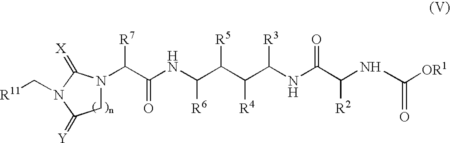
or a pharmaceutically acceptable salt form, stereoisomer, ester, salt of an ester, prodrug, salt of a prodrug, or combination thereof, wherein:
X is O, S or NH;
Y is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹⁶ is hydrogen or R¹⁵;
R¹⁵ is 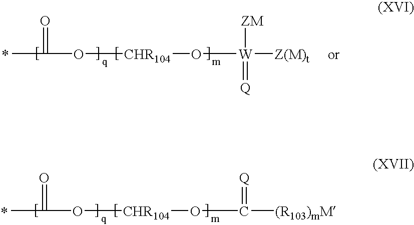
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each K and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

For example, the present invention provides a compound of formula (V) wherein X is O and Y is O.

For example, the present invention provides a compound of formula (V) wherein X is O, Y is O, R⁴ is H and R⁵ is OR¹⁶.

For example, the present invention provides a compound of formula (V) wherein X is O, Y is O, R⁴ is OR¹⁶ and R⁵ is H.

For example, the present invention provides a compound of formula (V) wherein X is O, Y is O, R⁴ is H, R⁵ is OR¹⁶, and R² is alkyl.

For example, the present invention provides a compound of formula (V) X is O, Y is O, R⁴ is O OR¹⁶, R⁵ is H, and R² is alkyl.

For example, the present invention provides a compound of formula (V) wherein X is O, Y is O, R⁴ is H, R⁵ is OR¹⁶, R² is alkyl, and R³ is arylalkyl.

For example, the present invention provides a compound of formula (V) wherein X is O, Y is O, R⁴ is OR¹⁶, R⁵ is H, R² is alkyl, and R³ is arylalkyl.

For example, the present invention provides a compound of formula (V) wherein X is O, Y is O, R⁴ is H, R⁵ is OR¹⁶, R² is alkyl, and R³ is arylalkyl substituted with R^{3a}.

For example, the present invention provides a compound of formula (V) wherein X is O, Y is O, R⁴ is OR¹⁶, R⁵ is H, R² is alkyl, and R³ is arylalkyl substituted with R^{3a}.

For example, the present invention provides a compound of formula (V) wherein X is O, Y is O, R⁴ is H, R¹ is OR¹⁶, R² is alkyl, R³ is arylalkyl substituted with R^{3a} and R^{3a} is aryl or heteroaryl.

For example, the present invention provides a compound of formula (V) wherein X is O, Y is O, R⁴ is OR¹⁶, R⁵ is H, R² is alkyl, R³ is arylalkyl substituted with R^{3a} and R^{3a} is aryl or heteroaryl.

For example, the present invention provides a compound of formula (V) wherein X is O, Y is O, R⁴ is H, R⁵ is OR¹⁶, R² is alkyl, R³ is arylalkyl substituted with R^{3a}, R¹¹ is aryl or heteroaryl and R^{3a} is aryl or heteroaryl.

For example, the present invention provides a compound of formula (V) wherein X is O, Y is O, R⁴ is OR¹⁶, R⁵ is H, R² is alkyl, R³ is aralkyl substituted with R^{3a}, R¹¹ is aryl or heteroaryl and R^{3a} is aryl or heteroaryl.

For example, the present invention provides a compound of formula (V) wherein X is O, Y is O, R⁴ is H, R⁵ is OR¹⁶, R² is alkyl, R³ is arylalkyl substituted with R^{3a}, R¹¹ is aryl or heteroaryl, R⁷ is alkyl and R^{3a} is aryl or heteroaryl.

For example, the present invention provides a compound of formula (V) wherein X is O, Y is O, R⁴ is OR¹⁶, R⁵ is H, R² is alkyl, R³ is arylalkyl substituted with R^{3a}, R¹¹ is aryl or heteroaryl, R⁷ is alkyl and R³, is aryl or heteroaryl.

For example, the present invention provides a compound of formula (V) wherein X is O, Y is O, R⁴ is H, R⁵ is OR¹⁶, R² is alkyl, R³ is arylalkyl substituted with R^{3a}, R¹¹ is aryl or heteroaryl, R⁷ is alkyl, R⁶ is arylalkyl and R^{3a} is aryl or heteroaryl.

For example, the present invention provides a compound of formula (V) wherein X is O, Y is O, R⁴ is OR¹⁶, R⁵ is H, R² is alkyl, R³ is arylalkyl substituted with R^{3a}, R¹¹ is aryl or heteroaryl, R⁷ is alkyl, R⁶ is arylalkyl and R^{3a} is aryl or heteroaryl.

For example, the present invention provides a compound of formula (V) wherein X is O, Y is O, R⁴ is H, R⁵ is OR¹⁶, R² is alkyl, R³ is arylalkyl substituted with R^{3a}, R¹¹ is aryl or heteroaryl, R⁷ is alkyl, R⁶ is arylalkyl, R¹ is alkyl and R^{3a} is aryl or heteroaryl.

For example, the present invention provides a compound of formula (V) wherein X is O, Y is O, R⁴ is OR¹⁶, R⁵ is H, R² is alkyl, R³ is arylalkyl substituted with R^{3a}, R¹¹ is aryl or heteroaryl, R⁷ is alkyl, R⁶ is arylalkyl, R¹ is alkyl and R^{3a} is aryl or heteroaryl.

For example, the present invention provides a compound of formula (V) wherein X is O, Y is O, R⁴ is H, R⁵ is OR¹⁶, R² is C1, C2, C3, C4 or C5 alkyl, R³ is arylalkyl substituted with Rₐ, R¹¹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R⁷ is C1, C2, C3, C4 or C5 alkyl, R⁶ is arylalkyl, R¹ is alkyl and R^{3a} is aryl or heteroaryl.

For example, the present invention provides a compound of formula (V) wherein X is O, Y is O, R⁴ is OR¹⁶, R⁵ is H, R² is C1, C2, C3, C4, C4 or C5 alkyl, R³ is arylalkyl substituted with R^{3a}, R¹¹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R⁷ is C1, C2, C3, C4 or C5 alkyl, R⁶ is arylalkyl, R¹ is alkyl and R^{3a} is aryl or heteroaryl.

For example, the present invention provides a compound of formula (V) wherein X is O, Y is O, R⁴ is H, R⁵ is OR¹⁶, R² is C1, C2, C3, C4 or C5 alkyl, R³ is phenylmethyl substituted with R^{3a}, R¹¹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R⁷ is C1, C2, C3, C4 or C5 alkyl, R⁶ is phenylmethyl and R¹ is methyl and R^{3a} is aryl or heteroaryl.

For example, the present invention provides a compound of formula (V) wherein X is O, Y is O, R⁴ is OR¹⁶, R⁵ is H, R² is C1, C2, C3, C4, C4 or C5 alkyl, R³ is phenylmethyl substituted with R^{3a}, R¹¹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R⁷ is C1, C2, C3, C4 or C5 alkyl, R^{3a} is phenylmethyl, R¹ is methyl and R^{3a} is aryl or heteroaryl.

For example, the present invention provides a compound of formula (V) wherein X is O, Y is O, R⁴ is H, R⁵ is OR¹⁶, R² is isopropyl, tert-butyl or 1-methylpropyl, R³ is phenylmethyl substituted with R^{3a}, R¹¹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R⁷ is isopropyl, tert-butyl or 1-methylpropyl, R⁶ is phenylmethyl, R¹ is methyl and R^{3a} is pyridyl.

For example, the present invention provides a compound of formula (V) wherein X is O, Y is O, R⁴ is OR¹⁶, R⁵ is H, R² is isopropyl, tert-butyl or 1-methylpropyl, R³ is phenylmethyl substituted with R^{3a}, R¹¹ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R⁷ is isopropyl, tert-butyl or 1-methylpropyl, R⁶ is phenylmethyl, R¹ is methyl and R^{3a} is pyridyl.

Exemplary compounds of the present invention of formula (V) include, but not limited to, the following:
methyl(1S)-1-[({(1S,3S,4S)-3-hydroxy-4-[((2S)-3-methyl-2-{3-[(6-methyl-2-pyridinyl)methyl]-2,4-dioxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1S,2S,4S)-2-hydroxy-4-[((2S)-3-methyl-2-{3-[(6-methyl-2-pyridinyl)methyl]-2,4-dioxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1R,3S,4S)-4-[((2S)-3,3-dimethyl-2-{3-[(6-methyl-2-pyridinyl)methyl]-2,4-dioxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(6-methyl-2-pyridinyl)methyl]-2,4-dioxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2,4-dioxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl(1S)-1-[({[(1S,3S,4S)-3-hydroxy-4-[((2S)-2-{3-[(2-isopropyl-1,3-thiazol-4-yl)methyl]-2,4-dioxo-1-imidazolidinyl}-3-methylpentanoyl)amino]-5-phenyl-1-[4-(2-pyridinyl)benzyl]pentyl}amino)carbonyl]-2,2-dimethylpropylcarbamate; and
methyl(1S)-1-[({(1S,3S,4S)-4-{[(2S)-2-(2,4-dioxo-3-{[2-(3-pyridinyl)-1,3-thiazol-4-yl]methyl}—1 imidazolidinyl)-3-methylpentanoyl]amino}-3-hydroxy-5-phenyl-1-[4-(2-pyridinyl)benzyl]pentyl}amino)carbonyl]-2,2-dimethylpropylcarbamate; or a pharmaceutically acceptable salt form, ester, salt of an ester, prodrug, salt of a prodrug, or combination thereof.

In a sixth embodiment the present invention provides a compound of formula (VI) 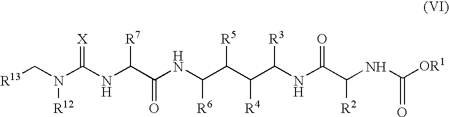
or a pharmaceutically acceptable salt form, stereoisomer, ester, salt of an ester, prodrug, salt of a prodrug, or combination thereof, wherein:
X is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁶ is hydrogen or R¹⁵;
R¹⁵ is 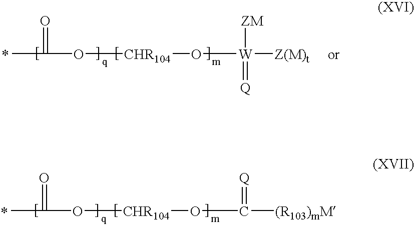
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c}; and
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂.

For example, the present invention provides a compound of formula (VI) wherein R⁴ is H and R⁵ is OR¹⁶.

For example, the present invention provides a compound of formula (VI) wherein R⁴ is OR¹⁶ and R⁵ is H.

For example, the present invention provides a compound of formula (VI) wherein R⁴ is H, R⁵ is OR¹⁶, and R³ is arylalkyl.

For example, the present invention provides a compound of formula (VI) wherein R⁴ is OR¹⁶, R⁵ is H, and R³ is arylalkyl.

For example, the present invention provides a compound of formula (VI) wherein R⁴ is H, R⁵ is OR¹⁶, and R³ is arylalkyl substituted with R³.

For example, the present invention provides a compound of formula (VI) wherein R⁴ is OR¹⁶, R⁵ is H, and R³ is arylalkyl substituted with R^{3a}.

For example, the present invention provides a compound of formula (VI) wherein R⁴ is H, R⁵ is OR¹⁶, R³ is arylalkyl substituted with R^{3a} and R^{3a} is aryl or heteroaryl.

For example, the present invention provides a compound of formula (VI) wherein R⁴ is OR¹⁶, R⁵ is H, R³ is arylalkyl substituted with R^{3a} and R^{3a} is aryl or heteroaryl.

For example, the present invention provides a compound of formula (VI) wherein R⁴ is H, R⁵ is OR¹⁶, R³ is arylalkyl substituted with R^{3a}, R¹² is alkyl and R^{3a} is aryl or heteroaryl.

For example, the present invention provides a compound of formula (VI) wherein R⁴ is OR¹⁶, R⁵ is H, R³ is arylalkyl substituted with R^{3a}, R¹² is alkyl and R^{3a} is aryl or heteroaryl.

For example, the present invention provides a compound of formula (VI) wherein R⁴ is H, R⁵ is OR¹⁶, R³ is arylalkyl substituted with R^{3a}, R¹² is alkyl, R³ is aryl or heteroaryl; and R^{3a} is aryl or heteroaryl.

For example, the present invention provides a compound of formula (VI) wherein R⁴ is OR¹⁶, R⁵ is H, R³ is arylalkyl substituted with R³, R¹² is alkyl, R³ is aryl or heteroaryl and R^{3a} is aryl or heteroaryl.

For example, the present invention provides a compound of formula (VI) wherein R⁴ is H, R⁵ is OR¹⁶, R³ is arylalkyl substituted with R³—, R¹² is alkyl, R¹³ is aryl or heteroaryl, R² is alkyl and R^{3a} is aryl or heteroaryl.

For example, the present invention provides a compound of formula (VI) wherein R⁴ is OR¹⁶, R⁵ is H, R³ is arylalkyl substituted with R^{3a}, R¹² is alkyl, R¹³ is aryl or heteroaryl, R² is alkyl and R^{3a} is aryl or heteroaryl.

For example, the present invention provides a compound of formula (VI) wherein R⁴ is H, R⁵ is OR¹⁶, R³ is arylalkyl substituted with R^{3a}, R¹² is alkyl, R³ is aryl or heteroaryl, R² is alkyl, R⁷ is alkyl and R^{3a} is aryl or heteroaryl.

For example, the present invention provides a compound of formula (VI) wherein R⁴ is OR¹⁶, R⁵ is H, R³ is arylalkyl substituted with R^{3a}, R¹² is alkyl, R¹³ is aryl or heteroaryl, R² is alkyl, R⁷ is alkyl and R^{3a} is aryl or heteroaryl.

For example, the present invention provides a compound of formula (VI) wherein R⁴ is H, R⁵ is OR¹⁶, R³ is arylalkyl substituted with R^{3a}, R¹² is alkyl, R³ is aryl or heteroaryl, R² is alkyl, R⁷ is alkyl, R⁶ is arylalkyl and R^{3a} is aryl or heteroaryl.

For example, the present invention provides a compound of formula (VI) wherein R⁴ is OR¹⁶, R⁵ is H, R³ is arylalkyl substituted with R^{3a}, R¹² is alkyl, R¹³ is aryl or heteroaryl, R² is alkyl, R⁷ is alkyl, R⁶ is arylalkyl and R^{3a} is aryl or heteroaryl.

For example, the present invention provides a compound of formula (VI) wherein R⁴ is H, R⁵ is OR¹⁶, R³ is arylalkyl substituted with R^{3a}, R¹² is alkyl, R¹³ is aryl or heteroaryl, R² is alkyl, R⁷ is alkyl, R¹ is alkyl, R⁶ is arylalkyl and R^{3a} is aryl or heteroaryl.

For example, the present invention provides a compound of formula (VI) wherein R⁴ is OR¹⁶, R⁵ is H, R³ is arylalkyl substituted with R³, R¹² is alkyl, R¹³ is aryl or heteroaryl, R² is alkyl, R⁷ is alkyl, R¹ is alkyl, R⁶ is arylalkyl and R^{3a} is aryl or heteroaryl.

For example, the present invention provides a compound of formula (VI) wherein R⁴ is H, R⁵ is OR¹⁶, R³ is phenylmethyl substituted with R^{3a}, R¹² is methyl or ethyl, R¹³ is aryl or heteroaryl, R² is C1, C2, C3, C4 or C5 alkyl, R⁷ is C1, C2, C3, C4 or C5 alkyl, R¹ is methyl, R⁶ is phenylmethyl and R^{3a} is aryl or heteroaryl.

For example, the present invention provides a compound of formula (VI) wherein R⁴ is OR¹⁶, R³ is H, R³ is phenylmethyl substituted with R^{3a}, R¹² is methyl or ethyl, R³ is aryl or heteroaryl, R² is C1, C2, C3, C4 or C5 alkyl, R⁷ is C1, C2, C3, C4 or C5 alkyl, R¹ is methyl, R⁶ is phenylmethyl and R^{3a} is aryl or heteroaryl.

For example, the present invention provides a compound of formula (VI) wherein R⁴ is H, R⁵ is OR¹⁶, R³ is phenylmethyl substituted with Rₐ, R¹² is methyl or ethyl, R³ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R² is C1, C2, C3, C4 or C5 alkyl, R⁷ is C1, C2, C3, C4 or C5 alkyl, R¹ is methyl, R⁶ is phenylmethyl, and R^{3a} is thienyl, furanyl, pyrrolyl, thiazolyl, oxazolyl, imidazolyl, pyrazolyl, isothiazolyl, isoxazolyl, pyridyl or phenyl.

For example, the present invention provides a compound of formula (VI) wherein R⁴ is OR¹⁶, R⁵ is H, R³ is phenylmethyl substituted with R^{3a}, R¹² is methyl or ethyl, R¹³ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R² is C1, C2, C3, C4 or C5 alkyl, R⁷ is C1, C2, C3, C4 or C5 alkyl, R¹ is methyl, R⁶ is phenylmethyl, and R^{3a} is thienyl, furanyl, pyrrolyl, thiazolyl, oxazolyl, imidazolyl, pyrazolyl, isothiazolyl, isoxazolyl, pyridyl or phenyl.

For example, the present invention provides a compound of formula (VI) wherein R⁴ is H, R⁵ is OR¹⁶, R³ is phenylmethyl substituted with R^{3a}, R¹² is methyl or ethyl, R¹³ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R² is C1, C2, C3, C4 or C5 alkyl, R¹ is methyl, R⁷ is C1, C2, C3, C4 or C5 alkyl, R⁶ is phenylmethyl and R^{3a} is pyridyl.

For example, the present invention provides a compound of formula (VI) wherein R⁴ is OR¹⁶, R⁵ is H, R³ is phenylmethyl substituted with R^{3a}, R¹² is methyl or ethyl, R¹³ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R² is C1, C2, C3, C4 or C5 alkyl, R¹ is methyl, R⁷ is C1, C2, C3, C4 or C5 alkyl, R⁶ is phenylmethyl, and R^{3a} is pyridyl.

For example, the present invention provides a compound of formula (VI) wherein R⁴ is H, R⁵ is OR¹⁶, R³ is phenylmethyl substituted with R^{3a}, R¹² is methyl or ethyl, R¹³ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R² is isopropyl, tert-butyl or 1-methylpropyl, R¹ is methyl, R⁷ is isopropyl, tert-butyl or 1-methylpropyl, R⁶ is phenylmethyl, and R^{3a} is pyridyl.

For example, the present invention provides a compound of formula (VI) wherein R⁴ is OR¹⁶, R⁵ is H, R³ is phenylmethyl substituted with R^{3a}, R¹² is methyl or ethyl, R¹³ is thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl or indazolyl, R² is isopropyl, tert-butyl or 1-methylpropyl, R¹ is methyl, R⁷ is isopropyl, tert-butyl or 1-methylpropyl, R⁶ is phenylmethyl, and R^{3a} is pyrdiyl.

Exemplary compounds of the present invention of formula (VI) include, but not limited, to the following:
methyl(1S,4S,6S,7S,10S)-7-benzyl-1,10-ditert-butyl-6-hydroxy-13-methyl-2,9,12-trioxo-14-phenyl-4-[4-(2-pyridinyl)benzyl]-3,8,11,13-tetraazatetradec-1-ylcarbamate;
methyl(1S,4R,6S,7S,10S)-7-benzyl-1,10-ditert-butyl-6-hydroxy-13-methyl-2,9,12-trioxo-14-phenyl-4-[4-(2-pyridinyl)benzyl]-3,8,11,13-tetraazatetradec-1-ylcarbamate;
methyl(1S,4S,6S,7S,10S)-7-benzyl-10-sec-butyl-1-tert-butyl-6-hydroxy-13-methyl-14-(2-methyl-1,3-thiazol-4-yl)-2,9,12-trioxo-4-[4-(2-pyridinyl)benzyl]-3,8,11,13-tetraazatetradec-1-ylcarbamate; and
methyl(1S,4S,5S,7S,10S)-7-benzyl-10-sec-butyl-1-tert-butyl-5-hydroxy-13-methyl-14-(2-methyl-1,3-thiazol-4-yl)-2,9,12-trioxo-4-[4-(2-pyridinyl)benzyl]-3,8,11,13-tetraazatetradec-1-ylcarbamate; or a pharmaceutically acceptable salt form, streoisomer, ester, salt of an ester, prodrug, salt of a prodrug, or combination thereof.

In a seventh embodiment, the present invention provides a compound of formula (VII) 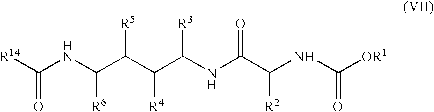
or a pharmaceutically acceptable salt form, stereoisomer, ester, salt of an ester, prodrug, salt of a prodrug, or combination thereof, wherein:
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R¹⁴ is —ORₐ or -alkylORₐ;
R¹⁶ is hydrogen or R¹⁵;
R⁵ is 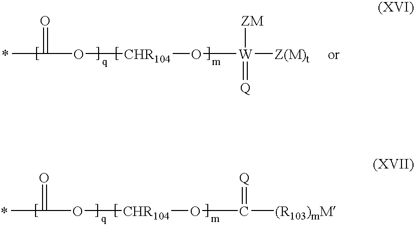
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂ or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀ or —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c}; and
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂.

For example, the present invention provides a compound of formula (VII) wherein R⁴ is H, R⁵ is OR¹⁶, and R² is alkyl.

For example, the present invention provides a compound of formula (VII) wherein R⁴ is OR¹⁶, R⁵ is H, and R² is alkyl.

For example, the present invention provides a compound of formula (VII) wherein R⁴ is H, R⁵ is OR¹⁶, R² is alkyl and R³ is arylalkyl.

For example, the present invention provides a compound of formula (VII) wherein R⁴ is OR¹⁶, R⁵ is H, R² is alkyl and R³ is arylalkyl.

For example, the present invention provides a compound of formula (VII) wherein R⁴ is H, R⁵ is OR¹⁶, R² is alkyl and R³ is arylalkyl substituted with R^{3a}.

For example, the present invention provides a compound of formula (VII) wherein R⁴ is OR¹⁶, R⁵ is H, R² is alkyl and R³ is arylalkyl substituted with R^{3a}.

For example, the present invention provides a compound of formula (VII) wherein R⁴ is H, R⁵ is OR¹⁶, R² is alkyl, R³ is arylalkyl substituted with R^{3a}, and R^{3a} is aryl or heteroaryl.

For example, the present invention provides a compound of formula (VII) wherein R⁴ is OR¹⁶, R⁵ is H, R² is alkyl, R³ is arylalkyl substituted with R^{3a}, and R^{3a} is aryl or heteroaryl.

For example, the present invention provides a compound of formula (VII) wherein R⁴ is H, R⁵ is OR¹⁶, R² is alkyl, R³ is arylalkyl substituted with R^{3a}, R⁴ is ORₐ or -alkylORₐ, R^{3a} is aryl or heteroaryl, and Rₐ is aryl or heterocycle.

For example, the present invention provides a compound of formula (VII) wherein R⁴ is OR¹⁶, R⁵ is H, R² is alkyl, R³ is arylalkyl substituted with R^{3a}, R¹⁴ is ORₐ or -alkylORₐ, R^{3a} is aryl or heteroaryl, and Rₐ is aryl or heterocycle.

For example, the present invention provides a compound of formula (VII) wherein R⁴ is H, R⁵ is OR¹⁶, R² is alkyl, R³ is arylalkyl substituted with R^{3a}, R¹⁴ is ORₐ or -alkylORₐ, R⁷ is alkyl, R^{3a} is aryl or heteroaryl, and Rₐ is aryl or heterocycle.

For example, the present invention provides a compound of formula (VII) wherein R⁴ is OR¹⁶, R⁵ is H, R² is alkyl, R³ is arylalkyl substituted with R^{3a}, R¹⁴ is ORₐ or -alkylORₐ, R⁷ is alkyl, R^{3a} is aryl or heteroaryl, and Rₐ is aryl or heterocycle.

For example, the present invention provides a compound of formula (VII) wherein R⁴ is H, R⁵ is OR¹⁶, R² is alkyl, R³ is arylalkyl substituted with R^{3a}, R¹⁴ is ORₐ or -alkylORₐ, R⁷ is alkyl, R⁶ is arylalkyl, R^{3a} is aryl or heteroaryl, and Rₐ is aryl or heterocycle.

For example, the present invention provides a compound of formula (VII) wherein R⁴ is OR¹⁶, R⁵ is H, R² is alkyl, R³ is arylalkyl substituted with R^{3a}, R¹⁴ is ORₐ or -alkylORₐ, R⁷ is alkyl, R⁶ is arylalkyl, R^{3a} is aryl or heteroaryl, and Rₐ is aryl or heterocycle.

For example, the present invention provides a compound of formula (VII) wherein R⁴ is H, R⁵ is OR¹⁶, R² is alkyl, R³ is arylalkyl substituted with R^{3a}, R⁴ is ORₐ or -alkylORₐ, R⁷ is alkyl, R⁶ is arylalkyl, R¹ is alkyl, R^{3a} is aryl or heteroaryl, and Rₐ is aryl or heterocycle.

For example, the present invention provides a compound of formula (VII) wherein R⁴ is OR¹⁶, R⁵ is H, R² is alkyl, R³ is arylalkyl substituted with R^{3a}, R¹⁴ is ORₐ or -alkylORₐ, R⁷ is alkyl, R⁶ is arylalkyl, R¹ is alkyl, R^{3a} is aryl or heteroaryl, and Rₐ is aryl or heterocycle.

For example, the present invention provides a compound of formula (VII) wherein R⁴ is H, R⁵ is OR¹⁶, R² is C1, C2, C3, C4 or C5 alkyl, R³ is arylalkyl substituted with R^{3a}, R¹⁴ is ORₐ or -alkylORₐ, R⁷ is alkyl, R⁶ is arylalkyl, R¹ is alkyl, R^{3a} is aryl or heteroaryl, and Rₐ is aryl or heterocycle.

For example, the present invention provides a compound of formula (VII) wherein R⁴ is OR¹⁶, R⁵ is H, R² is C1, C2, C3, C4 or C5 alkyl, R³ is arylalkyl substituted with Rₐ, R¹⁴ is ORₐ or -alkylORₐ, R⁷ is alkyl, R⁶ is arylalkyl, R¹ is alkyl, R^{3a} is aryl or heteroaryl, and Rₐ is aryl or heterocycle.

For example, the present invention provides a compound of formula (VII) wherein R⁴ is H, R⁵ is OR¹⁶, R² is C1, C2, C3, C4 or C5 alkyl, R³ is phenylmethyl substituted with R^{3a}, R¹⁴ is ORₐ or -alkylORₐ, R⁷ is C1, C2, C3, C4 or C5 alkyl, R⁶ is arylalkyl, R¹ is alkyl, R^{3a} is heteroaryl, and Rₐ is aryl or heterocycle.

For example, the present invention provides a compound of formula (VII) wherein R⁴ is OR¹⁶, R⁵ is H, R² is C1, C2, C3, C4 or C5 alkyl, R³ is phenylmethyl substituted with R^{3a}, R¹⁴ is ORₐ or -alkylORₐ, R⁷ is C1, C2, C3, C4 or C5 alkyl, R⁶ is arylalkyl, R¹ is alkyl, R^{3a} is heteroaryl, and Rₐ is aryl or heterocycle.

For example, the present invention provides a compound of formula (VII) wherein R⁴ is H, R⁵ is OR¹⁶, R² is C1, C2, C3, C4 or C5 alkyl, R³ is phenylmethyl substituted with R^{3a}, R¹⁴ is ORₐ or -alkylORₐ, R⁷ is C1, C2, C3, C4 or C5 alkyl, R⁶ is phenylmethyl, R¹ is alkyl, R^{3a} is heteroaryl, and Rₐ is aryl or heterocycle.

For example, the present invention provides a compound of formula (VII) wherein R⁴ is OR¹⁶, R⁵ is H, R² is C1, C2, C3, C4 or C5 alkyl, R³ is phenylmethyl substituted with R^{3a}, R⁴ is ORₐ or -alkylORₐ, R⁷ is C1, C2, C3, C4 or C5 alkyl, R⁶ is phenylmethyl, R¹ is alkyl, R^{3a} is heteroaryl, and Rₐ is aryl or heterocycle.

For example, the present invention provides a compound of formula (VII) wherein R⁴ is H, R⁵ is OR¹⁶, R² is C1, C2, C3, C4 or C5 alkyl, R³ is phenylmethyl substituted with R^{3a}, R¹⁴ is ORₐ or -alkylORₐ, R⁷ is C1, C2, C3, C4 or C5 alkyl, R⁶ is phenylmethyl, R¹ is methyl, R^{3a} is pyridyl, and Rₐ is phenyl or hexahydrofurofuranyl.

For example, the present invention provides a compound of formula (VII) wherein R⁴ is OR¹⁶, R⁵ is H, R² is C1, C2, C3, C4 or C5 alkyl, R³ is phenylmethyl substituted with R³, R¹⁴ is ORₐ or -alkylORₐ, R⁷ is C1, C2, C3, C4 or C5 alkyl, R⁶ is phenylmethyl, k is methyl, R^{3a} is pyridyl, and Rₐ is phenyl or hexahydrofurofuranyl.

For example, the present invention provides a compound of formula (VII) wherein R⁴ is H, R⁵ is OR¹⁶, R² is isopropyl, 1-methylpropyl or tert-butyl, R³ is phenylmethyl substituted with R^{3a}, R¹⁴ is ORₐ or -alkylORₐ, R⁷ is isopropyl, 1-methylpropyl or tert-butyl, R⁶ is phenylmethyl, R¹ is methyl, R^{3a} is pyridyl, and Rₐ is phenyl or hexahydrofurofuranyl.

For example, the present invention provides a compound of formula (VII) wherein R⁴ is OR¹⁶, R⁵ is H, R² is isopropyl, 1-methylpropyl or tert-butyl, R³ is phenylmethyl substituted with R^{3a}, R¹⁴ is ORₐ or -alkylORₐ, R⁷ is isopropyl, 1-methylpropyl or tert-butyl, R⁶ is phenylmethyl, R¹ is methyl, R^{3a} is pyridyl, and Rₐ is phenyl or hexahydrofurofuranyl.

Exemplary compounds of the present invention of formula (VII) include, but not limited to, the following:
1:1 mixture of (3R,3aS,6aR)-hexahydrofuro[2,3-b]furan-3-yl(1S,3S,4S)-1-benzyl-3-hydroxy-4-({(2S)-2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl(1S,3S,4S)-1-benzyl-3-hydroxy-4-({(2S)-2-[(methoxycarbonyl)amino]-3,3-dimethylbutanoyl}amino)-5-[4-(2-pyridinyl)phenyl]pentylcarbamate;
1:1 mixture of (3R,3aS,6aR)-hexahydrofuro[2,3-b]furan-3-yl(1S,2S,4S)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl(1S,2S,4S)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate;
methyl(1S)-1-[({(1S,3S,4S)-4-{[(2,6-dimethylphenoxy)acetyl]amino}-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-{[(2,6-dimethylphenoxy)acetyl]amino}-2-hydroxy-5-phenyl-1-[4-(2-pyridinyl)benzyl]pentyl}amino)carbonyl]-2,2-dimethylpropylcarbamate;
1:1 mixture of (3R,3aS,6aR)-hexahydrofuro[2,3-b]furan-3-yl(1S,2S,4R)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl(1S,2S,4R)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate; and
methyl(1S)-1-[({(1R,3S,4S)-4-{[(2,6-dimethylphenoxy)acetyl]amino}-3-hydroxy-5-phenyl-1-[4-(2-pyridinyl)benzyl]pentyl}amino)carbonyl]-2,2-dimethylpropylcarbamate; or a pharmaceutically acceptable salt form, stereoisomer, ester, salt of an ester, prodrug, salt of a prodrug, or combination thereof.

In an eighth embodiment the present invention provides a compound of formula (VIII) 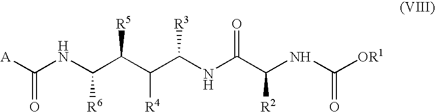
or a pharmaceutically acceptable salt form, stereoisomer, ester, salt of an ester, prodrug, salt of a prodrug, or combination thereof, wherein:
A is 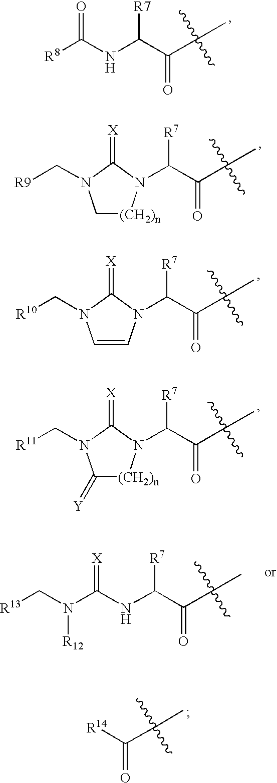
X is O, S or NH;
Y is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and Rₐ;
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁸ is —ORₐ or -alkylORₐ;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁴ is —ORₐ or -alkylORₐ;
R¹⁶ is hydrogen or R¹⁵;
R¹⁵ is 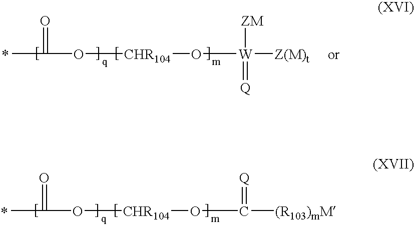
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆-SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

For example, the present invention provides a compound of formula (VIII) wherein X is O and Y is O.

For example, the present invention provides a compound of formula (VIII) wherein X is O, Y is O, R⁴ is H, R⁵ is OR¹⁶ and R² is alkyl.

For example, the present invention provides a compound of formula (VIII) wherein X is O, Y is O, R⁴ is H, R⁵ is OR¹⁶, R² is alkyl and R³ is arylalkyl.

For example, the present invention provides a compound of formula (VIII) wherein X is O, Y is O, R⁴ is H, R⁵ is OR¹⁶, R² is alkyl and R³ is arylalkyl substituted with R^{3a}.

For example, the present invention provides a compound of formula (VIII) wherein X is O, Y is O, R⁴ is H, R⁵ is OR¹⁶, R² is alkyl, R³ is arylalkyl substituted with R^{3a}, and R^{3a} is aryl or heteroaryl.

For example, the present invention provides a compound of formula (VIII) wherein X is O, Y is O, R⁴ is H, R⁵ is, OR¹⁶, R² is alkyl, R³ is arylalkyl substituted with R^{3a}, R¹ is alkyl, and R^{3a} is aryl or heteroaryl.

For example, the present invention provides a compound of formula (VIII) wherein X is O, Y is O, R⁴ is H, R⁵ is OR¹⁶, R² is alkyl, R³ is arylalkyl substituted with R³, R¹ is alkyl, R⁶ is arylalkyl, and R^{3a} is aryl or heteroaryl.

For example, the present invention provides a compound of formula (VIII) wherein X is O, Y is O, R⁴ is H, R⁵ is OR¹⁶, R² is alkyl, R³ is arylalkyl substituted with R^{3a}, R¹ is alkyl, R⁶ is arylalkyl, and R^{3a} is aryl or hetroaryl.

In a ninth embodiment the present invention provides a compound of formula (IX) 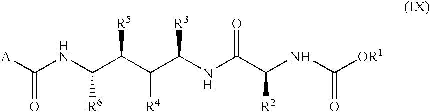
or a pharmaceutically acceptable salt form, stereoisomer, ester, salt of an ester, prodrug, salt of a prodrug, or combination thereof, wherein:
A is 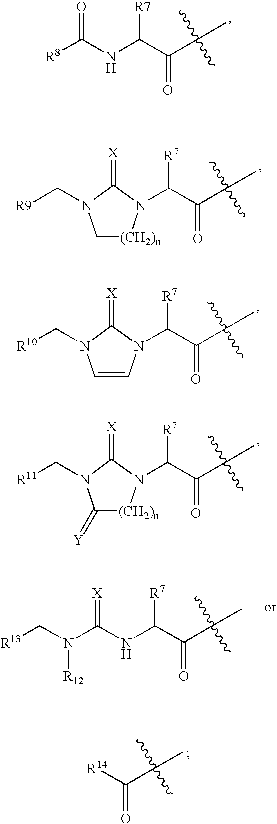
X is O, S or NH;
Y is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁸ is —ORₐ or -alkylORₐ;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁴ is —ORₐ or -alkylORₐ;
R¹⁶ is hydrogen or R¹⁵;
R¹⁵ is 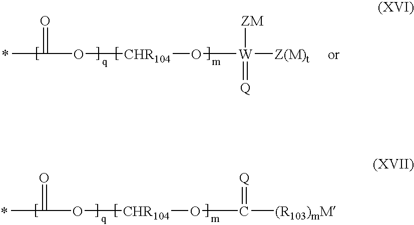
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl-4-s optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅)—OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

For example, the present invention provides a compound of formula (IX) wherein X is O and Y is O.

For example, the present invention provides a compound of formula (IX) wherein X is O, Y is O, R⁴ is H, R⁵ is OR¹⁶ and R² is alkyl.

For example, the present invention provides a compound of formula (IX) wherein X is O, Y is O, R⁴ is H, R⁵ is OR¹⁶, R² is alkyl and R³ is arylalkyl.

For example, the present invention provides a compound of formula (IX) wherein X is O, Y is O, R⁴ is H, R⁵ is OR¹⁶, R² is alkyl and R³ is arylalkyl substituted with R^{3a}.

For example, the present invention provides a compound of formula (IX) wherein X is O, Y is O, R⁴ is H, R⁵ is OR¹⁶, R² is alkyl, R³ is arylalkyl substituted with R^{3a}, and R^{3a} is aryl or heteroaryl.

For example, the present invention provides a compound of formula (IX) wherein X is O, Y is O, R⁴ is H, R⁵ is OR¹⁶, R² is alkyl, R³ is arylalkyl substituted with R^{3a}, R¹ is alkyl, and R^{3a} is aryl or heteroaryl.

For example, the present invention provides a compound of formula (IX) wherein X is O, Y is O, R⁴ is H, R⁵ is OR¹⁶, R² is alkyl, R³ is arylalkyl substituted with R^{3a}, R¹ is alkyl, R⁶ is arylalkyl, R^{3a} is aryl or heteroaryl.

For example, the present invention provides a compound of formula (IX) wherein X is O, Y is O, R⁴ is H, R⁵ is OR¹⁶, R² is alkyl, R³ is alkyl substituted with R^{3a}, R¹ is alkyl, R⁶ is arylalkyl, R⁷ is alkyl, and R^{3a} is aryl or heteroaryl.

In a tenth embodiment, the present invention provides a compound of formula (X) 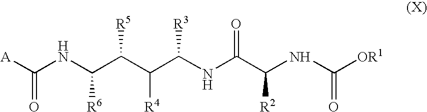
or a pharmaceutically acceptable salt form, stereoisomer, ester, salt of an ester, prodrug, salt of a prodrug, or combination thereof, wherein:
A is 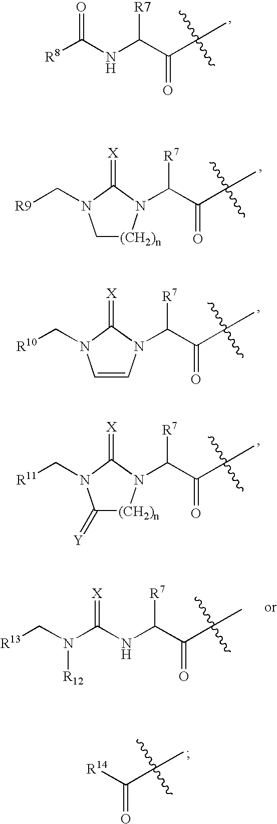
X is O, S or NH;
Y is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁸ is —ORₐ or -alkylORₐ;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R⁶)C(O)NRₐR_{b}, -alkylN(R_{b})SO₂Rₐ, -alkylC(O)ORₐ, -alkylC(O)Rₐ, -alkylC(O)NRₐR_{b} and R₁₀ₐ;
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁴ is —ORₐ or -alkylORₐ;
R¹⁶ is hydrogen or R¹⁵;
R¹⁵ is 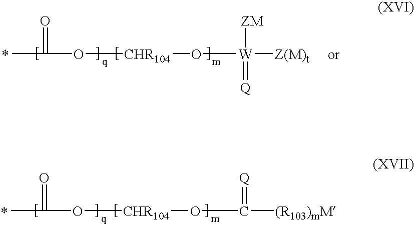
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆-SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl are heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

For example, the present invention provides a compound of formula (X) wherein X is O and Y is O.

For example, the present invention provides a compound of formula (X) wherein X is O, Y is O, R⁴ is H, R⁵ is OR¹⁶ and R² is alkyl.

For example, the present invention provides a compound of formula (X) wherein X is O, Y is O, R⁴ is H, R⁵ is OR¹⁶, R² is alkyl and R³ is arylalkyl.

For example, the present invention provides a compound of formula (X) wherein X is O, Y is O, R⁴ is H, R⁵ is OR¹⁶, R² is alkyl and R³ is arylalkyl substituted with R^{3a}.

For example, the present invention provides a compound of formula (X) wherein X is O, Y is O, R⁴ is H, R⁵ is O, R² is alkyl, R³ is arylalkyl substituted with R^{3a}, and R^{3a} is aryl or heteroaryl.

For example, the present invention provides a compound of formula (X) wherein X is O, Y is O, R⁴ is H, R¹ is OR¹⁶, R² is alkyl, R³ is arylalkyl substituted with R^{3a}, R¹ is alkyl, and R^{3a} is aryl or heteroaryl.

For example, the present invention provides a compound of formula (X) wherein X is O, Y is O, R⁴ is H, R⁵ is OR¹⁶, R² is alkyl, R³ is arylalkyl substituted with R^{3a}, R¹ is alkyl, R⁶ is arylalkyl, and R^{3a} is aryl or heteroaryl.

For example, the present invention provides a compound of formula (X) wherein X is O, Y is O, R⁴ is H, R⁵ is OR¹⁶, R² is alkyl, R³ is arylalkyl substituted with R^{3a}, R¹ is alkyl, R⁶ is arylalkyl, R⁷ is alkyl, and R^{3a} is aryl or heteroaryl.

In an eleventh embodiment, the present invention provides a compound of formula (XI) 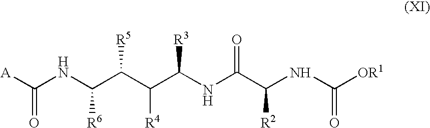
or a pharmaceutically acceptable salt form, stereoisomer, ester, salt of an ester, prodrug, salt of a prodrug, or combination thereof, wherein:
A is 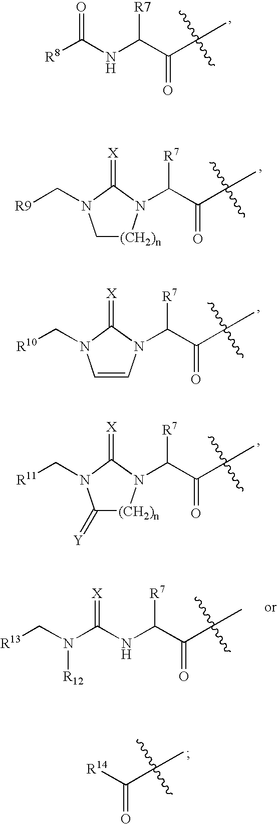
X is O, S or NH;
Y is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl; heterocycle, aryl or heteroaryl; wherein each R^{1a} is substituted with 0, 1, 2, 3 or 4 substituents selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl);
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —N_{b}C(O)Rₐ —N(R_{b})C(O)Ok, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁸ is —ORₐ or -alkylORₐ;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁴ is —ORₐ or -alkylORₐ;
R¹⁶ is hydrogen or R¹⁵;
R¹⁵ is 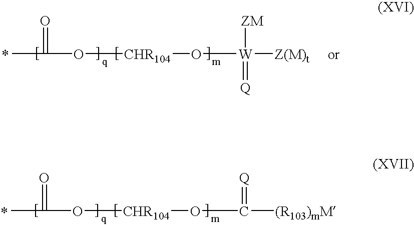
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₄₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆-SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alky, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each t and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

For example, the present invention provides a compound of formula (XI) wherein X is O and Y is O.

For example, the present invention provides a compound of formula (XI) wherein X is O, Y is O, R⁴ is H, R⁵ is OR¹⁶ and R² is alkyl.

For example, the present invention provides a compound of formula (XI) wherein X is O, Y is O, R⁴ is H, R¹ is OR¹⁶, R² is alkyl and R³ is arylalkyl.

For example, the present invention provides a compound of formula (XI) wherein X is O, Y is O, R⁴ is H, R⁵ is OR¹⁶, R² is alkyl and R³ is arylalkyl substituted with R^{3a}.

For example, the present invention provides a compound of formula (XI) wherein X is O, Y is O, R⁴ is H, R⁵ is OR¹⁶, R² is alkyl, R³ is arylalkyl substituted with R^{3a} and R^{3a} is aryl or heteroaryl.

For example, the present invention provides a compound of formula (XI) wherein X is O, Y is O, R⁴ is H, R⁵ is OR¹⁶, R² is alkyl, R³ is arylalkyl substituted with R^{3a}, R¹ is alkyl and R^{3a} is aryl or heteroaryl.

For example, the present invention provides a compound of formula (XI) wherein X is O, Y is O, R⁴ is H, R⁵ is OR¹⁶, R² is alkyl, R³ is arylalkyl substituted with R^{3a}, R¹ is alkyl, R⁶ is arylalkyl, and R^{3a} is aryl or heteroaryl.

For example, the present invention provides a compound of formula (XI) wherein X is O, Y is O, R⁴ is H, R⁵ is OR¹⁶, R² is alkyl, R³ is arylalkyl substituted with R^{3a}, R¹ is alkyl, R⁶ is arylalkyl, R⁷ is alkyl, and R^{3a} is aryl or heteroaryl.

In a twelfth embodiment the present invention provides a compound of formula (XII) 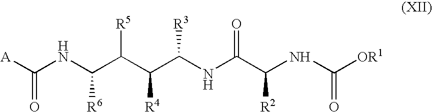
or a pharmaceutically acceptable salt form, stereoisomer, ester, salt of an ester, prodrug, salt of a prodrug, or combination thereof, wherein:
A is 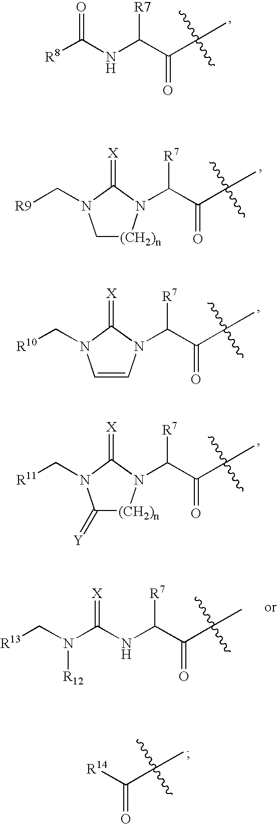
X is O, S or NH;
Y is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ, and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl; -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁵ is H and R⁴ is OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁸ is —ORₐ or -alkylORₐ;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl; cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁴ is —ORₐ or -alkylORₐ;
R¹⁶ is hydrogen or R¹⁵;
R¹⁵ is 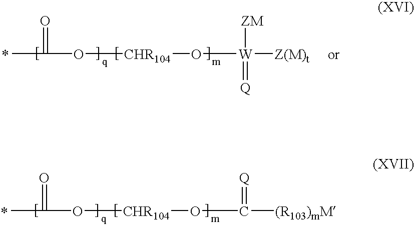
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆-SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₄₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

For example, the present invention provides a compound of formula (XII) wherein X is O and Y is O.

For example, the present invention provides a compound of formula (XII) wherein X is O, Y is O, R⁴ is OR¹⁶, R⁵ is H and R² is alkyl.

For example, the present invention provides a compound of formula (XII) wherein X is O, Y is O, R⁴ is OR¹⁶, R⁵ is H, R² is alkyl and R³ is arylalkyl.

For example, the present invention provides a compound of formula (XII) wherein X is O, Y is O, R⁴ is OR¹⁶, R⁵ is H, R² is alkyl and R³ is arylalkyl substituted with R³.

For example, the present invention provides a compound of formula (XII) wherein X is O, Y is O, R⁴ is OR¹⁶, R⁵ is H, R² is alkyl, R³ is arylalkyl substituted with R^{3a}, and R^{3a} is aryl or heteroaryl.

For example, the present invention provides a compound of formula (XII) wherein X is O, Y is O, R⁴ is OR¹⁶, R⁵ is H, R² is alkyl, R³ is arylalkyl substituted with R^{3a}, R¹ is alkyl, and R^{3a} is aryl or heteroaryl.

For example, the present invention provides a compound of formula (XII) wherein X is O, Y is O, R⁴ is OR¹⁶, R⁵ is H, R² is alkyl, R³ is arylalkyl substituted with R^{3a}, R¹ is alkyl, R⁶ is arylalkyl, and R^{3a} is aryl or heteroaryl.

For example, the present invention provides a compound of formula (XII) wherein X is O, Y is O, R⁴ is OR¹⁶, R⁵ is H, R² is alkyl, R³ is arylalkyl substituted with R^{3a}, R¹ is alkyl, R⁶ is arylalkyl, R⁷ is alkyl, and R^{3a} is aryl or heteroaryl.

In a thirteenth embodiment the present invention provides a compound of formula (XIII) 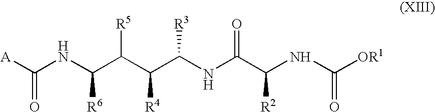
or a pharmaceutically acceptable salt form, stereoisomer, ester, salt of an ester, prodrug, salt of a prodrug, or combination thereof, wherein:
A is 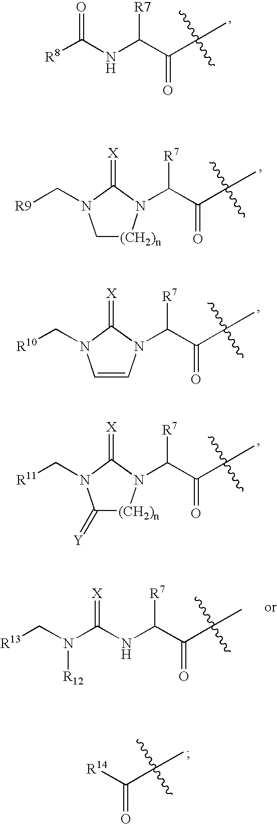
X is O, S or NH;
Y is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents indepentdently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁵ is H and R⁴ is OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁸ is —ORₐ or -alkylORₐ;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁴ is —ORₐ or -alkylORₐ;
R¹⁶ is hydrogen or R¹⁵;
R¹⁵ is 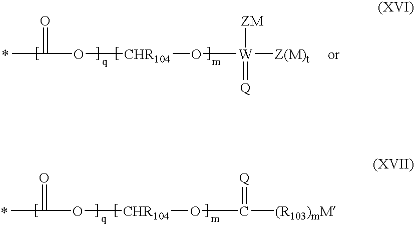
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

For example, the present invention provides a compound of formula (XIII) wherein X is O and Y is O.

For example, the present invention provides a compound of formula (XIII) wherein X is O, Y is O, R⁴ is OR¹⁶, R⁵ is H, and R² is alkyl.

For example, the present invention provides a compound of formula (XIII) wherein X is O, Y is O, R⁴ is OR¹⁶, R⁵ is H, R² is alkyl and R³ is arylalkyl.

For example, the present invention provides a compound of formula (XIII) wherein X is O, Y is O, R⁴ is OR¹⁶, R⁵ is H, R² is alkyl and R³ is arylalkyl substituted with R^{3a}.

For example, the present invention provides a compound of formula (XIII) wherein X is O, Y is O, R⁴ is OR¹⁶, R⁵ is H, R² is alkyl, R³ is arylalkyl substituted with R^{3a}, and R^{3a} is aryl or heteroaryl.

For example, the present invention provides a compound of formula (XIII) wherein X is O, Y is O, R⁴ is OR¹⁶, R⁵ is H, R² is alkyl, R³ is arylalkyl substituted with R^{3a}, R¹ is alkyl, and R^{3a} is aryl or heteroaryl.

For example, the present invention provides a compound of formula (XIII) wherein X is O, Y is O, R⁴ is OR¹⁶, R⁵ is H, R² is alkyl, R³ is arylalkyl substituted with R^{3a}, R¹ is alkyl, R⁶ is arylalkyl, and R^{3a} is aryl or heteroaryl.

For example, the present invention provides a compound of formula (XIII) wherein X is O, Y is O, R⁴ is OR¹⁶, R⁵ is H, R² is alkyl, R³ is arylalkyl substituted with R^{3a}, R¹ is alkyl, R⁶ is arylalkyl, R⁷ is alkyl, and R^{3a} is aryl or heteroaryl.

In a fourteenth embodiment the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, salt of an ester, prodrug, salt of a prodrug, or combination thereof, and a pharmaceutically acceptable carrier.

In a fifteenth embodiment the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt, stereoisomer, ester, salt of an ester, prodrug, salt of a prodrug, or combination thereof, and a pharmaceutically acceptable carrier.

In a sixteenth embodiment the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt, stereoisomer, ester, salt of an ester, prodrug, salt of a prodrug, or combination thereof, and a pharmaceutically acceptable carrier.

In a seventeenth embodiment the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, streoisomer, ester, salt of an ester, prodrug, salt of a prodrug, or combination thereof, one, two, three, four, five or six second HIV protease inhibitors, and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt, stereoisomer, ester, salt of an ester, prodrug, salt of a prodrug, or combination thereof, one, two, three, four, five or six second HIV protease inhibitors and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt, stereoisomer, ester, salt of an ester, prodrug, salt of a prodrug, or combination thereof, one, two, three, four, five or six second HIV protease inhibitors and a pharmaceutically acceptable carrier.

For example, the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, salt of an ester, prodrug, salt of a prodrug, or combination thereof, one, two, three, four, five or six second HIV protease inhibitors selected from the group consisting of ritonavir, lopinavir, saquinavir, amprenavir, fosamprenavir, nelfinavir, tipranavir, indinavir, atazanavir, TMC-126, TMC-114, mozenavir (DMP-450), JE-2147 (AG1776), L-756423, RO0334649, KNI-272, DPC-681, DPC-684 and GW640385X, and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt, stereoisomer, ester, salt of an ester, prodrug, salt of a prodrug, or combination thereof, one, two, three, four, five or six second HIV protease inhibitors selected from the group consisting of ritonavir, lopinavir, saquinavir, amprenavir, fosamprenavir, nelfinavir, tipranavir, indinavir, atazanavir, TMC-126, TMC-114, mozenavir (DMP-450), JE-2147 (AG1776), L-756423, RO0334649, KNI-272, DPC-681, DPC-684 and GW640385X, and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo 1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six second HIV protease inhibitors selected from the group consisting of ritonavir, lopinavir, saquinavir, amprenavir, fosamprenavir, nelfinavir, tipranavir, indinavir, atazanavir, TMC-126, TMC-114, mozenavir (DMP-450), JE-2147 (AG1776), L-756423, RO0334649, KNI-272, DPC-681, DPC-684 and GW640385X, and a pharmaceutically acceptable carrier.

In an eighteenth embodiment the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, salt of an ester, prodrug, salt of a prodrug, or combination thereof, one, two, three, four, five or six HIV reverse transcriptase inhibitors, and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methyl ethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV reverse transcriptase inhibitors and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV reverese transcriptase inhibitors and a pharmaceutically acceptable carrier.

For example, the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, salt of an ester, prodrug, salt of a prodrug, or combination thereof, one, two, three, four, five or six HIV reverse transcriptase inhibitors selected from the group consisting of lamivudine, stavudine, zidovudine, abacavir, zalcitabine, didanosine, tenofovir, emtricitabine, amdoxovir, elvucitabine, alovudine, MIV-210, Racivir (±-FTC), D-D4FC (Reverset, DPC-817), SPD754, nevirapine, delavirdine, efavirenz, capravirine, emivirine, calanolide A, GW5634, BMS-56190 (DPC-083), DPC-961, MIV-150, TMC-120 and TMC-125, and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV reverse transcriptase inhibitors selected from the group consisting of lamivudine, stavudine, zidovudine, abacavir, zalcitabine, didanosine, tenofovir, emtricitabine, amdoxovir, elvucitabine, alovudine, MIV-210, Racivir (±-FTC), D-D4FC (Reverset, DPC-817), SPD754, nevirapine, delavirdine, efavirenz, capravirine, emivirine, calanolide A, GW5634, BMS-56190 (DPC-083), DPC-961, MIV-150, TMC-120 and TMC-125, and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV reverse transcriptase inhibitors selected from the group consisting of lamivudine, stavudine, zidovudine, abacavir, zalcitabine, didanosine, tenofovir, emtricitabine, amdoxovir, elvucitabine, alovudine, MIV-210, Racivir (±-FTC), D-D4FC (Reverset, DPC-817), SPD754, nevirapine, delavirdine, efavirenz, capravirine, emivirine, calanolide A, GW5634, BMS-56190 (DPC-083), DPC-961, MIV-150, TMC-120 and TMC-125, and a pharmaceutically acceptable carrier.

In a nineteenth embodiment the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, salt of an ester, prodrug, salt of a prodrug, or combination thereof, one, two, three, four, five or six HIV entry/fusion inhibitors and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV entry/fusion inhibitors and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV entry/fusion inhibitors and a pharmaceutically acceptable carrier.

For example, the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, salt of an ester, prodrug, salt of a prodrug, or combination thereof, one, two, three, four, five or six HIV entry/fusion inhibitors selected from the group consisting of enfuvirtide (T-20), T-1249, PRO 2000, PRO 542, PRO 140, AMD-3100, BMS-806, FP21399, GW873140, Schering C (SCH-C), Schering D (SCH-D), TNX-355 and UK-427857, and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV entry/fusion inhibitors selected from the group consisting of enfuvirtide (T-20), T-1249, PRO 2000, PRO 542, PRO 140, AMD-3100, BMS-806, FP21399, GW873140, Schering C (SCH-C), Schering D (SCH-D), TNX-355 and UK-427857, and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV entry/fusion inhibitors selected from the group consisting of enfuvirtide (T-20), T-1249, PRO 2000, PRO 542, PRO 140, AMD-3100, BMS-806, FP21399, GW873140, Schering C (SCH-C), Schering D (SCH-D), TNX-355 and UK-427857, and a pharmaceutically acceptable carrier.

In a twentieth embodiment the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, salt of an ester, prodrug, salt of a prodrug, or combination thereof, one, two, three, four, five or six HIV integrase inhibitors and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV integrase inhibitors and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV integrase inhibitors and a pharmaceutically acceptable carrier.

For example, the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, prodrug, or combination thereof, one, two, three or four HIV integrase inhibitors selected from the group consisting of S-1360, zintevir (AR-177), L-870812 and L-870810, and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three or four HIV integrase inhibitors selected from the group consisting of S-1360, zintevir (AR-177), L-870812 and L-870810, and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate; or a pharmaceutically acceptable salt form, stereoisomer, ester, salt of an ester, prodrug, salt of a prodrug, or combination thereof, one, two, three or four HIV integrase inhibitors selected from the group consisting of S-1360, zintevir (AR-177), L-870812 and L-870810, and a pharmaceutically acceptable carrier.

In a twenty-first embodiment the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, salt of an ester, prodrug, salt of a prodrug, or combination thereof, one, two, three, four, five or six HIV budding/maturation inhibitors and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV budding/maturation inhibitors and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two, three, four, five or six HIV budding/maturation inhibitors and a pharmaceutically acceptable carrier.

For example, the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, salt of an ester, prodrug, salt of a prodrug, or combination thereof, PA-457, and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, PA-457, and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, PA-457, and a pharmaceutically acceptable carrier.

In a twenty-second embodiment the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, prodrug, or combination thereof, one, two or three second HIV protease inhibitor, one, two or three HIV reverese transcriptase inhibitor and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two or three second HIV protease inhibitor, one, two or three HIV reverse transcriptase inhibitor and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two or three second HIV protease inhibitor, one, two or three HIV reverse transcriptase inhibitor and a pharmaceutically acceptable carrier.

For example, the present invention provides a pharmaceutical composition comprising a therapeutically effective amount of a compound, or combination of compounds of formulae (I), (II), (III), (IV), (V), (VI), (VII), (VIII), (IX), (X), (XI), (XII) or (XIII), or a pharmaceutically acceptable salt form, stereoisomer, ester, prodrug, or combination thereof, one, two or three second HIV protease inhibitors selected from the group consisting of ritonavir, lopinavir, saquinavir, amprenavir, fosamprenavir, nelfinavir, tipranavir, indinavir, atazanavir, TMC-126, TMC-114, mozenavir (DMP-450), JE-2147 (AG1776), L-756423, RO0334649, KNI-272, DPC-681, DPC-684 and GW640385X, one, two or three HIV reverse transcriptase inhibitors selected from the group consisting of lamivudine, stavudine, zidovudine, abacavir, zalcitabine, didanosine, tenofovir, emtricitabine, amdoxovir, elvucitabine, alovudine, MIV-210, Racivir (±-FTC), D-D4FC (Reverset, DPC-817), SPD754, nevirapine, delavirdine, efavirenz, capravirine, emivirine, calanolide A, GW5634, BMS-56190 (DPC-083), DPC-961, MIV-150, TMC-120 and TMC-125, and a pharmaceutical acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two or three second HIV protease inhibitors selected from the group consisting of ritonavir, lopinavir, saquinavir, amprenavir, fosamprenavir, nelfinavir, tipranavir, indinavir, atazanavir, TMC-126, TMC-114, mozenavir (DMP-450), JE-2147 (AG1776), L-756423, RO0334649, KNI-272, DPC-681, DPC-684 and GW640385X, one, two or three HIV reverse transcriptase inhibitors selected from the group consisting of lamivudine, stavudine, zidovudine, abacavir, zalcitabine, didanosine, tenofovir, emtricitabine, amdoxovir, elvucitabine, alovudine, MIV-210, Racivir (±-FTC), D-D4FC (Reverset, DPC-817), SPD754, nevirapine, delavirdine, efavirenz, capravirine, emivirine, calanolide A, GW5634, BMS-56190 (DPC-083), DPC-961, MIV-150, TMC-120 and TMC-125, and a pharmaceutical acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two or three second HIV protease inhibitors selected from the group consisting of ritonavir, lopinavir, saquinavir, amprenavir, fosamprenavir, nelfinavir, tipranavir, indinavir, atazanavir, TMC-126, TMC-114, mozenavir (DMP-450), JE-2147 (AG1776), L-756423, RO0334649, KNI-272, DPC-681, DPC-684 and GW640385X, one, two or three HIV reverse transcriptase inhibitors selected from the group consisting of lamivudine, stavudine, zidovudine, abacavir, zalcitabine, didanosine, tenofovir, emtricitabine, amdoxovir, elvucitabine, alovudine, MIV-210, Racivir (i-FTC), D-D4FC (Reverset, DPC-817), SPD754, nevirapine, delavirdine, efavirenz, capravirine, emivirine, calanolide A, GW5634, BMS-56190 (DPC-083), DPC-961, MIV-150, TMC-120 and TMC-125, and a pharmaceutical acceptable carrier.

In a twenty-third embodiment the present invention provides a pharmaceutical composition comprising a therapeutically effective amount of a compound, or combination of compounds of formula (I), (II), (III), (IV), (V), (VI), (VII), (VIII), (IX), (X), (XI), (XII) or (XIII), or a pharmaceutically acceptable salt form, stereoisomer, ester, salt of an ester, prodrug, salt of a prodrug, or combination thereof, one, two or three second HIV protease inhibitor, one, two or three HIV entry/fusion inhibitor and a pharmaceutically acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two or three second HIV protease inhibitor, one, two or three HIV entry/fusion inhibitor and a pharmaceutically acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two or three second HIV protease inhibitor, one, two or three HIV entry/fusion inhibitor and a pharmaceutically acceptable carrier.

For example, the present invention provides a pharmaceutical composition comprising a therapeutically effective amount of a compound, or combination of compounds of formulae (I), (II), (III), (IV), (V), (VI), (VII), (VIII), (IX), (X), (XI), (XII) or (XIII), or a pharmaceutically acceptable salt form, stereoisomer, ester, salt of an ester, prodrug, salt of a prodrug, or combination thereof, one, two or three, second HIV protease inhibitors selected from the group consisting of ritonavir, lopinavir, saquinavir, amprenavir, fosamprenavir, nelfinavir, tipranavir, indinavir, atazanavir, TMC-126, TMC-114, mozenavir (DMP-450), JE-2147 (AG1776), L-756423, RO0334649, KNI-272, DPC-681, DPC-684 and GW640385X, one, two or three HIV entry/fusion inhibitors selected from the group consisting of enfuvirtide (T-20), T-1249, PRO 2000, PRO 542, PRO 140, AMD-3100, BMS-806, FP21399, GW873140, Schering C (SCH-C), Schering D (SCH-D), TNX-355 and UK-427857, and a pharmaceutical acceptable carrier.

For example, the present invention provides a pharmaceutical composition comprising a therapeutically effective amount of methyl(1S)-1-[({(1R,3S,4S)-3-hydroxy-4-{[(2S)-2-(3-{[6-(1-hydroxy-1-methyl ethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof, one, two or three second HIV protease inhibitors selected from the group consisting of ritonavir, lopinavir, saquinavir, amprenavir, fosamprenavir, nelfinavir, tipranavir, indinavir, atazanavir, TMC-126, TMC-114, mozenavir (DMP-450), JE-2147 (AG 1776), L-756423, RO0334649, KNI-272, DPC-681, DPC-684 and GW640385X, one, two or three HIV entry/fusion inhibitors selected from the group consisting of enfuvirtide (T-20), T-1249, PRO 2000, PRO 542, PRO 140, AMD-3100, BMS 806, FP21399, GW873140, Schering C (SCH-C), Schering D (SCH-D), TNX-355 and UK-427857, and a pharmaceutical acceptable carrier.

For example, the present invention provides a pharmaceutical composition comprising a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereo isomer, ester, salt of an ester, prodrug, salt of a prodrug, or combination thereof, one, two or three second HIV protease inhibitors selected from the group consisting of ritonavir, lopinavir, saquinavir, amprenavir, fosamprenavir, nelfinavir, tipranavir, indinavir, atazanavir, TMC-126, TMC-114, mozenavir (DMP-450), JE-2147 (AG1776), L-756423, RO0334649, KNI-272, DPC-681, DPC-684 and GW640385X, one, two or three HIV entry/fusion inhibitors selected from the group consisting of enfuvirtide (T-20), T-1249, PRO 2000, PRO 542, PRO 140, AMD-3100, BMS-806, FP21399, GW873140, Schering C (SCH-C), Schering D (SCH-D), TNX-355 and UK-427857, and a pharmaceutical acceptable carrier.

In a twenty-fourth embodiment the present invention provides a method of inhibiting the replication of an HIV virus comprising contacting said virus with a therapeutically effective amount of a compound or combination of compounds of formula (I), (II), (III), (IV), (V), (VI), (VII), (VIII), (IX), (X), (XI), (XII) or (XIII), or a pharmaceutically acceptable salt form, streoisomer, ester, prodrug, or combination thereof.

For example, the present invention provides a method of inhibiting the replication of an HIV virus comprising contacting said virus with a therapeutically effective amount of methyl (1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof.

For example, the present invention provides a method of inhibiting the replication of an HIV virus comprising contacting said virus with a therapeutically effective amount of methyl (1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof.

In a twenty-fifth embodiment the present invention provides a method of inhibiting HIV protease comprising contacting said HIV protease with a therapeutically effective amount of a compound or combination of compounds of formula (I), (II), (III), (IV), (V), (VI), (VII), (VIII), (IX), (X), (XI), (XII) or (XIII), or a pharmaceutically acceptable salt form, streoisomer, ester, prodrug, or combination thereof.

For example, the present invention provides a method of inhibiting HIV protease comprising contacting said HIV protease with a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof.

For example, the present invention provides a method of inhibiting HIV protease comprising contacting said HIV protease with a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof.

In a twenty-sixth embodiment the present invention provides a method of treating or preventing an HIV infection comprising administering to a patient in need of such treatment a therapeutically effective amount of a compound or combination of compounds of formula (I), (II), (III), (IV), (V), (VI), (VII), (VIII), (IX), (X), (XI), (XII) or (XIII), or a pharmaceutically acceptable salt form, streoisomer, ester, salt of an ester, prodrug, or combination thereof.

For example, the present invention provides a method of treating or preventing an HIV infection comprising administering to a patient in need of such treatment a therapeutically effective amount of methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof.

For example, the present invention provides a method of treating or preventing an HIV infection comprising administering to a patient in need of such treatment a therapeutically effective amount of methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof.

In a twenty-seventh embodiment the present invention provides a method of inhibiting the replication of an HIV virus comprising contacting said virus with any one of the pharmaceutical compositions disclosed hereinabove.

In a twenty-eighth embodiment the present invention provides a method of inhibiting HIV protease comprising contacting said HIV protease with any one of the pharmaceutical compositions disclosed hereinabove.

In a twenty-ninth embodiment the present invention provides a method of treating or preventing an HIV infection comprising administering to a patient in need of such treatment any one of the pharmaceutical compositions disclosed hereinabove.

In a thirtieth embodiment the present invention provides an HIV protease inhibiting compound comprising a substituent of the formula (XIV): 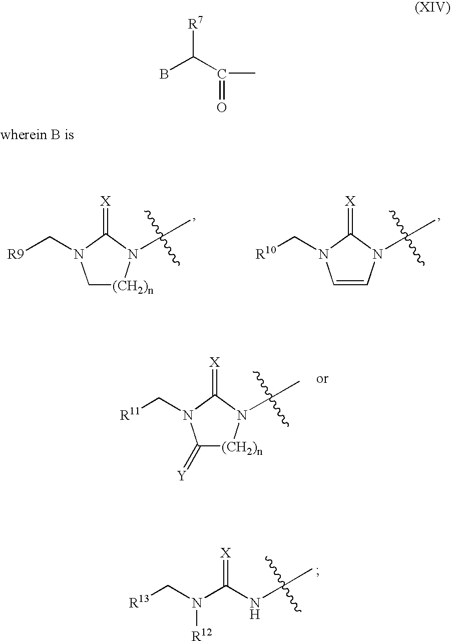
X is O, S or NH;
Y is O, S or NH;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

For example, the present invention provides a compound comprising a substituent of formula (XIV) wherein X is O and Y is O.

For example, the present invention provides a compound comprising a substituent of formula (XIV) wherein X is O, Y is O, and R⁷ is alkyl.

For example, the present invention provides a compound comprising a substituent of formula (XIV) wherein X is O, Y is O, R⁷ is alkyl and R¹² is alkyl.

For example, the present invention provides a compound comprising a substituent of formula (XIV) wherein X is O, Y is O, R⁷ is alkyl, R¹² is alkyl, and R⁹, R¹⁰, R¹¹ and R¹³ are independently selected from the group consisting of aryl and heteroaryl.

For example, the present invention provides a compound comprising a substituent of formula (XIV) wherein X is O, Y is O, R⁷ is alkyl, R¹² is alkyl and R⁹, R¹⁰, R¹¹ and R¹³ are independently selected from the group consisting of thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl and indazolyl.

For example, the present invention provides a compound comprising a substituent of formula (XIV) wherein X is O, Y is O, R⁷ is C1, C2, C3, C4 or C5 alkyl, R¹² is alkyl, and R⁹, R¹⁰, R¹¹ and R¹³ are independently selected from the group consisting of thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl and indazolyl.

For example, the present invention provides a compound comprising a substituent of formula (XIV) wherein X is O, Y is O, R⁷ is tert-butyl, 1-methylpropyl or isopropyl, R¹² is alkyl, and R⁹, R¹⁰, R¹¹ and R¹³ are independently selected from the group consisting of thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl and indazolyl.

For example, the present invention provides a compound comprising a substituent of formula (XIV) wherein X is O, Y is O, R⁷ is tert-butyl, 1-methylpropyl or isopropyl, R¹² is methyl or ethyl, and R⁹, R¹⁰, R¹¹ and R¹³ are independently selected from the group consisting of thienyl, furanyl, pyrrolyl, thiazolyl, oxazolyl, imidazolyl, pyrazolyl, isothiazolyl, isoxazolyl, isoquinolinyl, quinolinyl, pyridyl, phenyl, pyridoimidazolyl, benzimiazolyl, benzothienyl, benzthiazolyl and indazolyl.

HIV protease inhibiting compounds comprising a substituent of the formula (XIV) can be prepared by coupling a suitable intermediate or precursor molecule having an amino group (—NH₂ or —NHR* wherein R* is alkyl), a hydroxy group (—OH) or a thiol group (—SH) to the compound of formula (XV) or a salt or an activated ester derivative thereof: 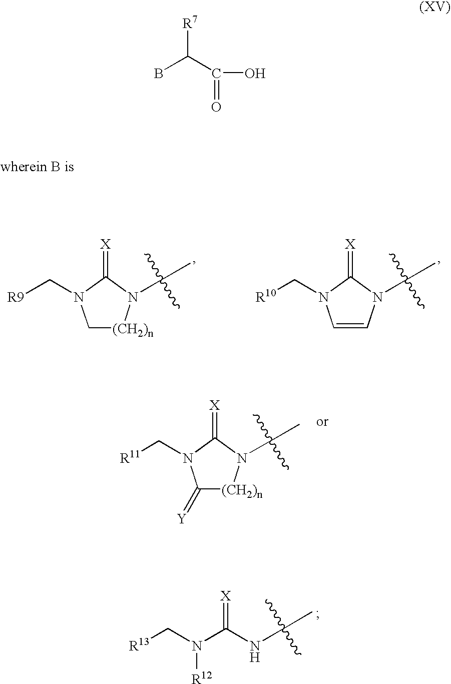
X is O, S or NH;
Y is O, S or NH;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

The term “N-protecting group” or “N-protected” as used herein refers to those groups intended to protect the N-terminus of an amino acid or peptide or to protect an amino group against undesirable reactions during synthetic procedures. Commonly used N-protecting groups are disclosed in T. H. Greene and P. G. M. Wuts, Protective Groups in Organic Synthesis, 2nd edition, John Wiley & Sons, New York (1991). N-protecting groups comprise acyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, benzoyl, 4-chlorobenzoyl, 4-bromobenzoyl, 4-nitrobenzoyl, and the like; sulfonyl groups such as benzenesulfonyl, p-toluenesulfonyl and the like; sulfenyl groups such as phenylsulfenyl (pheny-S—), triphenylmethylsulfenyl (trityl-S—) and the like; sulfinyl groups such as p-methylphenylsulfinyl (p-methylphenyl-S(O)—), t-butylsulfinyl (t-Bu-S(O)—) and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2-trichloro-ethoxy-carbonyl, phenoxycarbonyl, 4-nitro-phenoxycarbonyl, fluorenyl-9-methoxycarbonyl, cyclopentyloxycarbonyl, adamantyloxycarbonyl, cyclohexyloxycarbonyl, phenylthiocarbonyl and the like; alkyl groups such as benzyl, p-methoxybenzyl, triphenylmethyl, benzyloxymethyl and the like; p-methoxyphenyl and the like; and silyl groups such as trimethylsilyl and the like. Preferred N-protecting groups include formyl, acetyl, benzoyl, pivaloyl, t-butylacetyl, phenylsulfonyl, benzyl, t-butyloxycarbonyl (Boc) and benzyloxycarbonyl (Cbz).

As used herein, the terms “S” and “R” configuration are as defined by the IUPAC 1974 Recommendations for Section E, Fundamental Stereochemistry, Pure Appl. Chem. (1976) 45, 13-30.

The compounds of the invention can comprise asymmetrically substituted carbon atoms. As a result, all stereoisomers of the compounds of the invention are meant to be included in the invention, including racemic mixtures, mixtures of diastereomers, as well as individual optical isomers, including, enantiomers and single diastereomers of the compounds of the invention substantially free from their enantiomers or other diastereomers. By “substantially free” is meant greater than about 80% free of other enantiomers or diastereomers of the compound, more preferably greater than about 90% free of other enantiomers or diastereomers of the compound, even more preferably greater than about 95% free of other enantiomers or diastereomers of the compound, even more highly preferably greater than about 98% free of other enantiomers or diastereomers of the compound and most preferably greater than about 99% free of other enantiomers or diastereomers of the compound.

In addition, compounds comprising the possible geometric isomers of carbon-carbon double bonds and carbon-nitrogen double are also meant to be included in this invention.

Individual stereoisomers of the compounds of this invention can be prepared by any one of a number of methods which are within the knowledge of one of ordinary skill in the art. These methods include stereospecific synthesis, chromatographic separation of diastereomers, chromatographic resolution of enantiomers, conversion of enantiomers in an enantiomeric mixture to diastereomers and then chromatographically separating the diastereomers and regeneration of the individual enantiomers, enzymatic resolution and the like.

Stereospecific synthesis involves the use of appropriate chiral starting materials and synthetic reactions which do not cause racemization or inversion of stereochemistry at the chiral centers.

Diastereomeric mixtures of compounds resulting from a synthetic reaction can often be separated by chromatographic techniques which are well-known to those of ordinary skill in the art.

Chromatographic resolution of enantiomers can be accomplished on chiral chromatography resins. Chromatography columns containing chiral resins are commercially available. In practice, the racemate is placed in solution and loaded onto the column containing the chiral stationary phase. The enantiomers are then separated by HPLC.

Resolution of enantiomers can also be accomplished by converting the enantiomers in the mixture to diastereomers by reaction with chiral auxiliaries. The resulting diastereomers can then be separated by column chromatography. This technique is especially useful when the compounds to be separated contain a carboxyl, amino or hydroxyl group that will form a salt or covalent bond with the chiral auxiliary. Chirally pure amino acids, organic carboxylic acids or organosulfonic acids are especially useful as chiral auxiliaries. Once the diastereomers have been separated by chromatography, the individual enantiomers can be regenerated. Frequently, the chiral auxiliary can be recovered and used again.

Enzymes, such as esterases, phosphatases and lipases, can be useful for resolution of derivatives of the enantiomers in an enantiomeric mixture. For example, an ester derivative of a carboxyl group in the compounds to be separated can be prepared. Certain enzymes will selectively hydrolyze only one of the enantiomers in the mixture. Then the resulting enantiomerically pure acid can be separated from the unhydrolyzed ester.

In addition, solvates and hydrates of the compounds of Formula (I), (II), (III), (IV), (V), (VI), (VII), (VIII), (IX), (X), (XI), (XII) or (XIII) are meant to be included in this invention.

When any variable (for example A, B, R¹, R², R³, R⁴, R⁵, R⁶, R⁷, R⁸, R⁹, R¹⁰, R¹¹, R¹², R¹³, R¹⁴, R¹⁵, Rₐ, R_{b}, R_{c}, n, etc.) occurs more than one time in any substituent or in the compound of formula (I), (II), (III), (IV), (V), (VI), (VII), (VIII), (IX), (X), (XI), (XII), (XIII), (XIV), (XV), (XVI), (XVII) or any other formula herein, its definition on each occurrence is independent of its definition at every other occurrence. In addition, combinations of substituents are permissible only if such combinations result in stable compounds. Stable compounds are compounds which can be isolated in a useful degree of purity from a reaction mixture.

The compounds of the present invention can be used in the form of salts derived from inorganic or organic acids. These salts include but are not limited to the following: 4-acetamido-benzoate, acetate, adipate, alginate, carbonate, 4-chlorobenzenesulfonate, citrate, aspartate, benzoate, benzenesulfonate, bisulfate, butyrate, camphorate, camphorsulfonate, cholate, digluconate, cyclopentanepropionate, dichloroacetate, dodecylsulfate, ethanedisulfonate, ethanesulfonate, ethylsuccinate, formate, fumarate, galactarate, D-gluconate, D-glucuronate, glucoheptanoate, glutarate, lycerophosphate, glycolate, hemisulfate, heptanoate, hexanoate, hydrochloride, hydrobromide, hydroiodide, 2-hydroxyethanesulfonate (isethionate), 3-hydroxy-2-naphthoate, 1-hydroxy-2-naphthoate, lactate, lactobionate, laurate, maleate, malonate, mandelate, methanesulfonate, nicotinate, 1,5-naphthalene-disulfonate, 2-naphthalenesulfonate, oleate, oxalate, pamoate, palmitate, pectinate, persulfate, 3-phenylpropionate, picrate, pivalate, propionate, L-pyroglutamate, sebacate, stearate, succinate, tartrate, terephthalate, thiocyanate, p-toluenesulfonate, undecanoate, undecylenoate and valerate. Also, the basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. Other salts include salts with alkali metals or alkaline earth metals, such as aluminum, sodium, lithium, potassium, calcium, magnesium or zinc or with organic bases such as diethylethanolamine, diethanolamine, ethylenediamine, guanidine, meglumine, olamine (ethnolamine), piperazine, piperidine, triethylamine, tromethamine, benzathine, benzene-ethanamine, adenine, cytosine, diethylamine, glucosamine, guanine, nicotinamide, hydrabamine, tributylamine, deanol, epolamine or triethanolamine.

Representative salts of the compounds of the present invention include, but not limited to, hydrochloride, methanesulfonate, sulfonate, phosphonate, isethionate and trifluoroacetate.

The compounds of the present invention can also be used in the form of prodrugs. Examples of such prodrugs include compounds wherein one, two or three hydroxy groups in the compound of this invention are functionalized with R¹⁵ wherein R¹⁵ is 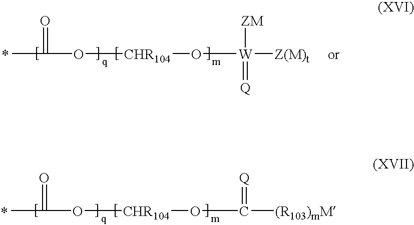
wherein
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring system is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1; and
t is 0 or 1.

Representative examples of R¹⁵ of formula (XVI) or (XVII) that can be utilized for the functionalization of the hydroxy groups in the compound of the present invention include, but not limited to, the following:

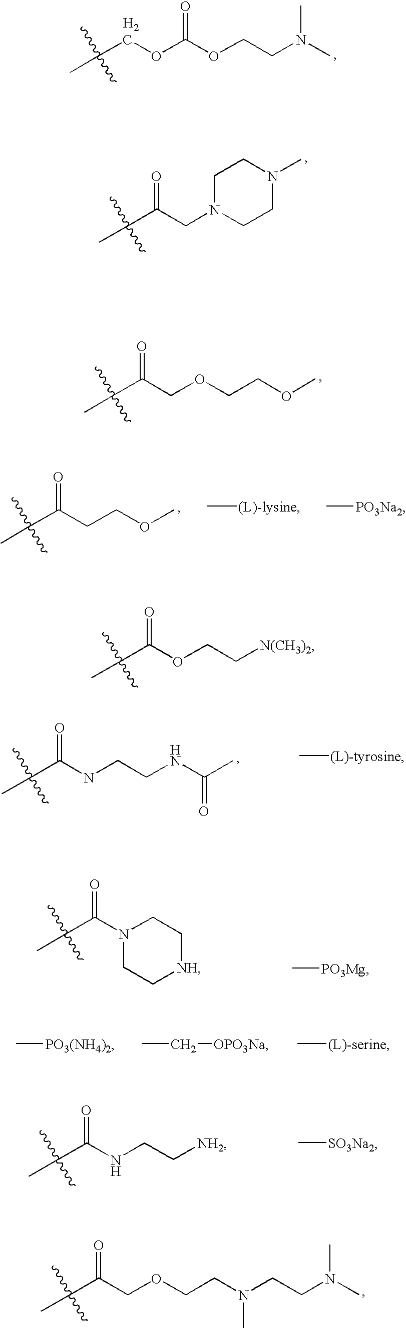

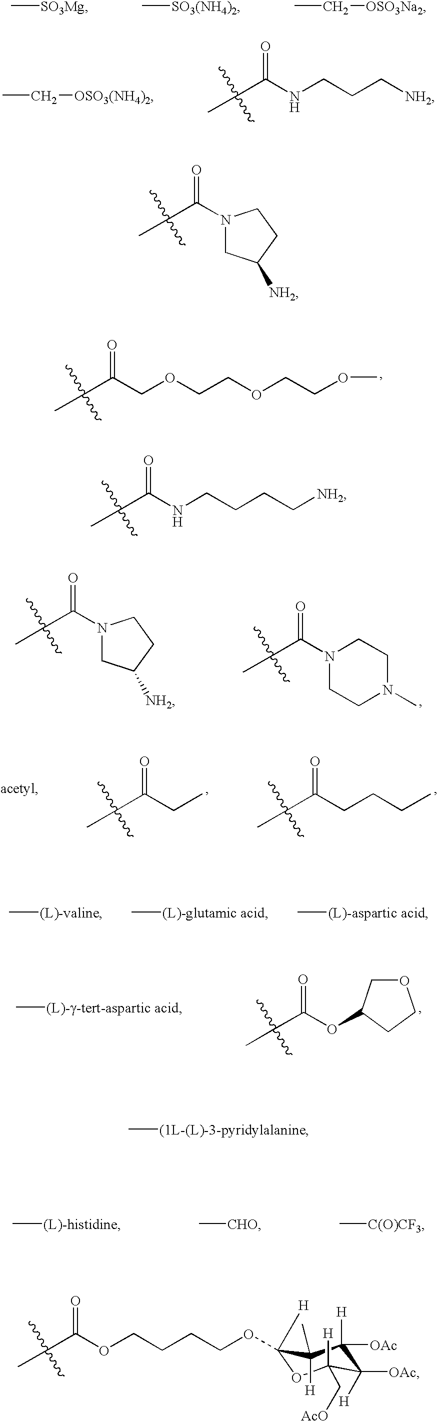

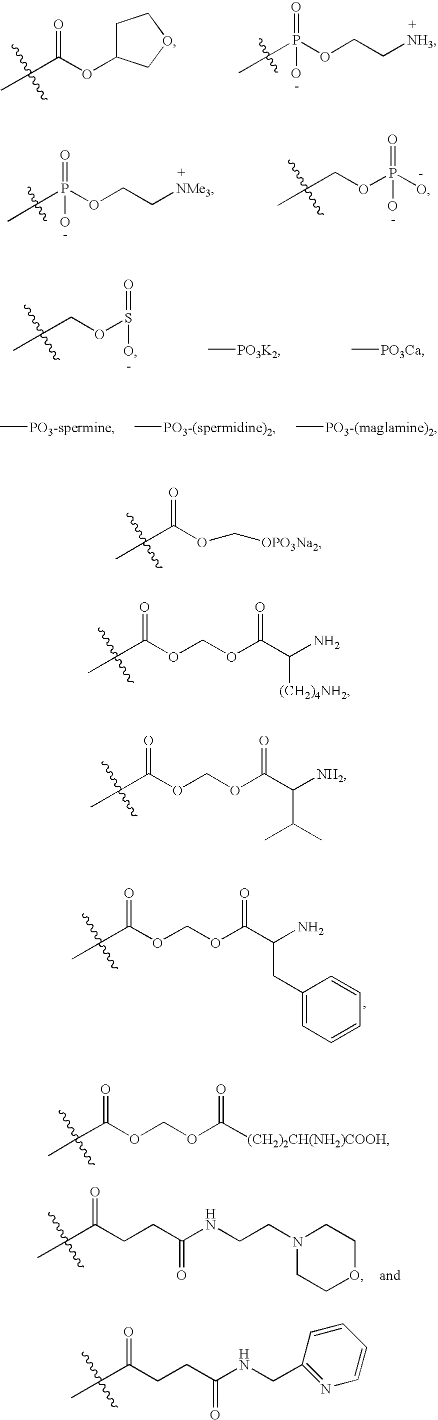

It will be understood by those of skill in the art that component M or M′ in the formulae set forth herein will have either a covalent, a covalent/zwitterionic, or an ionic association with either Z or R₁₀₃ depending upon the actual choice for M or M′. When M or M′ is hydrogen, alkyl, alkenyl or R₁₀₆ then M or M′, is covalently bound to —R₁₀₃ or Z. If M is a mono or bivalent metal or other charged species (i.e. NH₄⁺), there is an ionic interaction between M and Z and the resulting compound is a salt.

These prodrugs of the compound of the present invention serve to increase the solubility of these compounds in the gastrointestinal tract. These prodrugs also serve to increase solubility for intravenous administration of the compound. These prodrugs may be prepared by using conventional synthetic techniques. One of skill in the art would be well aware of conventional synthetic reagents to convert one or more of the hydroxy groups of the compounds of the present invention to a desired prodrug, functionalized by the substituents of formula (XVI) or (XVII) as defined above.

The prodrugs of this invention are metabolized in vivo to provide the compound of this invention.

The compounds of the invention are useful for inhibiting retroviral protease, in particular HIV protease, in vitro or in vivo (especially in mammals and in particular in humans). The compounds of the present invention are also useful for the inhibition of retroviruses in vivo, especially human immunodeficiency virus (HIV). The compounds of the present invention are also useful for the treatment or prophylaxis of diseases caused by retroviruses, especially acquired immune deficiency syndrome or an HIV infection in a human or other mammal.

Total daily dose administered to a human or other mammal host in single or divided doses may be in amounts, for example, from 0.001 to 300 mg/kg body weight daily and more usually 0.1 to 20 mg/kg body weight daily. Dosage unit compositions may contain such amounts of submultiples thereof to make up the daily dose.

The amount of active ingredient that may be combined with the carrier materials to produce a single dosage form will vary, depending upon the host treated and the particular mode of administration.

It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy.

The compounds of the present invention may be administered orally, parenterally, sublingually, by inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous injections, intravenous, intramuscular, intrasternal injection, or infusion techniques.

Injectable preparations, for example, sterile injectable aqueous or oleagenous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols which are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically aceptable and metabolizable lipid capabale of forming liposomes can be used. The present compositions in liposome form can contain, in addition to the compound of the present invention, stabilizers, preservatives, excipients, and the like. The preferred lipids are the phospholipids and phosphatidyl cholines (lecithins), both natureal and synthetic.

Methods to form liposomes are known in the art. See, for example, Prescott, Ed., Methods in Cell Biology, Volume XIV, Academic Press, New York, N.Y. (1976), p. 33.

While the compound of the invention can be administered as the sole active pharmaceutical agent, it can also be used in combination with one or more immunomodulators, antiviral agents, other antiinfective agents or vaccines. Other antiviral agents to be administered in combination with a compound of the present invention include AL-721, beta interferon, polymannoacetate, reverse transcriptase inhibitors (for example, BCH-189, AzdU, carbovir, ddA, d4C, d4T (stavudine), 3TC (lamivudine) DP-AZT, FLT (fluorothymidine), BCH-9189, 5-halo-3′-thia-dideoxycytidine, PMEA, bis-POMPMEA, zidovudine (AZT), MSA-300, trovirdine, R82193, L-697,661, BI-RG-587 (nevirapine), abacavir, zalcitabine, didanosine, tenofovir, emtricitabine, amdoxovir, elvucitabine, alovudine, MIV-210, Racivir (±-FTC), D-D4FC (Reverset, DPC-817), SPD754, nevirapine, delavirdine, efavirenz, capravirine, emivirine, calanolide A, GW5634, BMS-56190 (DPC-083), DPC-961, MIV-150, TMC-120, and TMC-125 and the like), retroviral protease inhibitors (for example, HIV protease inhibitors such as ritonavir, lopinavir, saquinavir, amprenavir (VX-478), fosamprenavir, nelfinavir (AG1343), tipranavir, indinavir, atazanavir, TMC-126, TMC-114, mozenavir (DMP-450), JE-2147 (AG1776), L-756423, RO0334649, KNI-272, DPC-681, DPC-684, GW640385X, SC-52151, BMS 186,318, SC-55389a, BILA 1096 BS, DMP-323, KNI-227, and the like), HEPT compounds, L,697,639, R82150, U-87201E and the like), HIV integrase inhibitors (S-1360, zintevir (AR-177), L-870812 L-870810 and the like), TAT inhibitors (for example, RO-24-7429 and the like), trisodium phosphonoformate, HPA-23, eflonithine, Peptide T, Reticulose (nucleophosphoprotein), ansamycin LM 427, trimetrexate, UA001, ribavirin, alpha interferon, oxetanocin, oxetanocin-G, cylobut-G, cyclobut-A, ara-M, BW882C87, foscarnet, BW256U87, BW348U87, L-693,989, BV ara-U, CMV triclonal antibodies, FIAC, HOE-602, HPMPC, MSL-109, TI-23, trifluridine, vidarabine, famciclovir, penciclovir, acyclovir, ganciclor, castanosperminem rCD4/CD4-IgG, CD4-PE40, butyl-DNJ, hypericin, oxamyristic acid, dextran sulfate and pentosan polysulfate. Other agents that can be administered in combination with the compound of the present invention include HIV entry/fusion inhibitor (for example, enfuvirtide (T-20), T-1249, PRO 2000, PRO 542, PRO 140, AMD-3100, BMS-806, FP21399, GW873140, Schering C (SCH-C), Schering D (SCH-D), TNX-355, UK-427857, and the like) and HIV budding/maturation inhibitor such as PA-457. Immunomodulators that can be administered in combination with the compound of the present invention include bropirimine, Ampligen, anti-human alpha interferon antibody, colony stimulting factor, CL246,738, Imreg-1, Imreg-2, diethydithiocarbamate, interleukin-2, alpha-interferon, inosine pranobex, methionine enkephalin, muramyl-tripeptide, TP-5, erythropoietin, naltrexone, tumor necrosis factor, beta interferon, gamma interferon, interleukin-3, interleukin-4, autologous CD8+ infusion, alpha interferon immunoglobulin, IGF-1, anti-Leu-3A, autovaccination, biostimulation, extracorporeal photophoresis, cyclosporin, rapamycin, FK-565, FK-506, GCSF, GM-CSF, hyperthermia, isopinosine, IVIG, HIVIG, passive immunotherapy and polio vaccine hyperimmunization. Other antiinfective agents that can be administered in combination with the compound of the present invention include pentamidine isethionate. Any of a variety of HIV or AIDS vaccines (for example, gp120 (recombinant), Env 2-3 (gp120), HIVAC-1e (gp120), gp160 (recombinant), VaxSyn HIV-1 (gp160), Immuno-Ag (gp160), HGP-30, HIV-Immunogen, p24 (recombinant), VaxSyn HIV-1 (p24)) can be used in combination with the compound of the present invention.

Other agents that can be used in combination with the compound of this invention are ansamycin LM 427, apurinic acid, ABPP, Al-721, carrisyn, AS-101, avarol, azimexon, colchicine, compound Q, CS-85, N-acetyl cysteine, (2-oxothiazolidine-4-carboxylate), D-penicillamine, diphenylhydantoin, EL-10, erythropoieten, fusidic acid, glucan, HPA-23, human growth hormone, hydroxchloroquine, iscador, L-ofloxacin or other quinolone antibiotics, lentinan, lithium carbonate, MM-1, monolaurin, MTP-PE, naltrexone, neurotropin, ozone, PAI, panax ginseng, pentofylline, pentoxifylline, Peptide T, pine cone extract, polymannoacetate, reticulose, retrogen, ribavirin, ribozymes, RS-47, Sdc-28, silicotungstate, THA, thymic humoral factor, thymopentin, thymosin fraction 5, thymosin alpha one, thymostimulin, UA001, uridine, vitamin B12 and wobemugos.

Other agents that can be used in combination with the compound of this invention are antifungals such as amphotericin B, clotrimazole, flucytosine, fluconazole, itraconazole, ketoconazole and nystatin and the like.

Other agents that can be used in combination with the compound of this invention are antibacterials such as amikacin sulfate, azithromycin, ciprofloxacin, tosufloxacin, clarithromycin, clofazimine, ethambutol, isoniazid, pyrazinamide, rifabutin, rifampin, streptomycin and TLC G-65 and the like.

Other agents that can be used in combination with the compound of this invention are anti-neoplastics such as alpha interferon, COMP (cyclophosphamide, vincristine, methotrexate and prednisone), etoposide, mBACOD (methotrexate, bleomycin, doxorubicin, cyclophosphamide, vincristine and dexamethasone), PRO-MACE/MOPP (prednisone, methotrexate (w/leucovin rescue), doxorubicin, cyclophosphamide, taxol, etoposide/mechlorethamine, vincristine, prednisone and procarbazine), vincristine, vinblastine, angioinhibins, pentosan polysulfate, platelet factor 4 and SP-PG and the like.

Other agents that can be used in combination with the compound of this invention are drugs for treating neurological disease such as peptide T, ritalin, lithium, elavil, phenyloin, carbamazipine, mexitetine, heparin and cytosine arabinoside and the like.

Other agents that can be used in combination with the compound of this invention are anti-protozoals such as albendazole, azithromycin, clarithromycin, clindamycin, corticosteroids, dapsone, DIMP, eflornithine, 566C80, fansidar, furazolidone, L,671,329, letrazuril, metronidazole, paromycin, pefloxacin, pentamidine, piritrexim, primaquine, pyrimethamine, somatostatin, spiramycin, sulfadiazine, trimethoprim, TMP/SMX, trimetrexate and WR 6026 and the like.

For example, a compound of this invention can be administered in combination with ritonavir. Such a combination is especially useful for inhibiting HIV protease in a human. Such a combination is also especially useful for inhibiting or treating an HIV infection in a human. When used in such a combination the compound of this invention and ritonavir can be administered as separate agents at the same or different times or they can be formulated as a single composition comprising both compounds.

When administered in combination with a compound, or combination of compounds of this invention, ritonavir causes an improvement in the pharmacokinetics (i.e., increases half-life, increases the time to peak plasma concentration, increases blood levels) of the compound of this invention.

Another combination can comprise of a compound, or combination of compounds of the present invention with ritonavir and one or more reverse transcriptase inhibitors (for example, lamivudine, stavudine, zidovudine, abacavir, zalcitabine, didanosine, tenofovir, emtricitabine, amdoxovir, elvucitabine, alovudine, MIV-210, Racivir (±-FTC), D-D4FC (Reverset, DPC-817), SPD754, nevirapine, delavirdine, efavirenz, capravirine, emivirine, calanolide A, GW5634, BMS-56190 (DPC-083), DPC-961, MIV-150 TMC-120, TMC-125 and the like). Yet another combination can comprise of a compound, or combination of compounds of the present invention with ritonavir and one or more HIV entry/fusion inhibitors. Such combinations are useful for inhibiting or treating an HIV infection in a human. When used in such a combination the compound or combination of compounds of the present invention and ritonavir and one or more reverse transcriptase inhibitors or HIV entry/fusion inhibitors can be administered as separate agents at the same or different times or they can be formulated as compositions comprising two or more of the compounds.

It will be understood that agents which can be combined with the compound of the present invention for the inhibition, treatment or prophylaxis of AIDS or an HIV infection are not limited to those listed above, but include in principle any agents useful for the treatment or prophylaxis of AIDS or an HIV infection.

When administered as a combination, the therapeutic agents can be formulated as separate compositions which are given at the same time or different times, or the therapeutic agents can be given as a single composition.

Antiviral Activity

### Determination of Activity Against Wild-Type HIV or the Passaged Variants

MT4 cells were infected with 0.003 multiplicity of infection (MOI) of wild-type HIV-1 or the passaged mutant variants at 1×10⁶ cells/mL for 1 h, washed twice to remove unabsorbed virus and resuspended to 1×10⁵ cells/mL of medium, seeded in a 96-well plate at 100 μL/well, and treated with an equal volume of solution of inhibitor in a series of half log dilutions in RPMI 1640 (Rosewell Park Memorial Institute) media (Gibco) containing 10% fetal bovine serum (FBS), in triplicate. The final concentration of DMSO in all wells was 0.5%. The virus control culture was treated in an identical manner except no inhibitor was added to the medium. The cell control was incubated in the absence of inhibitor or virus. Plates were incubated for 5 days in a CO₂ incubator at 37° C. On day 5, stock solution of 3-[4,5-dimethylthiazol-2-yl]-2,5-diphenyltetrazolium bromide (MTT) (4 mg/mL in PBS, Sigma cat. # M 5655) was added to each well at 25 μL per well. Plates were further incubated for 4 hrs, then treated with 20% sodium dodecyl sulfate (SDS) plus 0.02 N HCl at 50 μL per well to lyse the cells. After an overnight incubation, optical density (O.D.) was measured by reading the plates at 570/650 nm wavelengths on a Bio-Tek microtitre plate reader. Percent cytopathic effect (CPE) reduction was calculated from the formula below:
((O.D. test well−O.D. infected control well)/(O.D. uninfected control well−O.D. infected control well))×100

EC₅₀ values were determined from the plot of log (Fa/Fu) vs. log (compound concentration) using the median-effect equation (Chou, 1975, Proc. Int. Cong. Pharmacol. 6^{th} p. 619) wherein Fa is the fraction inhibited by the compound, and Fu is the fraction uninhibited (1-Fa).

When tested by the above method, the compounds of the present invention exhibit EC₅₀ in the range of 0.7 nM to 300 nM.

### Determination of Anti-HIV Activity in the Presence of Human Serum

The above antiviral assay was performed in 96-well tissue culture plates containing 50% human serum (HS) (Sigma) plus 10% FBS (Gibco/BRL, Grand Island, N.Y.). Compounds were dissolved in DMSO, diluted at half log concentrations in DMSO, then transferred to media without serum at four times the final concentration. These solutions were added to 96-well plates at 50 μL per well, in triplicate. Cells were separately infected with 0.003 MOI of HIV-1 at 1×10⁶ cells/mL for 1 hour, washed twice to remove unadsorbed virus and resuspended to 2×10⁵ cells/mL of media without serum. The cell suspension (50 μL) was seeded at 1×10⁴ cells per well. Uninfected cells were included as control. Final DMSO concentration in all wells was 0.5% including uninfected and infected control wells. Cultures were incubated for 5 days in a CO₂ incubator at 37° C. EC₅₀ values were measured using MTT uptake as described above.

When tested by the above method, compounds of the present invention exhibit EC₅₀ in the range of 5 nM to >1000 nM.

### Generation of HIV-1 Resistant to ABT-378/r (A17) by In Vitro Passage

MT4 cells (2×10⁶) were infected with pNL4-3 at an MOI of 0.03 for 2 h, washed, then cultured in the presence of ABT-378 and ritonavir at concentration ratio of 5:1. The concentration of ABT-378 and ritonavir used in the initial passage was 1 nM and 0.2 nM respectively. Viral replication was monitored by determination of p24 antigen levels in the culture supernatant (Abbott Laboratories), as well as by observation for any cytopathic effect (CPE) present in the cultures. When p24 antigen levels were positive, the viral supernatant was harvested for the proceeding passage. Following each passage, the drug concentrations in the subsequent passage were gradually increased. After 5 months of selection, 1.5 μM of ABT-378 can be used in the final passage. The A17 virus was generated after 17 passages of pNL43 in the presence of ABT-378 and ritonavir at concentration ratio of 5:1.

When tested by the above method, compounds of the present invention inhibit the A17 virus with EC₅₀ in the range of 0.3 nM to >1000 nM.

Synthetic Methods

Abbreviations which have been used in the descriptions of the schemes and the examples that follow are: DMF is N,N-dimethylformamide, DMSO is dimethylsulfoxide, THF is tetrahydrofuran, NMMO is 4-methylmorpholine N-oxide, HOBT is 1-hydroxybenzotriazole hydrate, DCC is 1,3-dicyclohexylcarbodiimide, EDAC is 1-(3-dimethylaminopropyl)-3-ethylcarbodiimide hydrochloride, DMAP is 4-(dimethylamino)pyridine, TFA is trifluoroacetic acid, DEPBT is 3-(diethoxyphosphoryloxy)-1,2,3-benzotriazin-4(3H)-one, DPPA is diphenylphosphine azide, NMM is N-methylmorpholine, DIBAL is diisobutyl aluminum hydride, EtOAc is ethyl acetate and TBAF is tetrabutyl ammonium fluoride.

The compounds and processes of the present invention will be better understood in connection with the following synthetic schemes which illustrate the methods by which the compounds of the invention may be prepared. Starting materials can be obtained from commercial sources or prepared by well-established literature methods known to those of ordinary skill in the art. The groups A, R¹, R², R³, R⁴, R⁵, R⁶, R⁷, R⁸, R⁹, R¹⁰, R¹¹, R¹², R¹³, R¹⁴, Rₐ, R_{b}, R_{c} and n are as defined above unless otherwise noted below.

This invention is intended to encompass compounds having formula (I), (II), (III), (IV), (V), (VI), (VII), (VIII), (IX), (X), (XI), (XII) or (XIII), when prepared by synthetic processes or by metabolic processes. Preparation of the compounds of the invention by metabolic processes includes those occurring in the human or animal body (in vivo) or processes occurring in vitro.

Compounds of the invention can be prepared according to the methods described in Schemes 1-6 as shown below.

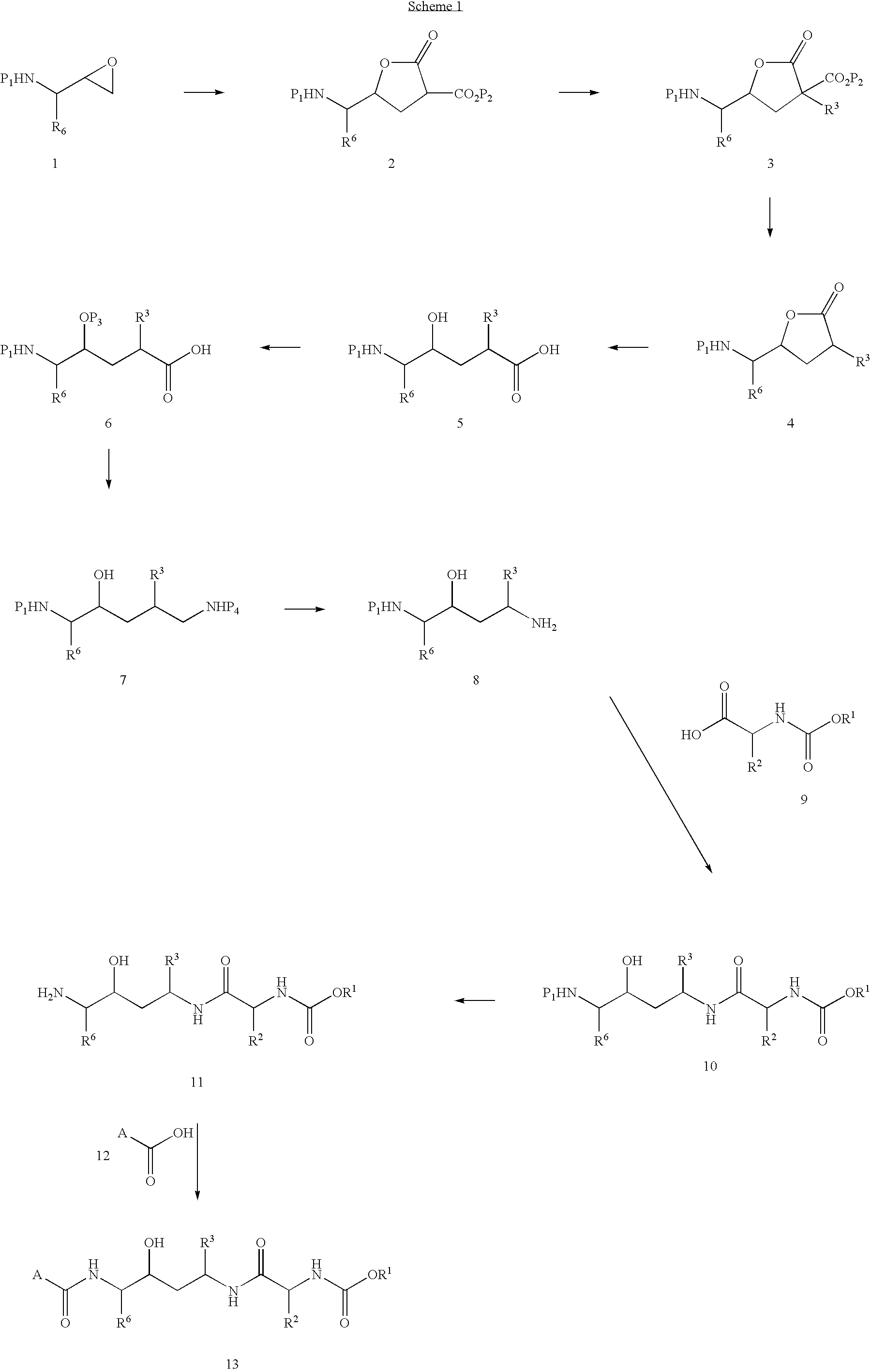

Compounds of formula (1) wherein P₁ is an N-protecting group, for example 1-tert-butyloxycarbonyl or benzyloxycarbonyl, can be treated with a dialkyl malonate and a base in an alcoholic solvent such as, but not limited to, methanol or ethanol, at a temperature of about −15° C. to about 30° C. to give compounds of formula (2), wherein P₂ is a carboxyl protecting group, for example ethyl, methyl, benzyl, tert-butyl, and the like. Examples of the dialkyl malonate are, but are not limited to, diethyl malonate, dimethyl malonate or dibenzyl malonate. Examples of the base include, but are not limited to, sodium methoxide, sodium ethoxide and sodium tert-butoxide.

Compounds of formula (2) can be isolated or reacted in-situ with an alkylating agent of formula R³X, wherein X is F, Br, Cl or I, and the like, in the presence of a base, in a solvent such as ethanol, methanol, THF, dioxane, DMF, or mixtures thereof, at a temperature from about 25° C. to about 80° C., to give compounds of formula (3). Examples of the base include, but are not limited to, sodium methoxide, sodium ethoxide and sodium tert-butoxide, NaNH₂, lithium bis(trimethylsilyl)amide and lithium diisopropylamide.

Compounds of formula (3) can be converted to compounds of formula (4) by (a) reacting compounds of formula (3) with a base, in a solvent such as, but not limited to, THF, DMF, methanol, ethanol or water, and mixtures thereof, at a temperature from about 25° C. to about 100° C., and (b) heating the product of step (a) at reflux in a high boiling solvent such as, but not limited to, benzene, toluene, xylene, DMF or acetic acid. Examples of the base include, but are not limited to, lithium hydroxide, sodium hydroxide, potassium hydroxide and potassium carbonate.

Transformation of compounds of formula (4) to compounds of formula (6), wherein P₃ is a hydroxyl protecting group (for example, tert-butyldimethyl silyl) can be achieved in a one-step or stepwise manner by (a) contacting compounds of formula (4) with a first base in a solvent such as, but not limited to, N-methylpyrrolidinone, DMF, THF, dioxane at a temperature from about 0° C. to about 50° C., and (b) contacting the product of step (b) with a silylating agent and a second base in an inert solvent such as, but not limited to, ethyl acetate, THF, dichloromethane, DMF, NMP, acetonitrile, isopropyl acetate or toluene, and the like, at a temperature from about −10° C. to about 60° C. Examples of the first base include, but are not limited to, inorganic bases such as sodium hydroxide, lithium hydroxide, potassium hydroxide, and the like, optionally in the presence of 4-N,N-dimethylamino pyridine (DMAP). Examples of the second base include, but are not limited to, organic amine bases such as imidazole, 1-methylimidazole, 2-methylimidazole, 2-isopropylimidazole, 4-methylimidazole, 4-nitroimidazole, pyridine, N,N-dimethylaminopyridine, 2,6-lutidine, 1,2,4-triazole, pyrrole, 3-methylpyrrole, triethylamine or N-methylmorpholine and the like. Examples of the silylating agent include, but are not limited to, trimethylsilyl chloride, trimethylsilyl triflate, tert-butyldimethylsilyl chloride, and tert-butyldimethylsilyl triflate.

Compounds of formula (6) can be converted to compounds of formula (7), wherein P₄ is an N-protecting group (for example benzyloxy carbonyl), in a one-step or stepwise manner, by (a) treating compounds of formula (6) with diphenylphosphoryl azide and a base such as, but not limited to, triethylamine, diisoproylethylamine, N-methylmorpholine, and the like in a solvent, or mixture of solvents, such as xylene, toluene, benzene or DMF, and the like, at a temperature from about 80° C. to about 150° C., (b) treating the product of step (b) with an alcohol at a temperature from about 80° C. to about 150° C., in a solvent, in a solvent, or mixture of solvents, such as xylene, toluene, benzene or DMF, and the like, and (c) treating the product of step (b) with a desilylating agent in a solvent, or mixture of solvents, such as THF, DMF, ethyl acetate, dichloromethane, acetone, acetonitrile, methanol or diethyl ether, and the like, at a temperature from about 0° C. to about 50° C. Examples of the alcohol include, but are not limited to, tert-butyl alcohol and benzyl alcohol. Examples of the desilylating agent include, but are not limited to, tetrabutyl ammonium fluoride, acetic acid, formic acid, HCl, HF and citric acid.

Removal of the P₄ benzyloxy carbonyl group of (7) (for example, using hydrogen and a hydrogenation catalyst or Pd/C and a formic acid salt (for example, ammonium formate and the like) or Pd/C and formic acid and the like) provides (8). Examples of the hydrogenation catalyst include, but are not limited to, Pd/C, Raney nickel, platinum metal and its oxides.

Compounds of formula (8) are reacted with carboxylic acids of formula (9) and an activating agent, optionally in the presence of 1-Hydroxy-7-azabenzotriazole (HOAT), 1-hydroxybenzotriazole hydrate (HOBT) or 3-hydroxy-1,2,3-benzotriazin-4(3H)-one (HOOBT), and optionally in the presence of an inorganic base (for example, sodium bicarbonate, sodium carbonate, potassium bicarbonate, potassium carbonate, sodium hydroxide, potassium hydroxide, and the like) in an inert solvent (for example, 1:1 ethyl acetate/water or isopropyl acetate/water or toluene/water or THF/water and the like) at about room temperature, or an organic amine base (for example, imidazole, 1-methylimidazole, 2-methylimidazole, 2-isopropylimidazole, 4-methylimidazole, 4-nitroimidazole, pyridine, N,N-dimethylaminopyridine, 1,2,4-triazole, pyrrole, 3-methylpyrrole, triethylamine or N-methylmorpholine and the like) in an inert solvent (for example, ethyl acetate, isopropyl acetate, THF, toluene, acetonitrile, DMF, dichloromethane and the like) at a temperature from about 0° C. to about 50° C. to provide compound (10). Examples of the activating agent include, but are not limited to, 1,1′-carbonyldiimidazole (CDI), 1,3-dicyclohexylcarbodiimide (DCC), 1,3-diisopropylcarbodiimide, 1-(3-dimethylaminopropyl)-3-ethylcarbodiimide hydrochloride (EDAC), DEPBT (3-(diethoxyphosphoryloxy)-1,2,3-benzotriazin-4(3H)-one), benzotriazol-1-yl-oxy-tris-pyrrolidino-phosphoniumhexafluorophosphate (PyBOP), and 1,3-di-tert-butylcarbodiimide. Alternatively, a salt or an activated ester derivative of acid (9) (for example, the acid chloride, prepared by reaction of the carboxylic acid with thionyl chloride in ethyl acetate or THF or oxalyl chloride in toluene/DMF) can be reacted with (8).

Removal of tert-butoxycabonyl group can be accomplished by treating compounds of formula (10) with an acid (for example, trifluoroacetic acid, hydrochloric acid, methanesulfonic acid, toluenesulfonic acid, sulfuric acid, aluminum chloride and the like) in an inert solvent (for example, dioxane, dichloromethane, chloroform, methanol, THF, acetonitrile and the like) at a temperature from about 0° C. to about room temperature, to provide (11).

Compounds of formula (11) can be reacted with acids of formula (12), or its salts, using the conditions for the transformation of (8) to (10), to provide compounds of formula (13).

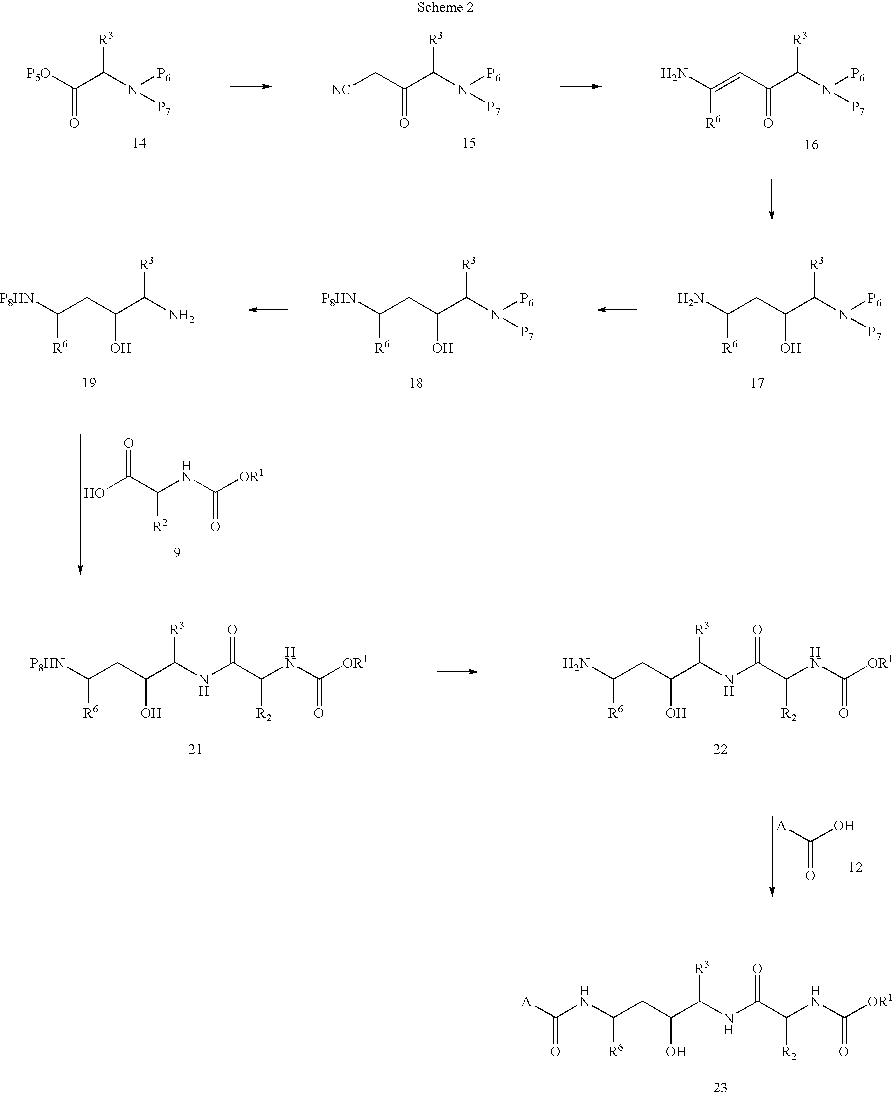

Protected amino acids of formula (14), wherein P₅ is lower alkyls, P₆ and P₇ are N— protecting groups (preferably, P₆ and P₇ are benzyl) is reacted with sodioacetonitrile, (formed in-situ from acetonitrile and a base such as NaNH₂, lithium bis(trimethylsilyl)amide, or lithium diisopropylamide, and the like) in a solvent, or mixtures of solvents, such as acetonitrile or THF, and the like, at a temperature of about −40° C. to provide ketonitrile (15). Addition of an organometallic reagent of formula R⁶MX, wherein M is a metal such as magnesium, and X is Cl, Br or I, in an inert solvent such as, but not limited to, dichloromethane, THF, diethyl ether, methyl tert-butyl ether, at a temperature from about 0° C. to about room temperature. Examples of the organometallic reagent include, but are not limited to, benzyl magnesium chloride and methylmagnesium bromide. Reduction of (16) to compounds of formula (17) can be accomplished by reaction with a reducing agent in an inert solvent, or mixtures of solvents, such as ethyl acetate, THF, dichloromethane, ethyl acetate, diethyl ether and the like, at a temperature from about −10° C. to about room temperature. Examples of reducing agents include, but are not limited to, hydrogen in the presence of a catalyst (for example, Pd/C, Raney nickel, platinum metal or its oxides and the like), metallic hydrides such as lithium aluminum hydride and sodium borohydride. The amino group can subsequently be protected to provide compound (18), wherein P₈ is tert-butoxycarbonyl, by conditions that are well known in the art.

N-Debenzylation of compounds of formula (18) wherein P₆ and P₇ are benzyl to provide compounds of formula (19) can be achieved using the conditions for the transformation of compounds of formula (7) to compounds of formula (8).

Conversion of compounds of formula (19) to compounds of formula (23) can be achieved using the conditions for the transformation of compounds of formula (8) to compounds of formula (13)

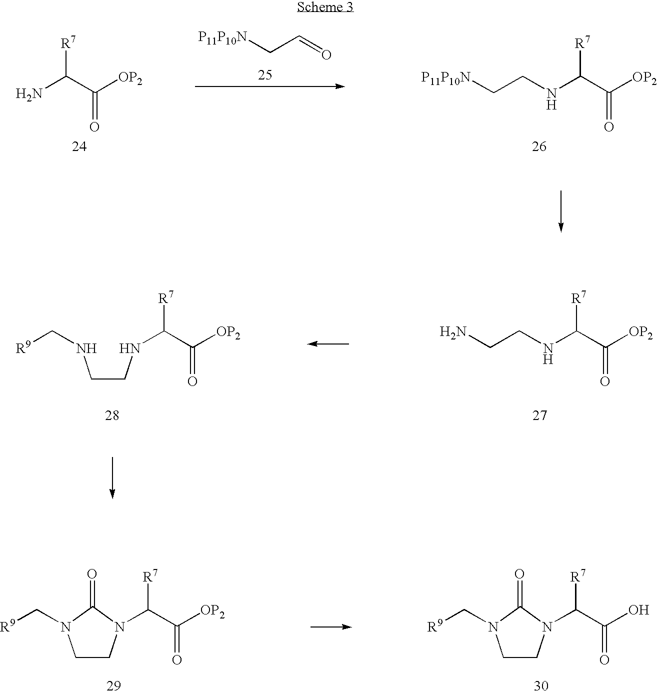

Amino acid esters of formula (24), wherein P₂ is lower alkyls (for example methyl, ethyl, tert-butyl and the like), can be treated with a suitably protected aldehyde of formula (2S) (for example, P₁₀ and P₁₁ together with the nitrogen atom they are attached, form a phthalimido group) in the presence of a reducing agent, optionally under acidic conditions (for example, in the presence of acetic acid or hydrochloric acid), in an inert solvent, or mixture of solvents, such as methyl sulfoxide, methanol, dichloromethane, and the like, at a temperature from about room temperature to about 50° C., to provide compounds of formula (26). Examples of the reducing agent include, but are not limited to, sodium triacetoxyborohydride, sodium borohydride, sodium cyanoborohydride, and BH₃-pyridine.

Removal of the phthalimido group can be achieved by treatment with hydrazine in a suitable solvent such as ethanol and the like, at a temperature of about room temperature to about 100° C., to provide compounds of formula (27).

Compounds of formula (27) can be converted to compounds of formula (28) by (a) treating compounds of formula (27) with an aldehyde having formula R⁹CHO, optionally in the presence of a drying agent (for example, magnesium sulfate, silica gel and the like) in an inert solvent, or mixture of solvents, such as dichloromethane, benzene, toluene, methanol, ethanol, methyl sulfoxide, and the like, at a temperature from about room temperature to about 100° C., and (b) reacting the product of step (a) with a reducing agent at about room temperature. Examples of the reducing agent include, but are not limited to, sodium triacetoxyborohydride, sodium borohydride, sodium cyanoborohydride, and BH₃-pyridine.

The diamine of formula (28) can be treated with a carbonylating agent in an inert solvent, or mixture of solvents, such as dichloromethane, 1,2 dichloroethane, toluene, acetonitrile, and the like, at a temperature from about room temperature to about 100° C., to provide compounds of formula (29). Examples of the carbonylating agent include, but not are limited to, 4-nitrophenyl carbonate, phosphene, diphosgene, triphosgene, carbonyl diimidazole and disuccinimidyl carbonate.

Conversion of compounds of formula (29) to the corresponding acids having formula (30) can be achieved by acid hydrolysis (for example acetic acid, trifluoroacetic acid, toluenesulfonic acid, formic acid, hydrochloric acid and the like) or base hydrolysis (for example sodium hydroxide, potassium hydroxide, lithium hydroxide, cesium carbonate, and the like) in a solvent, or mixture of solvents such as DMF, toluene, benzene, dichloromethane, ethyl acetate, water, methanol and the like, at a temperature from about 0° C. to about 100° C.

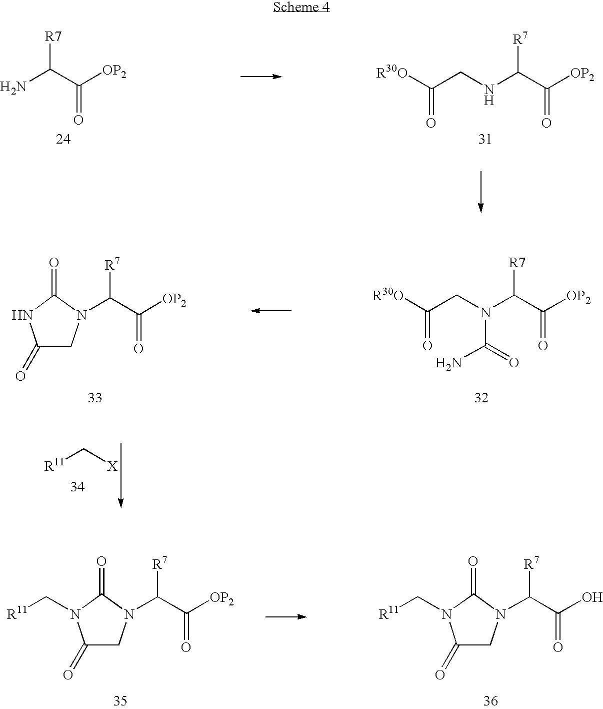

Amino acid esters having formula (24), wherein P₂ is lower alkyls (for example, methyl, ethyl, tert-butyl and the like) can be treated with compounds of formula R³⁰OC(O)CH₂X, wherein R³⁰ is lower alkyls and X is Br, Cl, or I, in an inert solvent, or mixture of solvents, such as DMF, dichloromethane, 1,2-dichloroethane, acetonitrile, toluene, benzene, diethyl ether and the like, at a temperature of about room temperature to about 50° C., to provide (31).

Compounds of formula (31) can be converted to compounds of formula (32) by (a) treating with compounds of formula XSO₂NCO (for example chlorosulfonyl isocyanate), wherein X is Br, Cl, or I, in an inert solvent, or mixture of solvents, such as dichloromethane, 1,2-dichloroethane, dioxane, toluene, DMF, THF diethyl ether and the like, at a temperature from about −10° C. to about room temperature, and (b) treating the product of step (a) with water at about room temperature. Alternatively, (31) can be reacted with a carbonylating agent such as, but not limited to, 4-nitrophenyl carbonate, phosphene, diphosgene, triphosgene, carbonyl diimidazole, disuccinimidyl carbonate, followed by reaction with ammonia.

Cyclization of the compounds of formula (32) to provide compounds of formula (33) can be achieved be treating with an organic amine base such as triethyl amine, diisopropylethyl amine, imidazole, pyridine, N-methylmorpholine and the like, or an inorganic base such as sodium bicarbonate, sodium carbonate, cesium-carbonate and the like, in an inert solvent, or mixture of solvents, such as methanol, ethanol, DMF, dioxane, xylene, THF and the like, at a temperature from about room temperature to about 100° C.

Imides of formula (33) can be converted to compounds of formula (35) by (a) deprotonation with a base in an inert solvent, or mixture of solvents, such as DMF, THF, diethyl ether, tert-butyl methyl ether, and the like, at a temperature from about −78° C. to about 0° C., and (b) treating product of step (a) with an alkyl halide of formula (34), wherein X is Cl, Br or I, at a temperature from about room temperature to about 100° C. Examples of the base include, but are not limited to, sodium hydride, potassium hydride, lithium diisopropyl amide, lithium bis(trimethylsilyl)amide.

Alternatively, compounds of formula (33) can be converted to compounds of formula (35) by treating with an alcohol having formula R¹¹CH₂OH, in the presence of triphenylphosphine and diethyl azodicarboxylate, in an inert solvent such as dichloromethane, THF, dioxane or DMF, at a temperature of about 0° C. to about 25° C.

Conversion of compounds of formula (35) to compounds of formula (36) can be achieved by using the conditions for the transformation of compounds of formula (29) to compounds of formula (30).

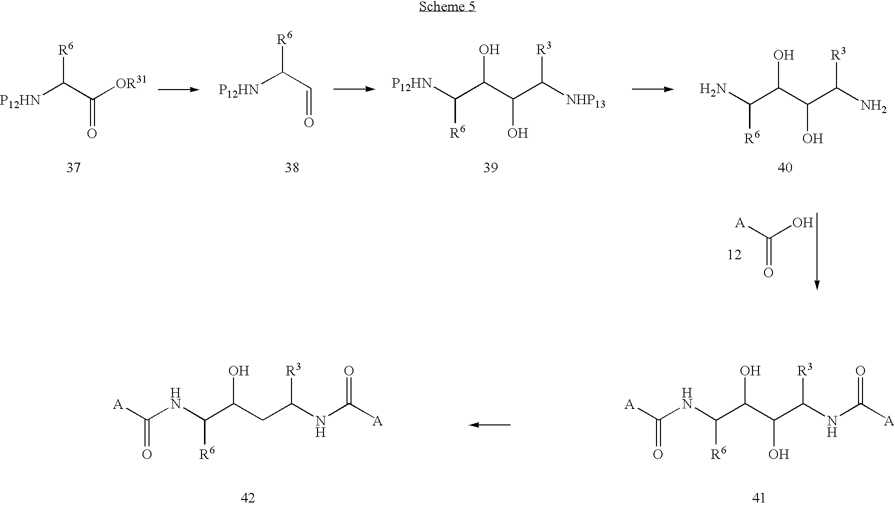

Protected amino acids of formula (37), wherein P₁₂ is an N-protecting group (for example benzyloxycarbonyl, benzyl, tert-butyloxycarbonyl, and the like) and R³¹ is hydrogen or lower alkyls (for example, methyl, ethyl and the like), can be converted to compounds of formula (38) by (a) treating with a reducing agent in an inert solvent such as dichloromethane, di ethyl ether, THF, tert-butyl methyl ether, and the like, at a temperature from about −78° C. to about room temperature, and (b) treating the product of step (a) with an oxidizing agent in an inert solvent, such as dichloromethane, diethyl ether, THF, tert-butyl methyl ether, and the like, at a temperature from about 0° C. to about room temperature. Examples of the reducing agent include, but are not limited to, lithium aluminum hydride, lithium borohydride, sodium borohydride and diisobutylaluminum hydride. Examples of the oxidizing agent include, but are not limited to, oxalyl chloride/methyl sulfoxide/triethylamine, Jones reagent, Cr(VI) reagents such as pyridinium chlorochromate, SO₃/pyridine, MnO₂ and KMnO₄.

Compounds of formula (38) can condense with itself, or an aldehyde of formula P₁₃N(H)CH(R³)CHO (prepared from the corresponding carboxylic acids or esters using the conditions for the transformation of (37) to (38)), wherein P₁₃ is a N-protecting group, and may be the same as or different from P₁₂, to give a diols having formula (39). The transformation can be accomplished with vanadium(III) chloride-THF complex and zinc at about room temperature in an inert solvent, such as dichloromethane, THF, diethyl ether, 1,2-dichloroethane, and the like.

N-Deprotection of compounds of formula (39) can be performed in a stepwise manner (if P₁₂ is different from P₁₃) or in one step (if P₁₂ is the same as P₁₃) using the conditions for the transformation of (7) to (8), if the N-protecting groups are benzyl or tert-benzyloxycarbonyl, or using the conditions for the transformation of (10) to (11), if the N-protecting groups are tert-butyloxycarbonyl.

The compounds of formula (41) can be prepared from (40) and carboxylic acids of formula (12), or its salt, using standard peptide coupling conditions (see the conditions for the transformation of (8) to (10)). The compounds of formula (41) can be converted to compounds of formula (42) by (a) treating with a thiocarbonylating agent in an inert solvent such as THF, dichloromethane, 1,2-dichloroethane, diethyl ether, toluene, xylene, and the like, at a temperature from about room temperature to about 100° C., and (b) treating products of step (b) with tributyltin hydride and 2,2′ azobisisobutyronitrile in an inert solvent, such as THF, dichloromethane, 1,2-dichloroethane, diethyl ether, toluene, xylene, and the like, at a temperature from about room temperature to about 150° C. Examples of the thiocarbonylating agent include, but are not limited to, thiocarbonyldiimidazole, and thiophosgene/4-dimethylaminopyridine.

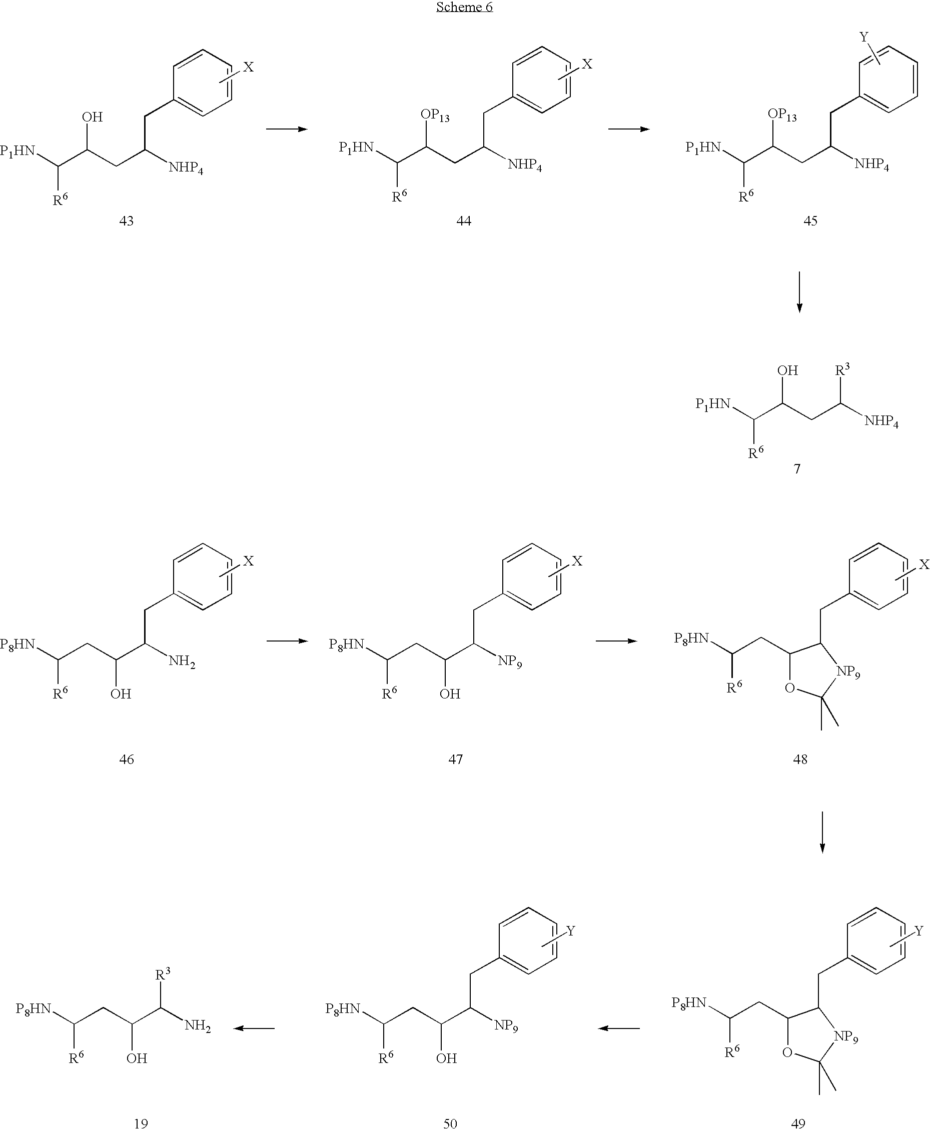

Compounds of formula (43) wherein X is Br, I, Cl or triflate can be converted to compounds of formula (44), wherein P₁₃ is a hydroxyl protecting group (for example, trialkyl silyl, methoxymethyl, and the like) by using the conditions for the transformation of (5) to (6). Treatment of compounds of formula (44) with compounds of formula Y—X¹, wherein Y is aryl or heteroaryl, and X¹ is Br, I, Cl, B(OH)₂, or Sn(lower alkyl)₃, and a palladium catalyst, optionally in the presence of a base (for example cesium carbonate, triethylamine, and the like), and optionally in the presence of CuI, provide compounds of formula (45). Examples of the palladium catalyst include, but are not limited to, tetrakis(triphenylphosphine)Pd(0), dichlorobis(triphenylphosphine)Pd(II), Pd on carbon, Pd(OAc), tris(dibenzylideneacetone)dipalladium(0), or any of the above with additional phosphine ligands such as, 2-(dicyclohexylphosphino)biphenyl or 2{di-tert-butylphosphino)biphenyl. Compounds of formula (45) can be converted to compounds of formula (7), wherein R³ is arylalkyl and wherein the aryl moiety of the arylalkyl is substituted by aryl or heteroaryl, by treatment with a desilylating agent such as, but not limited to, tetrabutyl ammonium fluoride, acetic acid, formic acid, HCl, HF and citric acid in a solvent, or mixture of solvents, such as THF, DMF, ethyl acetate, dichloromethane, acetone, acetonitrile, methanol or diethyl ether, and the like, at a temperature from about 25° C. to about 50° C.

Compounds of formula (47) wherein P₉ is tert-benzyloxycarbonyl, can be obtained from compounds of formula (46) using conditions well known in the art. The compounds of formula (47) can be converted to compounds of formula (48) by treatment with excess 2,2-dimethoxypropane in the presence of an acid (for example, toluenesulfonic acid, acetic acid, sulfuric acid, and the like) at a temperature from about 0° C. to about room temperature, optionally in the presence of an inert solvent such as dichloromethane, toluene, benzene, acetone, and the like. Transformation of (48) to compounds of formula (49), wherein Y is aryl or heteroaryl, can be accomplished by the conditions for the conversion of (44) to (45). Compounds of formula (49) can be converted to compounds of formula (50) by acid hydrolysis (for example acetic acid, trifluoroacetic acid, toluenesulfonic acid, formic acid, hydrochloric acid and the like) in solvent, or mixture of solvents, such as water, methanol, isopropyl alcohol, ethanol, dichloromethane, THF, acetonitrle, toluene, benzene, 1,2-dichloroethane, ethyl acetate, and the like, at a temperature from about room temperature to about 100° C. Compounds of formula (50) can be de-protected by employing the conditions for the conversion of (7) to (8) as illustrated in scheme 1, to provide compounds of formula (19) wherein R³ is arylalkyl and wherein the aryl moiety of the arylalkyl is substituted with aryl or heteroaryl.

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purpose of illustration of certain preferred embodiments and are presented to provide what is believed to be the most useful and readily understood description of its procedures and conceptual aspects.

Compounds of the invention were named by ACD/ChemSketch version 4.01 (developed by Advanced Chemistry Development, Inc., Toronto, ON, Canada) or were given names consistent with ACD nomenclature.

### EXAMPLE 1A

### tert-butyl(1S)-1-{(2S)-5-oxo-4-[4-(2-pyridinyl)benzyl]tetrahydro-2-furanyl}-2-phenylethylcarbamate

A solution of tert-Butyl(1S)-1-[(2R)-oxiran-2-yl]-2-phenylethylcarbamate (10.0 g, 38.0 mmol) and diethyl malonate (5.8 ml, 38.2 mmol) in ethanol (30 mL) at 0° C. was treated with sodium ethoxide (17 mL, 21% in ethanol) over 10 minutes. The reaction was warmed to 25° C. and stirred for 2 hours, treated with additional diethyl malonate (0.58 mL, 3.4 mmol) and stirred for 1 hour. The reaction was cooled to 0° C., and solid 2-[4-(bromomethyl)phenyl]pyridine (9.43 g, 38.0 mmol) was added in four increments over 10 minutes. To this suspension was added ethanol (20 mL) and the mixture was stirred at 25° C. for 16 hours. The reaction mixture was treated with LiOH monohydrate (4.6 g, 109.6 mmol) solution in water (30 mL), stirred at 25° C. for 16 hours, cooled to 0° C., adjusted to pH 5 by addition of 4N HCl and partitioned between dichloromethane and water. The organic phase was washed with brine and dried over MgSO₄, filtered and concentrated. A solution of the concentrate in toluene (100 mL) was heated at reflux for 16 hours, cooled to 25° C. and concentrated to afford the title compound (21.4 g).

### EXAMPLE 1B

### (4S,5S)-5-[(tert-butoxycarbonyl)amino]-4-{[tert-butyl(dimethyl)silyl]oxy}-6-phenyl-2-[4-(2-pyridinyl)benzyl]hexanoic Acid

A solution of the product from Example 1A (21.4 g) in dioxane (100 mL) was treated with sodium hydroxide solution (57 mL, 1N), stirred at 25° C. for 30 minutes and concentrated. The concentrate was cooled to 0° C., and acidified to pH 5 with 4N HCl. The mixture was partitioned between dichloromethane and water, and the organic phase layer was washed with brine, dried over MgSO₄, filtered and concentrated. A solution of the residue in N,N-dimethylformamide (100 mL) was treated with imidazole (21 g, 308.5 mmol) and t-butyldimethylsilyl chloride (23 g, 152.6 mmol), stirred at 25° C. for 16 hours and concentrated. The residue was combined with ice and acidified with 4N HCl to pH 3. Ethyl acetate (50 mL) was added to permit stirring during the acidification. The mixture was extracted with ethyl acetate, washed with brine, dried over MgSO₄, filtered, and concentrated. The residue was chromatographed on silica gel eluting with a gradient of 20%-100% ethyl acetate in chloroform, followed by elution with 5% methanol in ethyl acetate to give the title product (11.3 g, 49% yield).

### EXAMPLE 1C

### Benzyl(1S,3S,4S)-4-[(tert-butoxycarbonyl)amino]-3-hydroxy-5-phenyl-1-[4-(2-pyridinyl)benzyl]pentylcarbamate

A solution of the product from Example 1B (11.3 g, 18.7 mmol) in toluene (190 mL) was treated with DPPA (8.1 mL, 37.6 mmol) and triethylamine (5.2 mL, 37.3 mmol), heated at reflux for 2 hours, treated with benzyl alcohol (5.8 mL, 56.0 mmol), heated at reflux for an additional 16 hours, cooled to 25° C. and concentrated. The residue was treated with a solution of TBAF in THF (94 mL, 1N), stirred at 25° C. for 40 hours and concentrated. The mixture was partitioned between ethyl acetate and water. The organic phase was washed with brine, dried over MgSO₄, filtered and concentrated. The residue was chromatographed on silica gel eluting with 50% ethyl acetate in chloroform to give 4.2 g (38% yield) of the lower Rf product by TLC (35% ethyl acetate in dichloromethane).

### EXAMPLE 1D

### Benzyl(1R,3S,4S)-4-[(tert-butoxycarbonyl)amino]-3-hydroxy-5-phenyl-1-[4-(2-pyridinyl)benzyl]pentylcarbamate

A solution of the product from Example 1B (11.3 g, 18.7 mmol) in toluene (190 mL) was treated with DPPA (8.1 mL, 37.6 mmol) and triethylamine (5.2 mL, 37.3 mmol), heated at reflux for 2 hours, treated with benzyl alcohol (5.8 mL, 56.0 mmol), heated at reflux for an additional 16 hours, cooled to 25° C. and concentrated. The residue was treated with a solution of tetrabutylammonium fluoride in THF (94 mL, 1N), stirred at 25° C. for 40 hours and concentrated. The mixture was partitioned between ethyl acetate and water. The organic phase was washed with brine, dried over MgSO₄, filtered and concentrated. The residue was chromatographed on silica gel eluting with 50% ethyl acetate in chloroform to give 2.6 g (23% yield) of the higher Rf product by TLC (35% ethyl acetate in dichloromethane).

### EXAMPLE 1E

### tert-butyl(1S,2S,4R)-4-amino-1-benzyl-2-hydroxy-5-[4-(2-pyridinyl)phenyl]pentylcarbamate

A solution of the product from Example 1D (2.6 g, 4.4 mmol) in a mixture of methanol (22 mL) and ethyl acetate (22 mL) was treated with Pd(OH)₂ on carbon (0.8 g, 20% Pd by wt.) and HCl solution (1.0 mL, 4N in dioxane), stirred under a hydrogen atmosphere (balloon pressure) at 25° C. for 16 hours, filtered through celite®, rinsed with methanol and concentrated to give the title product (1.7 g) as the hydrochloride salt.

### EXAMPLE 1F

### (2S)-2-[(methoxycarbonyl)amino]-3,3-dimethylbutanoic Acid

A solution of L-tert-Leucine (25 g, 190.58 mmol) in a mixture of dioxane (100 mL) and aqueous NaOH solution (315 mL, 2N) was treated dropwise with methyl chloroformate (29.3 mL, 379.19 mmol), keeping the internal temperature below 50° C. The mixture was warmed to 60° C. and stirred for 18 hours, cooled to 25° C. and extracted with dichloromethane. The aqueous phase was cooled to 0° C. and the pH was adjusted to about 1-2 with concentrated HCl. The mixture was partitioned between ethyl acetate and water. The combined organic extracts were washed with brine, dried over MgSO₄, filtered and concentrated. A solution of the concentrate in ether was treated with hexanes to afford the crystalline product (33.22 g, 92% yield), which was collected by filtration.

### EXAMPLE 1G

### tert-butyl(1S,2S,4R)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate

A solution of the product of Example 1E (1.7 g) in THF (33 mL) was treated with the product of Example 1F (0.81 g, 4.3 mmol), DEPBT (1.5 g, 5.0 mmol), and N,N-diisopropylethylamine (2.9 mL, 16.6 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (1.55 g, 74% yield).

### EXAMPLE 1H

### methyl(1S)-1-[({(1R,3S,4S)-4-amino-3-hydroxy-5-phenyl-1-[4-(2-pyridinyl)benzyl]pentyl}amino)carbonyl]-2,2-dimethylpropylcarbamate

A solution of the product of Example 1G (1.55 g, 2.45 mmol) in dichloromethane (12.5 mL) was treated with trifluoroacetic acid (12.5 mL), stirred at 25° C. for 1 hour and concentrated. The concentrate was partitioned between ethyl acetate and saturated NaHCO₃ solution. The organic phase extract was washed with brine, dried over MgSO₄, filtered and concentrated to give the title compound (1.4 g) which was used without further purification.

### EXAMPLE 1I

### methyl(1S,4R,6S,7S,10S)-7-benzyl-1,10-ditert-butyl-6-hydroxy-2,9,12-trioxo-4-[4-(2-pyridinyl)benzyl]-13-oxa-3,8,11-triazatetradec-1-ylcarbamate

A solution of the product of Example 1H (0.18 g, 0.33 mmol) in THF (3.3 mL) was treated with the product of Example 1F (0.11 g, 0.60 mmol), DEPBT (0.15 g, 0.50 mmol), and N,N-diisopropylethylamine (0.29 mL, 1.66 mmol), stirred at 25° C. for 16 hours, and partitioned between ethyl acetate and 10% Na₂CO₃ solution. The organic phase phase was washed with additional 10% Na₂CO₃ solution and brine, dried over MgSO₄, filtered and concentrated. The residue was chromatographed on silica gel eluting with 0%-75% ethyl acetate in chloroform to give the title product (0.19 g, 81% yield). ¹H NMR (300 MHz, DMSO-d6), δ ppm 0.75 (s, 9H), 0.78 (s, 9H), 1.28 (m, 2H), 1.55 (m, 1H), 2.70 (m, 4H), 3.55 (d, J=1.77 Hz, 6H), 3.85 (m, 3H), 4.15 (m, 1H), 4.80 (d, J=5.15 Hz, 1H), 6.75 (d, J=9.19 Hz, 1H), 6.86 (d, J=9.56 Hz, 1H), 7.13 (m, 5H), 7.22 (d, J=8.46 Hz, 2H), 7.32 (m, 1H), 7.52 (d, J=8.82 Hz, 1H), 7.88 (m, 5H), 8.64 (d, J=4.41 Hz, 1H).

### EXAMPLE 2A

### tert-butyl(1S,2S,4S)-4-amino-1-benzyl-2-hydroxy-5-[4-(2-pyridinyl)phenyl]pentylcarbamate

A solution of the product of Example 1C (4.2 g, 7.0 mmol) in a mixture of methanol (35 mL) and ethyl acetate (35 mL) was treated with Pd(OH)₂ on carbon (1.4 g, 20% Pd by wt.) and HCl solution (1.8 mL, 4N in dioxane), and the reaction was stirred under a hydrogen atmosphere (balloon pressure) for 16 hours at 25° C. The reaction mixture was filtered through a bed of celite®, rinsed with methanol and concentrated to give the title compound as the hydrochloride salt (3.7 g).

### EXAMPLE 2B

### tert-butyl(1S,2S,4S)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate

A solution of the product of Example 2A (3.7 g, 7.4) in THF (75 mL) was treated with the product from Example 1F (1.39 g, 7.4 mmol), DEPBT (3.3 g, 11.0 mmol), and N,N-diisopropylethylamine (6.4 mL, 36.7 mmol), stirred at 25° C. for 16 hours, and partitioned between ethyl acetate and 10% Na₂CO₃ solution. The organic phase phase was washed with additional 10% Na₂CO₃ solution and brine, dried over MgSO₄, filtered and concentrated. The residue was chromatographed on silica gel eluting with 33%-100% ethyl acetate in chloroform to give the title compound (3.5 g, 75% yield).

### EXAMPLE 2C

### methyl(1S)-1-[({(1S,3S,4S)-4-amino-3-hydroxy-5-phenyl-1-[4-(2-pyridinyl)benzyl]pentyl}amino)carbonyl]-2,2-dimethylpropylcarbamate

A solution of the product of Example 2B (3.5 g, 5.5 mmol) in dichloromethane (40 mL) was treated with trifluoroacetic acid (20 mL), stirred at 25° C. for 1 hour, and concentrated. The concentrate was partitioned between ethyl acetate and saturated NaHCO₃ solution. The organic phase phase was washed with brine, dried over MgSO₄, filtered and concentrated to afford the crude product (3.19 g).

### EXAMPLE 2D

### methyl(1S,4S,5S,7S,10S)-4-benzyl-1,10-ditert-butyl-5-hydroxy-2,9,12-trioxo-7-[4-(2-pyridinyl)benzyl]-13-oxa-3,8,11-triazatetradec-1-ylcarbamate

A solution of the product of Example 2C (1.6 g, 3.0 mmol) in THF (30 mL) was treated with the product of Example 1F (0.57 g, 3.0 mmol), DEPBT (1.35 g, 4.5 mmol), and N,N-diisopropylethylamine (2.6 mL, 14.9 mmol), stirred at 25° C. for 3 hours and partitioned between ethyl acetate and 10% Na₂CO₃ solution. The organic phase phase was washed with additional 10% Na₂CO₃ solution and brine, dried over MgSO₄, filtered and concentrated. The residue was chromatographed on silica gel eluting with 50% ethyl acetate in chloroform, followed by 5% methanol in chloroform to give the title compound (1.59 g, 75% yield). ¹H NMR (300 MHz, DMSO-d₆), δ ppm 0.79 (s, 9H), 0.82 (s, 9H), 1.51 (m, 2H), 2.72 (m, 3H), 3.49 (s, 3H), 3.55 (s, 3H), 3.63 (m, 1H), 3.82 (d, J=9.93 Hz, 1H), 3.90 (d, J=9.56 Hz, 1H), 4.04 (m, 3H), 4.86 (d, J=5.88 Hz, 1H), 6.60 (d, J=9.93 Hz, 1H), 6.78 (d, J=9.19 Hz, 1H), 7.16 (m, 7H), 7.31 (m, 1H), 7.54 (d, J=8.46 Hz, 1H), 7.83 (m, 5H), 8.63 (d, J=4.78 Hz, 1H).

### EXAMPLE 3A

### 9H-fluoren-9-ylmethyl(1S,2S,4S)-1-benzyl-2-hydroxy-4-[(tert-butoxycarbonyl)amino]-5-phenylpentylcarbamate

A solution of the product of Example 126 (1.0 g, 2.0 mmol) in a mixture of dioxane (15 mL) and water (5 mL) was treated with sodium bicarbonate (0.37 g, 4.4 mmol) and N-(9-fluorenylmethyloxycarbonyloxy)-succinimide (0.74 g, 2.2 mmol), stirred at 25° C. for 16 hours and partitioned between ethyl acetate and diluted sodium bicarbonate solution. The organic phase phase was washed with brine and dried over MgSO₄, filtered and concentrated to give the title compound (1.37 g), which was used without further purification.

### EXAMPLE 3B

### 9H-fluoren-9-ylmethyl(1S,2S,4S)-4-amino-1-benzyl-2-hydroxy-5-phenylpentylcarbamate

A solution containing the product of Example 3A (0.92 g, 1.5 mmol) in dioxane (5 mL) was treated with HCl solution (15 mL, 4. N in dioxane) at 0° C., stirred at 25° C. for 1 hour and concentrated. The residue was triturated with hexanes to give the title compound as the hydrochloride salt (0.82 g).

### EXAMPLE 3C

### methyl(1S)-1-{[((1S,3S,4S)-1-benzyl-4-{[(9H-fluoren-9-ylmethoxy)carbonyl]amino}-3-hydroxy-5-phenylpentyl)amino]carbonyl}-2,2-dimethylpropylcarbamate

A solution of the product of Example 3B (0.150 g, 0.276 mmol) in DMF (3 mL) were treated with the product of Example 1F (0.052 g, 0.275 mmol), EDAC (0.080 g, 0.417 mmol), HOBT (0.055, 0.407 mmol), and NMM (0.090 mL, 0.819 mmol) at 0° C., stirred at 25° C. for 2 hours, and partitioned between ethyl acetate and water. The organic phase was washed with 10% citric acid, diluted sodium bicarbonate, and brine, dried over MgSO₄, filtered and concentrated. The concentrate was purified by reversed phase chromatography on a C18 column, eluting with a gradient starting with 5%-100% acetonitrile in water (0.1% TFA) to give the title compound (0.130 g, 70% yield).

### EXAMPLE 3D

### methyl(1S)-1-({[(1S,3S,4S)-4-amino-1-benzyl-3-hydroxy-5-phenylpentyl]amino}carbonyl)-2,2-dimethylpropylcarbamate

A solution of the product of Example 3C (0.130 g, 0.192 mmol) in DMF (6 mL) was treated with diethylamine (1.5 mL), stirred at 25° C. for 1 hour, and partitioned between ethyl acetate and wate. The organic phase was washed with brine, dried over MgSO₄, filtered and concentrated. The residue was chromatographed on silica gel eluting with a gradient starting with ethyl acetate and ending with methanol to give the title compound (0.52 g, 60% yield).

### EXAMPLE 3E

### (1,3-dioxo-1,3-dihydro-2H-isoindol-2-yl)acetaldehyde

A solution of phthalimidoacetaldehyde diethyl acetal (39.6 g, 150.4 mmol) in a mixture of THF (80 mL) and aqueous HCl (50 mL, 10%) was heated at 75° C. for 5 hours, cooled to 25° C. and partitioned between ethyl acetate and half-saturated NaHCO₃. The organic phase phase was washed with brine, dried over MgSO₄, filtered and concentrated to give the title compound (36.81 g), which was used without further purification.

### EXAMPLE 3F

### tert-butyl(2S,3S)-2-{[2-(1,3-dioxo-1,3-dihydro-2H-isoindol-2-yl)ethyl]amino}-3-methylpentanoate

A solution of the product of Example 3E (36.81 g) in methanol (50 mL) was treated with L-iso-leucine tert-butyl ester hydrochloride (30 g, 134 mmol), sodium cyanoborohydride (16.9 g, 268 mmol), and acetic acid (4.6 ml, 80.4 mmol), stirred at 25° C. for 3 hours and concentrated. The concentrate was partitioned between dichloromethane and saturated NaHCO₃. The organic phase phase was washed with brine and dried over MgSO₄, filtered and concentrated. The residue was chromatographed on silica gel, eluting with a gradient starting with 10%-066% ethyl acetate in hexanes to give the title compound (28.44 g, 59% yield).

### EXAMPLE 3G

### tert-butyl(2S,3S)-2-[(2-aminoethyl)amino]-3-methylpentanoate

A solution of the product of Example 3F (28.44 g, 78.9 mmol) in ethanol (400 mL) was treated with hydrazine hydrate (25 mL, 789 mmol), stirred at 70° C. for 2 hours, cooled to 25° C. The solid precipitate was dissolved by addition of aqueous NaOH solution (200 mL, 1 N). The reaction was partitioned between dichloromethane and water. The aqueous was extracted three times with dichloromethane. The combined organic extracts were dried over MgSO₄, filtered and concentrated to give the title compound (15.4 g, 85% yield), which was used without further purification.

### EXAMPLE 3H

### 2-pyridinecarbothioamide

A solution of pyridine-2-carboxamide (3.1 g, 25.4 mmol) in toluene (25 mL) was treated with Laweson's reagent (5.1 g, 12.6 mmol), heated at 85° C. for 64 hours, cooled to 25° C., and partitioned between ethyl acetate and water. The organic phase phase was washed with brine, dried over MgSO₄, filtered and concentrated to give the title compound, which was used without further purification.

### EXAMPLE 3I

### Ethyl 2-(2-pyridinyl)-1,3-thiazole-4-carboxylate

A solution of the product of Example 3H (25.4 mmol) in ethanol (50 mL) was treated with ethyl bromopyruvate (3 mL, 23.9 mmol) and molecular sieves (10 g, 3. A), heated at reflux for 16 hours, cooled to 25° C., filtered and concentrated. The concentrate was partitioned between ethyl acetate and saturated NaHCO₃, and the organic phase phase was washed with brine, dried over MgSO₄, filtered and concentrated. The residue was chromatographed on silica gel eluting with 0-25% ethyl acetate in dichloromethane to give the title compound (1.98 g, 33% yield).

### EXAMPLE 3J

### 2-(2-pyridinyl)-1,3-thiazole-4-carbaldehyde

A solution containing the product of Example 31 (0.91 g, 3.9 mmol) in dichloromethane (13 mL) was treated dropwise with DIBAL (7.4 mL, 1 M in dichloromethane) at −78° C., stirred at −78° C. for 1 hour, treated with acetic acid (0.8 mL) and warmed to 25° C. A 10% solution of aqueous sodium potassium tartrate was treated with and the mixture was stirred vigorously for 1 hour. The reaction mixture was partitioned between chloroform and water, and the organic phase phase was washed with brine, dried over MgSO₄, filtered and concentrated. The residue was chromatographed on silica gel eluting with with 0-10% ethyl acetate in dichloromethane to give the title compound (0.39 g, 53% yield).

### EXAMPLE 3K

### tert-butyl(2S,3S)-3-methyl-2-(2-oxo-3-{[2-(2-pyridinyl)-1,3-thiazol-4-yl]methyl}-1-imidazolidinyl)pentanoate

A solution containing the product of Example 3G (0.30 g, 1.30 mmol) in a mixture of benzene (3 mL) and ethanol (3 mL) was treated with the product of Example 3J (0.25 g, 1.31 mmol), heated at 70° C. for 16 hours, cooled to 25° C., treated with sodium borohydride (0.15 g, 3.97 mmol), stirred at 25° C. for 3 hours, quenched with sodium bicarbonate solution and partitioned between ethyl acetate and water. The organic phase phase was washed with brine, dried over MgSO₄, filtered and concentrated. A solution of the residue (1.3 mmol) in 1,2-dichloroethane (50 mL) was treated with bis(4-nitrophenyl)carbonate (0.425 g, 1.40 mmol) and triethylamine (0.225 ml, 1.83 mmol), heated at 70° C. for 16 hours, and partitioned between ethyl acetate and saturated NaHCO₃. The organic phase phase was washed with brine, dried over MgSO₄, filtered and concentrated. The residue was chromatographed on silica gel eluting with 0-35% ethyl acetate in dichloromethane to give the title compound (0.214 g, 38% yield).

### EXAMPLE 3L

### (2S,3S)-3-methyl-2-(2-oxo-3-{[2-(2-pyridinyl)-1,3-thiazol-4-yl]methyl}-1-imidazolidinyl)pentanoic Acid Trifluoroacetate

A solution containing the product of Example 3K (0.214 g, 0.50 mmol) in dichloromethane (2-mL) was treated with trifluoracetic acid (2 mL), was stirred at 25° C. for 1 hour and concentrated. The residue was chromatographed on silica gel eluting with 0-15% methanol in dichloromethane to give the title compound (0.24 g) as the trifluoroacetic acid salt.

### EXAMPLE 3M

### methyl(1S)-1-{[((1S,3S,4S)-1-benzyl-3-hydroxy-4-{[(2S)-3-methyl-2-(2-oxo-3-{[2-(2-pyridinyl)-1,3-thiazol-4-yl]methyl}-1-imidazolidinyl)pentanoyl]amino}-5-phenylpentyl)amino]carbonyl}-2,2-dimethylpropylcarbamate

A solution containing the product from Example 3D (0.025 g, 0.055 mmol) in DMF (0.5 mL) was treated with the product from Example 3L (0.021 g, 0.056 mmol), EDAC (0.020 g, 0.104 mmol), HOBT (0.015 g, 0.111 mmol), and NMM (0.020 mL, 0.182 mmol) at (0° C., stirred at 25° C. for 16 hours, and partitioned between ethyl acetate and water. The organic phase phase was washed with 10% citric acid, diluted sodium bicarbonate, and brine, dried over MgSO₄, filtered and concentrated. The residue was purified by reversed phase chromatography on a C18 column, eluting with 5-100% acetonitrile in water (0.1% TFA) to give the title compound (0.028 g, 63% yield). ¹H NMR (300 MHz, DMSO-d₆), δ ppm 0.79 (m, 15H), 0.95 (m, 1H), 1.29 (m, 1H), 1.49 (m, 2H), 1.80 (m, 1H), 2.68 (m, 4H), 3.03 (m, 1H), 3.17 (m, 2H), 3.55 (s, 3H), 3.63 (m, 3H), 3.94 (d, J=11.03 Hz, 2H), 4.12 (m, 2H), 4.47 (m, 2H), 6.62 (d, J=9.93 Hz, 1H), 7.07 (m, 10H), 7.25 (d, J=9.56 Hz, 1H), 7.48 (m, 1H), 7.57 (s, 1H), 7.75 (d, 8.46 Hz, 1H), 7.93 (n, 1H), 8.10 (d, J=8.09 Hz, 1H), 8.62 (d, J=4.04 Hz, 1H).

### EXAMPLE 4A

### (2S,3S)-3-methyl-2-[2-oxo-3-(4-quinolinylmethyl)-1-imidazolidinyl]pentanoic Acid Trifluoroacetate

A solution containing the product from Example 3G (2.0 g, 8.69 mmol) in a mixture of benzene (40 mL) and ethanol (40 mL) was treated with 4-quinolinecarboxaldehyde (1.4 g, 8.91 mmol), heated at 70° C. for 2 hours, cooled to 25° C., treated with sodium borohydride (1.0 g, 26.75 mmol), stirred at 25° C. for 16 hours, quenched with sodium bicarbonate solution and partitioned between ethyl acetate and water. The organic phase phase was washed with brine, dried over MgSO₄, filtered and concentrated. A solution of the residue (8.69 mmol) in 1,2-dichloroethane (300 mL) was treated with bis(4-nitrophenyl)carbonate (3.0 g, 9.86 mmol), heated at 70° C. for 16 hours, and partitioned between ethyl acetate and saturated NaHCO₃. The organic phase phase was washed with brine, dried over MgSO₄ filtered and concentrated. A solution of the residue (8.69 mmol) in dichloromethane (40 mL) was treated with trifluoracetic acid (40 mL), stirred at 25° C. for 1 hour and concentrated. The residue was chromatographed on silica gel eluting with 0-5% methanol in dichloromethane. A seond purification using reversed phase chromatography on a C18 column, eluting with 5-100% acetonitrile in water (0.1% TFA) afforded the title compound (1.91 g, 48% yield).

### EXAMPLE 4B

### methyl(1S)-1-({[(1S,3S,4S)-1-benzyl-3-hydroxy-4-({(2S)-3-methyl-2-[2-oxo-3-(4-quinolinylmethyl)-1-imidazolidinyl]pentanoyl}amino)-5-phenylpentyl]amino}carbonyl)-2,2-dimethylpropylcarbamate

A solution containing the product from Example 3D (0.078 g, 0.171 mmol) in DMF (0.5 mL) was treated with the product from Example 4A (0.070 g, 0.205 mmol), EDAC (0.050 g, 0.261 mmol), HOBT (0.035 g, 0.259 mmol), and NMM (0.060 mL, 0.546 mmol) at 0° C., stirred at 25° C. for 16 hours and partitioned between ethyl acetate and water. The organic phase phase was washed with 10% citric acid, diluted sodium bicarbonate, and brine, dried over MgSO₄, filtered and concentrated. The residue was purified by reversed phase chromatography on a C18 column, eluting with 5-100% acetonitrile in water (0.1% TFA) to give the title compound (0.030 g, 23% yield). ¹H NMR (300 MHz, DMSO-d6), δ ppm 0.79 (m, 15H), 1.00 (m, 1H), 1.25 (m, 1H), 1.51 (m, 2H), 1.82 (m, 1H), 2.64 (m, 5H), 3.02 (m, 3H), 3.55 (s, 3H), 3.63 (m, 1H), 3.82 (d, J=9.93 Hz, 1H), 3.99 (d, J=11.03 Hz, 1H), 4.13 (m, 2H), 4.64 (d, J=7.72 Hz, 1H), 4.80 (m, 2H), 6.62 (d, J=9.93 Hz, 1H), 6.98 (m, 5H), 7.14 (m, 5H), 7.32 (d, J=9.56 Hz, 1H), 7.41 (d, J=4.41 Hz, 1H), 7.62 (t, J=7.54 Hz, 1H), 7.76 (m, 2H), 8.06 (d, J=7.72 Hz, 1H), 8.30 (d, J=8.46 Hz, 1H), 8.88 (d, J=4.41 Hz, 1H).

### EXAMPLE 5A

### (2S,3S)-2-[(methoxycarbonyl)amino]-3-methylpentanoic Acid

A solution of L-iso-Leucine (7.43 g, 56.6 mmol) in a mixture of dioxane (28 mL) and aqueous NaOH solution (93.5 mL, 2N) was treated with methyl chloroformate (8.75 mL, 113.3 mmol) dropwise, not allowing the internal temperature to rise above 50° C. The mixture was warmed to 60° C. and stirred for 18 hours, cooled to 25° C., and extracted with dichloromethane. The aqueous phase was cooled to 0° C., adjusted its pH to 1-2 with HCl (4 N). The mixture was partitioned between ethyl acetate and water, and the organic phase was washed with brine, dried over Na₂SO₄, filtered and concentrated to give the crude product (10 g).

### EXAMPLE 5B

### methyl(1S,2S)-1-{[((1S,3S,4S)-1-benzyl-4-{[(9H-fluoren-9-ylmethoxy)carbonyl]amino}-3-hydroxy-5-phenylpentyl)amino]carbonyl}-2-methylbutylcarbamate

A solution containing the product from Example 3B (0.150 g, 0.276 mmol) in DMF (3 mL) was treated with the product from Example 5A (0.063 g, 0.333 mmol), EDAC (0.080 g, 0.417 mmol), HOBT (0.055, 0.407 mmol), and NMM (0.090 mL, 0.819 mmol) at 0° C., stirred at 25° C. for 16 hours, partitioned between ethyl acetate and water. The organic phase was washed with 10% citric acid, dilute sodium bicarbonate solution, and brine, dried over MgSO₄, filtered and concentrated. The concentrate was purified by reversed phase chromatography on a C18 column, eluting with 5%-100% acetonitrile in water (0.1% TFA) to give the title compound (0.107 g, 57% yield).

### EXAMPLE 5C

### methyl(1S,2S)-1-({[(1S,3S,4S)-4-amino-1-benzyl-3-hydroxy-5-phenylpentyl]amino}carbonyl)-2-methylbutylcarbamate

A solution containing the product from Example 5B (0.107 g, 0.158 mmol) in DMF (6 mL) was treated with diethylamine (1.5 mL), stirred at 25° C. for 1 hour, and partitioned between ethyl acetate and water. The organic phase phase was washed with brine, dried over MgSO₄, filtered and concentrated to give the title compound, which was used without further purification.

### EXAMPLE 5D

### methyl(1S)-1-({[(1S,3S,4S)-1-benzyl-3-hydroxy-4-({(2S)-3-methyl-2-[2-oxo-3-(4-quinolinylmethyl)-1-imidazolidinyl]pentanoyl}amino)-5-phenylpentyl]amino 3 carbonyl)-2-methylbutylcarbamate

A solution containing the product from Example 5C (0.078 g, 0.171 mmol) in DMF (0.5 mL) was treated with the product from Example 4A (0.070 g, 0.205 mmol), EDAC (0.050 g, 0.261 mmol), HOBT (0.035 g, 0.259 mmol), and NMM (0.060 mL, 0.546 mmol) at (0° C., stirred at 25° C. for 16 hours, and partitioned between ethyl acetate and water. The organic phase was washed with 10% citric acid, diluted sodium bicarbonate, and brine, dried over MgSO₄, filtered and concentrated. The residue was purified by reversed phase chromatography on a C18 column eluting with 5-100% acetonitrile in water (0.1% TFA) to give the title compound (0.030 g, 23% yield). ¹H NMR (300 MHz, DMSO-d₆), δ ppm 0.76 (m, 12H), 1.00 (m, 2H), 1.29 (m, 2H), 1.49 (m, 3H), 1.81 (m, 1H), 2.65 (m, 5H), 3.01 (m, 3H), 3.54 (s, 3H), 3.61 (m, 1H), 3.75 (t, J=8.82 Hz, 1H), 3.99 (d, J=11.03 Hz, 1H), 4.13 (m, 2H), 4.62 (d, J=7.35 Hz, 1H), 4.80 (m, 2H), 6.89 (d, J=9.56 Hz, 1H), 6.98 (m, 5H), 7.15 (m, 5H), 7.32 (d, J=9.93 Hz, 1H), 7.41 (d, J=4.41 Hz, 1H), 7.66 (m, 2H), 7.77 (t, J=6.99 Hz, 1H), 8.06 (d, J=7.72 Hz, 1H), 8.30 (d, J=8.09 Hz, 1H), 8.88 (d, J=4.41 Hz, 1H).

### EXAMPLE 6A

### tert-butyl(1S,3S,4S)-4-amino-1-benzyl-3-hydroxy-5-phenylpentylcarbamate

The product from Example 126 (20 g, 39.8 mmol) was partitioned between ethyl acetate and saturated NaHCO₃ solution with stirring for 30 minutes. The solid white amine was collected by filtration and the aqueous was extracted twice with portions of ethyl acetate. The solid material collected was dissolved in warm ethyl acetate and this solution was combined with the organic phase extracts, dried over sodium sulfate, filtered and concentrated to give the free amine (14.15 g).

### EXAMPLE 6B

### 2-methoxyethanethioamide

A solution containing methoxyacetyl chloride (10 g, 92.15 mmol) and ammonium acetate (7.1 g, 92.11 mmol) in acetone (250 mL) was stirred at 25° C. for 16 hours, treated with phosphorous pentasulfide (4.1 g, 9.22 mmol), stirred at 25° C. for 64 hours, concentrated and partitioned between ethyl acetate and water. The organic phase phase was washed with brine, dried over MgSO₄, filtered and concentrated to give the title compound (7.0 g, 72% yield), which was used without further purification.

### EXAMPLE 6C

### Ethyl 2-(methoxymethyl)-1,3-thiazole-4-carboxylate

A solution containing the product from Example 6B (7 g, 66.6 mmol) in acetone (270 mL) was treated with ethyl bromopyruvate (8.4 mL, 66.6 mmol) and magnesium sulfate (7.9 g, 66.6 mmol), heated at reflux for 16 hours, cooled to 25° C., filtered and concentrated. The residue was chromatographed on silica gel eluting with chloroform to give the title compound (7.6 g, 57% yield).

### EXAMPLE 6D

### 2-(methoxymethyl)-1,3-thiazole-4-carbaldehyde

A solution containing the product from Example 6C (7.4 g, 36.8 mmol) in dichloromethane (40 mL) was treated with DIBAL (73.6 mL, 1 M in dichloromethane) dropwise at −78° C. over 2 hours, stirred at −78° C. for 2 hours, treated with acetic acid (10 mL) at −78° C. and warmed to 25° C. A 10% solution of aqueous sodium potassium tartrate was treated with and the mixture was stirred vigorously for 1 hour. The reaction mixture was partitioned between chloroform and water. The organic phase phase was washed with brine, dried over MgSO₄, filtered and concentrated. The residue was chromatographed on silica gel eluting with 0-100% ethyl acetate/dichloromethane to give the title compound (5.78 g, 71% yield).

### EXAMPLE 6E

### tert-butyl(2S)-2-{[2-(1,3-dioxo-1,3-dihydro-2H-isoindol-2-yl)ethyl]amino}-3,3-dimethylbutanoate

A solution of the product of Example 3E (9.34 g, 49.4 mmol) in methanol (33 mL) was treated with L-tert-leucine tert-butyl ester hydrochloride (10 g, 44.9 mmol), sodium cyanoborohydride (5.6 g, 89.8 mmol), and acetic acid (1.5 ml, 26.2 mmol), stirred at 25° C. for 4 hours, and partitioned between chloroform and saturated NaHCO₃. The organic phase phase was washed with brine, dried over MgSO₄, filtered and concentrated. The residue was chromatographed in silica gel, eluting with first with 66% chloroform in hexanes and then with 33% ethyl acetate in chloroform to give the title compound (10.5 g, 59% yield).

### EXAMPLE 6F

### tert-butyl(2S)-2-[(2-aminoethyl)amino]-3,3-dimethylbutanoate

A solution of the product from Example 6E (10.5 g, 29.1 mmol) in ethanol (290 mL) was treated with hydrazine hydrate (9 mL, 290 mmol), heated at 70° C. for 2 hours and cooled to 25° C. The solid precipitate was dissolved by addition of aqueous NaOH solution (150 mL, 1 N). The reaction mixture was partitioned between chloroform and water, and the aqueous was extracted three times with chloroform. The combined organic extracts were dried over MgSO₄, filtered and concentrated to give the diamine (7.0 g, quantitative), which was used without further purification.

### EXAMPLE 6G

### tert-butyl(2S)-2-(3-{[2-(methoxymethyl)-1,3-thiazol-4-yl]methyl}-2-oxo-1-imidazolidinyl)-3,3-dimethylbutanoate

A solution containing the product from Example 6F (1.0 g, 4.34 mmol) in a mixture of benzene (12 mL) and ethanol (12 mL) was treated with the product from Example 6D (0.682 g, 4.34 mmol), heated at 50° C. for 1.5 hours, cooled to 25° C., treated with sodium borohydride (0.329 g, 8.68 mmol), stirred at 25° C. for 1.5 hours, quenched with sodium bicarbonate solution, and partitioned between ethyl acetate and water. The organic phase phase was washed with brine, dried over MgSO₄, filtered and concentrated. A solution of the residue (4.34 mmol) in toluene (25 mL) was treated with bis(4-nitrophenyl)carbonate (1.58 g, 5.21 mmol), heated at 60° C. for 16 hours, and partitioned between ethyl acetate and saturated NaHCO₃. The organic phase phase was washed with brine, dried over MgSO₄, filtered and concentrated to give the title compound (1.28 g, 74% yield), which was used without further purification.

### EXAMPLE 6H

### (2S)-2-(3-{[2-(methoxymethyl)-1,3-thiazol-4-yl]methyl}-2-oxo-1-imidazolidinyl)-3,3-dimethylbutanoic Acid Trifluoroacetate

A solution containing the product from Example 6G (1.28 g, 3.2 mmol) in dichloromethane (10 mL) was treated with trifluoracetic acid (5 mL), stirred at 25° C. for 4 hours and concentrated. The residue was chromatographed on silica gel eluting with 0-5% methanol in dichloromethane to give the title compound (1.2 g) as the trifluoroacetic acid salt.

### EXAMPLE 6I

### tert-butyl(1S,3S,4S)-1-benzyl-3-hydroxy-4-{[(2S)-2-(3-{[2-(methoxymethyl)-1,3-thiazol-4-yl]methyl}-2-oxo-1-imidazolidinyl)-3,3-dimethylbutanoyl]amino}-5-phenylpentylcarbamate

A solution of the product from Example 6A (0.034 g, 0.089 mmol) in THF (0.9 mL) was treated with the product from Example 6H (0.035 g, 0.103 mmol), DEPBT (0.040 g, 0.134 mmol), and N,N-diisopropylethylamine (0.075 mL, 0.431 mmol), stirred at 25° C. for 16 hours, and partitioned between ethyl acetate and 10% Na₂CO₃ solution. The organic phase phase was washed with additional 10% Na₂CO₃ solution and brine, dried over MgSO₄, filtered and concentrated. The residue was purified by reversed phase chromatography using C18 column, eluting with 5-100% acetonitrile in water (0.1% TFA) to give the title compound (0.047 g, 75% yield).

### EXAMPLE 6J

### methyl(1S)-1-{[((1S,3S,4S)-1-benzyl-3-hydroxy-4-{[(2S)-2-(3-{[2-(methoxymethyl)-1,3-thiazol-4-yl]methyl}-2-oxo-1-imidazolidinyl)-3,3-dimethylbutanoyl]amino}-5-phenylpentyl)amino]carbonyl}-2,2-dimethylpropylcarbamate

A solution containing the product from Example 61 (0.047 g, 0.066 mmol) in dichloromethane (1 mL) was treated with trifluoroacetic acid (1 mL), stirred at 25° C. for 1 hour, concentrated, and partitioned between ethyl acetate and saturated NaHCO₃. The organic phase phase was washed with brine and dried over MgSO₄, filtered and concentrated. A solution of the residue (0.030 g, 0.049 mmol) in DMF (0.5 mL) was treated with the product from Example 1F (0.010 g, 0.053 mmol), EDAC (0.020 g, 0.104 mmol), HOBT (0.015 g, 0.111 mmol), and NMM (0.016 mL, 0.146 mmol) at 0° C., stirred at 25° C. for 16 hours, and partitioned between ethyl acetate and water. The organic phase phase was washed with 10% citric acid, diluted sodium bicarbonate, and brine, and dried over MgSO₄, filtered and concentrated. The residue was purified by reversed phase chromatography on a C18 column, eluting with 5-100% acetonitrile in water (0.1% TFA) to give the title compound (0.031 g, 79% yield). ¹H NMR (300 MHz, DMSO-d₆), δ ppm 0.82 (s, 9H), 0.89 (s, 9H), 1.25 (m, 1H), 1.50 (m, 2H), 2.37 (m, 1H), 2.65 (d, J=7.35 Hz, 2H), 2.73 (d, J=9.56 Hz, 1H), 3.02 (m, 2H), 3.19 (m, 1H), 3.38 (s, 3H), 3.55 (s, 3H), 3.85 (m, 3H), 4.08 (m, 3H), 4.38 (m, 2H), 4.68 (s, 2H), 6.61 (d, J=9.93 Hz, 1H), 7.08 (m, 10H), 7.43 (m, 2H), 7.74 (d, J=8.46 Hz, 1H).

### EXAMPLE 7A

### tert-butyl(2S,3S)-3-methyl-2-{3-[(6-methyl-2-pyridinyl)methyl]-2-oxo-1-imidazolidinyl}pentanoate

A solution containing the product from Example 3G (6.18 g, 26.9 mmol) in dichloromethane (160 mL) was treated with 6-methyl-2-pyridinecarboxaldehyde (3.25 g, 26.8 mmol) and magnesium sulfate (16.3 g, 135.4 mmol) g), stirred at 25° C. for 18 hours, filtered and concentrated. A solution of the residue in methanol (160 mL) was treated with sodium borohydride (1.2 g, 31.7 mmol), stirred at 25° C. for 1 hour, quenched with water, stirred for 15 minutes, and followed by evaporation of the solvent. The concentrate was partitioned between ethyl acetate and saturated NaHCO₃, and the organic phase was washed with brine and dried over MgSO₄, filtered and concentrated. A solution of the residue (9.1 g, 26.8 mmol) in 1,2-dichloroethane (550 mL) was treated with N,N-disuccinimidyl carbonate (8.24 g, 32.2 mmol) and triethylamine (3.7 mL, 26.5 mmol), stirred at 25° C. for 68 hours, partitioned with 10% Na₂CO₃, and the organic phase was washed with additional 10% Na₂CO₃ solution and brine, dried over MgSO₄, filtered and concentrated. The residue was chromatographed on silica gel eluting with 0-100% ethyl acetate/dichloroform to give the title compound (6.15 g, 63% yield).

### EXAMPLE 7B

### (2S,3S)-3-methyl-2-{3-[(6-methyl-2-pyridinyl)methyl]-2-oxo-1-imidazolidinyl}pentanoic Acid

A solution containing the product from Example 7A (6.15 g, 17.0 mmol) in dichloromethane (150 mL) was treated with trifluoracetic acid (50 mL), stirred at 25 C for 2 hours and concentrated. The residue was purified by reversed phase chromatography on a C18 column, eluting with 5-100% acetonitrile in water (0.1% TFA) to give the title compound (6.0 g, 84% yield) as the trifluoroacetic acid salt.

### EXAMPLE 7C

### tert-butyl(1S,3S,4S)-1-benzyl-3-hydroxy-4-[((2S,3S)-3-methyl-2-{3-[(6-methyl-2-pyridinyl)methyl]-2-oxo-1-imidazolidinyl}pentanoyl)amino]-5-phenylpentylcarbamate

A solution of the product from Example 6A (0.046 g, 0.119 mmol) in THF (0.9 mL) was treated with the product from Example 7B (0.050 g, 0.119 mmol), EDAC (0.035 g, 0.183 mmol), HOBT (0.025 g, 0.185 mmol), and NMM (0.040 mL, 0.364 mmol) at 0° C., stirred at 25° C. for 16 hours, and partitioned between ethyl acetate and water. The organic phase phase was washed with 10% citric acid, dilute sodium bicarbonate solution, and brine, dried over MgSO₄, filtered and concentrated. The residue was purified by reversed phase chromatography on a C18 column, eluting with 5-100% acetonitrile in water (0.1% TFA) to give the title compound (0.080 g, 100% yield).

### EXAMPLE 7D

### methyl(1S)-1-[({(1S,3S,4S)-1-benzyl-3-hydroxy-4-[((2S)-3-methyl-2-{3-[(6-methyl-2-pyridinyl)methyl]-2-oxo-1-imidazolidinyl}pentanoyl)amino]-5-phenylpentyl}amino)carbonyl]-2,2-dimethylpropylcarbamate

A solution containing the product from Example 7C (0.080 g, 0.119 mmol) in dichloromethane (1 mL) was treated with trifluoroacetic acid (1 mL), stirred at 25 C for 1 hour and concentrated. The concentrate was partitioned between ethyl acetate and saturated NaHCO₃. The organic phase phase was washed with brine, dried over MgSO₄, filtered and concentrated. A solution of the residue (0.056 g, 0.098 mmol) in DMF (1 mL) was treated with the product from Example 1F (0.020 g, 0.106 mmol), EDAC (0.030 g, 0.156 mmol), HOBT (0.020 g, 0.148 mmol), and NMM (0.030 mL, 0.273 mmol) at 0° C., stirred at 25° C. for 16 hours, and partitioned between ethyl acetate and water. The organic phase phase was washed with 10% citric acid, dilute sodium bicarbonate, and brine, and dried over MgSO₄ filtered and concentrated. The residue was purified by reversed phase chromatography on a C18 column, eluting with 5-100% acetonitrile in water (0.1% TFA) to give the title compound (0.049 g, 67% yield). ¹H NMR (300 MHz, DMSO-d₆), δ ppm 0.79 (m, 15H), 0.93 (m, 2H), 1.31 (m, 1H), 1.50 (m, 2H), 1.82 (m, 1H), 2.45 (s, 3H), 2.67 (m, 4H), 3.06 (m, 3H), 3.55 (s, 3H), 3.64 (m, 1H), 3.82 (d, J=9.93 Hz, 1H), 3.93 (d, J=11.03 Hz, 1H), 4.13 (m, 2H), 4.35 (s, 2H), 4.64 (d, J=7.35 Hz, 1H), 6.62 (d, J=9.93 Hz, 1H), 7.02 (d, J=7.72 Hz, 1H), 7.13 (m, 11H), 7.26 (d, J=9.93 Hz, 1H), 7.66 (t, J=7.72 Hz, 1H), 7.75 (d, J=8.46 Hz, 1H).

### EXAMPLE 8A

### methyl(1S)-1-[({(1S,2S,4S)-1-benzyl-4-[(tert-butoxycarbonyl)amino]-2-hydroxy-5-phenylpentyl}amino)carbonyl]-2,2-dimethylpropylcarbamate

A solution of the product from Example 1F (7.0 g, 37.0 mmol), EDAC (8.5 g, 44.3 mmol), HOBT (6.0 g, 44.4 mmol), and NMM (8.0 mL, 72.8 mmol) in DMF (30 mL) was stirred at 25° C. for 1 hour, treated with a solution of the product from Example 6A (14.15 g, 36.8 mmol) in DMF (30 mL), stirred at 25° C. for 16 hours, concentrated, and partitioned between ethyl acetate and saturated NaHCO₃. The organic phase was washed with saturated NaHCO₃ and brine, and concentrated. The solution of the residue in hot methanol (20 mL) and water (10 mL) was allowed to cool and stand for 16 hours. The solids were collected by filtration and rinsed several times with hexanes, followed by drying under vacuum to give the title compound (16.97 g, 77% yield).

### EXAMPLE 8B

### methyl(1S)-1-({[(1S,2S,4S)-4-amino-1-benzyl-2-hydroxy-5-phenylpentyl]amino}carbonyl)-2,2-dimethylpropylcarbamate

A solution containing the product from Example 8A (16.97 g, 30.6 mmol) in THF (150 mL) was treated with HCl solution (50 mL, 4 N in dioxane), stirred at 60° C. for 2 hours, cooled and adjusted to pH 8 with 10% NaOH solution. The reaction mixture was partitioned between ethyl acetate and water, and the organic phase phase was washed with brine and concentrated to give the title compound (13.74 g).

### EXAMPLE 8C

### methyl(1S)-1-[({(1S,2S,4S)-1-benzyl-2-hydroxy-4-[((2S)-3-methyl-2-{3-[(6-methyl-2-pyridinyl)methyl]-2-oxo-1-imidazolidinyl}pentanoyl)amino]-5-phenylpentyl}amino)carbonyl]-2,2-dimethylpropylcarbamate

A solution containing the product from Example 8B (5.67 g, 12.5 mmol) in THF (124 mL) was treated with the product from Example 7B (3.8 g, 12.5 mmol), DEPBT (5.59 g, 18.7 mmol), and N,N-diisopropylethylamine (10.8 mL, 62.0 mmol), stirred at 25° C. for 16 hours, and partitioned between ethyl acetate and 10% Na₂CO₃ solution. The organic phase phase was washed with additional 10% Na₂CO₃ solution and brine, dried over MgSO₄, filtered and concentrated. The residue was chromatographed on silica gel eluting with a gradient starting with dichloromethane and ending with acetone to give the title compound (4.42 g, 48% yield). ¹H NMR (300 MHz, DMSO-d₆), δ ppm 0.68 (d, J=6.25 Hz, 3H), 0.82 (m, 14H), 0.92 (m, 1H), 1.30 (m, 1H), 1.50 (m, 2H), 1.79 (m, 1H), 2.43 (m, 3H), 2.68 (m, 3H), 2.89 (m, 1H), 3.10 (m, 3H), 3.57 (m, 3H), 3.89 (m, 2H), 4.13 (m, 2H), 4.34 (s, 2H), 4.79 (d, J=5.52 Hz, 1H), 6.80 (d, J=9.56 Hz, 1H), 7.11 (m, 12H), 7.50 (d, J=8.82 Hz, 1H), 7.66 (t, J=7.54 Hz, 1H), 7.83 (d, J=9.19 Hz, 1H).

### EXAMPLE 9A

### methyl(1S,2S)-1-[({(1S,2S,4S)-1-benzyl-4-[(tert-butoxycarbonyl)amino]-2-hydroxy-5-phenylpentyl}amino)carbonyl]-2-methylbutylcarbamate

A solution of the product from Example 6A (0.50 g, 1.30 mmol) in THF (13 mL) was treated with the product from Example 5A (0.30 g, 1.59 mmol), DEPBT (0.45 g, 1.50 mmol), and N,N-diisopropylethylamine (1.1 mL, 6.31 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated to give the title compound, used without further purification.

### EXAMPLE 9B

### methyl(1S,2S)-1-({[(1S,2S,4S)-4-amino-1-benzyl-2-hydroxy-5-phenylpentyl]amino}carbonyl)-2-methylbutylcarbamate

A solution containing the crude product from Example 9A in THF (150 mL) was treated with an HCl solution (5 mL, 4 N in dioxane), and the mixture was heated at 60° C. for 2 hours, cooled to 25° C. and concentrated. The concentrate was partitioned between ethyl acetate and saturated NaHCO₃. The organic phase was washed with brine, dried over MgSO₄, filtered and concentrated to give the title compound (0.36 g, 61% yield).

### EXAMPLE 9C

### methyl(1S)-1-[({(1S,2S,4S)-1-benzyl-2-hydroxy-4-[((2S)-3-methyl-2-{3-[(6-methyl-2-pyridinyl)methyl]-2-oxo-1-imidazolidinyl}pentanoyl)amino]-5-phenylpentyl}amino)carbonyl]-2-methylbutylcarbamate

A solution containing the product from Example 9B (0.36 g, 0.79 mmol) in THF (8 mL) was treated with the product from Example 7B (0.33 g, 0.79 mmol), DEPBT (0.355 g, 1.19 mmol), and N,N-diisopropylethylamine (0.70 mL, 4.02 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-50% acetone/dichloromethane to give the title compound (0.264 g, 45% yield). ¹H NMR (300 MHz, DMSO-d₆), δ ppm 0.75 (m, 12H), 0.94 (m, 2H), 1.29 (m, 2H), 1.46 (m, 2H), 1.62 (m, 1H), 1.80 (m, 1H), 2.43 (m, 4H), 2.68 (m, 3H), 2.88 (m, 1H), 3.08 (m, 3H), 3.56 (m, 4H), 3.84 (m, 2H), 4.15 (m, 2H), 4.34 (s, 2H), 4.84 (d, J=5.88 Hz, 1H), 7.03 (m, 7H), 7.15 (m, 6H), 7.39 (d, J=9.19 Hz, 1H), 7.66 (t, J=7.72 Hz, 1H), 7.83 (d, J=8.82 Hz, 1H).

### EXAMPLE 10A

### tert-butyl(2S)-3,3-dimethyl-2-[(2-{[(6-methyl-2-pyridinyl)methyl]amino}ethyl)amino]butanoate

A solution containing the product from Example 6F (2.0 g, 8.68 mmol) in dichloromethane (40 mL) was treated with 6-methyl-2-pyridinecarboxaldehyde (1.04 g, 8.59 mmol) and magnesium sulfate (6.0 g, 49.85 mmol), stirred at 25° C. for 4 hours, filtered and concentrated. A solution of the residue in methanol (40 mL) at 0° C. was treated with sodium borohydride (0.5 g, 13.22 mmol), stirred at 25° C. for 1.5 hours, and concentrated. The concentrate was partitioned between dichloromethane and water, and the aqueous was extracted three times with dichloromethane. The combined organic phase was dried over NaSO₄, filtered and concentrated. The residue was chromatographed on silica gel, eluting with 10% methanol in chloroform, to give the title compound (2.48 g, 85% yield).

### EXAMPLE 10B

### tert-butyl(2S)-3,3-dimethyl-2-{3-[(6-methyl-2-pyridinyl)methyl]-2-oxo-1-imidazolidinyl}butanoate

A solution containing the product from Example 10A (1.76 g, 5.25 mmol) in 1,2-dichloroethane (210 mL) was treated with N,N-disuccinimidyl carbonate (1.61 g, 6.28 mmol) and triethylamine (0.75 mL, 5.38 mmol), stirred at 25° C. for 16 hours, and partitioned with 10% Na₂CO₃. The aqueous phase was extracted with additional dichloromethane. The combined organic phase was dried over MgSO₄, filtered and concentrated. The residue was chromatographed on silica gel eluting with 0-25% methyl tert-butyl ether/dichloromethane to give the title compound (1.33 g, 70% yield).

### EXAMPLE 10C

### (2S)-3,3-dimethyl-2-{3-[(6-methyl-2-pyridinyl)methyl]-2-oxo-1-imidazolidinyl}butanoic Acid

A solution containing the product from Example 10B (1.33 g, 3.68 mmol) in dichloromethane (20 mL) was treated with trifluoracetic acid (20 mL), stirred at 25° C. for 2 hours and concentrated. The residue was purified by reversed phase chromatography on a C18 column, eluting with 0-100% acetonitrile/water (0.1% TFA) to give the title compound (1.44 g, 94% yield) as the trifluoroacetic acid salt.

### EXAMPLE 10D

### (2S)-3,3-dimethyl-2-{3-[(6-methyl-2-pyridinyl)methyl]-2-oxo-1-imidazolidinyl}butanoic Acid

A solution containing the product from Example 10B (10 g, 27.7 mmol) in dichloromethane (100 ml) at −5° C. was slowly treated with an HCl solution in dioxane (200 mL, 4 N), stirred at 40° C. for 6 hrs, stirred at 25° C. for 16 hours concentrated to give the title compound as a hydrochloride salt (10 g, quantitative).

### EXAMPLE 10E

### methyl(1S)-1-[({(1S,2S,4S)-1-benzyl-4-[((2S)-3,3-dimethyl-2-{3-[(6-methyl-2-pyridinyl)methyl]-2-oxo-1-imidazolidinyl}butanoyl)amino]-2-hydroxy-5-phenylpentyl}amino)carbonyl]-2,2-dimethylpropylcarbamate

A solution containing the product from Example 8B (1.36 g, 2.99 mmol) in THF (30 mL) was treated with the product from Example 10D (1.25 g, 2.98 mmol), DEPBT (1.34 g, 4.48 mmol), and N,N-diisopropylethylamine (2.6 mL, 14.9 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by elution with 5% methanol in ethyl acetate. The material obtained after concentration of all the desired fractions was re-chromatographed on silica gel eluting with 0-50% acetone/dichloromethane to give the title compound (1.57 g, 71% yield). ¹H NMR (300 MHz, DMSO-d₆), δ ppm 0.84 (s, 9H), 0.87 (s, 9H), 1.51 (m, 2H), 2.41 (m, 5H), 2.65 (dd, J=13.05, 2.76 Hz, 1H), 2.72 (d, J=7.35 Hz, 2H), 2.97 (m, 1H), 3.08 (q, 1=8.58 Hz, 1H), 3.24 (m, 1H), 3.58 (m, 3H), 3.91 (d, J=9.19 Hz, 1H), 3.97 (s, 1H), 4.16 (m, 2H), 4.34 (d, J=2.94 Hz, 2H), 4.80 (d, J=5.52 Hz, 1H), 6.81 (d, J=9.56 Hz, 1H), 7.07 (m, 6H), 7.16 (m, 7H), 7.50 (d, J=9.19 Hz, 1H), 7.68 (t, J=7.72 Hz, 1H), 7.89 (d, J=9.19 Hz, 1H).

### EXAMPLE 11A

### tert-butyl(1S,2S,4S)-4-amino-1-benzyl-2-hydroxy-5-phenylpentylcarbamate

A solution of the product from Example 127 (5 g, 17.6 mmol) in toluene (70 mL) was treated with phenyl boronic acid (2.14 g, 17.6 mmol), stirred at reflux until the theoretical amount of water (0.317 mL) was collected in a Dean-Stark trap. The reaction mixture was cooled to 25° C. and concentrated to dryness, treated with dichloromethane (70 mL) and di-tert-butyl-dicarbonate (4.0 mL, 17.6 mmol), stirred at 25° C. for 18 hours, treated with sodium hydroxide solution (35 mL, 1 N), and stirred for 10 minutes. The organic phase was washed with water, dried over Na₂SO₄, filtered and concentrated. The residue was chromatographed on silica gel, eluting with isopropyl amine in dichloromethane to give the title compound (2.23 g, 33% yield).

### EXAMPLE 11B

### 2-methyl-1,3-thiazole-4-carbaldehyde

A solution of ethyl 2-methylthiazole-4-carboxylate (1.00 g, 5.8 mmol) in toluene (18 mL) at −78° C. was treated dropwise with a diisobutyl aluminum hydride solution in dichloromethane (11.1 mL, 1 M) over 30 minutes, stirred at −78° C. for 4 hours, quenched with acetic acid (0.46 mL), warmed to 25° C. and concentrated. The concentrate was treated with dichloromethane and Rochelle's salt, stirred vigorously until a clear, two-phase solution formed (approximately 10 minutes). The layers were separated and organic layer was washed with 10% NaHCO₃, brine, dried over Na₂SO₄, filtered and concentrated. The residue was chromatographed on silica gel, eluting with 14% ethyl acetate in hexanes to give the title compound (0.28 g, 38% yield).

### EXAMPLE 11C

### tert-butyl(2S,3S)-3-methyl-2-{3-[(2-methyl-1,3-thiazol-4-yl)methyl]-2-oxo-1-imidazolidinyl}pentanoate

A solution containing the product from Example 3G (1.81 g, 7.9 mmol) in a mixture of benzene (8 mL) and methanol (8 mL) was treated with the product from Example 11B (1.0 g, 7.9 mmol), stirred at 50° C. for 1 hour, cooled to 25° C. and treated with sodium borohydride (0.60 g, 15.7 mmol), stirred at 25° C. for 1 hour, quenched with sodium bicarbonate solution and partitioned between ethyl acetate and water. The organic phase was washed with brine, dried over Na₂SO₄, filtered and concentrated. A solution of the residue (7.9 mmol) in toluene (16 mL) was treated with bis(4-nitrophenyl)carbonate (2.87 g, 9.4 mmol), stirred at reflux for 16 hours, cooled to 25° C. and partitioned between ethyl acetate and 10% K₂CO₃. The organic phase was washed with brine, dried over Na₂SO₄, filtered and concentrated. The residue was chromatographed on silica gel eluting with 1% methanol in chloroform to give the title compound (2.0 g, 69% yield).

### EXAMPLE 11D

### (2S,3S)-3-methyl-2-{3-[(2-methyl-1,3-thiazol-4-yl)methyl]-2-oxo-1-imidazolidinyl}pentanoic Acid

A solution containing the product from Example 1C (2.0 g, 5.4 mmol) in dichloromethane (14 mL) was treated with trifluoracetic acid (7 mL), stirred at 25° C. for 3 hours and concentrated to give the title compound as a trifluoroacetic acid salt.

### EXAMPLE 11E

### tert-butyl(1S,2S,4S)-1-benzyl-2-hydroxy-4-[((2S,3S)-3-methyl-2-{3-[(2-methyl-1,3-thiazol-4-yl)methyl]-2-oxo-1-imidazolidinyl}pentanoyl)amino]-5-phenylpentylcarbamate

A solution containing the product from Example 11A (0.42 g, 1.09 mmol) in THF (5 mL) was treated with the product from Example 11D (0.34 g, 1.09 mmol), DEPBT (0.65 g, 2.2 mmol), and N,N-diisopropylethylamine (0.57 mL, 3.3 mmol), stirred at 25° C. for 4 hours and partitioned between dichloromethane and 10% Na₂CO₃ solution. The organic phase was washed with additional 10% Na₂CO₃ solution and brine, dried over Na₂SO₄, filtered and concentrated. The residue was chromatographed on silica gel, eluting with 2% methanol in chloroform to give the title compound (0.27 g, 36% yield).

### EXAMPLE 11F

### (2S,3S)-N-[(1S,3S,4S)-4-amino-1-benzyl-3-hydroxy-5-phenylpentyl]-3-methyl-2-{3-[(2-methyl-1,3-thiazol-4-yl)methyl]-2-oxo-1-imidazolidinyl}pentanamide

A solution containing the product from Example 11E (0.27 g, 0.4 mmol) in THF (4 mL) was treated with an HCl solution (0.70 mL, 4 N in dioxane), heated at 60° C. for 3 hours, cooled to 25° C., concentrated to give the title compound as the hydrochloride salt.

### EXAMPLE 11G

### methyl(1S)-1-[({(1S,2S,4S)-1-benzyl-2-hydroxy-4-[((2S)-3-methyl-2-{3-[(2-methyl-1,3-thiazol-5-yl)methyl]-2-oxo-1-imidazolidinyl}pentanoyl)amino]-5-phenylpentyl}amino)carbonyl]-2,2-dimethylpropylcarbamate

A solution containing the product from Example 11F (0.23 g, 0.4 mmol) in THF (5 mL) was treated with the product from Example 1F (0.08 g, 0.4 mmol), DEPBT (0.24 g, 0.8 mmol), and N,N-diisopropylethylamine (0.21 mL, 1.2 mmol), stirred at 25° C. for 64 hours, and partitioned between chloroform and 10% Na₂CO₃ solution. The organic phase was washed with additional 10% Na₂CO₃ solution and brine, dried over Na₂SO₄, filtered and concentrated. The residue was chromatographed on silica gel, eluting with 2% methanol in chloroform to give the title compound (0.13 g, 44% yield). ¹H NMR (300 MHz, CDCl₃), δ ppm 0.77 (d, J=6.25 Hz, 3H), 0.85 (t, J=7.35 Hz, 4H), 0.92 (s, 10H), 1.00 (m, 1H), 1.41 (m, 1H), 2.01 (m, 1H), 2.68 (s, 3H), 2.71. (d, J=7.35 Hz, 2H), 2.82 (dd, J=7.35, 1.84 Hz, 2. H), 3.00 (m, 1H), 3. F (m, 3H), 3.66 (m, 6H), 3.77 (d, J=8.82 Hz, 1H), 3.94 (s, 1H), 4.07 (m, 2H), 4.40 (s, 2H), 5.23 (s, 1H), 6.05 (d, J=9.19 Hz, 1H), 6.48 (d, J=8.46 Hz, 1H), 6.91 (s, 1H), 7.15 (m, 11H).

### EXAMPLE 12

### methyl(1S)-1-{[((1S,2S,4S)-1-benzyl-2-hydroxy-4-{[(2S)-3-methyl-2-(2-oxo-3-{[2-(3-pyridinyl)-1,3-thiazol-4-yl]methyl}-1-imidazolidinyl)pentanoyl]amino}-5-phenylpentyl)amino]carbonyl}-2,2-dimethylpropylcarbamate

A solution containing the product from Example 8B (0.275 g, 0.56 mmol) in THF (6 mL) was treated with the product from example 3L (0.227 g, 0.61 mmol), DEPBT (0.275 g, 0.92 mmol), and N,N-diisopropylethylamine (0.55 mL, 3.16 mmol), stirred at 25° C. for 64 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by elution with 5% methanol in ethyl acetate to give the title compound (0.378 g, 77% yield).

¹H NMR (300 MHz, DMSO-d₆), δ ppm 0.67 (d, J=6.62 Hz, 3H), 0.80 (m, 14H), 0.94 (m, 1H), 1.29 (m, 1H), 1.49 (m, 2H), 1.78 (m, 1H), 2.42 (m, 1H), 2.68 (m, 3H), 2.86 (m, 1H), 3.13 (m, 4H), 3.58 (m, 4H), 3.89 (m, 2H), 4.12 (m, 2H), 4.47 (s, 2H), 4.79 (d, J=5.52 Hz, 1H), 6.80 (d, J=9.19 Hz, 1H), 7.07 (m, 7H), 7.52 (m, 2H), 7.57 (s, 1H), 7.82 (d, J=8.82 Hz, 1H), 8.29 (m, 1H), 8.66 (dd, J=4.78, 1.84 Hz, 1H), 9.13 (d, J=1.47 Hz, 1H).

### EXAMPLE 13A

### 6-methylnicotinaldehyde

A solution of methyl 6-methylnicotinate (0.5 g, 3.3 mmol) in THF (16 mL) at 0° C. was treated dropwise with lithium aluminum hydride in THF (6.6 mL, 1 M), stirred at 0° C. for 1.5 hours, treated with ethyl acetate (3 mL), stirred at 25° C. The reaction was partitioned between ethyl acetate and saturated NaHCO₃, and the organic phase was washed with brine and dried over MgSO₄, filtered and concentrated. A solution of the residue (0.395 g) in dichloromethane (16 mL) was treated with MnO₂ (2 g), stirred at 25° C. for 68 hours, filtered through celite® to give the title compound (0.326 g, 80% yield), which was used without further purification.

### EXAMPLE 13B

### tert-butyl(2S,3S)-3-methyl-2-{3-[(6-methyl-3-pyridinyl)methyl]-2-oxo-1-imidazolidinyl}pentanoate

A solution containing the product from Example 3G (0.55 g, 2.39 mmol) in a mixture of benzene (6 mL) and ethanol (6 mL) was treated with the product from Example 13A (0.265 g, 2.19 mmol), stirred at 70° C. for 2 hours, cooled to 25° C., treated with sodium borohydride (0.25 g, 6.61 mmol), stirred at 25° C. for 3 hours, and partitioned between ethyl acetate and saturated NaHCO₃. The organic phase was washed with brine, dried over MgSO₄, filtered and concentrated. A solution of the residue (2.19 mmol) in 1,2-dichloroethane (90 mL) was treated with N,N-disuccinimidyl carbonate (0.675 g, 2.63 mmol) and triethylamine (0.30 mL, 2.15 mmol), stirred at 25° C. for 16 hours, and partitioned with 10% Na₂CO₃. The aqueous phase was extracted with additional dichloromethane. The combined organic phase was dried over MgSO₄, filtered and concentrated. The residue was chromatographed on silica gel eluting with 0-100% ethyl acetate/dichloromethane to give the title compound (0.392 g, 49% yield).

### EXAMPLE 13C

### (2S,3S)-3-methyl-2-{3-[(6-methyl-3-pyridinyl)methyl]-2-oxo-1-imidazolidinyl}pentanoic Acid

A solution containing the product from Example 13B (0.39 g, 1.08 mmol) in dichloromethane (5 mL) was treated with trifluoracetic acid (5 mL), stirred at 25° C. for 2 hours and concentrated. The concentrate was purified by reversed phase chromatography on a C18 column, eluting with 0-100% acetonitrile/water (0.1% TFA) give the title compound (0.536 g, quantitative) as the trifluoroacetic acid salt.

### EXAMPLE 13D

### methyl(1S)-1-[({(1S,2S,4S)-1-benzyl-2-hydroxy-4-[((2S)-3-methyl-2-{3-[(6-methyl-3-pyridinyl)methyl]-2-oxo-1-imidazolidinyl}pentanoyl)amino]-5-phenylpentyl}amino)carbonyl]-2,2-dimethylpropylcarbamate

A solution containing the product from Example 8B (0.29 g, 0.64 mmol) in THF (6 mL) was treated with the product from Example 13C (0.27 g, 0.64 mmol), DEPBT (0.300 g, 1.00 mmol), and N,N-diisopropylethylamine (0.60 mL, 3.44 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0100% ethyl acetate/dichloromethane, followed by elution with 5% methanol in ethyl acetate to give the title compound (0.345 g, 73% yield).

¹H NMR (300 MHz, DMSO-d₆), δ ppm 0.67 (d, J=6.25 Hz, 3H), 0.85 (m, 13H), 1.21 (m, 1H), 1.49 (m, 2H), 1.77 (m, 1H), 2.41 (m, 4H), 2.68 (m, 3H), 2.84 (m, 1H), 2.93 (m, 1H), 3.01 (m, 2H), 3.57 (m, 3H), 3.89 (m, 3H), 4.11 (m, 2H), 4.28 (s, 2H), 4.79 (d, J=5.52 Hz, 1H), 6.80 (d, J=9.93 Hz, 1H), 7.03 (s, 5H), 7.17 (m, 6H), 7.52 (m, 2H), 7.83 (d, J=8.82 Hz, 1H), 8.35 (d, J=2.21 Hz, 1H).

### EXAMPLE 14A

### tert-butyl(2S)-3,3-dimethyl-2-{3-[(2-methyl-1,3-thiazol-4-yl)methyl]-2-oxo-1-imidazolidinyl}butanoate

A solution containing the product from Example 6F (0.82 g, 3.5 mmol) in a mixture of benzene (12 mL) and methanol (12 mL) was treated with the product from Example 11B (0.45 g, 3.5 mmol), heated at 50° C. for 1 hour, cooled to 25° C., treated with sodium borohydride (0.27 g, 7.1 mmol), stirred at 25° C. for 1 hour, quenched with sodium bicarbonate solution and partitioned between ethyl acetate and water. The organic phase was washed with brine and dried over Na₂SO₄, filtered and concentrated. A solution containing the residue (3.5 mmol) in toluene (20 mL) was treated with bis(4-nitrophenyl)carbonate (1.29 g, 4.2 mmol), heated at reflux for 16 hours, cooled to 25° C., and partitioned between ethyl acetate and 10% K₂CO₃. The organic phase was washed with brine and dried over Na₂SO₄, filtered and concentrated to give the title compound, which was used without further purification.

### EXAMPLE 14B

### (2S)-3,3-dimethyl-2-{3-[(2-methyl-1,3-thiazol-4-yl)methyl]-2-oxo-1-imidazolidinyl}butanoic Acid

A solution containing the product from Example 14A (3.5 mmol) in dichloromethane (4 mL) was treated with trifluoracetic acid (3 mL), stirred at 25° C. for 3 hours and concentrated. The residue was chromatographed on silica gel eluting with 2% methanol in chloroform to give the title compound as the trifluoroacetic acid salt (0.88 g, 80% yield).

### EXAMPLE 14C

### tert-butyl(1S,2S,4S)-1-benzyl-4-[((2S)-3,3-dimethyl-2-{3-[(2-methyl-1,3-thiazol-4-yl)methyl]-2-oxo-1-imidazolidinyl}butanoyl)amino]-2-hydroxy-5-phenylpentylcarbamate

A solution containing the product from Example 11A (0.37 g, 1.0 mmol) in THF (5 mL) was treated with the product of Example 14B (0.30 g, 1.0 mmol), DEPBT (0.58 g, 1.9 mmol), and N,N-diisopropylethylamine (0.57 mL, 3.3 mmol), stirred at 25° C. for 4 hours, and partitioned between dichloromethane and 10% Na₂CO₃ solution. The organic phase was washed with additional 10% Na₂CO₃ solution and brine, dried over Na₂SO₄, filtered and concentrated. The residue was chromatographed on silica gel, eluting with 2% methanol in chloroform to give the title compound (0.63 g, 97% yield).

### EXAMPLE 14D

### (2S)-N-[(1S,3S,4S)-4-amino-1-benzyl-3-hydroxy-5-phenylpentyl]-3,3-dimethyl-2-{3-[(2-methyl-1,3-thiazol-4-yl)methyl]-2-oxo-1-imidazolidinyl}butanamide

A solution containing the product from Example 14C (0.63 g, 0.9 mmol) in THF (5 mL) was treated with an HCl solution (0.1.6 mL, 4 N in dioxane), heated at 60° C. for 3 hours, cooled to 25° C. and concentrated. The residue was treated with ethanol (10 mL) and concentrated. This process was repeated an additional time to give the title compound as the hydrochloride salt.

### EXAMPLE 14E

### methyl(1S)-1-[({(1S,2S,4S)-1-benzyl-4-[((2S)-3,3-dimethyl-2-{3-[(2-methyl-1,3-thiazol-4-yl)methyl]-2-oxo-1-imidazolidinyl}butanoyl)amino]-2-hydroxy-5-phenylpentyl}amino)carbonyl]-2,2-dimethylpropylcarbamate

A solution containing the product from Example 14D (0.9 mmol) in THF (5 mL) was treated with the product from Example 1F (0.176 g, 0.9 mmol), DEPBT (0.556 g, 1.86 mmol), and N,N-diisopropylethylamine (0.486 mL, 2.79 mmol), stirred at 25° C. for 48 hours, and partitioned between dichloromethane and 10% Na₂CO₃ solution. The organic phase was washed with additional 10% Na₂CO₃ solution and brine, dried over Na₂SO₄, filtered and concentrated. The residue was chromatographed on silica gel, eluting with 2% methanol in chloroform to give the title compound (0.31 g, 44% yield). ¹H NMR (300 MHz, CDCl₃), δ ppm 0.94 (s, 9H), 1.00 (s, 9H), 2.74 (m, 9H), 3.13 (m, 2H), 3.40 (m, 1H), 3.63 (m, 1H), 3.68 (s, 3H), 3.78 (d, J=9.19 Hz, 1H), 3.83 (d, J=4.04 Hz, 1H), 3.96 (s, 1H), 4.10 (m, 2H), 4.44 (d, J=2.21 Hz, 2H), 5.27 (d, J=8.46 Hz, 1H), 6.05 (d, J=9.19 Hz, 1H), 6.14 (d, J=9.19 Hz, 1H), 6.93 (s, 1H), 7.16 (m, 11H).

### EXAMPLE 15A

### 2-methylnicotinaldehyde

A solution of methyl 2-methylnicotinate (0.5 g, 3.3 mmol) in THF (16 mL) at 0° C. was treated dropwise with lithium aluminum hydride in THF (6.6 mL, 1 M), stirred at 0° C. for 1.5 hours, treated with ethyl acetate (3 mL), warmed to 25° C., and partitioned between ethyl acetate and saturated NaHCO₃. The organic phase was washed with brine, dried over MgSO₄, filtered and concentrated. A solution of the residue (0.391 g) in dichloromethane (16 mL) was treated with MnO₂ (2 g), stirred at 25° C. for 68 hours, filtered through celite®, and the solvent was evaporated to give the title compound (0.303 g, 75% yield), which was used without further purification.

### EXAMPLE 15B

### tert-butyl(2S,3S)-3-methyl-2-{3-[(2-methyl-3-pyridinyl)methyl]-2-oxo-1-imidazolidinyl}pentanoate

A solution containing the product from Example 3G (2.4 g, 10.43 mmol) in dichloromethane (24 mL) was treated with the product from Example 15A (1.3 g, 10.74 mmol) and MgSO₄ (4.6 g, 38.21 mmol), stirred at 25° C. for 2.5 hours, filtered and concentrated. A solution of the residue in methanol (24 mL) at 0° C. was treated with sodium borohydride (0.5 g, 13.2 mmol), stirred at 25° C. for 3 hours. The solvent was concentrated and the reaction was partitioned between ethyl acetate and saturated NaHCO₃, and the organic phase was washed with brine and dried over MgSO₄, filtered and concentrated. A solution of the residue (3.4 g) in 1,2-dichloroethane (30 mL was treated with bis(4nitrophenyl)carbonate (3.8 g, 12.5 mmol), heated at 60° C. for 16 hours, and partitioned between ethyl acetate and saturated NaHCO₃. The organic phase was washed with brine, dried over MgSO₄, filtered and concentrated. The residue was chromatographed on silica gel eluting with ethyl acetate to give the title compound (2.31 g, 60% yield).

### EXAMPLE 15C

### (2S,3S)-3-methyl-2-{3-[(2-methyl-3-pyridinyl)methyl]-2-oxo-1-imidazolidinyl}pentanoic Acid

A solution containing the product from Example 15B (2.3 g, 6.37 mmol) in dichloromethane (15 mL) was treated with trifluoracetic acid (15 mL), stirred at 25° C. for 5.5 hours and concentrated to give the title compound (3.42 g) as the trifluoroacetic acid salt, which was used without further purification.

### EXAMPLE 15D

### methyl(1S)-1-[({(1S,2S,4S)-1-benzyl-2-hydroxy-4-[((2S)-3-methyl-2-{3-[(2-methyl-3-pyridinyl)methyl]-2-oxo-1-imidazolidinyl}pentanoyl)amino]-5-phenylpentyl}amino)carbonyl]-2,2-dimethylpropylcarbamate

A solution containing the product from Example 8B (2.0 g, 4.40 mmol) in DMF (10 mL) was treated with the product from Example 15C (1.34 g, 4.39 mmol), EDAC (1.01 g, 5.27 mmol), HOBT (0.7 g, 5.19 mmol), and NMM (0.96 mL, 8.72 mmol), stirred at 25° C. for 16 hours, treated with Example 15C (0.13 g), EDAC (0.5 g), HOBT (0.35 g), NMM (1 mL), and DMF (5 mL), stirred for 64 hours at 25° C. and concentrated. The concentrate was partitioned between ethyl acetate and saturated NaHCO₃. The organic phase was washed with brine, dried over Na₂SO₄, filtered and concentrated. The residue was chromatographed on silica gel eluting with 0-4% methanol/dichloromethane to give the title compound (1.76 g, 54% yield). ¹H NMR (300 MHz, DMSO-d₆), δ ppm 0.68 (d, J=6.62 Hz, 3H), 0.81 (m, 15H), 1.26 (m, 1H), 1.49 (m, 2H), 1.78 (m, 1H), 2.45 (m, 5H), 2.70 (m, 3H), 2.90 (m, 2H), 3.04 (m, 2H), 3.59 (m, 4H), 3.87 (m, 2H), 4.13 (m, 2H), 4.31 (s, 2H), 4.79 (d, J=5.52 Hz, 1H), 6.80 (d, J=9.19 Hz, 1H), 7.05 (s, 3H), 7.19 (m, 5H), 7.51 (m, 2H), 7.86 (d, J=8.82 Hz, 1H), 8.36 (d, J=3.68 Hz, 1H).

### EXAMPLE 16

### methyl(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 2C (1.0 g, 1.88 mmol) in THF (19 mL) was treated with the product from Example 10B (0.83 g, 1.98 mmol), DEPBT (0.84 g, 2.8 mmol), and N,N-diisopropylethylamine (1.6 mL, 9.2 mmol), stirred at 25° C. for 16 hours, and partitioned between a mixture of dichloromethane and ethyl acetate (2:1, respectively) and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by elution with 0-5% methanol in ethyl acetate to give the title compound (1.15 g, 75% yield). ¹H NMR (300 MHz, DMSO-d₆), δ ppm 0.83 (s, 9H), 0.90 (s, 9H), 1.55 (m, 2H), 2.38 (q, J=9.44 Hz, 1H), 2.46 (s, 3H), 2.57 (m, 1H), 2.67 (d, J=7.35 Hz, 2H), 2.79 (m, 1H), 2.97 (m, 1H), 3.09 (q, J=8.95 Hz, 1H), 3.21 (m, 1H), 3.50 (s, 3H), 3.67 (m, 1H), 3.85 (d, J=9.93 Hz, 1H), 4.12 (m, 3H), 4.35 (m, 2H), 4.54 (d, J=7.72 Hz, 1H), 6.63 (d, J=9.56 Hz, 1H), 7.09 (m, 7H), 7.22 (d, J=8.09 Hz, 2H), 7.31 (m, 1H), 7.49 (d, J=9.56 Hz, 1H), 7.69 (t, J=7.54 Hz, 1H), 7.86 (m, 5H), 8.63 (d, J=4.78 Hz, 1H).

### EXAMPLE 17A

### Methyl 6-(hydroxymethyl)-2-pyridinecarboxylate

A suspension of dimethyl 2,6-pyridine-dicarboxylate (50 g, 0.25 mol) in methanol (400 mL) and tetrahydrofuran (150 mL) was heated to dissolve and while the solution was still hot, it was treated in portions with sodium borohydride (9.1 g, 0.24 mol). The mixture was stirred for 1 hour after the addition, cooled to 25° C., quenched with 10% citric acid (80 mL), stirred for 15 minutes, filtered, and concentrated. A solution of the concentrate in dichloromethane was dried over sodium sulfate, filtered, and concentrated. A solution of the residue in hot ethyl acetate was allowed to stand for 16 hours at 25° C. The resulting precipitate (23 g) was collected by filtration. The mother liquor was concentrated and the resulted solid was purified by flash chromatography on silica gel eluting with 10% methanol in dichloromethane to give the crude white solid (24 g). The solid w was crystallized in ethyl acetate to give a total yield of the title compound (36 g, 84% yield).

### EXAMPLE 17B

### Methyl 6-formyl-2-pyridinecarboxylate

A solution of the product from Example 17A (8 g, 48 mmol) in dichloromethane (200 mL) was treated with electrolytic manganese dioxide (41.67 g, 0.48 mol). The mixture was stirred for 4 days at 25° C. and filtered through celite. The filtrate was concentrated under reduced pressure and the residue was purified by chromatography on silica gel eluting with 5% methanol in dichloromethane to give the title compound as a white solid (6.9 g, 87% yield).

### EXAMPLE 17C

### Methyl 6-{[(2-{[(1S)-1-(tert-butoxycarbonyl)-2,2-dimethylpropyl]amino}ethyl)amino]methyl}-2-pyridinecarboxylate

A suspension containing the product from Example 17B (6 g, 36.4 mmol), the product from Example 6F (8.37 g, 36.4 mmol), and magnesium sulfate (21.9 g, 0.18 mol) in dichloromethane (80 mL) was stirred at 25° C. for 4 hours, filtered, and concentrated. A solution of the residue in methanol (80 mL) was treated with sodium borohydride (1.58 g, 41.9 mmol) at 0° C., stirred 0.5 hour at 0° C., quenched with acetone (2 mL), concentrated, treated with 1M sodium bicarbonate and extracted with ethyl acetate. The organic phase layer was concentrated and the residue was chromatographed on silica gel eluting with 8% methanol in dichloromethane to give the title compound (10.27 g).

### EXAMPLE 17D

### Methyl 6-({3-[(18)-1-(tert-butoxycarbonyl)-2,2-dimethylpropyl]-2-oxo-1-imidazolidinyl}methyl)-2-pyridinecarboxylate

A solution of the product from Example 17C (10.27 g, 27.1 mmol), bis(4-nitrophenyl) carbonate (8.24 g, 27.1 mmol), in toluene (100 mL) was heated at 110° C. for 16 hours, cooled to 25° C., treated with 1M sodium bicarbonate, and extracted with ethyl acetate. The organic phase layer was concentrated and the residue was purified by flash chromatography on silica gel eluting with 60% ethyl acetate in hexane to give the title compound as a white solid (9.44 g, 64% yield).

### EXAMPLE 17E

### tert-butyl(2S)-2-(3-{[6-(1-hydroxy-1-methylethyl)-2-pyridinyl]methyl}-2-oxo-1-imidazolidinyl)-3,3-dimethylbutanoate

A solution of the product from Example 17D (9 g, 22.2 mmol) in tetrahydrofuran (200 mL) at 0° C. was treated with a solution of methylmagnesium bromide in diethyl ether (3M, 37 mL, 111 mmol), stirred for 1.5 hours at 0° C., quenched with 10% citric acid (20 mL), extracted with ethyl acetate. The organic phase layer was concentrated, and the residue was purified by flash chromatography on silica gel eluting with 20-70% ethyl acetate in hexane to give the title compound (7.2 g, 80% yield).

### EXAMPLE 17F

### (2S)-2-(3-{[6-(1-hydroxy-1-methylethyl)-2-pyridinyl]methyl}-2-oxo-1-imidazolidinyl)-3,3-dimethylbutanoic Acid

The product from Example 17E (7.2 g, 17.8 mmol) at 25° C. was treated with 90% trifluoroacetic acid in water (30 mL). The reaction mixture was stirred at 25° C. for 3 hours and concentrated. A solution of the residue in water (2 mL) was chromatographed on silica gel eluting with 5% methanol/dichloromethane to give the title compound as the trifluoroacetic acid salt (7.4 g, 89.9% yield).

### EXAMPLE 17G

### methyl(1S)-1-{[((1S,2S,4S)-1-benzyl-2-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenylpentyl)amino]carbonyl}-2,2-dimethylpropylcarbamate

A solution containing the product from Example 8B (1.7 g, 3.73 mmol) in THF (25 mL) was treated with the product from Example 17F (1.8 g, 3.88 mmol), DEPBT (2.32 g, 7.46 mmol), and triethylamine (1.35 mL, 9.32 mmol, stirred at 25° C. for 16 hours, quenched with sodium bicarbonate solution (1M), and extracted with ethyl acetate. The organic phase layer was decanted and concentrated. The residue was chromatographed on a silica gel column eluting with 2% methanol/ethyl acetate to give the title compound (1.73 g, 57% yield). ¹H NMR (300 MHz, CD₃OD) δ ppm 0.91 (s, 9H), 0.95 (s, 9H), 1.24 (m, 1H), 1.35 (m, 2H), 1.53 (s, 6H), 1.66 (m, 1H), 2.01 (s, 1H), 2.42 (m, 1H), 2.87 (m, 2H), 3.08 (m, 1H), 3.24 (m, 1H), 3.66 (s, 2H), 3.76 (m, 1H), 3.92 (s, 1H), 3.98 (s, 1H), 4.09 (m, 1H), 4.25 (dd, J=8.64, 7.17 Hz, 1H), 4.36 (m, J=8.82 Hz, 1H), 4.43 (s, 1H), 4.58 (s, 1H), 4.63(s, 1H), 7.14 (m, 11H), 7.53 (d, J=6.99 Hz, 1H), 7.77 (t, J=7.91 Hz, 1H).

### EXAMPLE 18

### methyl(1S)-1-[({(1S,3S,4S)-3-hydroxy-4-[((2S)-3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate

A solution containing the product from Example 2C (0.01 g, 0.019 mmol) in THF (0.2 mL) was treated with the product from Example 7B (0.009 g, 0.021 mmol), DEPBT (0.009 g, 0.030 mmol), and N,N-diisopropylethylamine (0.016 mL, 0.092 mmol), stirred at 25 C for 16 hours and partitioned between a mixture of dichloromethane, ethyl acetate (2:1, respectively) and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with a gradient starting with 5-100% acetonitrile in water (0.1% TFA). The product was partitioned between a mixture of dichloromethane and ethyl acetate (2:1, respectively) and saturated NaHCO₃ solution. The organic phase was washed with brine, dried over MgSO₄, filtered and concentrated to give the title compound (0.0076 g, 51% yield).

¹H NMR (300 MHz, DMSO-d₆), δ ppm 0.82 (m, 18H), 1.31 (m, 3H), 1.52 (m, 2H), 1.80 (m, 1H), 2.45 (s, 3H), 2.67 (m, 4H), 3.09 (m, 4H), 3.50 (s, 1H), 3.66 (m, 1H), 3.84 (d, J=9.93 Hz, 1H), 3.93 (d, J=11.03 Hz, 1H), 4.14 (m, 1H), 4.35 (s, 1H), 4.67 (d, J=7.35 Hz, 1H), 6.64 (d, J=9.93 Hz, 1H), 7.21 (m, 12H), 7.66 (t, J=7.72 Hz, 1H), 7.86 (m, 4H), 8.63 (d, J=4.78 Hz, 1H).

### EXAMPLE 19A

### Ethyl 2-(3-pyridinyl)-1,3-thiazole-4-carboxylate

A solution containing thionicotinamide (30 g, 217.1 mmol) in ethanol (540 mL) was treated with ethyl bromopyruvate (30.3 mL, 241.4 mmol), heated at 70° C. for 3 hours, cooled to 25° C., concentrated and partitioned between chloroform and saturated NaHCO₃. The organic phase was washed with brine, dried over MgSO₄, filtered and concentrated. The residue was chromatographed on silica gel eluting with chloroform and then with 15% methanol in chloroform containing 1% ammonium hydroxide to give the product (36.3 g, 71% yield).

### EXAMPLE 19B

### 2-(3-pyridinyl)-1,3-thiazole-4-carbaldehyde

A solution containing the product from Example 19A (20 g, 85.5 mmol) in dichloromethane (340 mL) was treated dropwise with DIBAL (86 mL, 1 M in dichloromethane) at −78° C., stirred at −78° C. for 2 hours, treated with DIBAL (43 mL, 1 M in dichloromethane), stirred at −78° C. for 1 hour, treated with methanol (20 mL) at −78° C., warmed to 25° C., treated with dichloromethane (250 mL), saturated aqueous sodium potassium tartrate (350 mL), and pH 7 buffer (300 mL), stirred vigorously with a mechanical stirrer for 16 hours, and filtered through celite®. The aqueous phase was washed with chloroform, and the combined organic phase were washed with brine and dried over MgSO₄, filtered and concentrated. The residue was chromatographed on silica gel eluting 0-4% methanol/chloroform to give the title compound (11.61 g, 71% yield).

### EXAMPLE 19C

### tert-butyl(2S)-3,3-dimethyl-2-(2-oxo-3-{[2-(3-pyridinyl)-1,3-thiazol-4-yl]methyl}-1-imidazolidinyl)butanoate

A solution containing the product from Example 6F (0.855 g, 3.72 mmol) in a mixture of benzene (10 mL) and ethanol (10 mL) was treated with the product from Example 19B (0.70 g, 3.72 mmol), heated at 70° C. for 1 hour, cooled to 25° C. and treated with sodium borohydride (0.422 g, 11.16 mmol), stirred at 25° C. for 2 hours, quenched with sodium bicarbonate solution and partitioned between ethyl acetate and water. The organic phase was washed with brine, dried over MgSO₄, filtered and concentrated. A solution of the residue (3.72 mmol) in toluene (85 mL) was treated with bis(4-nitrophenyl)carbonate (1.36 g, 4.47 mmol), heated at 100° C. for 24 hours, cooled to 25° C., and partitioned between ethyl acetate and 10% K₂CO₃. The organic phase was washed several times with 10% K₂CO₃, and with brine, dried over MgSO₄, filtered and concentrated. The residue was chromatographed on silica gel eluting with 40% chloroform in hexanes to give the title compound (0.61 g, 39% yield).

### EXAMPLE 19D

### (2S)-3,3-dimethyl-2-(2-oxo-3-{[2-(3-pyridinyl)-1,3-thiazol-4-yl]methyl}-1-imidazolidinyl)butanoic Acid

A solution containing the product from Example 19C (0.61 g, 1.42 mmol) in dichloromethane (7 mL) was treated with trifluoracetic acid (4 mL), stirred at 25° C. for 1 hour, concentrated, and azeotroped several times with tolune to give the title compound as the trifluoroacetic acid salt, which was used without further purification.

### EXAMPLE 19E

### methyl(1S)-1-{[((1S,2S,4S)-1-benzyl-4-{[(2S)-3,3-dimethyl-2-(2-oxo-3-{[2-(3-pyridinyl)-1,3-thiazol-4-yl]methyl}-1-imidazolidinyl)butanoyl]amino}-2-hydroxy-5-phenylpentyl)amino]carbonyl}-2,2-dimethylpropylcarbamate

A solution containing the product from Example 8B (1.4 g, 3.08 mmol) in THF (30 mL) was treated with the product from Example 19D (1.5 g, 3.07 mmol), DEPBT (1.4 g, 4.68 mmol), and N,N-diisopropylethylamine (2.75 mL, 15.78 mmol), stirred at 25° C. for 16 hours, and partitioned between dichloromethane and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate. The product was then purified by reversed phase chromatography on a C18 column eluting with a gradient starting with 5-100% acetonitrile in water (0.1% TFA). The product was partitioned between dichloromethane and saturated NaHCO₃ solution. The organic phase was washed brine, dried over MgSO₄, filtered and concentrated to give the title compound (1.49 g, 60% yield). ¹H NMR (300 MHz, DMSO-d₆), δ ppm 0.84 (s, 9H), 0.86 (s, 9H), 1.49 (m, 2H), 2.39(m, 2H), 2.62 (m, 1H), 2.72 (d, J=6.99 Hz, 2H), 3.16 (m, 3H), 3.57 (m, 4H), 3.91 (d, J=9.56 Hz, 1H), 3.98 (s, 1H), 4.17 (m, 2H), 4.48 (m, 2H), 4.80 (d, J=5.52 Hz, 1H), 6.81 (d, J=9.19 Hz, 1H), 6.94 (m, 1H), 7.04 (m, 4H), 7.15 (m, 5H), 7.52 (m, 2H), 7.59 (s, 1H), 7.88 (d, J=9.56 Hz, 1H), 8.30 (m, 1H), 8.66 (dd, J=4.78, 1.47 Hz, 1H), 9.14 (d, J=1.47 Hz, 1H).

### EXAMPLE 20A

### (2S)-3,3-dimethyl-2-[2-oxo-3-(3-pyridinylmethyl)-1-imidazolidinyl]butanoic Acid

A solution containing the product from Example 6F (0.10 g, 0.43 mmol) in a mixture of benzene (1.6 mL) and methanol (1.66 mL) was treated with pyridine-3-carboxaldehyde (0.041 mL, 0.43 mmol), stirred at 50° C. for 18 hours, cooled to 25° C., treated with sodium borohydride (0.033 g, 0.87 mmol), stirred at 25° C. for 1 hour, quenched with saturated NaHCO₃, stirred for 1 hour, and partitioned between ethyl acetate and saturated NaHCO₃. The organic phase was washed with brine, dried over MgSO₄, filtered and concentrated. A solution of the residue (0.127 g, 0.40 mmol) in 1,2-dichloroethane (7 mL) was treated with N,N-disuccinimidyl carbonate (0.134 g, 0.52 mmol) and triethylamine (0.07 mL, 0.50 mmol), stirred at 25° C. for 16 hours, diluted with chloroform and partitioned with 10% Na₂CO₃. The organic phase was washed with brine, dried over MgSO₄, filtered and concentrated. A solution of the residue (0.146 g) in dichloromethane (2 mL) was treated with trifluoracetic acid (2 mL), stirred at 25° C. for 2 hours, concentrated, and azeotroped with toluene several times to give the title compound (0.252 g), as the trifluoroacetic acid salt.

### EXAMPLE 20B

### methyl(1S)-1-({[(1S,2S,4S)-1-benzyl-4-({(2S)-3,3-dimethyl-2-[2-oxo-3-(3-pyridinylmethyl)-1-imidazolidinyl]butanoyl}amino)-2-hydroxy-5-phenylpentyl]amino}carbonyl)-2,2-dimethylpropylcarbamate

A solution containing the product from Example 8B (2.0 g, 4.40 mmol) in DMF (10 mL) was treated with the product from Example 20A (1.78 g, 4.39 mmol), EDAC (1.01 g, 5.27 mmol), HOBT (0.7 g, 5.19 mmol), and NMM (0.96 mL, 8.72 mmol), stirred at 25° C. for 16 hours, additional acid (0.17 g), EDAC (0.42 g), HOBT (0.3 g), NMM (0.5 mL), and DMF (5 mL) was added, stirred for 16 hours at 25° C., and concentrated. The reaction was partitioned between ethyl acetate and water. The organic phase was washed with brine and dried over Na₂SO₄, filtered and concentrated. The residue was chromatographed on silica gel eluting with 3% methanol in dichloromethane to give the title compound (2.0 g, 62% yield). ¹H NMR (300 MHz, DMSO-d₆), δ ppm 0.85 (d, J=1.47 Hz, 18H), 1.01 (m, 1H), 1.51 (m, 2H), 2.39 (m, 2H), 2.66 (m, 1H), 2.72 (d, J=6.99 Hz, 2H), 2.90 (m, 2H), 3.21 (m, 1H), 3.58 (m, 3H), 3.91 (d, J=9.93 Hz, 1H), 3.96 (s, 1H), 4.19 (m, 2H), 4.34 (d, J=2.94 Hz, 2H), 4.80 (d, J=5.52 Hz, 1H), 6.81 (d, J=9.93 Hz, 1H), 7.02 (m, 5H), 7.14 (m, 5H), 7.40 (dd, J=8.09, 4.78 Hz, 1H), 7.50 (d, J=9.19 Hz, 1H), 7.67 (m, 1H), 7.89 (d, J=9.19 Hz, 1H), 8.51 (d, JJ=2.94 Hz, 2H).

### EXAMPLE 21

### methyl(1S)-1-[({(1S,3S,4S)-3-hydroxy-4-[((2S)-3-methyl-2-{3-[(2-methyl-3-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate

A solution containing the product from Example 2C (1.1 g, 2.07 mmol) in THF (20 mL) was treated with the product from Example 15C (0.87 g, 2.07 mmol), DEPBT (0.93 g, 3.11 mmol), and N,N-diisopropylethylamine (1.8 mL, 10.33 mmol), stirred at 25° C. for 3 hours, and partitioned between ethyl acetate and 10% Na₂CO₃ solution. The organic phase was washed with additional 10% Na₂CO₃ solution and brine, dried over MgSO₄, filtered and concentrated. The residue was purified by chromatography on silica gel eluting with a gradient starting with 0-100% ethyl acetate/dichloromethane, followed by 0-10% methanol in ethyl acetate to give the title compound (1.17 g, 69% yield).

¹H NMR (300 MHz, DMSO-d₆), δ ppm 0.83 (m, 18H), 1.29 (m, 1H), 1.53 (m, 2H), 1.80 (m, 1H), 2.48 (s, 3H), 2.72 (m, 3H), 2.96 (m, 3H), 3.50 (s, 3H), 3.65 (m, 1H), 3.84 (d, J=9.93 Hz, 1H), 3.94 (d, J=11.03 Hz, 1H), 4.15 (m, 2H), 4.32 (s, 2H), 4.67 (d, J=7.35 Hz, 1H), 6.64 (d, J=9.93 Hz, 1H), 7.09 (m, 5H), 7.22 (m, 3H), 7.31 (m, 2H), 7.52 (dd, J=7.72, 1.47 Hz, 1H), 7.86 (m, 5H), 8.36 (dd, J=4.78, 1.47 Hz, 1H), 8.63 (d, J=4.41 Hz, 1H).

### EXAMPLE 22A

### tert-butyl(1S,3S,4S)-1-benzyl-4-[((2S)-3,3-dimethyl-2-{3-[(6-methyl-2-pyridinyl)methyl]-2-oxo-1-imidazolidinyl}butanoyl)amino]-3-hydroxy-5-phenylpentylcarbamate

A solution of the product from Example 6A (3.0 g, 7.81 mmol) in THF (80 mL) was treated with the product from Example 10D (2.93 g, 8.57 mmol), DEPBT (3.5 g, 11.71 mmol), and N,N-diisopropylethylamine (7 mL, 40.19 mmol) and the mixture was stirred at 25 C for 3 hours. The mixture was partitioned between ethyl acetate and 10% Na₂CO₃ solution. The organic phase was washed with additional 10% Na₂CO₃ solution and brine, dried over MgSO₄, filtered and concentrated. The product was used without further purification.

### EXAMPLE 22B

### methyl(1S)-1-[({(1S,3S,4S)-1-benzyl-4-[((2S)-3,3-dimethyl-2-{3-[(6-methyl-2-pyridinyl)methyl]-2-oxo-1-imidazolidinyl}butanoyl)amino]-3-hydroxy-5-phenylpentyl}amino)carbonyl]-2,2-dimethylpropylcarbamate

A solution of the product from Example 22A (7.81 mmol) in dichloromethane (20 mL) was treated with trifluoroacetic acid (20 mL), and the mixture was stirred at 25 C for 1 hour. The solvent was concentrated and the reaction was partitioned between ethyl acetate and 10% Na₂CO₃, and the organic phase was washed with brine and dried over MgSO₄, filtered and concentrated. A solution of the residue (7.81 mmol) in THF (80 mL) was treated with the product from Example 1F (1.6 g, 8.47 mmol, DEPBT (3.5 g, 11.71 mmol, and N,N-diisopropylethylamine (6.8 mL, 39.04 mmol) and the mixture was stirred at 25° C. for 5 hours. The mixture was partitioned between ethyl acetate and 10% Na₂CO₃ solution. The organic phase was washed with additional 10% Na₂CO₃ solution and brine, dried over MgSO₄, filtered and concentrated. The product was purified by chromatography on silica gel eluting with a gradient starting with 0-100% ethyl acetate/dichloromethane, followed by 0-10% methanol in ethyl acetate. The product was then purified by reversed phase chromatography on a C18 column eluting with a gradient starting with 5-100% acetonitrile in water (0.1% TFA). The product was partitioned between ethyl acetate and 10% Na₂CO₃ solution. The organic phase was washed brine, dried over MgSO₄, filtered and concentrated to give the title compound (1.32 g, 23% yield). ¹H NMR (300 MHz, DMSO-d₆), δ ppm 0.82 (s, 9H), 0.91 (s, 9H), 1.25 (m, 1H), 1.51 (m, 2H), 2.36 (m, 1H), 2.46 (s, 3H), 2.70(m, 3H), 2.96 (m, 1H), 3.09 (m, 1H), 3.23 (m, 1H), 3.55 (s, 3H), 3.65 (m, 1H), 3.83 (d, J=9.93 Hz, 1H), 4.14 (m, 3-H), 4.35 (m, 2H), 4.50 (d, J=7.72 Hz, 1H), 6.64 (d, J=9.93 Hz, 1H), 7.09 (m, 12H), 7.47 (d, J=9.19 Hz, 1H), 7.71 (m, 2H).

### EXAMPLE 23A

### benzyl(2S)-3-[4-(benzyloxy)phenyl]-2-(dibenzylamino)propanoate

A suspension of L-Tyrosine (20 g, 110.4 mmol) in a mixture of water and ethanol (2:1, respectively, 120 mL) was treated with potassium carbonate (76.3 g, 552.1 mmol) and benzyl chloride (63.5 mL, 551.8 mmol), and the mixture was heated at reflux for 68 hours. The reaction was cooled to 25° C., treated with a mixture of hexanes and THF (1:1, 500 mL), followed by water. The mixture was partitioned and the organic phase was washed two times with a mixture of water and methanol (2:1, respectively) and then with brine, dried over MgSO₄, filtered and concentrated. The crude product (53.5 g) was used without further purification.

### EXAMPLE 23B

### (4S)-5-[4-(benzyloxy)phenyl]-4-(dibenzylamino)-3-oxopentanenitrile

A solution of sodium bis(trimethylsilyl)amide (1 M in THF, 330 mL) at −45° C., was treated dropwise with acetonitrile (18.8 mL, 360 mmol) and the mixture was stirred for 15 minutes at −45° C. and then cooled to −78° C., treated dropwise with a solution of the product from Example 23A (53.5 g, 110 mmol) in THF (150 mL), warmed to −45° C., stirred for 1 hour, treated with solid NH₄Cl (40 g), warmed to 5° C., treated with water, warmed to 25° C. and partitioned between ethyl acetate and water. The organic phase was washed with brine and dried over MgSO₄, filtered and concentrated. Precipitation from ethanol gave the product (19.0 g, 36% yield).

### EXAMPLE 23C

### (2S)-5-amino-1-[4-(benzyloxy)phenyl]-2-(dibenzylamino)-6-phenyl-4-hexen-3-one

A solution containing the product from Example 23B (19.0 g, 40.1 mmol) in THF (48 mL) was treated dropwise with a solution of benzyl magnesium bromide (120 mL, 1 M in ether) at 0° C. The mixture was allowed to warm to 25° C. and was stirred for 16 hours. The reaction was cooled to 0° C. and quenched with 10% citric acid, followed by partitioning between ethyl acetate and water. The organic phase was washed with brine and dried over MgSO₄, filtered and concentrated to give the crude product (23.2 g), which was used without further purification.

### EXAMPLE 23D

### (2S,3S,5S)-5-amino-1-[4-(benzyloxy)phenyl]-2-(dibenzylamino)-6-phenyl-3-hexanol

(i) A suspension of NaBH₄ (6.07 g, 160.4 mmol) in THF (170 mL) at −10° C. was treated with methansulfonic acid (26.0 mL, 401.0 mmol) dropwise. After complete addition, a solution containing the product from Example 23C (23.2 g, 40.1 mmol) in a mixture of THF (60 mL) and water (6 mL) was added and the mixture was stirred at −10° C. for 18 hours.

(ii) A suspension of NaBH₄ (6.07 g, 160.4 mmol) in THF (170 mL) at 0° C. was treated dropwise with trifluoroacetic acid (15.4 mL, 200.5 mmol), stirred at 0° C. for 30 minutes, treated with the solution from step (i), warmed to 25° C., stirred for 3 hours, treated with a mixture of NaBH₄ (6.07 g, 160.4 mmol) and trifluoroacetic acid (15.4 mL, 200.5 mmol) prepared as described above, warmed to 25° C. and stirred for 2 hours. The reaction was cooled to 0° C. and quenched cautiously by slow addition of NaOH solution (300 mL, 3 N), followed by partitioning between tert-butyl methyl ether and water. The organic phase was washed with NaOH solution (0.5 N), NH₄Cl solution, and brine, dried over MgSO₄, filtered and concentrated to give the crude product (22.9 g), which was used without further purification.

### EXAMPLE 23E

### tert-butyl(1S,3S,4S)-1-benzyl-5-[4-(benzyloxy)phenyl]-4-(dibenzylamino)-3-hydroxypentylcarbamate

A solution containing the product from Example 23D (22.9 g, 40.1 mmol) in tert-butyl methyl ether (200 mL) was treated with 10% K₂CO₃ (95 mL) and di-tert-butyl dicarbonate (14.0 g, 64.2 mmol), and stirred at 25° C. for 2 hours. The organic phase layer was washed with water and brine, dried over MgSO₄, filtered and concentrated. The residue was chromatographed on silica gel eluting with 20% hexanes in chloroform and then with 10% ethyl acetate in chloroform to give the title compound (12.3 g, 46% yield).

### EXAMPLE 23F

### tert-butyl(1S,3S,4S)-4-amino-1-benzyl-3-hydroxy-5-(4-hydroxyphenyl)pentylcarbamate

A solution containing the product from Example 23E (12.3 g, 18.4 mmol) in THF (169 mL) was treated with 10% Pd on carbon (2.5 g) and ammonium formate (6.9 g, 109.4 mmol) and the mixture was heated at reflux for 1.5 hours. Additional 10% Pd on carbon (1.25 g) and NH₄CO₂H (3.45 g) was added and the mixture was heated at reflux for 2.5 hours. The reaction was concentrated and partitioned between chloroform and water and the solution was adjusted to pH 10 with NaHCO₃ solution. The organic phase was washed with brine and dried over MgSO₄, filtered and concentrated to give the title compound (6.1 g, 82% yield), which was used without further purification.

### EXAMPLE 23G

### benzyl(1S,2S,4S)-4-[(tert-butoxycarbonyl)amino]-2-hydroxy-1-(4-hydroxybenzyl)-5-phenylpentylcarbamate

A solution containing the product from Example 23F (6.1 g, 15.2 mmol) in THF (150 mL) was treated with N-benzyloxycarbonyloxy)succinimide (3.4 g, 13.6 mmol) and N,N-diisopropylethylamine (3.3 mL, 19.0 mmol), stirred at 25° C. for 68 hours and concentrated. The residue was chromatographed on silica gel eluting with 33% ethyl acetate in chloroform and then with 10% methanol in chloroform to give the title compound (5.1 g, 63% yield).

### EXAMPLE 23H

### 4-{(2S,3S,5S)-2-{[(benzyloxy)carbonyl]amino}-5-[(tert-butoxycarbonyl)amino]-3-hydroxy-6-phenylhexyl}phenyl Trifluoromethanesulfonate

A solution containing the product from Example 23G (5.1 g, 9.6 mmol) in dichloromethane (50 mL) was treated with N-Phenyltrifluoromethanesulfonimide (4.1 g, 11.5 mmol) and DMAP (1.4 g, 11.5 mmol), heated at reflux for 1 hour, cooled to 25° C. and chromatographed on silica gel eluting with 0-50% ethyl acetate/chloroform to give the title compound (4.7 g, 74% yield).

### EXAMPLE 23I

### benzyl(4S,5S)-5-{(2S)-2-[(tert-butoxycarbonyl)amino]-3-phenylpropyl}-2,2-dimethyl-4-{4-[(trifluoroacetyl)oxy]benzyl}-1,3-oxazolidine-3-carboxylate

A solution containing the product from Example 23H (4.7 g, 7.1 mmol) in 2,2-dimethoxypropane (70 mL) was treated with p-toluenesulfonic acid monohydrate (0.067 g, 0.35 mmol), and the mixture was stirred at 25° C. for 1 hour. Triethylamine (0.3 mL, 2.15 mmol) was added, and the reaction was partitioned between ethyl acetate and water. The organic phase was washed with brine and dried over MgSO₄, filtered and concentrated to give the title compound (4.83 g, 97% yield), which was used without further purification.

### EXAMPLE 23J

### benzyl(1S,2S,4S)-4-[(tert-butoxycarbonyl)amino]-2-hydroxy-5-phenyl-1-[4-(2-pyridinyl)benzyl]pentylcarbamate

A solution containing the product from Example 23I (2.65 g, 3.75 mmol) in DMF (20 mL) was treated with LiCl (1.6 g, 37.74 mmol), dichlorobis(triphenylphosphine)palladium(II) (0.5 g, 0.71 mmol), and 2-tri-n-butylstannylpyridine (2.6 mL, 11.30 mmol), heated at 100° C. for 16 hours, cooled and partitioned between ethyl acetate and water. The organic phase was washed with brine and dried over MgSO₄, filtered and concentrated. A solution of the residue in THF (30 mL) and aqueous HCl (30 mL, 1 N) was stirred at 50° C. for 48 hours, cooled to 0° C. and adjusted to pH 8 with 3 N NaOH solution. The reaction mixture was partitioned between ethyl acetate and water, and the organic phase was washed with brine and dried over MgSO₄, filtered and concentrated. A solution of the residue in THF (30 mL) was treated with triethylamine (1 mL, 13.6 mmol) and di-tert-butyl dicarbonate (0.82 g, 3.75 mmol), stirred at 25° C. for 16 hours, and partitioned between ethyl acetate and saturated NaHCO₃. The organic phase was washed with brine and dried over MgSO₄, filtered and concentrated. The residue was chromatographed on silica gel eluting with 0-100% ethyl acetate/dichloromethane to give the title compound (0.568 g, 25% yield).

### EXAMPLE 23K

### benzyl(2S)-3-(4-bromophenyl)-2-(dibenzylamino)propanoate

A suspension of L-p-bromophenylalanine (5 g, 20.5 mmol) in a mixture of water and ethanol (2:1, respectively, 20 mL) was treated with potassium carbonate (9.3 g, 67.3 mmol) and benzyl chloride (7.77 mL, 67.5 mmol), heated at reflux for 16 hours, cooled to 25° C. and treated with a mixture of hexanes and THF (1:1, 100 mL), followed by addition of water. The mixture was partitioned and the organic phase was washed two times with a mixture of water and methanol (2:1, respectively) and then with brine, dried over MgSO₄, filtered and concentrated. The crude product (11.23 g) was used without further purification.

### EXAMPLE 23L

### benzyl(2S)-2-(dibenzylamino)-3-[4-(2-pyridinyl)phenyl]propanoate

A solution containing the product from Example 23K (11.0 g, 20.5 mmol) in DMF (90 mL) was treated with LiCl (8 g, 188.7 mmol), tetrakis(triphenylphosphine)palladium(0) (5 g, 4.3 mmol), and 2-tri-n-butylstannylpyridine (22 g, 59.8 mmol), heated at 80° C. for 16 hours, cooled, filtered and concentrated. The residue was partitioned between ethyl acetate and water, and the organic phase was washed with brine and dried over MgSO₄, filtered and concentrated. The residue was purified by chromatography on silica gel eluting with a gradient 0-25% ethyl acetate/hexanes to give the title compound (7.6 g, 72% yield).

### EXAMPLE 23M

### (4S)-4-(dibenzylamino)-3-oxo-5-[4-(2-pyridinyl)phenyl]pentanenitrile

A solution of sodium bis(trimethylsilyl)amide (1 M in THF, 50 mL) at −45° C., was treated with a solution of acetonitrile (2.81 mL, 53.4 mmol) in THF (10 mL) dropwise and the mixture was stirred for 15 minutes at −45° C. and then cooled to −78° C., treated dropwise with a solution of the product from Example 23L (7.6 g, 14.8 mmol) in THF (20 mL), stirred at −45° C. for 1 hour, treated with solid NH₄Cl (10 g), warmed to 5° C., followed by the addition of water. The mixture was allowed to warm to 25° C. and was partitioned between ethyl acetate and water. The organic phase was washed with brine and dried over MgSO₄, filtered and concentrated. The residue was titurated with ethanol and the resulting solid was filtered and dried to give the title compound (4.3 g, 62% yield).

### EXAMPLE 23N

### (2S,4E)-5-amino-2-(dibenzylamino)-6-phenyl-1-[4-(2-pyridinyl)phenyl]-4-hexen-3-one

A solution containing the product from Example 23M (4.3 g, 9.65 mmol) in THF (15 mL) was treated dropwise with a solution of benzyl magnesium bromide (30 mL, 1 M in ether) at 0° C., warmed to 25 C, stirred for 16 hours, cooled to 0° C., quenched with 10% citric acid, and partitioned between ethyl acetate and water. The organic phase was washed with brine and dried over MgSO₄, filtered and concentrated to give the crude product (6.18 g), which was used without further purification.

### EXAMPLE 23O

### (2S,3S,5S)-5-amino-2-(dibenzylamino)-6-phenyl-1-[4-(2-pyridinyl)phenyl]-3-hexanol

(i) A suspension of NaBH₄ (1.75 g, 46.3 mmol) in THF (45 mL) at −10° C. was treated with methansulfonic acid (7.46 mL, 114.9 mmol) dropwise. After complete addition, a solution containing the product from Example 23N (6.18 g, 9.65 mmol) in a mixture of THF (16 mL) and water (1.6 mL) was added and the mixture was stirred at −10° C. for 16 hours.

(ii) A suspension of NaBH₄ (1.75 g, 46.3 mmol) in THF (45 mL) at 0° C. was treated with trifluoroacetic acid (4.4 mL, 57.1 mmol) dropwise, stirred at 0° C. for 30 minutes, treated with a solution of step (i), warmed to 25° C., stirred for 16 hours, treated with a suspension of NaBH₄ (1.75 g, 46.3 mmol) and trifluoroacetic acid (4.4 mL, 57.1 mmol) prepared as described above at 0° C., warmed to 25 C and stirred for 16 hours. The reaction mixture was cooled to 0° C. and quenched cautiously by slow addition of NaOH solution (65 mL, 3 N), followed by partitioning between tert-butyl methyl ether and water. The organic phase was washed with NaOH solution (0.5 N), NH₄Cl solution, and brine, dried over MgSO₄, filtered and concentrated to give the crude product, which was used without further purification.

### EXAMPLE 23P

### tert-butyl(1S,3S,4S)-1-benzyl-4-(dibenzylamino)-3-hydroxy-5-[4-(2-pyridinyl)phenyl]pentylcarbamate

A solution containing the product from Example 23O (9.65 mmol) in tert-butyl methyl ether (50 mL) was treated with 10% K₂CO₃ (23 mL) and di-tert-butyl dicarbonate (3.5 g, 16.0 mmol) and the mixture was stirred at 25° C. for 1 hour. The reaction mixture was diluted with tert-butyl methyl ether and the organic phase layer was washed with water and brine, dried over MgSO₄, filtered and concentrated. The residue was chromatographed on silica gel eluting with 0-50% ethyl acetate/hexanes to give the title compound (2.7 g, 43% yield).

### EXAMPLE 23Q

### tert-butyl(1S,3S,4S)-4-amino-1-benzyl-3-hydroxy-5-[4-(2-pyridinyl)phenyl]pentylcarbamate

Method 1 A solution containing the product from Example 23J (0.568 g, 0.95 mmol) in a mixture of ethyl acetate (5 mL) and methanol (5 mL) was treated with Pd(OH)₂ on carbon (0.2 g, 20% Pd by wt.) and HCl solution (0.25 mL, 4N in dioxane), stirred under a hydrogen atmosphere (balloon pressure) for 16 hours at 25° C., filtered through a bed of celite®, rinsed with a mixture of 50% ethyl acetate in methanol, and concentrated. The residue was partitioned between ethyl acetate and saturated NaHCO₃. The organic phase was washed with brine and dried over MgSO₄, filtered and concentrated to give the title compound (0.442 g), which was used without further purification.

Method 2 A solution containing the product from Example 23P (2.7 g, 4.21 mmol) in a mixture of methanol (20 mL) and ethyl acetate (20 mL) was treated with 20% Pd(OH)₂ on carbon (1 g) and an HCl solution in dioxane (2 mL, 4 N), stirred under an atmosphere of hydrogen (balloon pressure) for 16 hours at 25° C., heated to 60° C. for 6 hours. The reaction cooled and filtered through celite®, and concentrated. The residue was partitioned between dichloromethane and half-saturated NaHCO₃. The organic phase was dried over MgSO₄, filtered and concentrated to give the title compound, which was used without further purification.

### EXAMPLE 23R

### tert-butyl(1S,3S,4S)-1-benzyl-3-hydroxy-4-({(2S)-2-[(methoxycarbonyl)amino]-3,3-dimethylbutanoyl}amino)-5-[4-(2-pyridinyl)phenyl]pentylcarbamate

A solution containing the product from Example 23Q (0.442 g, 0.95 mmol) in THF (10 mL) was treated with the product from Example 1F (0.20 g, 1.06 mmol), DEPBT (0.45 g, 1.50 mmol), and N,N-diisopropylethylamine (0.85 mL, 4.88 mmol), stirred at 25 C for 1 hour, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-75% ethyl acetate/dichloromethane to give the title compound (0.34 g, 56% yield).

### EXAMPLE 23S

### methyl(1S)-1-[({(1S,2S,4S)-4-amino-2-hydroxy-5-phenyl-1-[4-(2-pyridinyl)benzyl]pentyl}amino)carbonyl]-2,2-dimethylpropylcarbamate

A solution containing the product from Example 23R (0.34 g, 0.54 mmol) in dichloromethane (5 mL) was treated with trifluoroacetic acid (5 mL), stirred at 25° C. for 1 hour, and concentrated. The residue was partitioned between dichloromethane and saturated NaHCO₃ solution. The organic phase was washed with brine, dried over MgSO₄, filtered and concentrated. The crude product (0.251 g) was used without further purification.

### EXAMPLE 23T

### methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate

A solution containing the product from Example 23S (0.075 g, 0.14 mmol) in THF (1.5 mL) was treated with the product from Example 10D (0.073 g, 0.21 mmol), DEPBT (0.09 g, 0.30 mmol), and N,N-diisopropylethylamine (0.125 mL, 0.72 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The concentrate was purified by reversed phase chromatography on a C18 column eluting with a gradient starting with 5-100% acetonitrile in water (0.1% TFA). The product was partitioned between dichloromethane and saturated NaHCO₃ solution. The organic phase was washed brine, dried over MgSO₄, filtered and concentrated. The residue was then chromatographed on silica gel eluting with 0-100% ethyl acetate/dichloromethane, followed by 0-10% methanol in ethyl acetate, to give the title compound (0.063 g, 54% yield). ¹H NMR (300 MHz, DMSO-d₆), δ ppm 0.83 (s, 9H), 0.86 (s, 9H), 1.53 (m, 2H), 2.39 (m, 1H), 2.45 (m, 3H), 2.48 (m, 1H), 2.66 (d, J=10.66 Hz, 1H), 2.78 (d, J=6.99 Hz, 2H), 2.96 (m, 1H), 3.07 (q, J=8.70 Hz, 1H), 3.23 (m, 1H), 3.50 (s, 3H), 3.62 (m, 1H), 3.95 (d, J=8.1 Hz, 1H), 3.96's, 1H), 4.19 (m, 2H), 4.34 (m, 2H), 4.83 (d, J=5.52 Hz, 1H), 6.79 (d, J=9.56 Hz, 1H), 7.05 (m, 6H), 7.15 (d, J=7.35 Hz, 1H), 7.30 (m, 3H), 7.58 (d, J=9.19 Hz, 1H), 7.68 (t, J=7.72 Hz, 1H), 7.86 (m, 5H), 8.63 (d, J=4.78 Hz, 1H).

### EXAMPLE 24A

### tert-butyl(1S,2S,4S)-1-benzyl-4-[((2S)-3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentylcarbamate

A solution containing the product from Example 2A (0.185 g, 0.37 mmol) in THF (3.5 mL) was treated with the product from Example 10D (0.127 g, 0.37 mmol), DEPBT (0.167 g, 0.56 mmol), and N,N-diisopropylethylamine (0.32 mL, 1.84 mmol) and the mixture was stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The product was purified by reversed phase chromatography on a C18 column eluting with 5-100% acetonitrile in water (0.1% TFA). The product was partitioned between dichloromethane and saturated NaHCO₃ solution. The organic phase was washed brine, dried over MgSO₄, filtered and concentrated. The residue was then chromatographed on silica gel eluting with 0-10% methanol/chloroform to give the title compound (0.129 g, 46% yield).

### EXAMPLE 24B

### methyl(1S)-1-[({(1S,2S,4S)-1-benzyl-4-[((2S)-3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentyl}amino)carbonyl]-2,2-dimethylpropylcarbamate

A solution containing the product from Example 24A (0.129 g, 0.17 mmol) dichloromethane (0.8 mL) was treated with trifluoroacetic acid (0.8 mL), stirred at 25° C. for 1 hour and concentrated. A solution of the residue (0.17 mmol) in THF (1.8 mL) was treated with the product from Example 1F (0.033 g, 0.17 mmol), DEPBT (0.077 g, 0.26 mmol), and N,N-diisopropylethylamine (0.30 mL, 1.72 mmol), stirred at 25° C. for 18 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-5% methanol/ethyl acetate to give the title compound (0.057 g, 40% yield). ¹H NMR (300 MHz, DMSO-d₆), δ ppm 0.85 (d, J=1.10 Hz, 18H), 1.55 (m, 2H), 2.43 (m, 5H), 2.72 (m, 4H), 2.95 (m, 1H), 3.19 (m, 1H), 3.58 (m, 4H), 3.93 (d, J=9.56 Hz, 1H), 3.99 (s, 1H), 4.28 (m, 4H), 4.84 (d, J=5.52 Hz, 1H), 6.82 (d, J=9.93 Hz, 1H), 6.97 (d, J=7.72 Hz, 1H), 7.16 (m, 8H), 7.29 (m, 1H), 7.52 (d, J=8.82 Hz, 1H), 7.61 (t, J=7.72 Hz, 1H), 7.79 (m, 4H), 7.95 (d, J=8.82 Hz, 1H), 8.61 (d, J=4.78 Hz, 1H).

### EXAMPLE 25

### methyl(1S,4S,5S,7S,10S)-7-benzyl-1,10-ditert-butyl-5-hydroxy-2,9,12-trioxo-4-[4-(2-pyridinyl)benzyl]-13-oxa-3,8,11-triazatetradec-1-ylcarbamate

A solution of the product from Example 23S (0.025 g, 0.047 mmol) in THF (0.5 mL) was treated with the product from Example 1F (0.010 g, 0.053 mmol), DEPBT (0.020 g, 0.067 mmol), and N,N-diisopropylethylamine (0.040 mL, 0.229 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.015 g, 45% yield). ¹H NMR (300 MHz, DMSO-d₆), δ ppm 0.77 (s, 9H), 0.83 (s, 9H), 1.48 (m, 2H), 2.74 (m, 3H), 3.50 (s, 3H), 3.54 (s, 3H), 3.64 (m, 1H), 3.79 (d, J=9.19 Hz, 1H), 3.93 (d, J=9.56 Hz, 1H), 4.09 (m, 2H), 4.85 (d, J=5.88 Hz, 1H), 6.60 (d, J=9.93 Hz, 1H), 6.75 (d, J=9.93 Hz, 1H), 7.11 (m, 5H), 7.31 (m, 3H), 7.59 (d, J=9.19 Hz, 1H), 7.74 (d, J=8.82 Hz, 1H), 7.86 (m, 4H), 8.63 (d, J=4.41 Hz, 1H).

### EXAMPLE 26A

### 1:1 Mixture of (3R,3aS,6aR)-hexahydrofuro[2,3-b]furan-3-yl 4-nitrophenyl carbonate and (3S,3aR,6aS)-hexahydrofuro[2,3-b]furan-3-yl 4-nitrophenyl carbonate

A solution of (3S,3aR,6aS)— and (3R,3aS,6aR)-3-hydroxy-4H-hexahydrofuro[2,3-b]furan (prepared as described in: Gosh, A. K.; Kincaid, J. F.; Walters, D. E.; Chen, Y.; Chaudhuri, N. C.; Thompson, W. J.; Culberson, C.; Fitzgerald, P. M. D.; Lee. H. Y.; McKee, S. P.; Munson, P. M.; Duong, T. T.; Darke, P. L.; Zugay, J. A.; Schleif, W. A.; Axel, M. G.; Lin, J.; Huff, J. R. Journal of Medicinal Chemistry 1996, 39, 3278-3290.) (1.5 g, 11.5 mmol) in dichloromethane (40 mL) at 0° C. was treated with NMM (1.9 mL, 17.3 mmol) and 4-nitrophenyl chloroformate (2.9 g, 14.4 mmol), stirred for 16 hours at 0° C. and concentrated. The residue was chromatographed on silica gel, eluting with 25% ethyl acetate in hexanes to give the title compound (2.91 g, 86% yield).

### EXAMPLE 26B

### 1:1 Mixture of (3R,3aS,6aR)-hexahydrofuro[2,3-b]furan-3-yl(1S,3S,4S)-1-benzyl-3-hydroxy-4-({(2S)-2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl(1S,3S,4S)-1-benzyl-3-hydroxy-4-({(2S)-2-[(methoxycarbonyl)amino]-3,3-dimethylbutanoyl}amino)-5-[4-(2-pyridinyl)phenyl]pentylcarbamate

A solution of the product from Example 23S (0.05 g, 0.094 mmol) in THF (0.5 mL) was treated with triethylamine (0.025 mL, 0.179 mmol) and the product from Example 26A (0.040 g, 0.135 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.050 g, 77% yield). ¹H NMR (300 MHz, DMSO-d₆), δ ppm 0.86 (d, J=4.78 Hz, 9H), 1.28 (m, 2H), 1.56 (m, 3H), 2.62 (m, 2H), 2.79 (m, 3H), 3.41 (m, 1H), 3.51 (s, 3H), 3.58 (m, 1H), 3.72 (m, 3H), 3.93 (m, 1H), 4.18 (m, 1H), 4.80 (m, 2H), 5.47 (d, J=4.78 Hz, 1H), 6.84 (t, J=9.93 Hz, 1H), 7.16 (m, 6H), 7.31 (d, J=8.46 Hz, 3H), 7.59 (d, J=9.19 Hz, 1H), 7.89 (m, 4H), 8.63 (d, J=4.78 Hz, 1H).

### EXAMPLE 27

### 1:1 Mixture of (3R,3aS,6aR)-hexahydrofuro[2,3-b]furan-3-yl(1S,2S,4S)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl(1S,2S,4S)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate

A solution of the product from Example 2C (0.025 g, 0.047 mmol) in THF (0.25 mL) was treated with triethylamine (0.013 mL, 0.093 mmol) and the product from Example 26A (0.020 g, 0.067 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.024 g, 74% yield).

¹H NMR (300 MHz, DMSO-d₆), δ ppm 0.84 (d, J=9.19 Hz, 9H), 1.44 (m, 5H), 2.73 (m, 5H), 3.49 (m, 3H), 3.73 (m, 6H), 4.19 (m, 1H), 4.68 (dd, J=17.65, 6.25 Hz, 1H), 4.82 (m, 1H), 5.49 (m, 1H), 6.70 (t, J=9.74 Hz, 1H), 6.86 (t, J=8.82 Hz, 1H), 7.19 (m, 7H), 7.31 (m, 1H), 7.88 (m, 5H), 8.63 (d, J=4.78 Hz, 1H).

### EXAMPLE 28

### methyl(1S)-1-[({(1S,2S,4S)-2-hydroxy-4-[((2S)-3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate

A solution containing the product from Example 23S (0.025 g, 0.047 mmol) in THF (0.5 mL) was treated with the product from Example 7B (0.025 g, 0.060 mmol), DEPBT (0.025 g, 0.084 mmol), and N,N-diisopropylethylamine (0.040 mL, 0.230 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by eluting with 0-10% methanol in ethyl acetate to give the title compound (0.021 g, 54% yield). ¹H NMR (300 MHz, DMSO-d₆), δ ppm 0.60 (d, J=6.62 Hz, 3H), 0.73 (t, J=7.17 Hz, 3H), 0.87 (m, 10H), 1.27 (m, 1H), 1.54 (m, 2H), 1.75 (m, 1H), 2.43 (m, 4H), 2.66 (m, 1H), 2.77 (d, J=6.99 Hz, 2H), 2.91 (m, 1H), 3.12 (m, 3H), 3.51 (s, 3H), 3.59 (m, 1H), 3.85 (d, J=11.03 Hz, 1H), 3.94 (d, J=9.56 Hz, 1H), 4.14 (m, 2H), 4.33 (s, 2H), 4.81 (d, J=5.15 Hz, 1H), 6.80 (d, J=9.56 Hz, 1H), 7.07 (m, 7H), 7.30 (d, J=7.72 Hz, 3H), 7.58 (d, J=8.82 Hz, 1H), 7.65 (t, J=7.54 Hz, 1H), 7.87 (m, 5H), 8.63 (d, J=4.04 Hz, 1H).

### EXAMPLE 29

### methyl(1S)-1-[({(1R,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 1H (0.176 g, 0.33 mmol) in THF (3.3 mL) was treated with the product from Example 10D (0.113 g, 0.33 mmol), DEPBT (0.148 g, 0.49 mmol), and N,N-diisopropylethylamine (0.29 mL, 1.66 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with a gradient starting with 5-100% acetonitrile in water (0.1% TFA). The product was partitioned between ethyl acetate and saturated NaHCO₃, and the organic phase was washed with brine and dried over MgSO₄, filtered and concentrated to give the title compound (0.176 g, 65% yield). ¹H NMR (300 MHz, DMSO-d₆), δ ppm 0.80 (s, 9H), 0.88 (s, 9H), 1.29 (m, 2H), 1.53 (m, 1H), 2.45 (s, 3H), 2.66 (m, 3H), 2.83 (dd, J=13.79, 6.07 Hz, 1H), 3.03 (m, 2H), 3.23 (m, 1H), 3.53 (m, 4H), 3.84 (d, J=9.56 Hz, 1H), 4.01 (m, 2H), 4.16 (m, 1H), 4.34 (m, 2H), 4.44 (d, J=6.99 Hz, 1H), 6.88 (d, J=9.56 Hz, 1H), 7.09 (m, 7H), 7.24 (d, J=8.09 Hz, 2H), 7.32 (m, 1H), 7.54 (d, J=9.56 Hz, 1H), 7.67 (t, J=7.72 Hz, 1H), 7.89 (m, 5H), 8.64 (d, J=4.04 Hz, 1H).

### EXAMPLE 30A

### (2R)-2-[(methoxycarbonyl)amino]-3-methyl-3-(methylsulfanyl)butanoic Acid

A solution of L-penicillamine (0.5 g, 3.35 mmol) in methanol (3.3 mL) at 0° C. was treated with aqueous NaOH solution (3.7 mL, 1 N) and methyl iodide (0.23 mL, 3.69 mmol), stirred at 0° C. for 16 hours, treated with additional aqueous NaOH solution (3.5 mL, 3 N) at 0° C., followed by methyl chloroformate (0.5 mL, 6.47 mmol), warmed to 25 C and stirred for 3 hours, and partitioned between ethyl acetate and water. The organic phase was washed with brine and dried over MgSO₄, filtered and concentrated to give the title compound (0.428 g, 58% yield), which was used without further purification.

### EXAMPLE 30B

### methyl(1R,4S,5S,7S,10S)-4-benzyl-10-tert-butyl-5-hydroxy-1-[1-methyl-1-(methylsulfanyl)ethyl]-2,9,12-trioxo-7-[4-(2-pyridinyl)benzyl]-13-oxa-3,8,14-triazatetradec-1-ylcarbamate

A solution containing the product from Example 2C (0.10 g, 0.18 mmol) in THF (2 mL) was treated with the product from Example 30A (0.05 g, 0.226 mmol), DEPBT (0.085 g, 0.28 mmol), and N,N-diisopropylethylamine (0.165 mL, 0.947 mmol), stirred at 25° C. for 1 hour, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.652 g, 38% yield). ¹H NMR (300 MHz, DMSO-d₆), δ ppm 0.80 (s, 9H), 1.10 (s, 3H), 1.21 (s, 3H), 1.54 (m, 2H), 1.98 (s, 3H), 2.57 (m, 1H), 2.75 (m, 3H), 3.49 (s, 3H), 3.56 (s, 3H), 3.64 (m, 1H), 3.82 (d, J=9.56 Hz, 1H), 4.17 (m, 3H), 4.83 (d, J=5.88 Hz, 1H), 6.61 (d, J=9.56 Hz, 1H), 6.90 (d, J=9.56 Hz, 1H), 7.15 (m, 7H), 7.31 (m, 1H), 7.84 (m, 6H), 8.63 (d, J=4.78 Hz, 1H).

### EXAMPLE 31

### methyl(1R,4S,5S,7S,10S)-4-benzyl-10-tert-butyl-5-hydroxy-1-[1-methyl-1-(methylsulfonyl)ethyl]-2,9,12-trioxo-7-[4-(2-pyridinyl)benzyl]-13-oxa-3,8,11-triazatetradec-1-ylcarbamate

A solution of the product from Example 30B (0.015 g, 0.020 mmol) in a mixture of acetone and water (3:1, respectively, 0.20 mL) and THF (0.10 mL) was treated with 4-methylmorpholine N-oxide (0.014 g, 0.120 mmol) and aqueous OsO₄ solution (0.033 mL, 4%), stirred at 25 C for 16 hours, and partitioned between ethyl acetate and water. The organic phase was washed with brine and dried over MgSO₄, filtered and concentrated. The residue was chromatographed on silica gel eluting with 0-100% ethyl acetate/dichloromethane to give the title compound (0.013 g, 83% yield). H NMR (300 MHz, DMSO-d₆), δ ppm 0.82 (s, 9H), 1.14 (s, 3H), 1.26 (s, 3H), 1.50 (m, 2H), 2.57 (m, 1H), 2.75 (m, 3H), 2.88 (s, 3H), 3.50 (s, 3H), 3.57 (s, 3H), 3.69 (m, 1H), 3.83 (d, J=10.30 Hz, 1H), 4.03 (m, 1H), 4.16 (m, 1H), 4.69 (d, J=10.30 Hz, 1H), 4.89 (d, J=5.52 Hz, 1H), 6.64 (d, J=9.56 Hz, 1H), 7.16 (m, 8H), 7.31 (m, 1H), 7.85 (m, 5H), 8.01 (d, J=9.19 Hz, 1H), 8.63 (d, J=4.41 Hz, 1H).

### EXAMPLE 32

### methyl(1R,4S,6S,7S,10S)-4-benzyl-10-tert-butyl-6-hydroxy-1-[1-methyl-1-(methylsulfanyl)ethyl]-2,9,12-trioxo-7-[4-(2-pyridinyl)benzyl]-13-oxa-3,8,11-triazatetradec-1-ylcarbamate

A solution containing the product from Example 23S (0.025 g, 0.047 mmol) in THF (0.5 mL) was treated with the product from Example 30A (0.0125 g, 0.056 mmol), DEPBT (0.021 g, 0.070 mmol), and N,N-diisopropylethylamine (0.040 mL, 0.230 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.024 g, 69% yield). ¹H NMR (300 MHz, DMSO-d₆), δ ppm 0.84 (s, 9H), 1.08 (s, 3H), 1.11 (s, 3H), 1.50 (m, 2H), 1.93 (s, 3H), 2.45 (m, 1H), 2.75 (m, 3H), 3.51 (s, 3H), 3.56 (s, 3H), 3.67 (m, 1H), 394 (d, J=9.56 Hz, 1H), 4.10 (d, J=10.30 Hz, 3H), 4.84 (d, J=5.88 Hz, 1H), 6.79 (dd, J=15.81, 9.93 Hz, 2H), 7.09 (m, 5H), 7.30 (d, J=7.35 Hz, 3H), 7.57 (s, 1H), 7.87 (m, 5H), 8.63 (d, J=4.41 Hz, 1H).

### EXAMPLE 33

### methyl(1R,4S,6S,7S,10S)-4-benzyl-10-tert-butyl-6-hydroxy-1-[1-methyl-1-(methylsulfonyl)ethyl]-2,9,12-trioxo-7-[4-(2-pyridinyl)benzyl]-13-oxa-3,8,11-triazatetradec-1-ylcarbamate

A solution of the product from Example 32 (0.015 g, 0.020 mmol) in a mixture of acetone and water (3:1, respectively, 0.20 mL) and THF (0.15 mL) was treated with 4-methylmorpholine N-oxide (0.014 g, 0.120 mmol) and aqueous OsO₄ solution (0.030 mL, 4%), stirred at 25° C. for 16 hours, and partitioned between ethyl acetate and water. The organic phase was washed with brine and dried over MgSO₄, filtered and concentrated. The residue was chromatographed on silica gel eluting with 0-100% ethyl acetate/dichloromethane, followed by 0-5% methanol in ethyl acetate, to give the title compound (0.012 g, 77% yield). ¹H NMR (300 MHz, DMSO-d₆), δ ppm 0.85 (s, 9H), 1.05 (s, 3H), 1.25 (s, 3H), 1.50 (m, 2H), 2.79 (m, 8H), 3.51 (s, 3H), 3.56 (s, 3H), 3.95 (d, J=9.19 Hz, 1H), 4.08 (m, 2H), 4.53 (d, J=10.30 Hz, 1H), 4.88 (d, J=5.88 Hz, 1H), 6.79 (d, J=9.93 Hz, 1H), 7.11 (m, 6H), 7.31 (m, 3H), 7.62 (d, J=9.56 Hz, 1H), 7.86 (m, 4H), 8.17 (d, J=8.82 Hz, 1H), 8.63 (d, J=4.41 Hz, 1H).

### EXAMPLE 34

### methyl(1S)-1-[({(1S,3S,4S)-4-{[(2S)-3,3-dimethyl-2-(2-oxo-3-{[2-(3-pyridinyl)-1,3-thiazol-4-yl]methyl}-1-imidazolidinyl)butanoyl]amino}-3-hydroxy-5-phenyl-1-[4-(2-pyridinyl)benzyl]pentyl}amino)carbonyl]-2,2-dimethylpropylcarbamate

A solution containing the product from Example 2C (0.025 g, 0.047 mmol) in THF (0.5 mL) was treated with the product from Example 19D (0.030 g, 0.061 mmol), DEPBT (0.020 g, 0.067 mmol), and N,N-diisopropylethylamine (0.040 mL, 0.230 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.029 g, 70% yield). ¹H NMR (300 MHz, DMSO-d₆), δ ppm 0.83 (s, 9H), 0.89 (s, 9H), 1.54 (m, 2H), 2.34 (m, 1H), 2.63 (m, 2H), 2.79 (m, 1H), 3.16 (m, 4H), 3.50 (s, 3H), 3.65 (m, 1H), 3.85 (d, J=9.93 Hz, 1H), 4.14 (m, 3H), 4.50 (m, 3H), 6.63 (d, J=9.56 Hz, 1H), 6.98 (m, 1H), 7.06 (m, 4H), 7.22 (d, J=8.09 Hz, 2H), 7.31 (m, 1H), 7.52 (m, 2H), 7.61 (s, 1H), 7.87 (m, 5H), 8.31 (m, 1H), 8.65 (m, 2H), 9.14 (d, J=1.84 Hz, 1H).

### EXAMPLE 35

### methyl(1S)-1-[({(1S,2S,4S)-4-{[(2S)-3,3-dimethyl-2-(2-oxo-3-{[2-(3-pyridinyl)-1,3-thiazol-4-yl]methyl}-1-imidazolidinyl)butanoyl]amino}-2-hydroxy-5-phenyl-1-[4-(2-pyridinyl)benzyl]pentyl}amino)carbonyl]-2,2-dimethylpropylcarbamate

A solution containing the product from Example 23S (0.025 g, 0.047 mmol) in THF (0.5 mL) was treated with the product from Example 19D (0.030 g, 0.061 mmol), DEPBT (0.020 g, 0.067 mmol), and N,N-diisopropylethylamine (0.040 mL, 0.230 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.021 g, 50% yield). ¹H NMR (300 MHz, DMSO-d₆), δ ppm 0.82 (s, 9H), 0.86 (s, 9H), 1.53 (m, 2H), 2.40 (m, 1H), 2.64 (d, J=13.97 Hz, 1H), 2.77 (d, J=6.62 Hz, 2H), 3.15 (m, 4H), 3.51 (s, 3H), 3.62 (m, 1H), 3.96 (m, 2H), 4.18 (m, 2H), 4.47 (m, 2H), 4.82 (d, J=5.52 Hz, 1H), 6.79 (d, J=9.56 Hz, 1H), 6.95 (m, 1H), 7.03 (m, 4H), 7.30 (m, 3H), 7.54 (m, 3H), 7.87 (m, 5H), 8.30 (m, 1H), 8.65 (m, 2H), 9.14 (d, J=1.47 Hz, 1H).

### EXAMPLE 36A

### tert-butyl(2S,3S)-2-[(2-ethoxy-2-oxoethyl)amino]-3-methylpentanoate

A solution of L-iso-leucine tert-butyl ester hydrochloride (5 g, 22.34 mmol) in DMF (30 mL) was treated with triethylamine (3.1 mL, 22.34 mmol), stirred for 1 hour at 25° C., filtered to remove solid salts, and the filtrate was treated with triethylamine (9.3 mL, 67.0 mmol) and ethyl bromoacetate (9.9 mL, 67.0 mmol), and the reaction was stirred for 3 hours at 25° C. The reaction mixture was partitioned between ethyl acetate and water, and the organic phase was washed with brine and dried over MgSO₄, filtered and concentrated to give the title compound (5.7 g, 93% yield), which was used without further purification.

### EXAMPLE 36B

### tert-butyl(2S,3S)-2-[(aminocarbonyl)(2-ethoxy-2-oxoethyl)amino]-3-methylpentanoate

A solution containing the product from Example 36A (5.7 g, 20.9 mmol) in dichloromethane (60 mL) at 0° C. was treated with chlorosulfonyl isocyanate (2.7 mL, 31.0 mmol) and the mixture was stirred at 0° C. for 16 hours. Water (60 mL) was added to the cold reaction and the mixture was warmed to 25° C. and stirred for 4 hours. The reaction was partitioned between dichloromethane and water, and the organic phase was washed with brine and dried over MgSO₄, filtered and concentrated to give the title compound (6.83 g), which was used without further purification.

### EXAMPLE 36C

### tert-butyl(2S,3S)-2-(2,4-dioxo-1-imidazolidinyl)-3-methylpentanoate

A solution containing the product from Example 36B (6.8 g, 20.9 mmol) in methanol (30 mL) was treated with triethylamine (5.6 mL, 40.2 mmol), stirred at 50° C. for 2 hours, and concentrated. The residue was chromatographed on silica gel eluting with 0-30% ethyl acetate/dichloromethane to give the title compound (2.53 g, 47% yield).

### EXAMPLE 36D

### tert-butyl(2S,3S)-3-methyl-2-{3-[(6-methyl-2-pyridinyl)methyl]-2,4dioxo-1-imidazolidinyl}pentanoate

A solution containing the product from Example 36C (0.107 g, 0.396 mmol) in dichloromethane (2 mL) at 0° C. Was treated with 6-methyl-2-pyridinemethanol (0.053 g, 0.435 mmol), triphenylphosphine (0.135 g, 9.515 mmol), followed by diethyl azodicarboxylate (0.080 mL, 0.515 mmol), stirred at 25° C. for 16 hours. Water (2 mL) was added and the reaction was stirred for 2 hours at 25° C. The reaction mixture was partitioned between dichloromethane and water, and the organic phase was washed with brine and dried over MgSO₄, filtered and concentrated. The residue was chromatographed on silica gel eluting with 0-30% ethyl acetate/dichloromethane to give the title compound (0.154 g, 94% yield).

### EXAMPLE 36E

### (2S,3S)-3-methyl-2-{3-[(6-methyl-2-pyridinyl)methyl]-2,4-dioxo-1-imidazolidinyl}pentanoic Acid

A solution containing the product from Example 36D (0.154 g, 0.410 mmol) in dichloromethane (3 mL) was treated with trifluoracetic acid (3 mL), stirred at 25° C. for 16 hours and concentrated. The residue was purified by reversed phase chromatography on a C18 column eluting with a gradient starting with 5-100% acetonitrile in water (0.1% TFA) to give the title compound (0.153 g) as the trifluoroacetic acid salt.

### EXAMPLE 36F

### methyl(1S)-1-[({(1S,3S,4S)-3-hydroxy-4-[((2S)-3-methyl-2-{3-[(6-methyl-2-pyridinyl)methyl]-2,4-dioxo-1-imidazolidinyl}pentanoyl)amino]-5-phenyl-1-[4-(2-pyridinyl)benzyl]pentyl}amino)carbonyl]-2,2-dimethylpropylcarbamate

A solution containing the product from Example 2C (0.025 g, 0.047 mmol) in THF (0.5 mL) was treated with the product from Example 36E (0.020 g, 0.061 mmol), DEPBT (0.021 g, 0.071 mmol), and N,N-diisopropylethylamine (0.041 mL, 0.235 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-10% methanol in ethyl acetate, to give the title compound (0.026 g, 68% yield). ¹H NMR (300 MHz, DMSO-d6), δ ppm 0.76 (m, 18H), 1.16 (m, 1H), 1.29 (m, 1H), 1.52 (m, 1H), 1.76 (s, 1H), 2.39 (s, 3H), 2.68 (m, 4H), 3.20 (m, 2H), 3.50 (s, 3H), 3.83 (m, 2H), 4.13 (m, 2H), 4.67 (m, 2H), 6.66 (d, J=9.56 Hz, 1H), 7.07 (m, 7H), 7.22 (d, J=8.09 Hz, 2H), 7.31 (m, 1H), 7.66 (t, J=7.72 Hz, 1H), 7.86 (n, 6H), 8.63 (d, J=4.04 Hz, 1H).

### EXAMPLE 37

### methyl(1S)-1-[({(1S,2S,4S)-2-hydroxy-4-[((2S)-3-methyl-2-{3-[(6-methyl-2-pyridinyl)methyl]-2,4-dioxo-1-imidazolidinyl}pentanoyl)amino]-5-phenyl-1-[4-(2-pyridinyl)benzyl]pentyl}amino)carbonyl]-2,2-dimethylpropylcarbamate

A solution containing the product from Example 23S (0.025 g, 0.047 mmol) in THF (0.5 mL) was treated with the product from Example 36E (0.020 g, 0.061 mmol), DEPBT (0.021 g, 0.071 mmol), and N,N-diisopropylethylamine (0.041 mL, 0.235 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-10% methanol in ethyl acetate, to give the title compound (0.025 g, 64% yield). ¹H NMR (300 MHz, DMSO-d₆), δ ppm 0.57 (d, J=6.62 Hz, 3H), 0.71 (t, J=7.17 Hz, 3H), 0.82 (m, 12H), 1.26 (m, 1H), 1.54 (m, 2H), 1.73 (m, 1H), 2.33 (m, 4H), 2.76 (m, 3H), 3.51 (s, 3H), 3.59 (m, 1H), 3.82 (d, J=18.38 Hz, 1H), 4.01 (m, 2H), 4.18 (s, 1H), 4.67 (m, 2H), 4.87 (d, J=5.15 Hz, 1H), 6.82 (d, J=9.56 Hz, 1H), 7.02 (m, 6H), 7.12 (d, J=7.72 Hz, 1H), 7.30 (d, J=8.46 Hz, 3H), 7.64 (m, 2H), 7.87 (m, 4H), 8.09 (d, J=9.19 Hz, 1H), 8.63 (d, J=4.04 Hz, 1H).

### EXAMPLE 38

### methyl(1S)-1-[({(1S,3S,4S)-4-{[(2,6-dimethylphenoxy)acetyl]amino}-3-hydroxy-5-phenyl-1-[4-(2-pyridinyl)benzyl]pentyl}amino)carbonyl]-2,2-dimethylpropylcarbamate

A solution containing the product from Example 2C (0.020 g, 0.038 mmol) in THF (0.4 mL) was treated with 2,6-dimethylphenoxy acetic acid (U.S. Pat. No. 5,914,332, see Example 1H) (0.008 g, 0.044 mmol), DEPBT (0.017 g, 0.057 mmol), and N,N-diisopropylethylamine (0.030 mL, 0.172 mmol), stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-80% ethyl acetate/chloroform to give the title compound (0.021 g, 77% yield).

¹H NMR (300 MHz, DMSO-d₆), δ ppm 0.83 (s, 9H), 1.52 (m, 2H), 2.08 (s, 6H), 2.72 (m, 2H), 2.80 (m, 2H), 3.51 (s, 3H), 3.72 (m, 1H), 3.85 (d, J=9.19 Hz, 1H), 4.01 (s, 2H), 4.22 (m, 2H), 5.05 (d, J=5.88 Hz, 1H), 6.71 (d, J=9.93 Hz, 1H), 6.92 (m, 3H), 7.26 (m, 8H), 7.45 (d, J=9.56 Hz, 1H), 7.85 (m, 5H), 8.62 (d, J=4.78 Hz, 1H).

### EXAMPLE 39

### methyl(1S)-1-[({(1S,2S,4S)-4-{[(2,6-dimethylphenoxy)acetyl]amino}-2-hydroxy-5-phenyl-1-[4-(2-pyridinyl)benzyl]pentyl}amino)carbonyl]-2,2-dimethylpropylcarbamate

A solution containing the product from Example 23S (0.020 g, 0.038 mmol) in THF (0.4 mL) was treated with 2,6-dimethylphenoxy acetic acid (U.S. Pat. No. 5,914,332, see Example 1H) (0.008 g, 0.044 mmol), DEPBT (0.017 g, 0.057 mmol), and N,N-diisopropylethylamine (0.030 mL, 0.172 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-80% ethyl acetate/chloroform to give the title compound (0.016 g, 61% yield). ¹H NMR (300 MHz, DMSO-d₆), δ ppm 0.86 (s, 9H), 1.63 (m, 2H), 2.07 (s, 6H), 2.78 (m, 4H), 3.51 (s, 3H), 3.62 (m, 1H), 3.94 (m, 3H), 4.25 (m, 2H), 4.87 (d, J=5.15 Hz, 1H), 6.89 (m, 4H), 7.19 (m, 5H), 7.31 (m, 3H), 7.61 (d, J=8.46 Hz, 1H), 7.84 (m, 5H), 8.62 (d, J=4.41 Hz, 1H).

### EXAMPLE 40A

### imidazo[1,5-a]pyridine-3-carbaldehyde

To imidazo[1,5-a]pyridine (2.337 g, 19.78 mmol) in tetrahydrofuran (40 mL) was added n-butyl lithium (2.5 M in hexane, 15.76 mL, 39.4 mmol) at −40° C. The mixture was stirred at −40° C. for 3.5 hours, followed by the addition of dimethylformamide (3.1 mL, 40 mmol). The reaction mixture was stirred at 25° C. overnight and quenched with water. The mixture was then partitioned between dichloromethane (80 mL) and water (15 mL). The organic phase layer was dried with anhydrous sodium sulfate, filtered and concentrated in vacuo. The crude material was purified by chromatography eluting with 0-50% ethyl acetate/dichloromethane to give the title compound (1.78 g, 62% yield).

### EXAMPLE 40B

### tert-butyl(2S)-2-[3-(imidazo[1,5-a]pyridin-3-ylmethyl)-2-oxo-1-imidazolidinyl]-3,3-dimethylbutanoate

A solution of the product from Example 40A (1.809 g, 12.38 mmol) and the product from Example 6F (2.85 g, 12.38 mmol) in ethanol (35 mL) and benzene (35 mL) was treated with molecular sieves (3 Å, 1.5 g). The mixture was stirred at 60° C. overnight and cooled to 25° C. To the reaction mixture was added sodium borohydride (1.407 g, 37.19 mmol) and then stirred for 3 hours at 25° C. The reaction mixture was quenched with an aqueous solution of saturated ammonium chloride at 0° C. The mixture was partitioned between water (50 mL) and ethyl acetate (100 mL). The organic phase layer was separated and washed with water and brine, dried with anhydrous sodium sulfate, filtered and concentrated. The residue was treated with 1,2-dichloroethane (247 mL), N,N-diisopropylethylamine (2.2 mL, 12.63 mmol) and N,N′-disuccinimidyl carbonate (3.823 g, 14.92 mmol). The solution was stirred at 25° C. overnight and then washed with a solution of 10% sodium carbonate (3×50 mL) and water (50 mL). The organic phase layer was dried with anhydrous sodium sulfate, filtered and concentrated in vacuo. The crude material was purified by chromatography eluting with 0-100% ethyl acetate/dichloromethane to give the title compound (3 g, 63% yield).

### EXAMPLE 40C

### (2S)-2-[3-(imidazo[1,5-a]pyridin-3-ylmethyl)-2-oxo-1-imidazolidinyl]-3,3-dimethylbutanoic Acid

A solution containing the product from Example 40B (0.039 g, 0.096 mmol) in dichloromethane (0.5 mL) was treated with trifluoracetic acid (0.5 mL), and the mixture was stirred at 25° C. for 2 hours. The solvent was concentrated and azeotroped with toluene to give the title compound as the trifluoroacetic acid salt, which was used without further purification.

### EXAMPLE 40D

### methyl(1S)-1-[({(1S,3S,4S)-3-hydroxy-4-({(2S)-2-[3-(imidazo[1,5-a]pyridin-3-ylmethyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate

A solution containing the product from Example 2C (0.021 g, 0.048 mmol) in THE (0.5 mL) was treated with the product from Example 40C (0.020 g, 0.061 mmol), DEPBT (0.021 g, 0.071 mmol), and N,N-diisopropylethylamine (0.042 mL, 0.235 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The reaction was partitioned between ethyl acetate and saturated NaHCO₃, and the organic phase was washed with brine and dried over MgSO₄, filtered and concentrated to give the title compound (0.018 g, 42% yield). ¹H NMR (300 MHz, DMSO-d₆), δ ppm 0.83 (s, 9H), 0.86 (s, 9H), 1.26 (m, 1H), 1.53 (m, 2H), 2.58 (m, 3H), 2.77 (m, 2H), 3.03 (m, 2H), 3.50 (s, 3H), 3.65 (m, 1H), 3.84 (d, J=9.93 Hz, 1H), 4.13 (m, 3H), 4.52 (m, 2H), 4.92 (d, J=15.44 Hz, 1H), 6.64 (t, J=7.54 Hz, 3H), 6.71 (m, 1H), 6.83 (m, 4H), 7.22 (d, J=8.09 Hz, 2H), 7.31 (m, 1H), 7.41 (m, 2H), 7.59 (d, J=9.19 Hz, 1H), 7.86 (m, 5H), 8.35 (d, J=7.35 Hz, 1H), 8.63 (d, J=4.78 Hz, 1H).

### EXAMPLE 41

### methyl(1S)-1-[({(1S,2S,4S)-2-hydroxy-4-({(2S)-2-[3-(imidazo[1,5-a]pyridin-3-ylmethyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate

A solution containing the product from Example 23S (0.021 g, 0.048 mmol) in THF (0.5 mL) was treated with the product from Example 40C (0.020 g, 0.061 mmol), DEPBT (0.021 g, 0.071 mmol), and N,N-diisopropylethylamine (0.042 mL, 0.235 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The reaction was partitioned between ethyl acetate and saturated NaHCO₃, and the organic phase was washed with brine and dried over MgSO₄, filtered and concentrated to give the title compound (0.019 g, 47% yield). ¹H NMR (300 MHz, DMSO-d₆), δ ppm 0.79 (s, 9H), 0.86 (s, 9H), 1.28 (m, 1H), 1.51 (m, 2H), 2.09 (m, 1H), 2.26 (m, 1H), 2.80 (m, 3H), 2.97 (m, 1H), 3.09 (m, 1H), 3.50 (s, 3H), 3.61 (m, 1H), 3.95 (m, 2H), 4.16 (m, 2H), 4.52 (d, J=15.44 Hz, 1H), 4.85 (m, 2H), 6.65 (m, 3H), 6.78 (m, 3H), 6.87 (d, J=6.99 Hz, 2H), 7.30 (m, 3H), 7.38 (s, 1H), 7.58 (d, J=9.19 Hz, 2H), 7.85 (m, 5H), 8.33 (d, J=6.99 Hz, 1H), 8.63 (d, J=4.41 Hz, 1H).

### EXAMPLE 42A

### tert-butyl(2S)-3,3-dimethyl-2-[2-oxo-3-(4-quinolinylmethyl)-1-imidazolidinyl]butanoate

A solution containing the product from Example 6F (0.367 g, 1.59 mmol) in a mixture of benzene (5 mL) and methanol (5 mL) was treated with 4quinolinecarboxaldehyde (0.25 g, 1.59 mmol), heated at 50° C. for 3 hours, cooled to 25° C. and treated with sodium borohydride (0.12 g, 3.18 mmol), stirred at 25° C. for 2 hours, quenched with sodium bicarbonate solution and partitioned between ethyl acetate and water. The organic phase was washed with brine and dried over MgSO₄, filtered and concentrated. A solution of the concentrate (1.59 mmol) in toluene (10 mL) was treated with bis(4-nitrophenyl)carbonate (0.58 g, 1.9 mmol), heated at 100° C. for 16 hours, cooled and partitioned between ethyl acetate and saturated Na₂CO₃. The organic phase was washed with brine and dried over MgSO₄, filtered and concentrated. The residue was chromatographed on silica gel eluting with 0-20% acetone/dichloromethane to give the title compound (0.355 g, 57% yield).

### EXAMPLE 42B

### (2S)-3,3-dimethyl-2-[2-oxo-3-(4-quinolinylmethyl)-1-imidazolidinyl]butanoic Acid

A solution containing the product from Example 42A (0.355 g, 0.89 mmol) in dichloromethane (4 mL) was treated with trifluoracetic acid (4 mL), and the mixture was stirred at 25° C. for 2 hours. The solvent was concentrated and azeotroped with toluene several times to give the crude product as the trifluoroacetic acid salt, which was used without further purification.

### EXAMPLE 42C

### methyl(1S)-1-[({(1S,3S,4S)-4-({(2S)-3,3-dimethyl-2-[2-oxo-3-(4-quinolinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 2C (0.025 g, 0.047 mmol) in THF (0.5 mL) was treated with the product from Example 42B (0.024 g, 0.070 mmol), DEPBT (0.021 g, 0.070 mmol), and N,N-diisopropylethylamine (0.041 mL, 0.235 mmol) and the mixture was stirred at 25° C. for 2 hours. The mixture was partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.027 g, 67% yield). ¹H NMR (500 MHz, DMSO-d₆), δ ppm 0.82(s, 9H), 0.89(s, 9H), 1.55 (m, 2H), 2.27(m, 1H), 2.65-2.60(m, 3H), 2.77(m, 1H), 2.85(m, 1H), 3.03(m, 1H), 3.17(m, 1H), 3.49(s, 3H), 3.65(m, 1H), 3.84(d, J=8.79 Hz, 1H), 4.08(d, J=33.69 Hz, 3H), 4.52(d, J=7.81 Hz, 1H), 4.79(dd, J=152.34, 15.63 Hz, 2H), 6.57(d, J=8.79 Hz, 1H), 6.81(t, J=7.32 Hz, 2H), 6.90(t, J=7.08 Hz, 1H), 6.96(d, J=6.84 Hz, 2H), 7.22(d, J=7.81 Hz, 2H), 7.29(m, 1H), 7.46-7.42(m, 2H), 7.63(t, J=7.57 Hz, 1H), 7.90-7.76(m, 5H), 8.06(d, J=7.81 Hz, 1H), 8.31(d, J=8.30 Hz, 1H), 8.62(d, J=3.91 Hz, 1H), 8.90(d, J=4.39 Hz, 1H).

### EXAMPLE 43

### methyl(1S)-1-[({(1S,2S,4S)-4-({(2S)-3,3-dimethyl-2-[2-oxo-3-(4-quinolinylmethyl)-1-imidazolidinyl]butanoyl}amino)-2-hydroxy-5-phenyl-1-[4-(2-pyridinyl)benzyl]pentyl}amino)carbonyl]-2,2-dimethylpropylcarbamate

A solution containing the product from Example 23S (0.025 g, 0.047 mmol) in THF (0.5 mL) was treated with the product from Example 42B (0.024 g, 0.070 mmol), DEPBT (0.021 g, 0.070 mmol), and N,N-diisopropylethylamine (0.041 mL, 0.235 mmol) and the mixture was stirred at 25° C. for 2 hours. The mixture was partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.015 g, 37% yield). ¹H NMR (500 MHz, DMSO-d₆), δ ppm 0.83(s, 9H), 0.85(s, 9H), 1.61-1.50(m, 2H), 2.41-2.31(m, 2H), 2.69-2.59(m, 1H), 2.78(bs, 2H), 2.88(m, 1H), 3.03-2.95(m, 1H), 3.23-3.14(m, 1H), 3.50(s, 3H), 3.61(m, 1H), 3.94(m, 1H), 4.00(s, 1H), 4.18(m, 2H), 4.81(bs, 1H), 4.92-4.64(dd, J=15.63, 126.95 Hz, 2H), 6.87-6.73(m, 4H), 6.96(m, 2H), 7.29(m, 3H), 7.41(bs, 1H), 7.61-7.54(m, 2H), 7.89-7.77(m, 5H), 8.05(d, J=7.81 Hz, 1H), 8.29(d, J=7.32 Hz, 1H), 8.62(bs, 1H), 8.89(bs, 1H).

### EXAMPLE 44

### methyl(1S)-1-[({(1S,2S,4S)-2-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate

A solution containing the product from Example 23S (0.025 g, 0.047 mmol) in THF (0.5 mL) was treated with the product from Example 17F (0.030 g, 0.066 mmol), DEPBT (0.021 g, 0.070 mmol), and N,N-diisopropylethylamine (0.041 mL, 0.235 mmol) and the mixture was stirred at 25° C. for 2 hours. The mixture was partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 05% methanol in ethyl acetate, to give the title compound (0.026 g, 64% yield). ¹H NMR (300 MHz, DMSO-d6), δ ppm 0.82 (s, 9H), 0.86 (s, 9H), 1.42 (d, J=4.78 Hz, 6H), 1.55 (m, 2H), 2.39 (m, 2H), 2.65 (d, J=13.24 Hz, 1H), 2.78 (d, J=6.25 Hz, 2H), 2.98 (m, 1H), 3.20 (m, 3H), 3.51 (s, 3H), 3.61 (m, 1H), 3.98 (m, 2H), 4.19 (m, 2H), 4.39 (m, 2H), 4.82 (d, J=5.52 Hz, 1H), 6.78 (d, J=9.19 Hz, 1H), 7.06 (m, 6H), 7.31 (m, 3H), 7.55 (m, 2H), 7.76 (t, J=7.72 Hz, 1H), 7.86 (m, 5H), 8.63 (d, J=4.04 Hz, 1H).

### EXAMPLE 45

### methyl(1S)-1-[({(1S,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate

A solution containing the product from Example 2C (0.025 g, 0.047 mmol) in THF (0.5 mL) was treated with the product from Example 17F (0.030 g, 0.066 mmol), DEPBT (0.021 g, 0.070 mmol), and N,N-diisopropylethylamine (0.041 mL, 0.235 mmol) and the mixture was stirred at 25° C. for 2 hours. The mixture was partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.035 g, 86% yield). ¹H NMR (300 MHz, DMSO-d6), δ ppm 0.83 (s, 9H), 0.90 (s, 9H), 1.43 (d, J=5.15 Hz, 6H), 1.53 (m, 2H), 2.36 (m, 1H), 2.65 (m, 3H), 2.79 (m, 1H), 2.99 (m, 1H), 3.20 (m, 3H), 3.50 (s, 3H), 3.65 (m, 1H), 3.85 (d, J=9.93 Hz, 1H), 4.05(m, 3H), 4.45 (m, 3H), 6.63 (d, J=9.93 Hz, 1H), 7.08 (m, 6H), 7.22 (d, J=8.09 Hz, 2H), 7.31 (m, 1H), 7.46 (d, J=9.56 Hz, 1H), 7.54 (d, J=7.72 Hz, 1H), 7.83 (m, 6H), 8.64 (d, J=4.78 Hz, 1H).

### EXAMPLE 46A

### tert-butyl(2S)-2-[(2-ethoxy-2-oxoethyl)amino]-3,3-dimethylbutanoate

A solution of L-tert-leucine tert-butyl ester hydrochloride (5 g, 22.34 mmol) in DMF (25 mL) was treated with triethylamine (3.1 mL, 22.34 mmol) and the mixture was stirred for 1 hour. The reaction was filtered to remove solid salts, and the filtrate was treated with triethylamine (9.3 mL, 67.0 mmol) and ethyl bromoacetate (9.9 mL, 67.0 mmol), and the reaction was stirred for 3 hours at 25° C. The solvent was concentrated and the reaction was partitioned between dichloromethane and water. The organic phase was washed with brine and dried over MgSO₄, filtered and concentrated. The residue was chromatographed on silica gel eluting with 0-20% ethyl acetate/hexane to give the title compound (5.47 g, 90% yield).

### EXAMPLE 46B

### tert-butyl(2S)-2-[(aminocarbonyl)(2-ethoxy-2-oxoethyl)amino]-3,3-dimethylbutanoate

A solution containing the product from Example 46A (5.74 g, 20.0 mmol) in dichloromethane (40 mL) at 0° C. was treated with chlorosulfonyl isocyanate (2.26 mL, 26.0 mmol) and the mixture was stirred at 0° C. for 2 hours. Water (40 mL) was added to the cold reaction and the mixture was warmed to 25° C. and stirred for 2 hours. The reaction mixture was partitioned between dichloromethane and water, and the organic phase was washed with brine and dried over MgSO₄, filtered and concentrated to give the title compound, which was used without further purification.

### EXAMPLE 46C

### tert-butyl(2S)-2-(2,4-dioxo-1-imidazolidinyl)-3,3-dimethylbutanoate

A solution containing the product from Example 46B (20.0 mmol) in methanol (30 mL) was treated with triethylamine (5.6 mL, 40.2 mmol), stirred at 50° C. for 2 hours, and concentrated. The residue was chromatographed on silica gel eluting with 0-30% ethyl acetate/dichloromethane to give the title compound (4.57 g, 85% yield).

### EXAMPLE 46D

### tert-butyl(2S)-3,3-dimethyl-2-{3-[(6-methyl-2-pyridinyl)methyl]-2,4-dioxo-1-imidazolidinyl}butanoate

A solution containing the product from Example 46C (0.112 g, 0.413 mmol) in dichloromethane (3 mL) at 0° C. was treated with 6-methyl-2-pyridinemethanol (0.056 g, 0.454 mmol), triphenylphosphine (0.141 g, 0.537 mmol), followed by diethyl azodicarboxylate (0.084 mL, 0.537 mmol), stirred at 25° C. for 16 hours, treated with water (3 mL), stirred for 2 hours at 25° C., and partitioned between dichloromethane and water. The organic phase was washed with brine and dried over MgSO₄, filtered and concentrated. The residue was chromatographed on silica gel eluting with 030% ethyl acetate/dichloromethane to give the title compound (0.151 g, 97% yield).

### EXAMPLE 46E

### (2S)-3,3-dimethyl-2-{3-[(6-methyl-2-pyridinyl)methyl]-2,4-dioxo-1-imidazolidinyl}butanoic Acid

A solution containing the product from Example 46D (0.151 g, 0.403 mmol) in dichloromethane (3 mL) was treated with trifluoracetic acid (3 mL), and the mixture was stirred at 25° C. for 16 hours. The solvent was concentrated and the product was purified by reversed phase chromatography on a C18 column eluting with a gradient starting with 5-100% acetonitrile in water (0.1% TFA) to give the title compound (0.141 g, 81% yield) as the trifluoroacetic acid salt.

### EXAMPLE 46F

### methyl(1S)-1-[({(1R,3S,4S)-4-[((2S)-3,3-dimethyl-2-{3-[(6-methyl-2-pyridinyl)methyl]-2,4-dioxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 1H (0.020 g, 0.038 mmol) in THF (0.4 mL) was treated with the product from Example 46E (0.020 g, 0.046 mmol), DEPBT (0.017 g, 0.057 mmol), and N,N-diisopropylethylamine (0.030 mL, 0.172 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.023 g, 73% yield). ¹H NMR (300 MHz, DMSO-d₆), δ ppm 0.82 (s, 9H), 0.87 (s, 9H), 1.32 (m, 1H), 1.53 (t, J=11.40 Hz, 1H), 2.41 (s, 3H), 2.63 (m, 3H), 2.85 (m, 1H), 3.16 (d, J=18.02 Hz, 1H), 3.60 (m, 5H), 3.90 (m, 3H), 4.19 (m, 1H), 4.35 (s, 1H), 4.68 (m, 2H), 6.90 (d, J=9.93 Hz, 1H), 7.03 (m, 6H), 7.16 (d, J=7.72 Hz, 1H), 7.25 (d, J=8.09 Hz, 2H), 7.34 (m, 1H), 7.69 (t, J=7.72 Hz, 1H), 7.93 (m, 6H), 8.65 (d, J=4.78 Hz, 1H).

### EXAMPLE 47

### 1:1 Mixture of (3R,3aS,6aR)-hexahydrofuro[2,3-b]furan-3-yl(1S,2S,4R)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl(1S,2S,4R)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate

A solution of the product from Example 1H (0.020 g, 0.038 mmol) in THF (0.25 mL) was treated with N,N-diisopropylethylamine (0.015 mL, 0.086 mmol) and the product from Example 26A (0.017 g, 0.058 mmol), stirred at 25° C. for 1 hour, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.018 g, 70% yield).

¹H NMR (300 MHz, DMSO-d₆), δ ppm 0.76 (d, J=3.31 Hz, 9H), 1.43 (m, 3H), 2.68 (m, 5H), 3.71 (m, 12H), 4.18 (m, 1H), 4.85 (m, 1H), 5.52 (m, 1H), 6.89 (m, 1H), 6.99 (m, 1H), 7.23 (m, 8H), 7.94 (m, 5H), 8.65 (d, J=4.78 Hz, 1H).

### EXAMPLE 48

### methyl(1S)-1-[({(1R,3S,4S)-4-{[(2S)-3,3-dimethyl-2-(2-oxo-3-{[2-(3-pyridinyl)-1,3-thiazol-4-yl]methyl}-1-imidazolidinyl)butanoyl]amino}-3-hydroxy-5-phenyl-1-[4-(2-pyridinyl)benzyl]pentyl}amino)carbonyl]-2,2-dimethylpropylcarbamate

A solution containing the product from Example 1H (0.020 g, 0.038 mmol) in THF (0.4 mL) was treated with the product from Example 19D (0.022 g, 0.045 mmol), DEPBT (0.017 g, 0.057 mmol), and N,N-diisopropylethylamine (0.030 mL, 172 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was purified by chromatography on silica gel eluting with 0-100% ethyl acetate/dichloromethane, followed by 0-7.5% methanol in ethyl acetate to give the title compound (0.026 g, 78% yield). ¹H NMR (300 MHz, DMSO-d₆), δ ppm 0.80 (s, 9H), 0.88 (s, 9H), 1.46 (m, 2H), 2.44 (d, J=8.82 Hz, 1H), 2.63 (m, 3H), 2.83 (m, 1H), 3.15 (m, 3H), 3.54 (m, 4H), 3.84 (d, J=9.56 Hz, 1H), 3.93 (m, 1H), 4.04 (s, 1H), 4.18 (m, 1H), 4.46 (m, 3H), 6.88 (d, J=9.56 Hz, 1H), 6.96 (m, 1H), 7.06 (m, 4H), 7.24 (d, J=8.46 Hz, 2H), 7.32 (m, 1H), 7.53 (m, 2H), 7.60 (s, 1H), 7.89 (m, 5H), 8.29 (m, 1H), 8.65 (m, 2H), 9.13 (d, J=1.47 Hz, 1H).

### EXAMPLE 49

### methyl(1S)-1-[({(1R,3S,4S)-4-{[(2,6-dimethylphenoxy)acetyl]amino}-3-hydroxy-5-phenyl-1-[4-(2-pyridinyl)benzyl]pentyl}amino)carbonyl]-2,2-dimethylpropylcarbamate

A solution containing the product from Example 1H (0.020 g, 0.038 mmol) in THF (0.4 mL) was treated with 2,6-dimethylphenoxy acetic acid (U.S. Pat. No. 5,914,332, see Example 1H) (0.008 g, 0.044 mmol), DEPBT (0.017 g, 0.057 mmol), and N,N-diisopropylethylamine (0.030 mL, 0.172 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.019 g, 73% yield). ¹H NMR (300 MHz, DMSO-d₆), δ ppm 0.76 (s, 9H), 1.41 (t, J=11.77 Hz, 1H), 1.58 (m, 1H), 2.10 (s, 6H), 2.77 (m, 4H), 3.57 (s, 3H), 3.65 (m, 1H), 3.81 (d, J=9.56 Hz, 1H), 4.07 (m, 4H), 5.02 (d, J=5.52Hz, 1H), 6.92 (m, 4H), 7.25 (m, 8H), 7.56 (d, J=9.56 Hz, 1H), 7.85 (m, 3H), 7.96 (d, J=8.46 Hz, 2H), 8.64 (d, J=4.41 Hz, 1H).

### EXAMPLE 50

### methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate

A solution containing the product of Example 1H (0.72 g, 1.35 mmol) in THF (12 mL) was treated with the product from Example 17F (0.54 g, 1.16 mmol), DEPBT (0.52 g, 1.74 mmol), and N,N-diisopropylethylamine (1.0 mL, 5.74 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 50-100% ethyl acetate in chloroform to give the title compound (0.84 g, 84% yield).

¹H NMR (300 MHz, DMSO-d₆), δ ppm 0.79 (s, 9H), 0.87 (s, 9H), 1.37 (m, 1H), 1.41 (s, 3H), 1.43 (s, 3H), 1.52 (m, 1H), 2.48 (m, 1H), 2.64 (m, 3H), 2.83 (dd, J=14.0, 6.6 Hz, 1H), 3.01 (m, 1H), 3.15 (m, 1H), 3.23 (m, 1H), 3.53 (m, 1H), 3.56 (s, 3H), 3.83 (d, J=9.56 Hz, 1H), 3.93 (m, 1H), 4.02 (s, 1H), 4.17 (m, 1H), 4.39 (m, 3H), 5.15 (s, 1H), 6.87 (d, J=10.30 Hz, 1H), 7.08 (m, 6H), 7.24 (d, J=8.46 Hz, 2H), 7.32 (m, 1H), 7.52 (m, 2H), 7.75 (t, J=7.72 Hz, 1H), 7.89 (m, 3H), 7.95 (d, J=8.46 Hz, 2H), 8.64 (d, J=4.78 Hz, 1H).

### EXAMPLE 51

### methyl(1S)-1-[({(1S,3S,4S)-4-[((2S)-3,3-dimethyl-2-{3-[(6-methyl-2-pyridinyl)methyl]-2,4-dioxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 2C (0.020 g, 0.038 mmol) in THF (0.4 mL) was treated with the product from Example 46E (0.020 g, 0.046 mmol), DEPBT (0.017 g, 0.057 mmol), and N,N-diisopropylethylamine (0.035 mL, 0.201 mmol), stirred at 25° C. for 1 hour, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.020 g, 64% yield). ¹H NMR (300 MHz, DMSO-d₆), δ ppm 0.85 (s, 9H), 0.88 (s, 9H), 1.54 (m, 2H), 2.41 (s, 3H), 2.65 (m, 4H), 3.08 (d, J=18.02 Hz, 1H), 3.51 (s, 3H), 3.72 (m, 1H), 3.89 (m, 2H), 4.17 (m, 2H), 4.39 (s, 1H), 4.67 (m, 3H), 6.66 (d, J=9.93 Hz, 1H), 7.06 (m, 7H), 7.23 (d, J=8.46 Hz, 2H), 7.31 (m, 1H), 7.66 (t, J=7.54 Hz, 1H), 7.87 (m, 6H), 8.64 (d, J=4.41 Hz, 1H).

### EXAMPLE 52

### methyl(1S)-1-[({(1S,2S,4S)-4-[((2S)-3,3-dimethyl-2-{3-[(6-methyl-2-pyridinyl)methyl]-2,4-dioxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate

A solution containing the product from Example 23S (0.020 g, 0.038 mmol) in THF (0.4 mL) was treated with the product from Example 46E (0.020 g, 0.046 mmol), DEPBT (0.017 g, 0.057 mmol), and N,N-diisopropylethylamine (0.035 mL, 0.201 mmol), stirred at 25° C. for 1 hour, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.021 g, 67% yield). ¹H NMR (300 MHz, DMSO-d₆), δ ppm 0.80 (s, 9H), 0.88 (s, 9H), 1.55 (m, 2H), 2.29 (m, 1H), 2.39 (s, 3H), 2.75 (m, 3H), 3.15 (d, J=18.38 Hz, 1H), 3.52 (s, 3H), 3.61 (m, 1H), 3.94 (m, 2H), 4.19 (m, 3H), 4.68 (d, J=10.30 Hz, 2H), 4.89 (d, J=5.52 Hz, 1H), 6.83 (d, J=9.93 Hz, 1H), 7.00 (m, 6H), 7.13 (d, J=7.72 Hz, 1H), 7.31 (m, 3H), 7.64 (m, 2H), 7.88 (m, 4H), 8.09 (d, J=9.19 Hz, 1H), 8.63 (d, J=4.78 Hz, 1H).

### EXAMPLE 53

### methyl(1S)-1-[({(1R,3S,4S)-4-({(2S)-3,3-dimethyl-2-[2-oxo-3-(4-quinolinylmethyl) 1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 1H (0.020 g, 0.038 mmol) in THF (0.4 mL) was treated with the product from Example 42B (0.020 g, 0.045 mmol), DEPBT (0.017 g, 0.057 mmol), and N,N-diisopropylethylamine (0.035 mL, 0.201 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-0.5% methanol in ethyl acetate, to give the title compound (0.021 g, 65% yield). ¹H NMR (300 MHz, DMSO-d₆), δ ppm 0.81 (s, 9H), 0.89 (s, 9H), 1.26 (m, 1H), 1.37 (m, 1H), 1.53 (m, 1H), 2.30 (m, 1H), 2.65 (m, 2H), 2.85 (m, 2H), 3.00 (m, 1H), 3.18 (m, 1H), 3.53 (m, 4H), 3.84 (d, J=9.56 Hz, 1H), 3.94 (m, 1H), 4.05 (m, 1H), 4.19 (m, 1H), 4.44 (d, J=7.35 Hz, 1H), 4.63 (d, J=15.44 Hz, 1H), 4.95 (d, J=15.44 Hz, 1H), 6.87 (m, 6H), 7.25 (d, J=8.46 Hz, 2H), 7.32 (m, 1H), 7.43 (d, J=4.41 Hz, 1H), 7.60 (m, 2H), 7.86 (m, 6H), 8.06 (d, J=7.72 Hz, 1H), 8.30 (d, J=8.09 Hz, 1H), 8.65 (m, 1H), 8.90 (d, J=4.04 Hz, 1H).

### EXAMPLE 54A

### tert-butyl(2S)-3,3-dimethyl-2-[(phenoxyacetyl)amino]butanoate

A solution of L-tert-Leucine tert-butyl ester hydrochloride (0.20 g, 0.90 mmol) in THF (9 mL) at 0° C. was treated with triethylamine (0.38 mL, 2.73 mmol) and phenoxyacetyl chloride (0.14 mL, 1.01 mmol), stirred at 0° C. for 15 minutes and then at 25° C. for 2 hours. The reaction mixture was partitioned between ethyl acetate and water. The organic phase was washed with brine and dried over MgSO₄, filtered and concentrated. The residue was chromatographed on silica gel eluting with 0-10% ethyl acetate/chloroform to give the title compound (0.23 g, 80% yield).

### EXAMPLE 54B

### (2S)-3,3-dimethyl-2-[(phenoxyacetyl)amino]butanoic Acid

A solution of the product from Example 54A (0.012 g, 0.038 mmol) in dichloromethane (0.2 mL) was treated with trifluoroacetic acid (0.2 mL) and the reaction was stirred at 25° C. for 1 hour and concentrated. The concentrate was azeotroped with toluene to give the title compound, which was used without further purification.

### EXAMPLE 54C

### methyl(1S)-1-[({(1S,3S,4S)-4-({(2S)-3,3-dimethyl-2-[(phenoxyacetyl)amino]butanoyl}amino)-3-hydroxy-5-phenyl-1-[4-(2-pyridinyl)benzyl]pentyl}amino)carbonyl]-2,2-dimethylpropylcarbamate

A solution containing the product from Example 2C (0.020 g, 0.038 mmol) in THF (0.4 mL) was treated with the product from Example 54B (0.038 mmol), DEPBT (0.016 g, 0.054 mmol), and N,N-diisopropylethylamine (0.032 mL, 0.184 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA) to give the title compound (0.011 g, 38% yield). ¹H NMR (300 MHz, DMSO-d₆), δ ppm 0.80 (d, J=2.94 Hz, 18H), 1.53 (m, 2H), 2.55 (m, 1H), 2.73 (m, 3H), 3.49 (s, 3H), 3.65 (m, 1H), 3.82 (d, J=9.93 Hz, 1H), 4.05 (m, 1H), 4.15 (m, 1H), 4.33 (d, J=9.56 Hz, 1H), 4.53 (m, 2H), 4.81 (d, J=5.88 Hz, 1H), 6.61 (d, J=9.56 Hz, 1H), 6.95 (m, 3H), 7.11 (m, 1H), 7.18 (m, 6H), 7.31 (m, 3H), 7.48 (d, J=9.56 Hz, 1H), 7.84 (m, 6H), 8.64 (d, J=4.41 Hz, 1H).

### EXAMPLE 55A

### tert-butyl(2S)-2-[(methoxyacetyl)amino]-3,3-dimethylbutanoate

A solution of L-tert-Leucine tert-butyl ester hydrochloride (0.20 g, 0.90 mmol) in THF (9 mL) at 0° C. was treated with triethylamine (0.38 mL, 2.73 mmol) and methoxyacetyl chloride (0.09 mL, 0.98 mmol), stirred at 0° C. for 15 minutes and then at 25° C. for 2 hours. The reaction was partitioned between ethyl acetate and water. The organic phase was washed with brine and dried over MgSO₄, filtered and concentrated. The residue was chromatographed on silica gel eluting with 0-33% ethyl acetate/chloroform to give the title compound (0:266 g).

### EXAMPLE 55B

### (2S)-2-[(methoxyacetyl)amino]-3,3-dimethylbutanoic Acid

A solution of the product from Example 55A (0.012 g, 0.038 mmol) in dichloromethane (0.2 mL) was treated with trifluoroacetic acid (0.2 mL), stirred at 25° C. for 1 hour and concentrated. The residue was azeotroped with toluene to give the title compound, which was used without further purification.

### EXAMPLE 55C

### methyl(1S,4S,6S,7S,10S)-7-benzyl-1,10-ditert-butyl-6-hydroxy-2,9,12-trioxo-4-[4-(2-pyridinyl)benzyl]-14-oxa-3,8,11-triazapentadec-1-ylcarbamate

A solution containing the product from Example 2C (0.020 g, 0.038 mmol) in THF (0.4 mL) was treated with the product from Example 55B (0.038 mmol), DEPBT (0.016 g, 0.054 mmol), and N,N-diisopropylethylamine (0.032 mL, 0.184 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA) to give the title compound (0.011 g, 38% yield). ¹H NMR (300 MHz, DMSO-d₆), δ ppm 0.81 (s, 9H), 0.84 (s, 9H), 1.52 (m, 2H), 2.56 (m, 1H), 2.74 (m, 3H), 3.26 (s, 3H), 3.49 (s, 3H), 3.66 (m, 1H), 3.82 (m, 3H), 4.03 (m, 1H), 4.16 (m, 1H), 4.31 (d, J=9.56 Hz, 1H), 4.83 (d, J=5.88 Hz, 1H), 6.62 (d, J=9.56 Hz, 1H), 7.14 (m, 8H), 7.31 (m, 1H), 7.85 (m, 6H), 8.63 (d, J=4.41 Hz, 1H).

### EXAMPLE 56A

### 2-methylpropanethioamide

A solution containing isobutyramide (10 g, 115 mmol) in THF (250 mL) was treated with phosphorous pentasulfide (4.1 g, 9.22 mmol), stirred at 25° C. for 64 hours, concentrated and partitioned between ethyl acetate and water. The organic phase was washed with brine and dried over MgSO₄, filtered and concentrated to give the title compound (8.6 g, 73% yield), which was used without further purification.

### EXAMPLE 56B

### Ethyl 2-isopropyl-1,3-thiazole-4-carboxylate

A solution containing the product from Example 56A (8.6 g, 83.5 mmol) in ethanol (250 mL) was treated with ethyl bromopyruvate (12.6 mL, 100 mmol), and the mixture was heated at 70° C. for 3 hours, cooled to 25° C., concentrated, and partitioned between dichloromethane and saturated NaHCO₃. The organic phase was washed with brine and dried over MgSO₄, filtered and concentrated to give the title compound (18 g, 57% yield), which was used without further purification.

### EXAMPLE 56C

### (2-isopropyl-1,3-thiazol-4-yl)methanol

A solution containing the product from Example 56B (18 g, 90.5 mmol) in dichloromethane (100 mL) was treated with diisobutyl aluminum hydride (150 mL, 1 M in dichloromethane) dropwise at −78° C. over 2 hours and the mixture was stirred at −78° C. for 2 hours. Acetic acid (10 mL) was added at −78° C. and the mixture was warmed to 25° C. A 10% solution of aqueous sodium potassium tartrate was added and the mixture was stirred vigorously for 1 hour. The reaction was partitioned between dichloromethane and water, and the organic phase was washed with brine and dried over MgSO₄, filtered and concentrated. The residue was chromatographed on silica gel eluting with 0-5% ethyl acetate/dichloromethane to give the title compound (3.84 g, 27% yield).

### EXAMPLE 56D

### tert-butyl(2S,3S)-2-{3-[(2-isopropyl-1,3-thiazol-4-yl)methyl]-2,4-dioxo-1-imidazolidinyl}-3-methylpentanoate

A solution containing the product from Example 36C (0.076 g, 0.281 mmol) in dichloromethane (2 mL) at 0° C. was treated with the product from Example 56C (0.049 g, 0.309 mmol), triphenylphosphine (0.096 g, 0.365 mmol), followed by diethyl azodicarboxylate (0.057 mL, 0.365 mmol), stirred at 25° C. for 16 hours. Water (3 mL) was added and the reaction was stirred for 2 hours at 25° C. The reaction mixture was partitioned between dichloromethane and water, and the organic phase was washed with brine and dried over MgSO₄, filtered and concentrated. The residue was purified by reversed phase chromatography on a C18 column eluting with 5-100% acetonitrile in water (0.1% TFA) to give the title compound (0.090 g, 781/o yield).

### EXAMPLE 56E

### (2S,3S)-2-{3-[(2-isopropyl-1,3-thiazol-4-yl)methyl]-2,4-dioxo-1-imidazolidinyl}-3-methylpentanoic Acid

A solution containing the product from Example 56D (0.090 g, 0.220 mmol) in dichloromethane (2 mL) was treated with trifluoracetic acid (2 mL), stirred at 25° C. for 16 hours, and concentrated. The residue was purified by reversed phase chromatography on a C18 column eluting with 5-100% acetonitrile in water (0.1% TFA) to give the title compound (0.1 g) as the trifluoroacetic acid salt.

### EXAMPLE 56F

### methyl(1S)-1-[({(1S,3S,4S)-3-hydroxy-4-[((2S)-2-{3-[(2-isopropyl-1,3-thiazol-4-yl)methyl]-2,4-dioxo-1-imidazolidinyl}-3-methylpentanoyl)amino]-5-phenyl-1-[4-(2-pyridinyl)benzyl]pentyl}amino)carbonyl]-2,2-dimethylpropylcarbamate

A solution containing the product from Example 2C (0.030 g, 0.056 mmol) in THF (0.5 mL) was treated with the product from Example 56E (0.026 g, 0.073 mmol), DEPBT (0.025 g, 0.085 mmol), and N,N-diisopropylethylamine (0.049 mL, 0.282 mmol), stirred at 25° C. for 1 hour, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by elution with 0-5% methanol in ethyl acetate to give the title compound (0.033 g, 67% yield). ¹H NMR (300 MHz, DMSO-d₆), δ ppm 0.66 (d, J=6.62 Hz, 3H), 0.73 (t, J=7.35 Hz, 3H), 0.88 (m, 12H), 1.26 (s, 3H), 1.29 (s, 3H), 1.50 (m, 2H), 1.73 (m, 1H), 2.69 (m, 4H), 3.10 (d, J=18.38 Hz, 1H), 3.50 (s, 3H), 3.78 (m, 3H), 4.17 (m, 3H), 4.66 (m, 3H), 6.67 (d, J=9.93 Hz, 1H), 6.99 (m, 3H), 7.07 (m, 2H), 7.23 (m, 3H), 7.31 (m, 1H), 7.85 (m, 6H), 8.63 (d, J=4.41 Hz, 1H).

### EXAMPLE 57A

### [2-(3-pyridinyl)-1,3-thiazol-4-yl]methanol

A solution containing the product from Example 19B (0.20 g, 1.05 mmol) in a mixture of THF (1.5 mL) and methanol (1.5 mL) was treated with NaBH4 (0.052 g, 1.37 mmol), stirred at 25° C. for 2 hours, quenched with saturated ammonium chloride solution and concentrated. The concentrate was partitioned between ethyl acetate and saturated NaHCO₃. The organic phase was washed with brine and dried over MgSO₄, filtered and concentrated to give the title compound (0.063 g, 31% yield).

### EXAMPLE 57B

### tert-butyl(2S,3S)-2-(2,4-dioxo-3-{[2-(3-pyridinyl)-1,3-thiazol-4-yl]methyl}-1-imidazolidinyl)-3-methylpentanoate

A solution containing the product from Example 36C (0.081 g, 0.30 mmol) in dichloromethane (2 mL) at 0° C. was treated with the product from Example 57A (0.063 g, 0.33 mmol), triphenylphosphine (0.103 g, 0.39 mmol), followed by diethyl azodicarboxylate (0.061 mL, 0.39 mmol), stirred at 25° C. for 16 hours. Water (3 mL) was added and the reaction was stirred for 2 hours at 25° C. The reaction mixture was partitioned between dichloromethane and water, and the organic phase was washed with brine and dried over MgSO₄, filtered and concentrated, to give the crude product, which was used without further purification.

### EXAMPLE 57C

### (2S,3S)-2-(2,4-dioxo-3-{[2-(3-pyridinyl)-1,3-thiazol-4-yl]methyl}-1-imidazolidinyl)-3-methylpentanoic Acid

A solution containing the product from Example 57B (0.090 g, 0.220 mmol) in dichloromethane (2 mL) was treated with trifluoracetic acid (2 mL), stirred at 25° C. for 16 hours, and concentrated. The residuet was purified by reversed phase chromatography on a C18 column eluting with 5-100% acetonitrile in water (0.1% TFA) to give the title compound (0.131 g, 90% yield) as the trifluoroacetic acid salt.

### EXAMPLE 57D

### methyl(1S)-1-[({(1S,3S,4S)-4-{[(2S)-2-(2,4-dioxo-3-{[2-(3-pyridinyl)-1,3-thiazol-4-yl]methyl}-1-imidazolidinyl)-3-methylpentanoyl]amino}-3-hydroxy-5-phenyl-1-[4-(2-pyridinyl)benzyl]pentyl}amino)carbonyl]-2,2-dimethylpropylcarbamate

A solution containing the product from Example 2C (0.030 g, 0.056 mmol) in THF (0.5 mL) was treated with the product from Example 57C (0.036 g, 0.073 mmol), DEPBT (0.025 g, 0.085 mmol), and N,N-diisopropylethylamine (0.049 mL, 0.282 mmol), stirred at 25° C. for 1 hour, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by elution with 0-5% methanol in ethyl acetate to give the title compound (0.044 g, 86% yield). ¹H NMR (300 MHz, DMSO-d₆), δ ppm 0.67 (d, J=6.62 Hz, 3H), 0.73 (t, J=7.35 Hz, 3H), 0.90 (m, 12H), 1.25 (m, 1H), 1.52 (m, 2H), 1.75 (m, 1H), 2.69 (m, 3H), 3.15 (m, 1H), 3.50 (s, 3H), 3.78 (m, 2H), 4.16 (m, 3H), 4.67 (d, =6.62 Hz, 1H), 4.78 (m, 2H), 6.67 (d, J=9.93 Hz, 1H), 6.96 (m, 3H), 7.07 (m, 2H), 7.22 (d, J=8.09 Hz, 2H), 7.31 (m, 1H), 7.51 (dd, J=7.91, 4.96 Hz, 1H), 7.64 (s, 1H), 7.86 (m, 6H), 8.24 (m, 1H), 8.64 (m, 2H), 9.08 (d, J=1.84 Hz, 1H).

### EXAMPLE 58A

### [(6-methyl-3-pyridinyl)oxy]acetic Acid

A solution containing ethyl 6-methyl-3-pyridyloxyacetate (0.026 g, 0.13 mmol) in a mixture of THF (0.5 mL) and water (0.5 mL) was treated with lithium hydroxide monohydrate (0.008 g, 0.19 mmol), stirred at 25° C. for 18 hours, and concentrated to give the crude product, which was used without purification.

### EXAMPLE 58B

### methyl(1S,4S,6S,7S,10S)-7-benzyl-1,10-ditert-butyl-6-hydroxy-14,14-dimethyl-2,9,12-trioxo-4-[4-(2-pyridinyl)benzyl]-13-oxa-3,8,11-triazapentadec-1-ylcarbamate

A solution containing the product from Example 2C (0.050 g, 0.094 mmol) in THF (0.5 mL) was treated with Boc-L-tert-leucine (0.022 g, 0.096 mmol), DEPBT (0.042 g, 0.140 mmol), and N,N-diisopropylethylamine (0.08 mL, 0.459 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with a gradient starting with 50-100% ethyl acetate/chloroform to give the title compound (0.058 g, 83% yield).

### EXAMPLE 58C

### methyl(1S)-1-[({(1S,3S,4S)-4-{[(2S)-2-amino-3,3-dimethylbutanoyl]amino}-3-hydroxy-5-phenyl-1-[4-(2-pyridinyl)benzyl]pentyl}amino)carbonyl]-2,2-dimethylpropylcarbamate

A solution containing the product from Example 58B (0.058 g, 0.078 mmol) in dichloromethane (0.5 mL) was treated with trifluoroacetic acid (0.5 mL), stirred at 25° C. for 1 hour, and concentrated. The residue was azeotroped with toluene to give the title compound as the trifluoroacetic acid salt, which was used without further purification.

### EXAMPLE 58D

### methyl(1S)-1-[({(1S,3S,4S)-4-{[(2S)-3,3-dimethyl-2-({[(6-methyl-3-pyridinyl)oxy]acetyl}amino)butanoyl]amino}-3-hydroxy-5-phenyl-1-[4-(2-pyridinyl)benzyl]pentyl}amino)carbonyl]-2,2-dimethylpropylcarbamate

A solution containing the product from Example 58C (0.03 g, 0.04 mmol) in THF (0.5 mL) was treated with the product from Example 58A (0.13 mmol), DEPBT (0.017 g, 0.12 mmol), and N,N-diisopropylethylamine (0.033 mL, 0.39 mmol), stirred at 25° C. for 18 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA) to give the title compound (0.016 g, 52% yield). ¹H NMR (300 MHz, DMSO-d₆), δ ppm 0.80 (s, 9H), 0.83 (s, 9H), 1.25 (m, 1H), 1.52 (m, 2H), 2.38 (s, 3H), 2.71 (m, 3H), 3.49 (s, 3H), 3.65 (m, 1H), 3.82 (d, J=9.93 Hz, 1H), 4.10 (m, 2H), 4.32 (d, J=9.56 Hz, 1H), 4.58 (m, 2H), 4.81 (d, J=5.88 Hz, 1H), 6.61 (d, J=9.56 Hz, 1H), 7.17 (m, 9H), 7.31 (m, 1H), 7.61 (d, J=9.56 Hz, 1H), 7.83 (m, 6H), 8.14 (d, J=2.94 Hz, 1H), 8.63 (d, J=4.41 Hz, 1H).

### EXAMPLE 59A

### 2,2-dimethoxy-N-[(1-methyl-1H-benzimidazol-2-yl)methyl]ethanamine

A solution of 1-methyl-2-formylbenzimidazole (1 g) in methanol (27 mL) and acetic acid (0.54 mL) was treated with aminoacetaldehyde diethylacetal (0.9 g, 1 eq.) and NaCNBH₃ (0.85 g, 2 eq.) at 25° C., stirred for 1 hour. The mixture was partitioned between water and ethyl acetate. The organic phase layer was separated, washed sequentially with saturated NaHCO₃ and brine, and concentrated. The residue was chromatographed on silica gel, eluting with 8% methanol/dichloromethane to give the title compound (1.2 g 64% yield).

### EXAMPLE 59B

### 9H-fluoren-9-ylmethyl 2,2-dimethoxyethyl[(1-methyl-1H-benzimidazol-2-yl)methyl]carbamate

A solution of the product of Example 59A (1.2 g) in dichloromethane (30 mL) was treated with 9-fluorenylmethyl succinimide (1.6 g, 1.05 eq.) at 0° C. for 16 hours. The mixture was partitioned between water and ethyl acetate. The organic phase layer was separated, washed sequentially with 10% NaHCO₃ and brine, dried over Na₂SO₄, filtered and concentrated. The residue was chromatographed on silica gel, eluting with ethyl acetate: dichloromethane (1:1) to give 1.83 g (84% yield) of the title compound.

### EXAMPLE 59C

### 9H-fluoren-9-ylmethyl(1-methyl-1H-benzimidazol-2-yl)methyl(2-oxoethyl)carbamate

A solution of the product of Example 59B (0.2 g) in tetrahydrofuran (0.2 mL) was treated with 30% HCl (0.2 mL), stirred at 75° C. for 6 hours, cooled to 25° C. and concentrated. The residue was partitioned between 10% NaHCO₃ and ethyl acetate, the organic phase layer was separated and washed with brine, dried over Na₂SO₄, filtered and concentrated to give the title compound (175 mg).

### EXAMPLE 59D

### tert-butyl(2S)-2-[(2-{[(9H-fluoren-9-ylmethoxy)carbonyl][(1-methyl-1H-benzimidazol-2-yl)methyl]amino}ethyl)amino]-3,3-dimethylbutanoate

A solution of the product of Example 59C (0.178 g) and (L)-methyl t-leucinate hydrochloride (76.1 mg, 1 eq.) in methanol (1.7 mL) and acetic acid (17 μL) was treated with NaCNBH₃ (54 mg, 2 eq.) at 25° C. for 3.5 hours. The mixture was partitioned between water and ethyl acetate. The organic phase layer was separated and washed with 1N NaHCO₃ and brine, and concentrated. The residue was chromatographed on silica gel, eluting with ethyl acetate:dichloromethane (3:1) to give 0.19 g (83% yield) of the title compound.

### EXAMPLE 59E

### tert-butyl(2S)-3,3-dimethyl-2-{3-[(1-methyl-1H-benzimidazol-2-yl)methyl]-2-oxo-1-imidazolidinyl}butanoate

A solution of the product of Example 59D (0.19 g) in N,N-dimethylformamide (3.5 mL) was treated with diethylamine (0.35 mL), stirred at 25° C. for 1.5 hours and concentrated. A solution of the residue in 1,2-dichloroethane (7 mL) was treated with bis(p-nitrophenyl) carbonate (0.128 g, 1.2 eq.), stirred at 60° C. for 16 hours and concentrated. The residue was chromatographed on silica gel, eluting with ethyl acetate:dichloromethane (3:2) to give 80 mg (64% yield) of the title compound.

### EXAMPLE 59F

### (2S)-3,3-dimethyl-2-{3-[(1-methyl-1H-benzimidazol-2-yl)methyl]-2-oxo-1-imidazolidinyl}butanoic Acid

A solution containing the product from Example 59E (0.025 g, 0.070 mmol) in a mixture of THF (0.3 mL) and water (0.3 mL) was treated with lithium hydroxide monohydrate (0.004 g, 0.094 mmol), and the mixture was stirred at 25° C. for 18 hours. The solvent was concentrated to give the crude product, which was used without purification.

### EXAMPLE 59G

### methyl(1S)-1-[({(1R,3S,4S)-4-[((2S)-3,3-dimethyl-2-{3-[(1-methyl-1H-benzimidazol-2-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 1H (0.025 g, 0.047 mmol) in THF (0.5 mL) was treated with the product from Example 59F (0.070 mmol), DEPBT (0.021 g, 0.070 mmol), and N,N-diisopropylethylamine (0.041 mL, 0.240 mmol) and the mixture was stirred at 25° C. for 2 hours. The mixture was partitioned between ethyl acetate and 10% Na₂CO₃ solution. The organic phase was washed with additional 10% Na₂CO₃ solution and brine, dried over MgSO₄, filtered and concentrated. The product was purified by reversed phase chromatography on a C18 column eluting with 5-100% acetonitrile in water (0.1% TFA). The reaction was partitioned between ethyl acetate and saturated NaHCO₃, and the organic phase was washed with brine and dried over MgSO₄, filtered and concentrated, to give the title compound (0.021 g, 50% yield). ¹H NMR (300 MHz, DMSO-d₆), δ ppm 0.81 (s, 9H), 0.88 (s, 9H), 1.38 (m, 1H), 1.53 (m, 1H), 2.40 (m, 1H), 2.64 (m, 3H), 2.83 (m, 1H), 3.12 (m, 4H), 3.54 (m, 4H), 3.82 (m, 3H), 3.95 (m, 1H), 4.03 (s, 1H), 4.18 (m, 1H), 4.43 (d, J=6.99 Hz, 1H), 4.60 (m, 2H), 6.92 (m, 4H), 7.04 (m, 2H), 7.21 (m, 4H), 7.32 (m, 1H), 7.58 (m, 3H), 7.89 (m, 5H), 8.65 (d, J=4.41 Hz, 1H).

### EXAMPLE 60

### methyl(1S)-1-[({(1R,3S,4S)-4-[((2S)-3,3-dimethyl-2-{3-[(2-methyl-1,3-thiazol-4-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 1H (0.030 g, 0.056 mmol) in THF (0.5 mL) was treated with the product from Example 14B (0.023 g, 0.073 mmol), DEPBT (0.025 g, 0.085 mmol), and N,N-diisopropylethylamine (0.049 mL, 0.282 mmol) and the mixture was stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by elution with 0-5% methanol in ethyl acetate to give the title compound (0.044 g, 94% yield). ¹H NMR (300 MHz, DMSO-d6), δ ppm 0.80 (s, 9H), 0.89 (m, 9H), 1.38 (m, 1H), 1.53 (m, 1H), 2.43 (m, 1H), 2.63 (m, 6H), 2.83 (m, 1H), 3.03 (m, 2H), 3.20 (m, 1H), 3.53 (m, 4H), 3.94 (m, 3H), 4.36 (m, 4H), 6.88 (d, J=9.56 Hz, 1H), 7.05 (m, 5H), 7.24 (m, 3H), 7.32 (m, 1H), 7.51 (d, J=9.56 Hz, 1H), 7.89 (m, 5H), 8.65 (d, J=4.78 Hz, 1H).

### EXAMPLE 61A

### tert-butyl(2S)-3,3-dimethyl-2-{[(3-pyridinylmethoxy)carbonyl]amino}butanoate

A solution containing L-tert-leucine tert-butyl ester hydrochloride (0.20 g, 0.90 mmol) in THF (9 mL) was treated with [(3-pyridinyl)methyl]-(4-nitrophenyl)carbonate (0.27 g, 0.99 mmol) and triethylamine (0.38 mL, 2.73 mmol), and the mixture was stirred at 25° C. for 16 hours. The reaction mixture was partitioned between ethyl acetate and saturated NaHCO₃, and the organic phase was washed with brine and dried over MgSO₄ filtered and concentrated. The residue was chromatographed on silica gel eluting 0-66% ethyl acetate in chloroform to give the title compound (0.080 g, 28% yield).

### EXAMPLE 61B

### (2S)-3,3-dimethyl-2-{[(3-pyridinylmethoxy)carbonyl]amino}butanoic Acid

A solution containing the product from Example 61A (0.017 g, 0.052 mmol) in dichloromethane (0.2 mL) was treated with trifluoroacetic acid (0.2 mL), and the mixture was stirred at 25° C. for 2 hours. The solvent was concentrated and the residue was dissolved in toluene and concentrated several times to give the title compound as the trifluoroacetic acid salt, which was used without further purification.

### EXAMPLE 61C

### 3-pyridinylmethyl(1S,4S,5S,7S,10S)-4-benzyl-1,10-ditert-butyl-5-hydroxy-2,9,12-trioxo-7-[4-(2-pyridinyl)benzyl]-13-oxa-3,8,11-triazatetradec-1-ylcarbamate

A solution containing the product from Example 2C (0.025 g, 0.047 mmol) in THF (0.5 mL) was treated with the product from Example 61B (0.052 mmol), DEPBT (0.021 g, 0.071 mmol), and N,N-diisopropylethylamine (0.041 mL, 0.235 mmol) and the mixture was stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-5% methanol in chloroform to give the title compound (0.024 g, 65% yield). ¹H NMR (300 MHz, DMSO-d₆) δ ppm 0.79(m, 9H), 0.83(m; 9H), 1.59-1.46(m, 2H), 2.80-2.70(m, 3H), 3.49(s, 3H), 3.69-3.60(m, 1H), 3.84-3.80(d, J=9.56 Hz, 1H), 3.96-3.93(d, J=9.93 Hz, 1H), 4.22-4.00(m, 2H), 4.88-4.86(d, J=5.52 Hz, 1H), 5.14-5.04(m, 2H), 6.62-6.59(d, J=9.56 Hz, 1H), 7.03-7.00(d, J=9.93 Hz, 1H), 7.20-7.06(m, 7H), 7.33-7.28(m, 1H), 7.43-7.39(m, 1H), 7.59-7.56(d, J=9.19 Hz, 1H), 7.82-7.77(m, 2H), 7.89-7.84(m, 4H), 8.54-8.53(m, 1H), 8.64-8.60(m, 2H).

### EXAMPLE 62

### benzyl(1S,4S,5S,7S,10S)-4-benzyl-1,10-ditert-butyl-5-hydroxy-2,9,12-trioxo-7-[4-(2-pyridinyl)benzyl]-13-oxa-3,8,11-triazatetradec-1-ylcarbamate

A solution containing the product from Example 58C (0.011 g, 0.014 mmol) in THF (0.2 mL) was treated with N-benzyloxycarbonyloxy)succinimide (0.005 g, 0.020 mmol) and triethylamine (0.006 mL, 0.043 mmol) and the mixture was stirred at 25° C. for 3 hours. The reaction was partitioned between ethyl acetate and saturated NaHCO₃, and the organic phase was washed with brine and dried over MgSO₄, filtered and concentrated. The residue was chromatographed on silica gel eluting with 0-10% methanol in chloroform to give the title compound (0.006 g, 55% yield).

¹H NMR (300 MHz, DMSO-d₆) δ ppm 0.80(m, 9H), 0.83(m, 9H), 1.59-1.46(m, 2H), 2.80-2.70(m, 3H), 3.49(s, 3H), 3.69-3.60(m, 1H), 3.84-3.80(d, J=9.56 Hz, 1H), 3.96-3.93(d, J=9.93 Hz, 1H), 4.22-4.00(m, 2H), 4.88-4.86(d, J=5.52 Hz, 1H), 5.05(s, 2H), 6.62-6.58(d, J=9.56 Hz, 1H), 6.96-6.93(d, J=9.93 Hz, 1H), 7.20-7.17(m, 8H), 7.38-7.29(m, 5H), 7.59-7.58(d, J=8.82 Hz, 1H), 7.82-7.78(m, 1H), 7.89-7.84(m, 4H), 8.64-8.63(m, 1H).

### EXAMPLE 63A

### methyl(2S)-3,3-dimethyl-2-{[(4-nitrophenoxy)carbonyl]amino}butanoate

A solution of L-tert-leucine methyl ester hydrochloride (0.300 g, 1.65 mmol) in dichloromethane (4 mL) at 0° C. was treated with 4-nitrophenyl chloroformate (0.366, 1.82 mmol) and N-methyl morpholine (0.380 mL, 3.46 mmol), and the mixture was stirred at 25° C. for 64 hours. The reaction was partitioned between dichloromethane and saturated NaHCO₃, and the organic phase was washed with brine and dried over MgSO₄, filtered and concentrated to give the title compound (0.562 g, quantitative), which was used without further purification.

### EXAMPLE 63B

### methyl(2S)-2-({[benzyl(methyl)amino]carbonyl}amino)-3,3-dimethylbutanoate

A solution containing the product from Example 63A (0.075 g, 0.242 mmol) in toluene (0.5 mL) was treated with N-benzylmethylamine (0.035 mL, 2.71 mmol), and the mixture was stirred at 80° C. for 1 hour. The reaction was partitioned between ethyl acetate and 10% Na₂CO₃, and the organic phase was washed with brine and dried over MgSO₄, filtered and concentrated. The residue was chromatographed on silica gel eluting with 0-20% ethyl acetate in dichloromethane to give the title compound (0.046 g, 65% yield).

### EXAMPLE 63C

### (2S)-2-({[benzyl(methyl)amino]carbonyl}amino)-3,3-dimethylbutanoic Acid

A solution containing the product from Example 63B (0.046 g, 0.057 mmol) in dioxane (1.6 mL) was treated with an aqueous solution of lithium hydroxide (0.63 mL, 0.5 N), and the mixture was stirred at 25° C. for 16 hours. An aqueous HCl solution (0.60 mL, 1N) was added, the reaction mixture was partitioned between ethyl acetate and water, and the organic phase was washed with brine and dried over MgSO₄, filtered and concentrated to give the crude product, which was used without further purification.

### EXAMPLE 63D

### methyl(1S,4S,6S,7S,10S)-7-benzyl-1,10-ditert-butyl-6-hydroxy-13-methyl-2,9,12-trioxo-14-phenyl-4-[4-(2-pyridinyl)benzyl]-3,8,11,13-tetraazatetradec-1-ylcarbamate

A solution containing the product from Example 2C (0.020 g, 0.038 mmol) in THF (0.4 mL) was treated with the product from Example 63C (0.013 g, 0.047 mmol), DEPBT (0.017 g, 0.057 mmol), and N,N-diisopropylethylamine (0.035 mL, 0.201 mmol) and the mixture was stirred at 25° C. for 45 minutes. The mixture was partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.017 g, 57% yield).

¹H NMR (300 MHz, DMSO-d₆) δ ppm 0.80(s, 9H), 0.81(s, 9H), 1.58-1.49(m, 2H), 2.74-2.72(m, 3H), 2.79(s, 3H), 3.49(s, 3H), 3.67-3.61(m, 1H), 3.84-3.81(d, J=9.93 Hz, 1H), 4.12-3.99(m, 1H), 4.16-4.13(d, J=8.82 Hz, 2H), 4.44(s, 2H), 4.82-4.80(d, J=5.88 Hz, 1H), 5.40-5.37(d, J=9.19 Hz, 1H), 6.62-6.59(d, J=9.93 Hz, 1H), 7.35-7.10(m, 13H), 7.67-7.64(d, J=8.82 Hz, 1H), 7.82-7.77(m, 1H), 7.88-7.84(m, 4H), 8.63 (d, J=4.41 Hz, 1H).

### EXAMPLE 64

### methyl(1S,4R,6S,7S,10S)-7-benzyl-1,10-ditert-butyl-6-hydroxy-13-methyl-2,9,12-trioxo-14-phenyl-4-[4-(2-pyridinyl)benzyl]-3,8,11,13-tetraazatetradec-1-ylcarbamate

A solution containing the product from Example 1H (0.020 g, 0.038 mmol) in THF (0.4 mL) was treated with the product from Example 63C (0.013 g, 0.047 mmol), DEPBT (0.017 g, 0.057 mmol), and N,N-diisopropylethylamine (0.035 mL, 0.201 mmol) and the mixture was stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give the title compound (0.022 g, 74% yield).

¹H NMR (300 MHz, DMSO-d₆) δ ppm 0.75(m, 9H), 0.78(m, 9H), 1.35-1.22(m, 1H), 1.65-1.54(m, 1H), 2.77-2.60(m, 4H), 2.79(s, 3H), 3.57(s, 3H), 3.83-3.77(m, 1H), 3.94-3.83(m, 1H), 4.09-4.06(d, J=8.82 Hz, 1H), 4.21-4.10(m, 1H), 4.51-4.38(m, 2H), 4.77-4.75(d, J=5.52 Hz, 1H), 5.43-5.40(d, J=8.82 Hz, 1H), 6.85-6.82(d, J=9.52 Hz, 1H), 7.26-7.10(m, 10H), 7.35-7.30(m, 3H), 7.60-7.57(d, J=9.19, 1H), 7.79-7.77(d, J=7.72 Hz, 1H), 7.93-7.82(m, 4H), 8.65-8.62(m, 1H).

### EXAMPLE 65A

### (2S)-3,3-dimethyl-2-[3-(2-methylbenzyl)-2-oxo-1-imidazolidinyl]butanoic Acid

A solution containing the product from Example 6F (0.150 g, 0.65 mmol) in a mixture of toluene (2.5 mL) and methanol (2.5 mL) was treated with o-tolualdehyde (0.081 mL, 0.687 mmol), and the mixture was stirred at 50° C. for 18 hours. The reaction was cooled to 25° C. and sodium borohydride (0.049 g, 1.29 mmol) was added and the reaction was stirred at 25° C. for 1 hour. The reaction mixture was quenched with 1N NaHCO₃, stirred for 1 hour, and partitioned between ethyl acetate and water. The organic phase was washed with brine and dried over MgSO₄, filtered and concentrated. A solution containing the residue (0.220 g) in 1,2-dichloroethane (10 mL) was treated with N,N-disuccinimidyl carbonate (0.20 g, 0.781 mmol) and triethylamine (0.11 mL, 0.789 mmol), stirred at 25° C. for 68 hours, and partitioned with 10% Na₂CO₃, and the aqueous was extracted with additional dichloromethane. The organic phase was dried over MgSO₄, filtered and concentrated. A solution containing the concentrate (0.245 g) in dichloromethane (2.5 in L) was treated with trifluoracetic acid (2.5 mL), stirred at 25° C. for 2 hours and concentrated to give the title compound, which was used without further purification.

### EXAMPLE 65B

### methyl(1S)-1-[({(1S,3S,4S)-4-({(2S)-3,3-dimethyl-2-[3-(2-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 2C (0.025 g, 0.047 mmol) in THF (0.5 mL) was treated with the product from Example 65A (0.014 g, 0.046 mmol), DEPBT (0.021 g, 0.071 mmol), and N,N-diisopropylethylamine (0.041 mL, 0.235 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The product was partitioned between ethyl acetate and saturated NaHCO₃ solution. The organic phase was washed brine, dried over MgSO₄, filtered and concentrated to give the title compound (0.020 g, 49% yield). ¹H NMR (300 MHz, DMSO-d₆) δ ppm 0.83(m, 9H), 0.89(m, 9H), 1.62-1.48(m, 2H), 2.31(s, 3H), 2.34-2.24(m, 1H), 2.62-2.53(m, 1H), 2.68-2.65(m, 2H), 2.84-2.73(m, 2H), 2.97-2.88(m, 1H), 3.22-3.12(m, 1H), 3.50(s, 3H), 3.70-3.62(m, 1H), 3.87-3.83(d, J=9.93 Hz, 1H), 4.08(s, 1H), 4.43-4.12(m, 4H), 4.55-4.52(d, J=7.72 Hz, 1H), 6.65-6.62(d, J=9.56 Hz, 1H), 7.01-6.99(m, 3H), 7.09-7.08(m, 2H), 7.24-7.20(m, 5H), 7.32-7.29(m, 1H), 7.49-7.46(d, J=9.56 Hz, 1H), 7.91-7.82(m, 5H), 8.64-8.63(d, J=4.41 Hz, 1H).

### EXAMPLE 66A

### (2S)-3,3-dimethyl-2-[3-(3-methylbenzyl)-2-oxo-1-imidazolidinyl]butanoic Acid

A solution containing the product from Example 6F (0.150 g, 0.65 mmol) in a mixture of toluene (2.5 mL) and methanol (2.5 mL) was treated with m-tolualdehyde (0.080 mL, 0.692 mmol), stirred at 50° C. for 18 hours, cooled to 25° C., treated with sodium borohydride (0.049 g, 1.29 mmol), stirred at 25° C. for 1 hour, quenched with 1N NaHCO₃, stirred for 1 hour, and partitioned between ethyl acetate and water. The organic phase was washed with brine and dried over MgSO₄, filtered and concentrated. A solution of the concentrate (0.211 g) in 1,2-dichloroethane (10 mL) was treated with N,N-disuccinimidyl carbonate (0.20 g, 0.781 mmol) and triethylamine (0.11 mL, 0.789 mmol), stirred at 25° C. for 68 hours, and partitioned with 10% Na₂CO₃. The aqueous was extracted with additional chloroform. The combined organic phase was dried over MgSO₄, filtered and concentrated. A solution of the concentrate (0.254 g) in dichloromethane (2.5 mL) was treated with trifluoracetic acid (2.5 mL), and the mixture was stirred at 25° C. for 2 hours. The solvent was concentrated to give the title compound, which was used without further purification.

### EXAMPLE 66B

### methyl(1S)-1-[({(1S,3S,4S)-4-({(2S)-3,3-dimethyl-2-[3-(3-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 2C (0.025 g, 0.047 mmol) in THF (0.5 mL) was treated with the product from Example 66A (0.014 g, 0.046 mmol), DEPBT (0.021 g, 0.071 mmol), and N,N-diisopropylethylamine (0.041 mL, 0.235 mmol) and the mixture was stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The product was purified by reversed phase chromatography on a C18 column eluting with 5-100% acetonitrile in water (0.1% TFA). The product was partitioned between ethyl acetate and saturated NaHCO₃ solution. The organic phase was washed brine, dried over MgSO₄, filtered and concentrated to give the title compound (0.018 g, 44% yield).

¹H NMR (300 MHz, DMSO-d₆) δ ppm 0.83(m, 9H), 0.89(m, 9H), 1.62-1.48(m, 2H), 2.31(s, 3H), 2.34-2.24(m, 1H), 2.62-2.53(m, 1H), 2.68-2.65(m, 2H), 2.97-2.73(m, 3H), 3.22-3.12(m, 1H), 3.50(s, 3H), 3.70-3.62(m, 1H), 3.87-3.83(d, J=9.93 Hz, 1H), 4.08(s, 1H), 4.33-4.11(m, 4H), 4.56-4.53(d, J=7.72 Hz, 1H), 6.65-6.62(d, J=9.56 Hz, 1H), 7.04-7.02(m, 3H), 7.11-7.07(m, 4H), 7.25-7.21(m, 4H), 7.33-7.28(m, 1H), 7.49-7.46(d, J=9.56 Hz, 1H), 7.91-7.82(m, 4H), 8.64-8.63(d, J=4.04 Hz, 1H).

### EXAMPLE 67A

### benzyl(4S,5S)-5-{(2S)-2-[(tert-butoxycarbonyl)amino]-3-phenylpropyl}-2,2-dimethyl-4-[4-(3-pyridinyl)benzyl]-1,3-oxazolidine-3-carboxylate

A solution containing the product from Example 23I (0.200 g, 0.283 mmol) in DMF (3 mL) was treated with LiCl (0.120 g, 2.83 mmol), dichlorobis(triphenylphosphine)palladium(II) (0.060 g, 0.085 mmol), and 3-tri-r-butylstannlypyridine (0.200 mL, 0.870 mmol), heated at 1001C for 16 hours, cooled and partitioned between ethyl acetate and water. The organic phase was washed with brine and dried over MgSO₄, filtered and concentrated. The residue was chromatographed on silica gel eluting with 0-25% ethyl acetate in dichloromethane to give the title compound (0.130 g, 72% yield).

### EXAMPLE 67B

### tert-butyl(1S,3S,4S)-4-amino-1-benzyl-3-hydroxy-5-[4-(3-pyridinyl)phenyl]pentylcarbamate

A solution containing the product from Example 67A (0.130 g, 0.205 mmol) in methanol (3 mL) was treated with Pd(OH)₂ on carbon (0.040 g, 20% Pd by wt.) and HCl solution (0.150 mL, 4N in dioxane), stirred under a hydrogen atmosphere (balloon pressure) for 2.5 hours at 25° C., filtered through a bed of celite® and rinsed with methanol. The filtrate was concentrated to give the title compound as the hydrochloride salt.

### EXAMPLE 67C

### tert-butyl(1S,3S,4S)-1-benzyl-3-hydroxy-4-({(2S)-2-[(methoxycarbonyl)amino]-3,3-dimethylbutanoyl}amino)-5-[4-(3-pyridinyl)phenyl]pentylcarbamate

A solution containing the product from Example 67B (0.205 mmol) in THF (2 mL) was treated with the product from Example 1F (0.046 g, 0.243 mmol), DEPBT (0.10 g, 0.334 mmol), and N,N-diisopropylethylamine (0.350 mL, 2.01 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated to give the title compound (0.073 g), which was used without further purification.

### EXAMPLE 67D

### methyl(1S)-1-[({(1S,2S,4S)-4-amino-2-hydroxy-5-phenyl-1-[4-(3-pyridinyl)benzyl]pentyl}amino)carbonyl]-2,2-dimethylpropylcarbamate

A solution containing the product from Example 67C (0.073 g) in dichloromethane (5 mL) was treated with trifluoroacetic acid (5 mL) and the mixture was stirred at 25° C. for 1 hour. The solvent was concentrated and the residue was purified by reversed phase chromatography on a C18 column eluting with 5-100% acetonitrile in water (0.1% TFA) to give the title compound as the trifluoroacetic acid salt (0.073 g, 47% yield).

### EXAMPLE 67E

### methyl(1S)-1-[({(1S,2S,4S)-4-[((2S)-3,3-dimethyl-2-{3-[(2-methyl-1,3-thiazol-4-yl)methyl]-2-oxo-1-imidazolidinyl}butanoyl)amino]-2-hydroxy-5-phenyl-1-[4-(3-pyridinyl)benzyl]pentyl}amino)carbonyl]-2,2-dimethylpropylcarbamate

A solution containing the product from Example 67D (0.025 g, 0.033 mmol) in THF (0.4 mL) was treated with the product from Example 14B (0.017 g, 0.039 mmol), DEPBT (0.017 g, 0.057 mmol), and N,N-diisopropylethylamine (0.060 mL, 0.344 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate to give the title compound (0.01 g). ¹H NMR (300 MHz, DMSO-d₆), δ ppm 0.81 (s, 9H), 0.86 (s, 9H), 1.53 (m, 2H), 2.39 (m, 2H), 2.66 (m, 4H), 2.77 (d, J=6.99 Hz, 2H), 3.00 (m, 2H), 3.19 (m, 1H), 3.49 (s, 3H), 3.61 (m, 1H), 3.93 (m, 2H), 4.32 (m, 4H), 4.83 (d, J=5.15 Hz, 1H), 6.81 (d, J=9.19 Hz, 1H), 7.03 (m, 5H), 7.21 (s, 1H), 7.32 (d, J=8.09 Hz, 2H), 7.46 (dd, J=7.72, 4.78 Hz, 1H), 7.55 (m, 3H), 7.87 (d, J=8.82 Hz, 1H), 8.01 (d, J=8.09 Hz, 1H), 8.53 (d, J=4.41 Hz, 1H), 8.83 (d, J=1.84 Hz, 1H).

### EXAMPLE 68A

### 2-methyl-6-(tributylstannyl)pyridine

A solution containing 2-bromo-6-methylpyridine (1.48 g, 8.63 mmol) in ether (15 mL) at −78° C. was treated with n-butyllithium (5.39 mL, 1.6 M in hexanes) dropwise, stirred at −78° C. for 1 hour, treated with tributyltin chloride (4.21 mL, 12.94 mmol), stirred at −78° C. for 4 hours, quenched with saturated ammonium chloride solution, and partitioned between ether and water. The organic phase was washed with brine and dried over MgSO₄, filtered and concentrated. The residue was purified by chromatography on neutral alumina eluting with 10% ethyl acetate in dichloromethane to give the title compound.

### EXAMPLE 68B

### benzyl(4S,5S)-5-{(2S)-2-[(tert-butoxycarbonyl)amino]-3-phenylpropyl}-2,2-dimethyl-4-[4-(6-methyl-2-pyridinyl)benzyl]-1,3-oxazolidine-3-carboxylate

A solution containing the product from Example 23I (0.113 g, 0.160 mmol) in DMF (1.5 mL) was treated with LiCl (0.068 g, 1.60 mmol), dichlorobis(triphenylphosphine)palladium(II) (0.034 g, 0.048 mmol), and the product from Example 68A (0.367 g, 0.961 mmol), heated at 110° C. for 16 hours, cooled and partitioned between ethyl acetate and water. The organic phase was washed with brine and dried over MgSO₄, filtered and concentrated. The residue was chromatographed on silica gel eluting with 0-50% ethyl acetate in dichloromethane to give the title compound (0.102 g, 98% yield).

### EXAMPLE 68C

### benzyl(1S,2S,4S)-4-amino-2-hydroxy-1-[4-(6-methyl-2-pyridinyl)benzyl]-5-phenylpentylcarbamate

A solution containing the product from Example 68B (0.07 g, 0.108 mmol) in a mixture of THF (0.5 mL), methanol (0.3 mL), and aqueous HCl (0.5 mL, 1 N) was stirred at 59C for 16 hours. The solvent was removed under reduced pressure to give the title compound as the hydrochloride salt, which was used without further purification.

### EXAMPLE 68D

### benzyl(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methyl-2-pyridinyl)benzyl]-5-phenylpentylcarbamate

A solution containing the product from Example 68C (0.108 mmol) in THF (0.5 mL) was treated with the product from Example 10D (0.048 g, 0.14 mmol), DEPBT (0.048 g, 0.162 mmol), and N,N-diisopropylethylamine (0.281 mL, 1.62 mmol), stirred at 25° C. for 3 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate to give the title compound (0.048 g, 56% yield).

### EXAMPLE 68E

### (2S)-N-{(1S,3S,4S)-4-amino-1-benzyl-3-hydroxy-5-[4-(6-methyl-2-pyridinyl)phenyl]pentyl}-3,3-dimethyl-2-{3-[(6-methyl-2-pyridinyl)methyl]-2-oxo-1-imidazolidinyl}butanamide

A solution containing the product from Example 68D (0.046 g, 0.058 mmol) in a mixture of ethyl acetate (0.25 mL) and methanol (0.25 mL) was treated with Pd(OH)₂ on carbon (0.012 g, 20% Pd by wt.) and HCl solution (0.058 mL, 4N in dioxane), stirred under a hydrogen atmosphere (balloon pressure) at 25° C. for 16 hours, filtered through a bed of celite® and rinsed with methanol. The solvent was concentrated to give the crude product as the hydrochloride salt, which was used without further purification.

### EXAMPLE 68F

### methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methyl-2-pyridinyl)benzyl]-5-phenylpentyl}amino)carbonyl]-2,2-dimethylpropylcarbamate

A solution containing the product from Example 68E (0.058 mmol) in THF (0.5 mL) was treated with the product from Example 1F (0.013 g, 0.069 mmol), DEPBT (0.026 g, 0.087 mmol), and N,N-diisopropylethylamine (0.100 mL, 0.577 mmol), stirred at 25° C. for 16 hours, and partitioned between ethyl acetate and 10% Na)CO₃ solution. The organic phase was washed with additional 10% Na₂CO₃ solution and brine, dried over MgSO₄, filtered and concentrated. The residue was purified by chromatography on silica gel eluting with 0-100% ethyl acetate/dichloromethane, followed by 0-5% methanol in ethyl acetate, to give the title compound (0.033 g, 69% yield).

¹H NMR (300 MHz, DMSO-d₆), δ ppm 0.84 (s, 9H), 0.87 (s, 9H), 1.53 (m, 3H), 2.42 (m, 5H), 2.74 (m, 4H), 3.05 (m, 2H), 3.24 (m, 2H), 3.60 (m, 4H), 3.97 (m, 2H), 4.19 (m, 2H), 4.34 (m, 2H), 4.81 (d, J=5.15 Hz, 1H), 6.78 (d, J=9.19 Hz, 1H), 7.02 (m, 6H), 7.16 (m, 2H), 7.29 (d, J=8.09 Hz, 2H), 7.69 (m, 4H), 7.89 (d, J=8.09 Hz, 3H).

### EXAMPLE 69

### methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-5-phenyl-1-[4-(3-pyridinyl)benzyl]pentyl}amino)carbonyl]-2,2-dimethylpropylcarbamate

A solution containing the product from Example 67D (0.025 g, 0.033 mmol) in THF (0.4 mL) was treated with the product from Example 10D (0.016 g, 0.047 mmol), DEPBT (0.017 g, 0.057 mmol), and N,N-diisopropylethylamine (0.060 mL, 0.344 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by elution with 0.5% methanol in ethyl acetate to give the title compound (0.015 g, 56% yield). ¹H NMR (300 MHz, DMSO-d₆) δ ppm 0.84(s, 9H), 0.86(s, 9H), 1.59-1.50(m, 2H), 2.48-2.35(m, 2H), 2.46(s, 3H), 2.70-2.62(m, 1H), 2.79-2.77(m, 2H), 3.00-2.92(m, 1H), 3.13-3.02(m, 1H), 3.28-3.18(m, 1H), 3.49(s, 3H), 3.67-3.58(m, 1H), 3.95-3.93(m, 1H), 3.97(s, 1H), 4.27-4.12(m, 2H), 4.40-4.26(m, 2H), 4.84-4.82(d, J=5.52 Hz, 1H), 6.83-6.80 (d, J=9.56 Hz, 1H), 7.05-7.02 (m, 5H), 7.16-7.14(d, J=7.72 Hz, 1H), 7.34-7.33(d, J=8.09 Hz, 2H), 7.49-7.44(dd, J=8.27, 4.96 Hz, 1H), 7.59-7.53(m, 3H), 7.70-7.65(t, J=7.54 Hz, 1H), 7.91-7.88(d, J=9.19 Hz, 1H), 8.03-7.99(m, J=6.07, 2.39 Hz, 1H), 8.55-8.53(dd, J=4.78, 1.47 Hz, 1H), 8.84-8.83(d, J=1.84 Hz, 1H).

### EXAMPLE 70A

### (2S)-2-(3-benzyl-2-oxo-1-imidazolidinyl)-3,3-dimethylbutanoic Acid

A solution containing the product from Example 6F (1.0 g, 4.35 mmol) in a mixture of benzene (10 mL) and ethanol (10 mL) was treated with benzaldehyde (0.46 mL, 4.55 mmol), stirred at 70° C. for 16 hours, cooled to 25° C., treated with sodium borohydride (0.50 g, 13.22 mmol), stirred at 25° C. for 3 hours, quenched with 1N NaHCO₃ and stirred for 1 hour, and partitioned between ethyl acetate and saturated NaHCO₃. The organic phase was washed with brine and dried over MgSO₄, filtered and concentrated. A solution of the concentrate (4.35 mmol) in 1,2-dichloroethane (175 mL) was treated with N,N-disuccinimidyl carbonate (1.34 g, 5.23 mmol) and triethylamine (0.60 mL, 4.30 mmol), stirred at 25° C. for 16 hours, and partitioned with 10% Na₂CO₃. The aqueous was extracted with additional dichloromethane. The combined organic phase was dried over MgSO₄, filtered and concentrated. A solution of the concentrate (4.35 mmol) in dichloromethane (25 mL) was treated with trifluoracetic acid (25 mL), and the mixture was stirred at 25° C. for 2 hours. The solvent was concentrated, and the residue was purified by reversed phase chromatography on a C18 column eluting with a gradient starting with 0-100% acetonitrile/water (0.1% TFA) to give the title compound (0.76 g, 60% yield).

### EXAMPLE 70B

### methyl(1S)-1-[({(1S,2S,4S)-4-{[(2S)-2-(3-benzyl-2-oxo-1-imidazolidinyl)-3,3-dimethylbutanoyl]amino}-2-hydroxy-5-phenyl-1-[4-(3-pyridinyl)benzyl]pentyl}amino)carbonyl]-2,2-dimethylpropylcarbamate

A solution containing the product from Example 67D (0.025 g, 0.033 mmol) in THF (0.4 mL) was treated with the product from Example 70A (0.014 g, 0.048 mmol), DEPBT (0.017 g, 0.057 mmol), and N,N-diisopropylethylamine (0.060 mL, 0.344 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by elution with 0.5% methanol in ethyl acetate to give the title compound (0.013 g, 49% yield). ¹H NMR (300 MHz, DMSO-d₆) δ ppm 0.83(s, 9H), 0.86(s, 9H), 1.58-1.49(m, 2H), 2.45-2.35(m, 2H), 2.70-2.60(m, 1H), 2.99-2.74(m, 4H), 3.24-3.15(m, 1H), 3.49(s, 3H), 3.67-3.58(m, 1H), 3.96-3.93(d, J=9.93 Hz, 1H), 3.97(s, 1H), 4.27-4.11(m, 2H), 4.30(s, 2H), 4.84-4.82(d, J=5.88 Hz, 1H), 6.83-6.80(d, J=9.19 Hz, 1H), 7.06-7.03(m, 5H), 7.40-7.25(m, 6H), 7.49-7.44(dd, J=8.27, 4.96 Hz, 1H), 7.58-7.52(m, 3H), 7.91-7.88(d, J=8.82 Hz, 1H), 8.03-7.99(m, 1H), 8.55-8.52(dd, J=4.78, 1.47 Hz, 1H), 8.84-8.83(d, J=2.21 Hz, 1H).

### EXAMPLE 71A

### (2S)-2-[3-(3-methoxybenzyl)-2-oxo-1-imidazolidinyl]-3,3-dimethylbutanoic Acid

A solution containing the product from Example 6F (0.150 g, 0.65 mmol) in a mixture of toluene (2.5 mL) and methanol (2.5 mL) was treated with m-anisaldehyde (0.083 mL, 0.68 mmol), stirred at 509C for 18 hours, cooled to 25° C., treated with sodium borohydride (0.049 g, 1.29 mmol), stirred at 25° C. for 1 hour, quenched with 1N NaHCO₃ and stirred for 1 hour, and partitioned between ethyl acetate and water. The organic phase was washed with brine and dried over MgSO₄, filtered and concentrated. A solution of the concentrate (0.242 g) in 1,2-dichloroethane (10 mL) was treated with N,N-disuccinimidyl carbonate (0.20 g, 0.781 mmol) and triethylamine (0.11 mL, 0.789 mmol), stirred at 25° C. for 68 hours, and partitioned with 10% Na₂CO₃. The aqueous was extracted with additional chloroform. The combined organic phase was dried over MgSO₄, filtered and concentrated. A solution of the concentrate (0.265 g) in dichloromethane (2.5 mL) was treated with trifluoracetic acid (2.5 mL), stirred at 25° C. for 2 hours, and concentrated to give the title compound, which was used without further purification.

### EXAMPLE 71B

### methyl(1S)-1-[({(1S,3S,4S)-3-hydroxy-4-({(2S)-2-[3-(3-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate

A solution containing the product from Example 2C (0.025 g, 0.047 mmol) in THF (0.5 mL) was treated with the product from Example 71A (0.020 g, 0.062 mmol), DEPBT (0.021 g, 0.071 mmol), and N,N-diisopropylethylamine (0.041 mL, 0.235 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with a gradient starting with 5-100% acetonitrile in water (0.1% TFA). The product was partitioned between ethyl acetate and saturated NaHCO₃ solution. The organic phase was washed brine, dried over MgSO₄, filtered and concentrated to give the title compound (0.024 g, 59% yield). ¹H NMR (300 MHz, DMSO-d₆), δ ppm 0.83 (s, 9H), 0.89 (s, 9H), 1.55 (m, 2H), 2.32 (m, 1H), 2.80 (m, 6H), 3.18 (m, 1H), 3.50 (s, 3H), 3.65 (m, 1H), 3.74 (s, 3H), 3.85 (d, J=9.93 Hz, 1H), 4.20 (m, 5H), 4.54 (d, J=7.72 Hz, 1H), 6.63 (d, J=9.93 Hz, 1H), 6.85 (m, 3H), 7.08 (m, 5H), 7.28 (m, 4H), 7.48 (d, J=9.56 Hz, 1H), 7.86 (m, 5H), 8.63 (d, J=4.78 Hz, 1H).

### EXAMPLE 72

### methyl(1S)-1-[({(1R,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate

A solution containing the product from Example 1H (0.040 g, 0.075 mmol) in THF (0.6 mL) was treated with the product from Example 70A (0.027 g, 0.092 mmol), DEPBT (0.034 g, 0.114 mmol), and N,N-diisopropylethylamine (0.066 mL, 0.379 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by elution with 0-5% methanol in ethyl acetate to give the title compound (0.045 g, 73% yield). ¹H NMR (300 MHz, DMSO-d₆), δ ppm 0.80 (s, 9H), 0.87 (s, 9H), 1.38 (t, J=111.58 Hz, 1H), 1.54 (m, 1H), 2.41 (m, 1H), 2.64 (m, 3H), 2.87 (m, 3H), 3.19 (m, 1H), 3.53 (m, 4H), 3.84 (d, J=9.56 Hz, 1H), 3.95 (m, 1H), 4.04 (s, 1H), 4.18 (m, 1H), 4.29 (m, 2H), 4.45 (d, J=7.35 Hz, 1H), 6.88 (d, J=9.56 Hz, 1H), 7.05 (m, 5H), 7.30 (m, 8H), 7.53 (d, J=9.56 Hz, 1H), 7.90 (m, 5H), 8.65 (d, J=4.41 Hz, 1H).

### EXAMPLE 73A

### benzyl(4S,5S)-5-{(2S)-2-[(tert-butoxycarbonyl)amino]-3-phenylpropyl}-2,2-dimethyl-4-[4-(4-pyridinyl)benzyl]-1,3-oxazolidine-3-carboxylate

A solution containing the product from Example 23I (0.64 g, 0.906 mmol) in DMF (10 mL) was treated with LiCl (0.384 g, 9.06 mmol), dichlorobis(triphenylphosphine)palladium(II) (0.19 g, 0.271 mmol), and 4(tri-n-butylstannyl)pyridine (1.0 g, 2.72 mmol), heated at 100° C. for 16 hours, cooled and partitioned between ethyl acetate and water. The organic phase was washed with brine and dried over MgSO₄, filtered and concentrated. The residue was chromatographed on silica gel eluting with 0-25% ethyl acetate in dichloromethane to give the title compound (0.28 g, 49% yield).

### EXAMPLE 73B

### benzyl(1S,2S,4S)-4-amino-2-hydroxy-5-phenyl-1-[4-(4-pyridinyl)benzyl]pentylcarbamate

A solution containing the product from Example 73A (0.28 g, 0.441 mmol) in a mixture of THF (5 mL), methanol (5 mL), and aqueous HCl (5 mL, 1 N) was stirred at 60° C. for 16 hours, and concentrated to give the title compound as the hydrochloride salt, which was used without further purification.

### EXAMPLE 73C

### benzyl(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-5-phenyl-1-[4-(4-pyridinyl)benzyl]pentylcarbamate

A solution containing the product from Example 73B (0.441 mmol) in THF (4.5 mL) was treated with the product from Example 10D (0.18 g, 0.526 mmol), DEPBT (0.20 g, 0.669 mmol), and N,N-diisopropylethylamine (0.75 mL, 4.31 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 7.5% methanol in ethyl acetate to give the title compound (0.095 g, 28% yield).

### EXAMPLE 73D

### (2S)-N-{(1S,3S,4S)-4-amino-1-benzyl-3-hydroxy-5-[4-(4-pyridinyl)phenyl]pentyl}-3,3-dimethyl-2-{3-[(6-methyl-2-pyridinyl)methyl]-2-oxo-1-imidazolidinyl}butanamide

A solution containing the product from Example 73C (0.095 g, 0.121 mmol) in methanol (1.5 mL) was treated with Pd(OH)₂ on carbon (0.075 g, 20% Pd by wt.) and HCl solution (0.090 mL, 4N in dioxane), stirred under a hydrogen atmosphere (balloon pressure) at 25° C. for 16 hours, filtered through a bed of celite® and rinsed with methanol. The solvent was concentrated to give the title compound as the hydrochloride salt, which was used without further purification.

### EXAMPLE 73E

### methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-5-phenyl-1-[4-(4-pyridinyl)benzyl]pentyl}amino)carbonyl]-2,2-dimethylpropylcarbamate

A solution containing the product from Example 73D (0.121 mmol) in THF (1.2 mL) was treated with the product from Example 1F (0.030 g, 0.159 mmol), DEPBT (0.055 g, 0.184 mmol), and N,N-diisopropylethylamine (0.225 mL, 2.35 mmol), stirred at 25° C. for 4 hours, and partitioned between ethyl acetate and 10% Na₂CO₃ solution. The organic phase was washed with additional 10% Na₂CO₃ solution and brine, dried over MgSO₄, filtered and concentrated. The residue was purified by chromatography on silica gel eluting with a 100% ethyl acetate/dichloromethane, followed by 0-10% methanol in ethyl acetate, to give the title compound (0.048 g, 48% yield). ¹H NMR (300 MHz, DMSO-d₆) δ ppm 0.83(s, 9H), 0.86(s, 9H), 1.59-1.50(m, 2H), 2.48-2.35(m, 2H), 2.46(s, 3H), 2.70-2.62(m, 1H), 2.79-2.77(m, 2H), 3.00-2.92(m, 1H), 3.13-3.02(m, 1H), 3.28-3.18(m, 1H), 3.49(s, 3H), 3.67-3.58(m, 1H), 3.95-3.93(m, 1H), 3.97(s, 1H), 4.27-4.12(m, 2H), 4.40-4.26(m, 2H), 4.84-4.82(m, 1H), 6.83-6.80(d, J=9.93 Hz, 1H), 7.09-7.00(m, 5H), 7.16-7.14(d, J=7.35 Hz, 1H), 7.36-7.33(d, J=8.09 Hz, 2H), 7.74-7.53(m, 6H), 7.92-7.89(d, J=9.19 Hz, 1H), 8.62-8.60(d, J=5.88 Hz, 2H).

### EXAMPLE 74A

### 5-methyl-2-(tributylstannyl)pyridine

A solution containing 2-bromo-5-methylpyridine (1.42 g, 8.23 mmol) in ether (15 mL) at −78° C. was treated with n-butyllithium (5.14 mL, 1.6 M in hexanes) dropwise, stirred at −78° C. for 1 hour, treated with tributyltin chloride (3.35 mL, 12.35 mmol), stirred at 0° C. for 4 hours, quenched with saturated ammonium chloride solution and partitioned between ether and water. The organic phase was washed with brine and dried over MgSO₄, filtered and concentrated. The residue was purified by chromatography on neutral alumina eluting with 10% ethyl acetate in dichloromethane to give the title compound.

### EXAMPLE 74B

### benzyl(4S,5S)-5-{(2S)-2-[(tert-butoxycarbonyl)amino]-3-phenylpropyl}-2,2-dimethyl-4-[4-(5-methyl-2-pyridinyl)benzyl]-1,3-oxazolidine-3-carboxylate

A solution containing the product from Example 23I (0.114 g, 0.162 mmol) in DMF (1.6 mL) was treated with LiCl (0.068 g, 1.60 mmol), dichlorobis(triphenylphosphine)palladium(II) (0.034 g, 0.048 mmol), and the product from Example 74A (0.367 g, 0.961 mmol), heated at 100° C. for 16 hours, cooled and partitioned between ethyl acetate and water. The organic phase was washed with brine and dried over MgSO₄, filtered and concentrated. The residue was chromatographed on silica gel eluting with 0-15% ethyl acetate in dichloromethane. The product was purified by reversed phase chromatography on a C18 column eluting with 5-100% acetonitrile in water (0.1% TFA) to give the title compound (0.044 g, 42% yield).

### EXAMPLE 74C

### benzyl(1S,2S,4S)-4-amino-2-hydroxy-1-[4-(5-methyl-2-pyridinyl)benzyl]-5-phenylpentylcarbamate

A solution containing the product from Example 74B (0.044 g, 0.068 mmol) in a mixture of THF (0.3 mL), methanol (0.2 mL), and aqueous HCl (0.4 mL, 1 N) was stirred at 50° C. for 16 hours. The solvent was removed under reduced pressure to give the title compound as the hydrochloride salt, which was used without further purification.

### EXAMPLE 74D

### benzyl(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(5-methyl-2-pyridinyl)benzyl]-5-phenylpentylcarbamate

A solution containing the product from Example 74C (0.068 mmol) in THF (0.5 mL) was treated with the product from Example 10D (0.030 g, 0.088 mmol), DEPBT (0.030 g, 0.102 mmol), and N,N-diisopropylethylamine (0.177 mL, 1.02 mmol), stirred at 25° C. for 3 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate to give the title compound (0.033 g, 61% yield).

### EXAMPLE 74E

### (2S)-N-{(1S,3S,4S)-4-amino-1-benzyl-3-hydroxy-5-[4-(5-methyl-2-pyridinyl)phenyl]pentyl}-3,3-dimethyl-2-{3-[(6-methyl-2-pyridinyl)methyl]-2-oxo-1-imidazolidinyl}butanamide

A solution containing the product from Example 74D (0.033 g, 0.041 mmol) in a mixture of ethyl acetate (0.25 mL) and methanol (0.25 mL) was treated with Pd(OH)₂ on carbon (0.009 g, 20% Pd by wt.) and HCl solution (0.041 mL, 4N in dioxane), stirred under a hydrogen atmosphere (balloon pressure) at 25° C. for 16 hours, filtered through a bed of celite® and rinsed with methanol. The solvent was concentrated to give the title compound as the hydrochloride salt, which was used-without further purification.

### EXAMPLE 74F

### methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(5-methyl-2-pyridinyl)benzyl]-5-phenylpentyl}amino)carbonyl]-2,2-dimethylpropylcarbamate

A solution containing the product from Example 74E (0.041 mmol) in THF (0.5 mL) was treated with the product from Example 1F (0.010 g, 0.053 mmol), DEPBT (0.018 g, 0.061 mmol), and N,N-diisopropylethylamine (0.071 mL, 0.408 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by chromatography on silica gel eluting with 0-1005 ethyl acetate/dichloromethane, followed by 0-5% methanol in ethyl acetate, to give the title compound (0.024 g, 70% yield). ¹H NMR (300 MHz, DMSO-d₆) δ ppm 0.83(m, 9H), 0.86(m, 9H), 1.60-1.47(m, 2H), 2.32(s, 3H), 2.46-2.39(m, 2H), 2.46(s, 3H), 2.68-2.64(m, 1H), 2.78-2.76(d, J=6.62 Hz, 2H), 2.99-2.91(m, 1H), 3.12-3.03(m, 1H), 3.27-3.18(m, 1H), 3.52(s, 3H), 3.65-3.57(m, 1H), 3.96-3.94(m, 2H), 4.26-4.11(m, 2H), 4.40-4.28(m, 2H), 4.84-4.82(d, J=5.52 Hz, 1H), 6.81-6.78(d, J=9.93 Hz, 1H), 7.05-7.0(m, 5H), 7.16-7.14(d, J=7.72 Hz, 1H), 7.30-7.27(d, J=8.09 Hz, 2H), 7.59-7.56(m, 1H), 7.70-7.65 (m, 2H), 7.79-7.77(d, J=8.46 Hz, 1H), 7.91-7.86(m, 3H) 8.46(bs, 1H).

### EXAMPLE 75A

### (2S)-2-[3-(2-methoxybenzyl)-2-oxo-1-imidazolidinyl]-3,3-dimethylbutanoic Acid

A solution containing the product from Example 6F (0.150 g, 0.65 mmol) in a mixture of toluene (2.5 mL) and methanol (2.5 mL) was treated with o-anisaldehyde (0.079 mL, 0.68 mmol), and the mixture was stirred at 50° C. for 18 hours. The reaction was cooled to 25° C. and sodium borohydride (0.049 g, 1.29 mmol) was added and the reaction was stirred at 25° C. for 1 hour. The reaction was quenched with 1N NaHCO₃ and stirred for 1 hour. The reaction was partitioned between ethyl acetate and water, and the organic phase was washed with brine and dried over MgSO₄, filtered and concentrated. A solution of the concentrate (0.261 g) in 1,2-dichloroethane (10 mL) was treated with N,N-disuccinimidyl carbonate (0.20 g, 0.781 mmol) and triethylamine (0.11 mL, 0.789 mmol), and the mixture was stirred at 25° C. for 18 hours. The reaction was partitioned with 10% Na₂CO₃, and the aqueous was extracted with additional chloroform. The organic phase was dried over MgSO₄, filtered and concentrated. A solution of the concentrate (0.319 g) in dichloromethane (2.5 mL) was treated with trifluoracetic acid (2.5 mL), and the mixture was stirred at 25° C. for 1 hour. The solvent was concentrated to give the title compound (0.371 g), which was used without further purification.

### EXAMPLE 75B

### methyl(1S)-1-[({(1S,3S,4S)-3-hydroxy-4-({(2S)-2-[3-(2-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate

A solution containing the product from Example 2C (0.025 g, 0.047 mmol) in THF (0.5 mL) was treated with the product from Example 75A (0.020 g, 0.062 mmol), DEPBT (0.021 g, 0.071 mmol), and N,N-diisopropylethylamine (0.041 mL, 0.235 mmol) and the mixture was stirred at 25° C. for 2 hours. The mixture was partitioned between ethyl acetate and 10% Na₂CO₃ solution. The organic phase was washed with additional 10% Na₂CO₃ solution and brine, dried over MgSO₄, filtered and concentrated. The product was purified by reversed phase chromatography on a C18 column eluting with 5-100% acetonitrile in water (0.1% TFA). The product was partitioned between ethyl acetate and saturated NaHCO₃ solution. The organic phase was washed brine, dried over MgSO₄, filtered and concentrated to give the title compound (0.024 g, 59% yield). ¹H NMR (300 MHz, DMSO-d₆), δ ppm 0.83 (s, 9H), 0.89 (s, 9H), 1.26 (m, 1H), 1.38 (m, 1H), 1.54 (m, 2H), 2.33 (m, 1H), 2.83 (m, 5H), 3.18 (m, 1H), 3.50 (s, 3H), 3.66 (m, 1H), 3.83 (m, 4H), 4.25 (m, 4H), 4.53 (d, J=7.72 Hz, 1H), 6.63 (d, J=9.93 Hz, 1H), 6.95 (t, J=6.99 Hz, 1H), 7.18 (m, 11H), 7.45 (d, J=9.19 Hz, 1H), 7.86 (m, 5H), 8.63 (d, J=4.41 Hz, 1H).

### EXAMPLE 76

### methyl(1S)-1-[({(1R,3S,4S)-4-({(2S)-3,3-dimethyl-2-[3-(2-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 1H (0.020 g, 0.038 mmol) in THF (0.4 mL) was treated with the product from Example 65A (0.023 g, 0.076 mmol), DEPBT (0.017 g, 0.057 mmol), and N,N-diisopropylethylamine (0.033 mL, 0.189 mmol) and the mixture was stirred at 25° C. for 2 hours. The mixture was partitioned between ethyl acetate and 10% Na₂CO₃ solution. The organic phase was washed with additional 10% Na₂CO₃ solution and brine, dried over MgSO₄, filtered and concentrated. The product was purified by reversed phase chromatography on a C18 column eluting with 5-100% acetonitrile in water (0.1% TFA). The product was partitioned between ethyl acetate and saturated NaHCO₃ solution. The organic phase was washed brine, dried over MgSO₄, filtered and concentrated to give the title compound (0.013 g, 42% yield). ¹H NMR (300 MHz, DMSO-dc, δ ppm 0.80 (s, 9H), 0.89 (m, 9H), 1.46 (m, 2H), 2.29 (s, 3H), 238 (m, 1H), 2.76 (m, 5H), 3.22 (m, 2H), 3.53 (m, 4H), 3.84 (d, J=9.93 Hz, 1H), 3.95 (m, 1H), 4.02 (s, 1H), 4.18 (m, 2H), 4.41 (m, 2H), 6.88 (d, J=9.56 Hz, 1H), 7.04 (m, 5H), 7.21 (m, 6H), 7.32 (m, 1H), 7.53 (d, J=9.56 Hz, 1H), 7.89 (m, 5H), 8.65 (d, J=4.04 Hz, 1H).

### EXAMPLE 77

### methyl(1S)-1-[({(1R,3S,4S)-4-({(2S)-3,3-dimethyl-2-[3-(3-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 1H (0.020 g, 0.038 mmol) in THF (0.4 mL) was treated with the product from Example 66A (0.023 g, 0.076 mmol), DEPBT (0.017 g, 0.057 mmol), and N,N-diisopropylethylamine (0.033 mL, 0.189 mmol) and the mixture was stirred at 25° C. for 2 hours. The mixture was partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5100% acetonitrile in water (0.1% TFA). The product was partitioned between ethyl acetate and saturated NaHCO₃ solution. The organic phase was washed brine, dried over MgSO₄, filtered and concentrated to give the title compound (0.013 g, 42% yield). ¹H NMR (300 MHz, DMSO-d), b ppm 0.80 (s, 9H), 0.89 (m, 9H), 1.47 (m, 2H), 2.28 (s, 3H), 2.39 (m, 1H), 2.78 (m, 6H), 3.22 (m, 1H), 3.54 (m, 4H), 384 (d, J=9.93 Hz, 1H), 3.93 (m, 1H), 4.04 (s, 1H), 4.25 (m, 3H), 4.45 (d, J=6.99 Hz, 1H), 6.88 (d, J=9.93 Hz, 1H), 7.05 (m, 7H), 7.24 (m, 4H), 7.32 (m, 1H), 7.54 (d, J=9.56 Hz, 1H), 7.89 (m, 5H), 8.65 (d, J=4.41 Hz, 1H).

### EXAMPLE 78

### methyl(1S)-1-[({(1R,3S,4S)-3-hydroxy-4-({(2S)-2-[3-(2-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate

A solution containing the product from Example 1H (0.020 g, 0.038 mmol) in THF (0.4 mL) was treated with the product from Example 75A (0.018 g, 0.056 mmol), DEPBT (0.017 g, 0.057 mmol), and N,N-diisopropylethylamine (0.033 mL, 0.189 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The product was partitioned between ethyl acetate and saturated NaHCO₃ solution. The organic phase was washed brine, dried over MgSO₄, filtered and concentrated to give the title compound (0.016 g, 47% yield). ¹H NMR (300 MHz, DMSO-d₆), δ ppm 0.80 (s, 9H), 0.89 (m, 9H), 1.26 (m, 1H), 1.39 (m, 1H), 1.54 (m, 1H), 2.41 (m, 1H), 2.65 (m, 2H), 2.89 (m, 3H), 3.20 (m, 1H), 3.54 (m, 4H), 3.81 (m, 4H), 3.93 (m, 1H), 4.02 (m, 1H), 4.28 (m, 3H), 4.44 (d, J=7.35 Hz, 1H), 6.91 (m, 2H), 7.04 (m, 6H), 7.14 (d, J=7.35 Hz, 1H), 7.28 (m, 4H), 7.51 (d, J=9.93 Hz, 1H), 7.90 (m, 5H), 8.65 (d, J=4.04 Hz, 1H).

### EXAMPLE 79

### methyl(1S)-1-[({(1R,3S,4S)-3-hydroxy-4-({(2S)-2-[3-(3-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate

A solution containing the product from Example 1H (0.020 g, 0.038 mmol in THF (0.4 mL) was treated with the product from Example 71A (0.018 g, 0.056 mmol), DEPBT (0.017 g, 0.057 mmol), and N,N-diisopropylethylamine (0.033 mL, 0.189 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The product was partitioned between ethyl acetate and saturated NaHCO₃ solution. The organic phase was washed brine, dried over MgSO₄, filtered and concentrated to give the title compound (0.018 g, 54% yield). ¹H NMR (300 MHz, DMSO-d₆), δ ppm 0.80 (s, 9H), 0.87 (s, 9H), 1.25 (m, 1H), 1.39 (m, 1H), 1.54 (m, 1H), 2.42 (m, 1H), 2.64 (m, 3H), 2.89 (m, 2H), 3.21 (m, 1H), 3.54 (m, 4H), 3.72 (s, 3H), 3.84 (d, J=9.56 Hz, 1H), 3.96 (m, 1H), 4.04 (s, 1H), 4.18 (m, 1H), 4.27 (s, 2H), 4.44 (d, J=6.99 Hz, 1H), 6.85 (m, 4H), 7.06 (m, 5H), 7.29 (m, 4H), 7.54 (d, J=9.56 Hz, 1H), 7.89 (m, 5H), 8.65 (d, J=4.41 Hz, 1H).

### EXAMPLE 80A

### 4-methyl-2-(tributylstannyl)pyridine

A solution containing 2-bromo-4-methylpyridine (1.46 g, 8.49 mmol) in ether (15 mL) at −78° C. was treated with n-butyllithium (5.57 mL, 1.6 M in hexanes) dropwise, stirred at −78° C. for 1 hour, treated with tributyltin chloride (3.45 mL, 12.74 mmol), stirred at 0° C. for 4 hours, quenched with saturated ammonium chloride solution and partitioned between ether and water. The organic phase was washed with brine and dried over MgSO₄, filtered and concentrated. The residue was purified by chromatography on neutral alumina eluting with 10% ethyl acetate in dichloromethane to give the title compound.

### EXAMPLE 80B

### benzyl(4S,5S)-5-{(2S)-2-[(tert-butoxycarbonyl)amino]-3-phenylpropyl}-2,2-dimethyl-4-[4-(4-methyl-2-pyridinyl)benzyl]-1,3-oxazolidine-3-carboxylate

A solution containing the product from Example 23I (0.183 g, 0.259 mmol) in DMF (2.6 mL) was treated with LiCl (0.110 g, 2.59 mmol), dichlorobis(triphenylphosphine)palladium(II) (0.055 g, 0.078 mmol), and the product from Example 80A (0.495 g, 1.29 mmol), heated at 100° C. for 16 hours, cooled and partitioned between ethyl acetate and water. The organic phase was washed with brine and dried over MgSO₄, filtered and concentrated. The residue was chromatographed on silica gel eluting with 0-10% ethyl acetate in dichloromethane, to give the title compound (0.065 g, 39% yield).

### EXAMPLE 80C

### benzyl(1S,2S,4S)-4-amino-2-hydroxy-1-[4-(4-methyl-2-pyridinyl)benzyl]-5-phenylpentylcarbamate

A solution containing the product from Example 80B (0.065 g, 0.100 mmol) in a mixture of THF (0.3 mL), methanol (0.3 mL), and aqueous HCl (0.5 mL, 1 N) was stirred at 51C for 16 hours. The solvent was removed under reduced pressure to give the title compound as the hydrochloride salt, which was used without further purification.

### EXAMPLE 80D

### benzyl(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(4-methyl-2-pyridinyl)benzyl]-5-phenylpentylcarbamate

A solution of the product from Example 80C (0.100 mmol) in THF (1 mL) was treated with the product from Example 10D (0.044 g, 0.13 mmol), DEPBT (0.044 g, 0.15 mmol), and N,N-diisopropylethylamine (0.261 mL, 1.5 mmol), stirred at 25° C. for 1.5 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-3% methanol in ethyl acetate to give the title compound (0.054 g, 68% yield).

### EXAMPLE 80E

### (2S)-N-{(1S,3S,4S)-4-amino-1-benzyl-3-hydroxy-5-[4-(4-methyl-2-pyridinyl)phenyl]pentyl}-3,3-dimethyl-2-{3-[(6-methyl-2-pyridinyl)methyl]-2-oxo-1-imidazolidinyl}butanamide

A solution containing the product from Example 80D (0.054 g, 0.068 mmol) in a mixture of ethyl acetate (0.3 mL) and methanol (0.3 mL) was treated with Pd(OH)₂ on carbon (0.014 g, 20% Pd by wt.) and HCl solution (0.068 mL, 4N in dioxane), stirred under a hydrogen atmosphere (balloon pressure) at 25° C. for 16 hours, filtered through a bed of celite® and rinsed with methanol. The solvent was concentrated to give the title compound as the hydrochloride salt, which was used without further purification.

### EXAMPLE 80F

### methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(4-methyl-2-pyridinyl)benzyl]-5-phenylpentyl}amino)carbonyl]-2,2-dimethylpropylcarbamate

A solution containing the product from Example 80E (0.068 mmol) in THF (0.65 mL) was treated with the product from Example 1F (0.017 g, 0.088 mmol), DEPBT (0.030 g, 0.102 mmol), and N,N-diisopropylethylamine (0.118 mL, 0.678 mmol), stirred at 25 C for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by chromatography on silica gel eluting with 0-100% ethyl acetate/dichloromethane, followed by 0-5% methanol in ethyl acetate, to give the title compound (0.036 g, 64% yield).

¹H NMR (300 MHz, DMSO-d₆), δ ppm 0.83 (s, 9H), 0.86 (s, 9H), 1.53 (m, 2H), 2.42 (m, 8H), 2.73 (m, 3H), 3.03 (m, 2H), 3.23 (m, 1H), 3.52 (s, 3H), 3.62 (m, 1H), 3.94 (m, 2H), 4.18 (m, 2H), 4.34 (m, 2H), 4.83 (d, J=5.88 Hz, 1H), 6.79 (d, J=9.56 Hz, 1H), 7.05 (m, 6H), 7.16 (m, 2H), 7.29 (d, J=8.09 Hz, 2H), 7.58 (d, J=8.46 Hz, 1H), 7.69 (m, 2H), 7.90 (d, J=8.46 Hz, 3H), 8.48 (d, J=4.78 Hz, 1H).

### EXAMPLE 81

### methyl(1S)-1-[({(1S,2S,4S)-4-{[(2S)-2-(3-benzyl-2-oxo-1-imidazolidinyl)-3,3-dimethylbutanoyl]amino}-2-hydroxy-5-phenyl-1-[4-(2-pyridinyl)benzyl]pentyl}amino)carbonyl]-2,2-dimethylpropylcarbamate

A solution containing the product from Example 23S (0.020 g, 0.038 mmol) in THF (0.4 mL) was treated with the product from Example 70A (0.013 g, 0.045 mmol), DEPBT (0.017 g, 0.057 mmol), and N,N-diisopropylethylamine (0.039 mL, 0.224 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by elution with 5% methanol in ethyl acetate to give the title compound (0.009 g, 30% yield). ¹H NMR (300 MHz, CDCl₃), δ ppm 0.96 (s, 9H), 1.00 (s, 9H), 1.27 (m, 1H), 2.62 (dd, J=13.79, 8.64 Hz, 1H), 2.85 (m, 5H), 3.03 (q, J=8.58 Hz, 1H), 3.39 (m, 1H), 3.64 (m, 4H), 3.82 (d, J=9.19 Hz, 1H), 3.94 (m, 1H), 4.00 (s, 1H), 4.11 (m, 2H), 4.35 (m, 2H), 5.31 (m, 1H), 6.13 (m, 2H), 7.10 (m, 5H), 7.21 (m, 2H), 7.33 (m, 7H), 7.74 (m, 2H), 7.89 (d, J=8.46 Hz, 2H), 8.68 (d, J=4.78 Hz, 1H).

### EXAMPLE 82A

### (2S)-3,3-dimethyl-2-{3-[(2-methyl-3-pyridinyl)methyl]-2-oxo-1-imidazolidinyl}butanoic Acid

A solution containing the product from Example 6F (0.150 g, 0.65 mmol) in a mixture of toluene (2.5 mL) and methanol (2.5 mL) was treated with the product from Example 15A (0.079 mL, 0.65 mmol), stirred at 50° C. for 18 hours, cooled to 25° C., treated with sodium borohydride (0.049 g, 1.29 mmol), stirred at 25° C. for 1 hour, quenched with 1N NaHCO₃, stirred for 1 hour, and partitioned between ethyl acetate and water. The organic phase was washed with brine and dried over MgSO₄, filtered and concentrated. A solution of the concentrate (0.214 g) in 1,2-dichloroethane (10 mL) was treated with N,N-disuccinimidyl carbonate (0.20 g, 0.781 mmol) and triethylamine (0.11 mL, 0.789 mmol), stirred at 25° C. for 16 hours, and partitioned with 10% Na₂CO₃. The aqueous was extracted with additional chloroform. The combined organic phase was dried over MgSO₄, filtered and concentrated. A solution of the concentrate (0.268 g) in dichloromethane (2.5 mL) was treated with trifluoracetic acid (2.5 mL), and the mixture was stirred at 25° C. for 2 hours. The solvent was concentrated to give the title compound (0.430 g) as the trifluoroacetic acid salt, which was used without further purification.

### EXAMPLE 82B

### methyl(1S)-1-[({(1S,3S,4S)-4-[((2S)-3,3-dimethyl-2-{3-[(2-methyl-3-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 2C (0.020 g, 0.038 mmol) in THF (0.4 mL) was treated with the product from Example 82A (0.025 g, 0.082 mmol), DEPBT (0.017 g, 0.057 mmol), and N,N-diisopropylethylamine (0.033 mL, 0.189 mmol) and the mixture was stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The product was partitioned between ethyl acetate and saturated NaHCO₃ solution. The organic phase was washed brine, dried over MgSO₄, filtered and concentrated to give the title compound (0.016 g, 52% yield). ¹H NMR (300 MHz, DMSO-d₆), δ ppm 0.83 (s, 9H), 0.89 (s, 9H), 1.26 (m, 2H), 1.54 (m, 2H), 2.32 (m, 1H), 2.61 (m, 4H), 2.81 (m, 2H), 2.96 (q, J=8.95 Hz, 1H), 3.21 (m, 1H), 3.50 (s, 3H), 3.66 (m, 1H), 3.85 (d, J=9.93 Hz, 1H), 4.08 (s, 1H), 4.21 (m, 3H), 4.42 (m, 1H), 4.55 (d, J=7.72 Hz, 1H), 6.65 (d, J=9.93 Hz, 1H), 7.00 (m, 3H), 7.10 (m, 2H), 7.24 (m, 3H), 7.31 (m, 1H), 7.54 (m, 2H), 7.87 (m, 5H), 8.38 (dd, J=4.78, 1.47 Hz, 1H), 8.64 (d, J=4.41 Hz, 1H).

### EXAMPLE 83A

### (2S)-3,3-dimethyl-2-{3-[(6-methyl-3-pyridinyl)methyl]-2-oxo-1-imidazolidinyl}butanoic Acid

A solution containing the product from Example 6F (0.150 g, 0.65 mmol) in a mixture of toluene (2.5 mL) and methanol (2.5 mL) was treated with the product from Example 13A (0.079 mL, 0.65 mmol), stirred at 50° C. for 16 hours, cooled to 25° C., treated with sodium borohydride (0.049 g, 1.29 mmol), stirred at 25° C. for 1 hour, quenched with 1N NaHCO₃ and stirred for 1 hour, and partitioned between ethyl acetate and water. The organic phase was washed with brine and dried over MgSO₄, filtered and concentrated. A solution of the concentrate (0.194 g) in 1,2-dichloroethane (10 mL) was treated with N,N-disuccinimidyl carbonate (0.20 g, 0.781 mmol) and triethylamine (0.11 mL, 0.789 mmol), stirred at 25° C. for 16 hours, and partitioned with 10% Na₂CO₃. The aqueous was extracted with additional chloroform. The combined organic phase was dried over MgSO₄, filtered and concentrated. A solution of the concentrate (0.223 g) in dichloromethane (2.5 mL) was treated with trifluoracetic acid (2.5 mL), and the mixture was stirred at 25° C. for 2 hours. The solvent was concentrated to give the title compound (0.379 g) as the trifluoroacetic acid salt, which was used without further purification.

### EXAMPLE 83B

### methyl(1S)-1-[({(1S,3S,4S)-4-[((2S)-3,3-dimethyl-2-{3-[(6-methyl-3-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 2C (0.020 g, 0.038 mmol) in THF (0.4 mL) was treated with the product from Example 83A (0.025 g, 0.082 mmol), DEPBT (0.017 g, 0.057 mmol), and N,N-diisopropylethylamine (0.033 mL, 0.189 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The product was partitioned between ethyl acetate and saturated NaHCO₃ solution. The organic phase was washed brine, dried over MgSO₄, filtered and concentrated to give the title compound (0.014 g, 45% yield). ¹H NMR (300 MHz, DMSO-d₆), δ ppm 0.83 (s, 9H), 0.88 (s, 9H), 1.25 (m, 1H), 1.54 (m, 2H), 2.32 (m, 1H), 2.45 (s, 3H), 2.63 (m, 2H), 2.89 (m, 3H), 3.18 (m, 1H), 3.50 (s, 3H), 3.66 (m, 1H), 3.85 (d, J=9.93 Hz, 1H), 4.13 (m, 3H), 4.30 (s, 2H), 4.55 (d, J=7.35 Hz, 1H), 6.65 (d, J=9.56 Hz, 1H), 7.07 (m, 5H), 7.28 (m, 4H), 7.54 (m, 2H), 7.86 (m, 5H), 8.37 (d, J=1.84 Hz, 1H), 8.63 (d, J=4.41 Hz, 1H).

### EXAMPLE 84

### methyl(1S)-1-[({(1S,3S,4S)-4-({(2S)-3,3-dimethyl-2-[2-oxo-3-(3-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 2C (0.015 g, 0.028 mmol) in THF (0.3 mL) was treated with the product from Example 20A (0.019 g, 0.047 mmol), DEPBT (0.013 g, 0.043 mmol), and N,N-diisopropylethylamine (0.025 mL, 0.144 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The product was partitioned between ethyl acetate and saturated NaHCO₃ solution. The organic phase was washed brine, dried over MgSO₄, filtered and concentrated to give the title compound (0.013 g, 55% yield). ¹H NMR (300 MHz, DMSO-d₆), δ ppm 0.83 (s, 9H), 0.88 (s, 9H), 1.26 (m, 1H), 1.54 (m, 2H), 2.32 (m, 1H), 2.77 (m, 5H), 3.18 (m, 1H), 3.50 (s, 3H), 3.66 (m, 1H), 3.85 (d, J=9.93 Hz, 1H), 4.28 (m, 5H), 4.55 (d, J=7.72 Hz, 1H), 6.65 (d, J=9.93 Hz, 1H), 7.03 (m, 2H), 7.10 (m, 2H), 7.22 (d, J=8.46 Hz, 2H), 7.31 (m, 1H), 7.41 (dd, J=7.72, 5.15 Hz, 1H), 7.51 (d, J=9.56 Hz, 1H), 7.68 (m, 1H), 7.86 (m, 6H), 8.52 (m, 2H), 8.63 (d, J=4.41 Hz, 1H).

### EXAMPLE 85A

### (2S)-3,3-dimethyl-2-[2-oxo-3-(4-pyridinylmethyl)-1-imidazolidinyl]butanoic Acid

A solution containing the product from Example 6F (0.10 g, 0.43 mmol) in a mixture of benzene (1.6 mL) and methanol (1.66 mL) was treated with pyridine-4-carboxaldehyde (0.041 mL, 0.43 mmol), stirred at 50° C. for 18 hours, cooled to 25° C., treated with sodium borohydride (0.033 g, 0.87 mmol), stirred at 25° C. for 1 hour, quenched with saturated NaHCO₃, stirred for 1 hour, and partitioned between ethyl acetate and saturated NaHCO₃. The organic phase was washed with brine and dried over MgSO₄, filtered and concentrated. A solution of the concentrate (0.43 mmol) in 1,2-dichloroethane (7 mL) was treated with N,N-disuccinimidyl carbonate (0.134 g, 0.52 mmol) and triethylamine (0.07 mL, 0.50 mmol), and the mixture was stirred at 25° C. for 16 hours. The reaction was diluted with chloroform and partitioned with 10% Na₂CO₃. The organic phase was washed with brine, dried over MgSO₄, filtered and concentrated. A solution containing the product from Example ii (0.43 mmol) in dichloromethane (2 mL) was treated with trifluoracetic acid (2 mL), and the mixture was stirred at 25° C. for 2 hours. The solvent was concentrated, and the product was dissolved in toluene and concentrated several times to give the title compound (0.259 g), as the trifluoroacetic acid salt.

### EXAMPLE 85B

### methyl(1S)-1-[({(1S,3S,4S)-4-({(2S)-3,3-dimethyl-2-[2-oxo-3-(4-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 2C (0.015 g, 0.028 mmol) in THF (0.3 mL) was treated with the product from Example 85A (0.019 g, 0.047 mmol), DEPBT (0.013 g, 0.043 mmol), and N,N-diisopropylethylamine (0.07 mL, 0.402 mmol) and the mixture was stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The product was partitioned between ethyl acetate and saturated NaHCO₃ solution. The organic phase was washed brine, dried over MgSO₄, filtered and concentrated to give the title compound (0.010 g, 44% yield). ¹H NMR (300 MHz, DMSO-d₆), δ ppm 0.83 (s, 9H), 0.90 (s, 9H), 1.27 (m, 1H), 1.55 (m, 2H), 2.41 (m, 1H), 2.83 (m, 5H), 3.24 (m, 1H), 3.50 (s, 3H), 3.67 (m, 1H), 3.85 (d, J=9.93 Hz, 1H), 4.18 (m, 5H), 4.56 (d, J=7.35 Hz, 1H), 6.65 (d, J=9.93 Hz, 1H), 7.11 (m, 5H), 7.28 (m, 5H), 7.54 (d, J=9.19 Hz, 1H), 7.87 (m, 5H), 8.56 (d, J=5.88 Hz, 2H), 8.64 (d, J=4.41 Hz, 1H).

### EXAMPLE 86A

### (2S)-3,3-dimethyl-2-[2-oxo-3-(2-pyridinylmethyl)-1-imidazolidinyl]butanoic Acid

A solution containing the product from Example 6F (0.10 g, 0.43 mmol) in a mixture of benzene (1.6 mL) and methanol (1.66 mL) was treated with pyridine-2-carboxaldehyde (0.041 mL, 0.43 mmol), stirred at 50° C. for 18 hours, cooled to 25° C., treated with sodium borohydride (0.033 g, 0.87 mmol), stirred at 25° C. for 1 hour, quenched with saturated NaHCO₃, stirred for 1 hour, and partitioned between ethyl acetate and saturated NaHCO₃. The organic phase was washed with brine and dried over MgSO₄, filtered and concentrated. A solution of the concentrate (0.43 mmol) in 1,2-dichloroethane (7 mL) was treated with N,N-disuccinimidyl carbonate (0.134 g, 0.52 mmol) and triethylamine (0.07 mL, 0.50 mmol), stirred at 25° C. for 16 hours, diluted with chloroform and partitioned with 10% Na₂CO₃. The organic phase was washed with brine, dried over MgSO₄, filtered and concentrated. A solution of the concentrate (0.43 mmol) in dichloromethane (2 mL) was treated with trifluoracetic acid (2 mL), stirred at 25° C. for 2 hours, concentrated, and azeotroped several times with toluene to give the title compound (0.201 g), as the trifluoroacetic acid salt.

### EXAMPLE 86B

### methyl(1S)-1-[({(1S,3S,4S)-4-({(2S)-3,3-dimethyl-2-[2-oxo-3-(2-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 2C (0.015 g, 0.028 mmol) in THF (0.3 mL) was treated with the product from Example 85A (0.019 g, 0.047 mmol), DEPBT (0.013 g, 0.043 mmol), and N,N-diisopropylethylamine (0.025 mL, 0.144 mmol) and the mixture was stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5100% acetonitrile in water (0.1% TFA). The product was partitioned between ethyl acetate and saturated NaHCO₃ solution. The organic phase was washed brine, dried over MgSO₄, filtered and concentrated to give the title compound (0.011 g, 48% yield). ¹H NMR (300 MHz, DMSO-d₆) δ ppm 0.83(s, 9H), 0.90(s, 9H), 1.60-1.51(m, 2H), 2.44-2.35(q, J=9.07 Hz, 1H), 2.61-2.54(m, 1H), 2.69-2.66(d, J=6.99 Hz, 2H), 2.81-2.76(m, 1H), 3.01-2.94(m, 1H), 3.14-3.05(m, 1H), 3.26-3.19(m, 1H), 3.50(s, 3H), 3.70-3.62(m, 1H), 3.86-3.83(d, J=9.56 Hz, 1H), 4.08(s, 1H), 4.25-4.11(m, 2H), 4.47-4.34(m, 2H), 4.57-4.54(d, J=7.72 Hz, 1H), 6.65-6.63(d, J=9.17 Hz, 1H), 7.10-7.06(m, 5H), 7.26-7.21(m, 3H), 7.32-7.28(m, 2H), 7.51-7.48(d, J=9.56 Hz, 1H), 7.91-7.79(m, 6H), 8.55-8.54(d, J=3.68 Hz, 1H), 8.64-8.63(d, J=4.41 Hz, 1H).

### EXAMPLE 87A

### 2-methyl-5-(tributylstannyl)pyridine

A solution containing 5-bromo-2-methylpyridine (1.2 g, 6.98 mmol) in ether (14 mL) at −78° C. was treated with n-butyllithium (5.2 mL, 1.6 M in hexanes) dropwise, stirred at −78° C. for 1 hour, treated with tributyltin chloride (2.25 mL, 8.30 mmol), stirred at −78° C. for 0.5 hours, and then at 0° C. for 0.5 hours. The reaction was quenched with saturated ammonium chloride solution and the reaction was partitioned between ether and water, and the organic phase was washed with brine and dried over MgSO₄, filtered and concentrated to give the title compound (2.97 g), which was used without further purification.

### EXAMPLE 87B

### benzyl(4S,5S)-5-{(2S)-2-[(tert-butoxycarbonyl)amino]-3-phenylpropyl}-2,2-dimethyl-4-[4-(6-methyl-3-pyridinyl)benzyl]-1,3-oxazolidine-3-carboxylate

A solution containing the product from Example 23I (0.25 g, 0.354 mmol) in DMF (3.5 mL) was treated with LiCl (0.15 g, 3.54 mmol), dichlorobis(triphenylphosphine)palladium(II) (0.075 g, 0.107 mmol), and the product from Example 87A (0.40 mL, 1.67 mmol), heated at 100° C. for 16 hours, cooled and partitioned between ethyl acetate and water. The organic phase was washed with brine and dried over MgSO₄, filtered and concentrated. The residue was chromatographed on silica gel eluting with 0-25% ethyl acetate in dichloromethane to give the title compound (0.193 g, 84% yield).

### EXAMPLE 87C

### benzyl(1S,2S,4S)-4-amino-2-hydroxy-1-[4-(6-methyl-3-pyridinyl)benzyl]-5-phenylpentylcarbamate

A solution containing the product from Example 87B (0.193 g, 0.297 mmol) in a mixture of THF (2 mL), methanol (2 mL), and aqueous HCl (2 mL, 1 N) was stirred at 60° C. for 16 hours. The solvent was removed under reduced pressure to give the title compound as the hydrochloride salt, which was used without further purification.

### EXAMPLE 87D

### (2S,3S,5S)-2,5-diamino-1-[4-(6-methyl-3-pyridinyl)phenyl]-6-phenyl-3-hexanol

A solution containing the product from Example 87C (0.086 g, 0.148 mmol) in methanol (1.5 mL) was treated with Pd(OH)₂ on carbon (0.020 g, 20% Pd by wt.) and HCl solution (0.11 mL, 4N in dioxane), stirred under a hydrogen atmosphere (balloon pressure) at 25° C. for 16 hours, filtered through a bed of celite® and rinsed with methanol. The solvent was concentrated to give the title compound as the hydrochloride salt, which was used without further purification.

### EXAMPLE 87E

### methyl(1S,4S,5S,7S,10S)-7-benzyl-1,10-ditert-butyl-5-hydroxy-4-[4-(6-methyl-3-pyridinyl)benzyl]-2,9,12-trioxo-13-oxa-3,8,11-triazatetradec-1-ylcarbamate

A solution containing the product from Example 87D (0.148 mmol) in THF (1.5 mL) was treated with the product from Example 1F (0.070 g, 0.370 mmol), DEPBT (0.14 g, 0.468 mmol), and N,N-diisopropylethylamine (0.26 mL, 1.49 mmol) and the mixture was stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The product was purified by chromatography on silica gel eluting with 0-100% ethyl acetate/dichloromethane, followed by 0-5% methanol in ethyl acetate, to give the title compound (0.060 g, 56% yield). ¹H NMR (300 MHz, DMSO-d₆) δ ppm 0.77(s, 9H), 0.83(s, 9H), 1.56-1.54(m, 2H), 2.77-2.69(m, 3H), 3.49(s, 3H), 3.54(s, 3H), 3.67-3.60(m, 1H), 3.81-3.78(d, J=9.93 Hz, 1H), 3.95-3.92(d, J=9.56 Hz, 1H), 4.16-4.02(m, 2H), 4.86-4.84(d, J=5.88 Hz, 1H), 6.64-6.61(d, J=9.93 Hz, 1H), 6.81-6.78(d, J=9.93 Hz, 1H), 7.15-7.07(m, 5H), 7.33-7.28(m, 3H), 7.52-7.49(d, J=8.09 Hz, 2H), 7.60-7.58(d, J=8.82 Hz, 1H), 7.76-7.73(d, J=8.09 Hz, 1H), 7.91-7.88(dd, J=8.09, 2.57 Hz, 1H), 8.70-8.69 (d, J=2.21 Hz, 1H).

### EXAMPLE 88A

### benzyl(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methyl-3-pyridinyl)benzyl]-5-phenylpentylcarbamate

A solution containing the product from Example 87C (0.086 g, 0.148 mmol) in THF (1.5 mL) was treated with the product from Example 10D (0.060 g, 0.176 mmol), DEPBT (0.067 g, 0.224 mmol), and N,N-diisopropylethylamine (0.26 mL, 1.49 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by chromatography on silica gel eluting with 0-100% ethyl acetate/dichloromethane; followed by 0-5% methanol in ethyl acetate, to give the title compound (0.079 g, 67% yield).

### EXAMPLE 88B

### (2S)-N-{(1S,3S,4S)-4-amino-1-benzyl-3-hydroxy-5-[4-(6-methyl-3-pyridinyl)phenyl]pentyl}-3,3-dimethyl-2-{3-[(6-methyl-2-pyridinyl)methyl]-2-oxo-1-imidazolidinyl}butanamide

A solution containing the product from Example 88A (0.079 g, 0.099 mmol) in methanol (1.5 mL) was treated with Pd(OH)₂ on carbon (0.040 g, 20% Pd by wt.) and HCl solution (0.075 mL, 4N in dioxane), stirred under a hydrogen atmosphere (balloon pressure) at 25° C. for 16 hours, filtered through a bed of celite® and rinsed with methanol. The solvent was concentrated to give the title compound as the hydrochloride salt, which was used without further purification.

### EXAMPLE 88C

### methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methyl-3-pyridinyl)benzyl]-5-phenylpentyl}amino)carbonyl]-2,2-dimethylpropylcarbamate

A solution containing the product from Example 88B (0.099 mmol) in THF (1 mL) was treated with the product from Example 1F (0.022 g, 0.116 mmol), DEPBT (0.045 g, 0.151 mmol), and N,N-diisopropylethylamine (0.175 mL, 1.00 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by chromatography on silica gel eluting with 0-100% yl acetate/dichloromethane, followed by 0-5% methanol in ethyl acetate, to give the title compound (0.064 g, 77% yield). ¹H NMR (300 MHz, DMSO-d₆) δ ppm 0.83(s, 9H), 0.86(s, 9H), 1.59-1.49(m, 2H), 2.46-2.34(m, 2H), 2.46(s, 3H), 2.49(s, 3H), 2.67-2.64(m, 1H), 2.77-2.75(d, J=6.99 Hz, 2H), 2.99-2.91(m, 1H), 3.12-3.03(m, 1H), 3.26-3.17(m, 1H), 3.50(s, 3H), 3.66-3.58(m, 1H), 3.96-3.93(m, 2H), 4.25-4.13(m, 2H), 4.40-4.28(m, 2H), 4.84-4.82(d, J=5.52 Hz, 1H), 6.84-6.81(d, J=9.56 Hz, 1H), 7057.01(m, 6H), 7.16-7.14(d, J=7.72 Hz, 1H), 7.33-7.29(dd, J=8.09, 4.04 Hz, 3H), 7.51-7.49(d, J=8.09 Hz, 2H), 7.59-7.56(d, J=8.45 Hz, 1H), 7.71-7.65(t, J=7.72 Hz, 1H), 7.91-7.88(m, 2H), 8.69(d, J=2.21 Hz, 1H).

### EXAMPLE 89A

### tert-butyl(1S,3S,4S)-4-amino-1-benzyl-3-hydroxy-5-[4-(5-methyl-2-pyridinyl)phenyl]pentylcarbamate

A solution containing the product from Example 74B (0.312 g, 0.48 mmol) in methanol (5 mL) was treated with Pd(OH)₂ on carbon (0.10 g, 20% Pd by wt.) and HCl solution (0.240 mL, 4N in dioxane), stirred under a hydrogen atmosphere (balloon pressure) at 25° C. for 16 hours, filtered through a bed of celite®(D, rinsed with methanol and concentrated. The residue was purified by reversed phase chromatography on a C18 column eluting with 5-100% acetonitrile in water (0.1% TFA) to give the title compound as the hydrochloride salt (0.178 g, 53% yield).

### EXAMPLE 89B

### tert-butyl(1S,3S,4S)-1-benzyl-3-hydroxy-4-({(2S)-2-[(methoxycarbonyl)amino]-3,3-dimethylbutanoyl}amino)-5-[4-(5-methyl-2-pyridinyl)phenyl]pentylcarbamate

A solution containing the product from Example 89A (0.178 g, 0.302 mmol) in THF (4 mL) was treated with the product from Example 1F (0.074 g, 0.393 mmol), DEPBT (0.136 g, 0.454 mmol), and N,N-diisopropylethylamine (0.527 mL, 3.02 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated to give the title compound, which was used without further purification.

### EXAMPLE 89C

### methyl(1S)-1-[({(1S,2S,4S)-4-amino-2-hydroxy-1-[4-(5-methyl-2-pyridinyl)benzyl]-5-phenylpentyl}amino)carbonyl]-2,2-dimethylpropylcarbamate

A solution containing the product from Example 89B (0.302 mmol) in dichloromethane (2.5 mL) was treated with trifluoroacetic acid (2.5 mL), stirred at 25° C. for 16 hours and concentrated. The residue was purified by reversed phase chromatography on a C18 column eluting with 5-100% acetonitrile in water (0.1% TFA). The product was partitioned between ether and dilute ammonium hydroxide, and the organic phase was dried over MgSO₄, filtered and concentrated to give the title compound (0.068 g, 42% yield).

### EXAMPLE 89D

### methyl(1S)-1-[({(1S,2S,4S)-4-[((2S)-3,3-dimethyl-2-{3-[(2-methyl-1,3-thiazol-4-yl)methyl]-2-oxo-1-imidazolidinyl}butanoyl)amino]-2-hydroxy-1-[4-(5-methyl-2-pyridinyl)benzyl]-5-phenylpentyl}amino)carbonyl]-2,2-dimethylpropylcarbamate

A solution containing the product from Example 89C (0.040 g, 0.073 mmol) in THF (0.7 mL) was treated with the product from Example 14B (0.030 g, 0.095 mmol), DEPBT (0.033 g, 0.110 mmol), and N,N-diisopropylethylamine (0.064 mL, 0.365 mmol) and the mixture was stirred at 25° C. for 16 hours. The mixture was partitioned between ethyl acetate and 10% Na₂CO₃ solution. The organic phase was washed with additional 10% Na₂ CO₃ solution and brine, dried over MgSO₄, filtered and concentrated. The residue was purified by chromatography on silica gel eluting with 0-100% ethyl acetate/dichloromethane, followed by 0-5% methanol in ethyl acetate, to give the title compound (0.037 g, 60% yield). ¹H NMR (300 MHz, DMSO-d₆) δ ppm 0.81(s, 9H), 0.86(s, 9H), 1.57-1.45(m, 2H), 2.32(s, 3H), 2.45-2.37(m, 2H), 2.64(s, 3H), 2.69-2.58(m, 1H), 2.77-2.75(m, 2H), 3.09-2.92(m, 2H), 3.26-3.17(m, 1H), 3.52(s, 3H), 3.66-3.54(m, 1H), 3.97-3.94(m, 2H), 4.23-4.07(m, 2H), 4.42-4.23(m, 2H), 4.82(bs, 1H), 6.58-6.54(J=8.09 Hz, 1H), 6.82-6.79(d, J=9.56 Hz, 1H), 7.08-6.95(m, 5H), 7.21(s, 1H), 7.29-7.27(d, J=8.09 Hz, 2H), 7.60-7.57(d, J=8.82 Hz, 1H), 7.68-7.64(dd, J=8.09, 2.21 Hz, 1H), 7.79-7.77(d, J=8.09 Hz, 1H), 7.89-7.86(d, J=8.46 Hz, 2H), 8.47-8.46(d, J=2.21 Hz, 1H).

### EXAMPLE 90

### methyl(1S,4S,5S,7S,10S)-7-benzyl-1,10-ditert-butyl-5-hydroxy-4-[4-(5-methyl-2-pyridinyl)benzyl]-2,9,12-trioxo-13-oxa-3,8,11-triazatetradec-1-ylcarbamate

A solution containing the product from Example 89C (0.032 g, 0.059 mmol) in THF (0.5 mL) was treated with the product from Example 1F (0.014 g, 0.076 mmol), DEPBT (0.034 g, 0.114 mmol), and N,N-diisopropylethylamine (0.066 mL, 0.38 mmol) and the mixture was stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The residue was purified by chromatography on silica gel eluting with 0-100% ethyl acetate/dichloromethane, followed by 0-5% methanol in ethyl acetate, to give the title compound (0.028 g, 61% yield). ¹H NMR (300 MHz, DMSO-d₆) δ ppm 0.77 (s, 9H), 0.83(s, 9H), 1.58-1.39(m, 2H), 2.32(s, 3H), 2.77-2.69(m, 3H), 3.51(s, 3H), 3.54(s, 3H), 3.67-3.60(m, 1H), 3.81-3.78(d, J=9.93 Hz, 1H), 3.95-3.92(d, J=9.93 Hz, 1H), 4.17-4.01(m, 2H), 4.86-4.84(d, J=5.52 Hz, 1H), 6.63-6.60(d, J=9.56 Hz, 1H), 6.78-6.75(d, J=9.93 Hz, 1H), 7.14-7.06(m, 5H), 7.29-7.26(d, J=8.46 Hz, 2H), 7.61-7.58(d, J=9.19 Hz, 1H), 7.68-7.65(m, 1H), 7.80-7.73(m, 2H), 7.90-7.87(d, J=8.09 Hz, 2H), 8.47(s, 1H).

### EXAMPLE 91A

### tert-butyl(1S,2S,4S)-1-benzyl-2-hydroxy-4-({(2S,3S)-2-[(methoxycarbonyl)amino]-3-methylpentanoyl}amino)-5-[4-(2-pyridinyl)phenyl]pentylcarbamate

A solution containing the product from Example 2A (0.030 g, 0.060 mmol) in THF (0.6 mL) was treated with the product from Example 5A (0.014 g, 0.074 mmol), DEPBT (0030 g, 0.100 mmol), and N,N-diisopropylethylamine (0.050 mL, 0.287 mmol) and the mixture was stirred at 25° C. for 4 hours. The mixture was partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 33-100% ethyl acetate in chloroform to give the title compound (0.025 g, 66% yield).

### EXAMPLE 91B

### methyl(1S,2S)-1-[({(1S,3S,4S)-4-amino-3-hydroxy-5-phenyl-1-[4-(2-pyridinyl)benzyl]pentyl}amino)carbonyl]-2-methylbutylcarbamate

A solution containing the product from Example 2B (0.025 g, 0.040 mmol) in dichloromethane (1 mL) was treated with trifluoroacetic acid (1 mL) and the mixture was stirred at 25 C for 1 hour. The solvent was concentrated and the mixture was partitioned between ethyl acetate and saturated NaHCO₃ solution. The organic phase was washed with brine, dried over MgSO₄, filtered and concentrated.

### EXAMPLE 91C

### methyl(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2-methylbutylcarbamate

A solution containing the product from Example 91B (0.040 mmol) in THF (0.4 mL) was treated with the product from Example 10D (0.016 g, 0.047 mmol), DEPBT (0.018 g, 0.060 mmol), and N,N-diisopropylethylamine (0.035 mL, 0.201 mmol) and the mixture was stirred at 25° C. for 16 hours. The mixture was partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 50% ethyl acetate in chloroform, followed by 5% methanol in chloroform to give the title compound (0.007 g, 22% yield). ¹H NMR (300 MHz, DMSO-d₆) δ ppm 0.77-0.69(m, 6H), 0.90(s, 9H), 1.06-0.80(m, 2H), 1.40-1.22(m, 1H), 1.67-1.48(m, 2H), 2.40-2.33(m, 1H), 2.46(s, 3H), 2.68-2.57(m, 3H), 2.82-2.70(m, 1H), 3.01-2.92(m, 1H), 3.13-3.04(m, 1H), 3.27-3.17(m, 1H), 3.52(s, 3H), 3.68-3.74(m, 1H), 3.80-3.74(m, 1H), 4.08(s, 1H), 4.25-4.11(m, 2H), 4.41-4.29(m, 2H), 4.53-4.51(d, J=7.72 Hz, 1H), 6.94-6.91(d, J=9.19 Hz, 1H), 7.17-7.03(m, 6H), 7.25-7.23(d, J=8.09 Hz, 2H), 7.33-7.29(m, 1H), 7.51-7.48(d, J=9.56 Hz, 1H), 7.71-7.66(t, J=7.72 Hz, 1H), 7.79-7.75(d, J=9.19 Hz, 1H), 7.94-7.85(m, 4H), 8.64-8.63(m, 1H).

### EXAMPLE 92A

### ethyl(5S)-3-(4-bromobenzyl)-5-{(1S)-1-[(tert-butoxycarbonyl)amino]-2-phenylethyl}-2-oxotetrahydro-3-furancarboxylate

A solution of tert-Butyl(1S)-1-[(2R)-oxiran-2-yl]-2-phenylethylcarbamate (10.0 g, 38.0 mmol) and diethyl malonate (5.8 ml, 38.2 mmol) in ethanol (30 mL) at 0° C. was treated with a solution of NaOEt (13.5 mL, 21% in ethanol) over 10 minutes. The reaction was warmed to 25° C. and stirred for 18 hours. The reaction was re-cooled to 0° C. and treated with a solution of 4-bromobenzyl bromide (9.5 g, 38.0 mmol) in ethanol (40 mL) was, stirred at 50° C. for 3 hours, cooled to 0° C. and adjusted to neutral pH by addition of 4N HCl. The ethanol was removed under reduced pressure and the residue was partitioned between chloroform and water. The organic phase was washed with brine and dried over MgSO₄, filtered and concentrated to give the title compound (22.4 g), which was used without further purification.

### EXAMPLE 92B

### tert-butyl(1S)-1-[(2S)-4-(4-bromobenzyl)-5-oxotetrahydro-2-furanyl]-2-phenylethylcarbamate

A solution of the product from Example 92A (22.4 g) in ethanol (120 mL) was treated with LiOH monohydrate (8.0 g, 190.7 mmol) solution in water (30 mL) and the mixture was stirred at 25° C. for 16 hours. The mixture was cooled to 0° C., adjusted to pH 5 by addition of 4N HCl and partitioned between dichloromethane and water. The organic phase was washed with brine and dried over MgSO₄, filtered and concentrated. A solution of the concentrate in toluene (400 mL) was then heated at reflux for 18 hours, cooled and concentrated to give the title compound (18.4 g), which was used without further purification.

### EXAMPLE 92C

### (4S,5S)-2-(4-bromobenzyl)-5-[(tert-butoxycarbonyl)amino]-4-{[tert-butyl(dimethyl)silyl]oxy}-6-phenylhexanoic Acid

A solution containing the product from Example 92B (18.4 g) in dioxane (190 mL) was treated with sodium hydroxide solution (45 mL, 1N) for 1 hour at 25° C. The mixture was cooled to 0° C., and acidified to pH 5 using 4N HCl, and concentrated under reduced pressure. The concentrate was partitioned between chloroform and water. The organic phase layer was washed with brine, dried over MgSO₄, and concentrated. A solution of the residue (22 g) in dioxane (115 mL) was treated with imidazole (19 g, 279 mmol) and t-butyldimethylsilyl chloride (35 g, 232 mmol), stirred at 25° C. for 18 hours and concentrated. The residue was combined with ice, acidified with 4N HCl to pH 3, and extracted with ethyl acetate. The organic phase was washed with brine, dried over MgSO₄, filtered, and concentrated. A solution of the residue in a mixture of THF (180 mL), acetic acid (180 mL), and water (60 mL) was stirred for 1 hour at 25° C. and concentrated. The residue was chromatographed on silica gel eluting with 0-50% ethyl acetate in chloroform to give the title compound (14.18 g, 60% yield).

### EXAMPLE 92D

### tert-butyl(1S,2S,4S)-1-benzyl-4-{[benzyloxycarbonyl]amino}-5-(4-bromophenyl)-2-{[tert-butyl(dimethyl)silyl]oxy}pentylcarbamate

A solution of the product from Example 92C (14.1 g, 23.3 mmol) in toluene (230 mL) was treated with DPPA (10.0 mL, 46.4 mmol) and triethylamine (6.5 mL, 46.6 mmol), heated at reflux for 2 hours, treated with benzyl alcohol (7.2 mL, 69.9 mmol), heated at reflux for an additional 16 hours, cooled and concentrated. The residue was purified by chromatography on silica gel, eluting with 20% ethyl acetate in hexanes to give the higher Rf product (2.94 g, 18% yield).

### EXAMPLE 92E

### tert-butyl(1S,2S,4R)-1-benzyl-4-{[(benzyloxy)carbonyl]amino}-5-(4-bromophenyl)-2-{[tert-butyl(dimethyl)silyl]oxy}pentylcarbamate

A solution of the product from Example 92C (14.1 g, 23.3 mmol) in toluene (230 mL) was treated with diphenylphosphine azide (10.0 mL, 46.4 mmol) and triethylamine (6.5 mL, 46.6 mmol), heated at reflux for 2 hours, treated with benzyl alcohol (7.2 mL, 69.9 mmol), heated at reflux for an additional 16 hours, cooled and concentrated. The residue was purified by chromatography on silica gel, eluting with 20% ethyl acetate in hexanes to give the lower Rf product (3.21 g, 19% yield).

### EXAMPLE 92F

### tert-butyl(1S,2S,4S)-1-benzyl-4-{[(benzyloxy)carbonyl]amino}-2-{[tert-butyl(dimethyl)silyl]oxy}-5-[4-(5-methyl-2-pyridinyl)phenyl]pentylcarbamate

A solution containing the product from Example 92D (0.50 g, 0.703 mmol) in DMF (7 mL) was treated with LiCl (0.30 g, 7.08 mmol), dichlorobis(triphenylphosphine)palladium(II) (0.15 g, 0.213 mmol), and the product from Example 74A (0.805 g, 2.11 mmol), heated at 100° C. for 16 hours, cooled, filtered through celite®(D, and partitioned between ethyl acetate and water. The organic phase was washed with brine and dried over MgSO₄, filtered and concentrated. The residue was chromatographed on silica gel eluting with 0-20% ethyl acetate in chloroform to give the title compound (0.374 g, 74% yield).

### EXAMPLE 92G

### tert-butyl(1S,2S,4S)-1-benzyl-4-{[(benzyloxy)carbonyl]amino}-2-hydroxy-5-[4-(5-methyl-2-pyridinyl)phenyl]pentylcarbamate

The product from Example 92F (0.374 g, 0.517 mmol) was treated with TBAF solution in THF (2 mL, 1N), stirred at 25° C. for 16 hours, concentrated and partitioned between ethyl acetate and water. The organic phase was washed with brined, dried over MgSO₄, filtered and concentrated. The residue was chromatographed on silica gel eluting with 0-20% ethyl acetate in chloroform, to give the title compound (0.198 g, 63% yield).

### EXAMPLE 92H

### tert-butyl(1S,2S,4S)-4-amino-1-benzyl-2-hydroxy-5-[4-(5-methyl-2-pyridinyl)phenyl]pentylcarbamate

A solution containing the product from Example 92G (0.198 g, 0.325 mmol) in a mixture of methanol (1.6 mL) and ethyl acetate (1.6 mL) was treated with Pd(OH)₂ on carbon (0.060 g, 20% Pd by wt.) and HCl solution (0.080 mL, 4N in dioxane), stirred under a hydrogen atmosphere (balloon pressure) at 25° C. for 18 hours, filtered through a bed of celite® and rinsed with methanol. The solvent was concentrated to give the title compound as the hydrochloride salt, which was used without further purification.

### EXAMPLE 92I

### tert-butyl(1S,2S,4S)-1-benzyl-2-hydroxy-4-({(2S)-2-[(methoxycarbonyl)amino]-3,3-dimethylbutanoyl}amino)-5-[4-(5-methyl-2-pyridinyl)phenyl]pentylcarbamate

A solution containing the product from Example 92H (0.325 mmol) in THF (3.3 mL) was treated with the product from Example 1F (0.068 g, 0.360 mmol), DEPBT (0.146 g, 0.488 mmol), and N,N-diisopropylethylamine (0.28 mL, 1.61 mmol), stirred at 259C for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-80% ethyl acetate in chloroform to give the title compound (0.120 g, 56% yield).

### EXAMPLE 92J

### methyl(1S)-1-[({(1S,3S,4S)-4-amino-3-hydroxy-1-[4-(5-methyl-2-pyridinyl)benzyl]-5-phenylpentyl}amino)carbonyl]-2,2-dimethylpropylcarbamate

A solution containing the product from Example 92I (0.120 g, 0.183 mmol) in dichloromethane (1 mL) was treated with trifluoroacetic acid (1 mL), stirred at 25° C. for 1 hour. The solvent was concentrated and the mixture was partitioned between chloroform and saturated NaHCO₃ solution. The organic phase was washed with brine, dried over MgSO₄, filtered and concentrated, and the crude product (0.098 g) was used without further purification.

### EXAMPLE 92K

### methyl(1S,4S,5S,7S,10S)-4-benzyl-1,10-ditert-butyl-5-hydroxy-7-[4-(5-methyl-2-pyridinyl)benzyl]-2,9,12-trioxo-13-oxa-3,8,11-triazatetradec-1-ylcarbamate

A solution containing the product from Example 92J (0.049 g, 0.090 mmol) in THF (1 mL) was treated with the product from Example 1F (0.018 g, 0.095 mmol), DEPBT (0.040 g, 0.133 mmol), and N,N-diisopropylethylamine (0.080 mL, 0.459 mmol), stirred at 25° C. for 3 days, and partitioned between ethyl acetate and 10% Na₂CO₃ solution. The organic phase was washed with additional 10% Na₂CO₃ solution and brine, dried over MgSO₄, filtered and concentrated. The reaction was purified by reversed phase chromatography on a C18 column eluting with 5-100% acetonitrile in water (0.1% TFA). The product was partitioned between ethyl acetate and 10% Na₂CO₃, and the organic phase was washed with brine and dried over MgSO₄, filtered and concentrated to give the title compound (0.047 g, 73% yield). ¹H NMR (300 MHz, DMSO-d₆) δ ppm 0.79(s, 9H), 0.82(s, 9H), 1.58-1.43(m, 2H), 2.32(s, 3H), 2.79-2.68(m, 3H), 3.49(s, 3H), 3.55(s, 3H), 3.67-3.59(m, 1H), 3.84-3.80(d, J=9.93 Hz, 1H), 3.92-3.89(d, J=9.93 Hz, 1H), 4.19-4.01(m, 2H), 4.87-4.85(d, J=5.88 Hz, 1H), 6.63-6.60(d, J=9.19 Hz, 1H), 6.81-6.77(d, J=9.56 Hz, 1H), 7.19-7.12(m, 5H), 7.56-7.53(d, J=8.82 Hz, 1H), 7.68-7.64(m, 1H), 7.81-7.76(m, 3H), 7.86-7.83(d, J=8.09 Hz, 2H), 8.47(bs, 1H).

### EXAMPLE 93

### methyl(1S)-1-[({(1S,3S,4S)-4-{[(2S)-2-(3-benzyl-2-oxo-1-imidazolidinyl)-3,3-dimethylbutanoyl]amino}-3-hydroxy-1-[4-(5-methyl-2-pyridinyl)benzyl]-5-phenylpentyl}amino)carbonyl]-2,2-dimethylpropylcarbamate

A solution containing the product from Example 92J (0.050 g, 0.092 mmol) in THF (1 mL) was treated with the product from Example 70A (0.028 g, 0.097 mmol), DEPBT (0.041 g, 0.137 mmol), and N,N-diisopropylethylamine (0.080 mL, 0.459 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA) The product was partitioned between ethyl acetate and saturated NaHCO₃, and the organic phase was washed with brine and dried over MgSO₄, filtered and concentrated to give the title compound (0.036 g, 48% yield). ¹H NMR (300 MHz, DMSO-d₆) δ ppm 0.83(s, 9H), 0.89(s, 9H), 1.60-1.49(m, 2H), 2.32(s, 3H), 2.60-2.53(m, 1H), 2.68-2.65(d, J=6.99 Hz, 2H), 2.88-2.75(m, 2H), 2.96-2.90(q, J=8.70 Hz, 1H), 3.24-3.15(m, 1H), 3.51(s, 3H), 3.71-3.61(m, 1H), 3.87-3.83(d, J=9.56 Hz, 1H), 4.09(s, 1H), 4.22-4.11 (m, 2H), 4.31(s, 2H), 4.56-4.53(d, J=7.72 Hz, 1H), 6.67-6.63(d, J=9.56 Hz, 1H), 7.08-7.02(m, 5H), 7.21-7.19(d, J=8.46 Hz, 2H), 7.31-7.26(m, 3H), 7.40-7.35(m, 2H), 7.50-7.46 (d, J=9.56 Hz, 1H), 7.68-7.64(dd, J=8.27, 2.02 Hz, 1H), 7.82-7.77(m, 2H), 7.88-7.85(d, J=8.09 Hz, 2H), 8.47-8.46(d, J=2.21 Hz, 1H).

### EXAMPLE 94A

### tert-butyl(1S,2S,4R)-1-benzyl-4-[((2S)-3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentylcarbamate

A solution containing the product from Example 1E (0.050 g, 0.101 mmol) in THF (1 mL) was treated with the product from Example 10D (0.034 g, 0.100 mmol), DEPBT (0.045 g, 0.151 mmol), and N,N-diisopropylethylamine (0.090 mL, 0.517 mmol), stirred at 25° C. for 4 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The product was partitioned between dichloromethane and saturated NaHCO₃ solution. The organic phase was washed brine, dried over MgSO₄, filtered and concentrated. The residue was then chromatographed on silica gel eluting with 0-10% methanol in chloroform, to give the title compound (0.042 g, 56% yield).

### EXAMPLE 94B

### methyl(1S)-1-[({(1S,2S,4R)-1-benzyl-4-[((2S)-3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentyl}amino)carbonyl]-2,2-dimethylpropylcarbamate

A solution containing the product from Example 94A (0.042 g, 0.056 mmol) dichloromethane (0.3 mL) was treated with trifluoroacetic acid (0.3 mL) and the mixture was stirred at 25° C. for 1 hour. The solvent was concentrated and the residue was dissolved in toluene and concentrated several times. A solution of the residue (0.056 mmol) in THF (0.6 mL) was treated with the product from Example 1F (0.011 g, 0.058 mmol), DEPBT (0.025 g, 0.083 mmol), and N,N-diisopropylethylamine (0.049 mL, 0.281 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The product was partitioned between ethyl acetate and saturated NaHCO₃, and the organic phase was washed with brine and dried over MgSO₄, filtered and concentrated to give the title compound (0.023 g, 48% yield). ¹H NMR (300 MHz, DMSO-d₆) δ ppm 0.77(s, 9H), 0.81(s, 9H), 1.41-1.31(m, 1H), 1.59-1.49(m, 1H), 2.43(s, 3H), 2.70-2.59(m, 3H), 2.88-2.77(m, 1H), 3.25-3.12(m, 2H), 3.53(s, 3H), 3.64-3.44(m, 2H), 3.94-3.84(m, 2H), 4.08(s, 1H), 4.19-4.10(m, 2H), 4.43-4.26(m, 2H), 6.77-6.73(d, J=9.56 Hz, 1H), 7.03-7.01(d, J=7.72 Hz, 1H), 7.18-7.09(m, 5H), 7.28-7.25(d, J=8.46 Hz, 2H), 7.34-7.30(m, 2H), 7.53-7.50(d, J=9.93 Hz, 1H), 7.65-7.60(t, J=7.72 Hz, 1H), 7.92-7.83(m, 2H), 7.97-7.95(d, J=8.09 Hz, 2H), 8.17-8.15(d, J=8.46 Hz, 1H), 8.65-8.64(d, J=4.78 Hz, 1H).

### EXAMPLE 95

### 1:1 Mixture of methyl(1R,4S,5S,7S,10S)-4-benzyl-10-tert-butyl-5-hydroxy-1-[1-methyl-1-((R)-methylsulfinyl)ethyl]-2,9,12-trioxo-7-[4-(2-pyridinyl)benzyl]-13-oxa-3,8,11-triazatetradec-1-ylcarbamate and methyl(1R,4S,5S,7S,10S)-4-benzyl-10-tert-butyl-5-hydroxy-1-[1-methyl-1-((S)-methylsulfinyl)ethyl]-2,9,12-trioxo-7-[4-(2-pyridinyl)benzyl]-13-oxa-3,8,11-triazatetradec-1-ylcarbamate

A solution containing the product from Example 30B (0.015 g, 0.020 mmol) in a mixture of THF (0.15 mL), acetone (0.15 mL), and water (0.05 mL) was treated with NMO (0.003 g, 0.026 mmol) and aqueous osmium tetroxide solution (0.030 mL, 4%), was stirred for 16 hours at 25° C., and partitioned between ethyl acetate and water. The organic phase was washed with brine and dried over MgSO₄, filtered and concentrated. The residue was chromatographed on silica gel eluting with 0-100% ethyl acetate/dichloromethane, followed by 0-10% methanol in dichloromethane to give the title compound (0.006 g, 39% yield). ¹H NMR (300 MHz, DMSO-d₆), ppm 0.81 (s, 9H), 1.02 (m, 7H), 1.50(m, 2H), 2.29 (s, 1H), 2.38 (s, 2H), 2.76 (m, 3H), 3.50 (s, 3H), 3.55 (s, 3H), 3.68 (m, 1H), 3.83 (d, J=9.93 Hz, 1H), 4.08 (m, 2H), 4.33 (m, 1H), 5.01 (d, J=5.15 Hz, 1H), 6.63 (d, J=9.56 Hz, 1H), 7.22 (m, 9H), 7.86 (m, 6H), 8.63 (d, J=4.41 Hz, 1H).

### EXAMPLE 96A

### tert-butyl(2S,3S)-2-[(2-{[(9H-fluoren-9-ylmethoxy)carbonyl][(1-methyl-1H-benzimidazol-2-yl)methyl]amino}ethyl)amino]-3-methylpentanoate

A solution of the product of Example 59C (0.81 mmol) and (L)-methyl iso-leucinate hydrochloride (0.182 g, 0.813 mmol) in methanol (3.2 mL) and acetic acid (0.032 mL) was treated with NaCNBH₃ (0.104 g, 1.65 mmol), stirred at 25° C. for 1 hour, and partitioned between water and dichloromethane. The organic phase layer was separated and washed with 1N NaHCO₃ and brine, dried over Na₂SO₄, filtered and concentrated. The crude product was used without further purification.

### EXAMPLE 96B

### tert-butyl(2S,3S)-3-methyl-2-{3-[(1-methyl-1H-benzimidazol-2-yl)methyl]-2-oxo-1-imidazolidinyl}pentanoate

A solution of the product of Example 96A (0.81 mmol) in N,N-dimethylformamide (5 mL) was treated with diethylamine (0.8 mL), stirred at 25° C. for 2 hours and concentrated. A solution of the residue in 1,2-dichloroethane (16 mL) was treated with bis-(p-nitrophenyl) carbonate (0.296 g, 0.973 mmol), stirred at 60° C. for 16 hours and concentrated. The residue was chromatographed on silica gel, eluting with 0-100% ethyl acetate/dichloromethane to give the title compound (0.192 g, 59% yield).

### EXAMPLE 96C

### (2S,3S)-3-methyl-2-{3-[(1-methyl-1H-benzimidazol-2-yl)methyl]-2-oxo-1-imidazolidinyl}pentanoic Acid

A solution containing the product from Example 96B (0.037 g, 0.093 mmol) in dichloromethane (0.45 mL) was treated with trifluoroacetic acid (0.45 mL), stirred for 2 hours at 25° C. The solvent was concentrated and the residue was dissolve in ethyl acetate and concentrated to give the title compound as the trifluoroacetic acid salt, which was used without purification.

### EXAMPLE 96D

### methyl(1S)-1-[({(1S,2S,4S)-2-hydroxy-4-[((2S)-3-methyl-2-{3-[(1-methyl-1H-benzimidazol-2-yl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate

A solution containing the product from Example 23S (0.020 g, 0.038 mmol) in THF (0.5 mL) was treated with the product from Example 96C (0.021 g, 0.046 mmol), DEPBT (0.017 g, 0.057 mmol), and N,N-diisopropylethylamine (0.035 mL, 0.201 mmol), stirred at 25° C. for 1 hour, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.022 g, 68% yield). ¹H NMR (300 MHz, DMSO-d), δ ppm 0.60 (d, J=6.25 Hz, 3H, 0.72 (t, J=7.35 Hz, 3H), 0.85 (m, 12H), 1.24 (m, 1H), 1.51 (m, 2H), 1.73 (m, 1H), 2.67 (m, 1H), 2.77 (d, J=6.62 Hz, 2H), 2.89 (m, 1H), 3.08 (m, 2H), 3.51 (s, 3H), 3.59 (m, 1H), 3.77 (s, 3H), 3.85 (d, J=11.03 Hz, 1H), 3.94 (d, J=9.93 Hz, 1H), 4.15 (m, 2H), 4.59 (s, 2H), 4.82 (d, J=5.52 Hz, 1H), 6.80 (d, J=10.30 Hz, 1H), 6.99 (m, 5H), 7.24 (m, 5H), 7.56 (m, 3H), 7.88 (m, 5H), 8.63 (d, J=4.41 Hz, 1H).

### EXAMPLE 97A

### 2-isopropyl-1,3-thiazole-4-carbaldehyde

A solution containing the product from Example 56B (18 g, 90.5 mmol) in dichloromethane (100 mL) was treated with DIBAL (150 mL, 1 M in dichloromethane) dropwise at −78° C. over 2 hours, stirred at −78° C. for 2 hours, treated with acetic acid (10 mL), warmed to 25° C., treated with 10% solution of aqueous sodium potassium tartrate, stirred vigorously for 1 hour, and partitioned between dichloromethane and water. The organic phase was washed with brine and dried over MgSO₄, filtered and concentrated. The residue was chromatographed on silica gel eluting 0-5% ethyl acetate in dichloromethane to give the title compound (5.24 g, 40% yield).

### EXAMPLE 97B

### tert-butyl(2S,3S)-2-{3-[(2-isopropyl-1,3-thiazol-4-yl)methyl]-2-oxo-1-imidazolidinyl}-3-methylpentanoate

A solution containing the product from Example 3G (1.304 g, 5.66 mmol) in a mixture of benzene (15 mL) and methanol (15 mL) was treated with the product from Example 97A (1.05 g, 6.79 mmol), was heated at 50° C. for 3 hours, cooled to 0° C., treated with sodium borohydride (0.428 g, 11.32 mmol), stirred at 25° C. for 16 hours, quenched with sodium bicarbonate solution, and partitioned between ethyl acetate and water. The organic phase was washed with brine and dried over MgSO₄, filtered and concentrated. A solution of the concentrate (5.66 mmol) in toluene (30 mL) was treated with bis(4nitrophenyl)carbonate (2.066 g, 6.79 mmol), heated at 100° C. for 16 hours, cooled and partitioned between ethyl acetate and saturated NaHCO₃. The organic phase was washed with brine and dried over MgSO₄ filtered and concentrated. The residue was chromatographed on silica gel eluting with 0-25% ethyl acetate in dichloromethane to give the title compound (1.68 g, 75% yield).

### EXAMPLE 97C

### (2S,3S)-2-{3-[(2-isopropyl-1,3-thiazol-4-yl)methyl]-2-oxo-1-imidazolidinyl}-3-methylpentanoic Acid

A solution containing the product from Example 97B (1.68 g, 4.25 mmol) in dichloromethane (14 mL) was treated with trifluoracetic acid (7 mL), was stirred at 25° C. for 2 hours, and concentrated to give the title compound as the trifluoroacetic acid salt, which was used without further purification.

### EXAMPLE 97D

### methyl(1S)-1-[({(1S,2S,4S)-2-hydroxy-4-[((2S)-2-{3-[(2-isopropyl-1,3-thiazol-4-yl)methyl]-2-oxo-1-imidazolidinyl}-3-methylpentanoyl)amino]-5-phenyl-1-[4-(2-pyridinyl)benzyl]pentyl}amino)carbonyl]-2,2-dimethylpropylcarbamate

A solution containing the product from Example 23S (0.020 g, 0.038 mmol) in THF (0.5 mL) was treated with the product from Example 97C (0.020 g, 0.044 mmol), DEPBT (0.017 g, 0.057 mmol), and N,N-diisopropylethylamine (0.035 mL, 0.201 mmol), stirred at 25° C. for 1 hour, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.024 g, 75% yield). ¹H NMR (300 MHz, DMSO-d@, δ ppm 0.59 (d, J=6.25 Hz, 3H), 0.72 (t, J=7.17 Hz, 3H), 0.86 (s, 10H), 1.28 (m, 7H), 1.52 (m, 2H), 1.72 (m, 1H), 2.41 (m, 1H), 2.65 (m, 1H), 2.80 (m, 3H), 3.07 (m, 4H), 3.51 (s, 3H), 3.59 (m, 1H), 3.83 (d, J=11.03 Hz, 1H), 3.94 (d, J=9.56 Hz, 1H), 4.14 (m, 2H), 4.33 (m, 2H), 4.80 (d, J=5.52 Hz, 1H), 6.79 (d, J=9.19 Hz, 1H), 7.04 (s, 5H), 7.22 (s, 1H), 7.31 (m, 3H), 7.58 (d, J=8.46 Hz, 1H), 7.86 (m, 5H), 8.63 (d, J=4.04 Hz, 1H).

### EXAMPLE 98

### methyl(1S)-1-[({(1R,3S,4S)-3-hydroxy-4-[((2S)-3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate

A solution containing the product from Example 1H (0.020 g, 0.038 mmol) in THF (0.4 mL) was treated with the product from Example 7B (0.020 g, 0.048 mmol), DEPBT (0.017 g, 0.057 mmol), and N,N-diisopropylethylamine (0.035 mL, 0.201 mmol), stirred at 25° C. for 1 hour, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.025 g, 81% yield). ¹H NMR (300 MHz, DMSO-d₆), δ ppm 0.83 (m, 16H), 1.29 (m, 2H), 1.55 (m, 1H), 1.80 (m, 1H), 2.44 (s, 3H), 2.71 (m, 5H), 3.09 (m, 3H), 3.54 (m, 4H), 3.85 (m, 3H), 4.18 (m, 1H), 4.33 (s, 2H), 4.56 (d, J=6.99 Hz, 1H), 6.88 (d, J=9.56 Hz, 1H), 7.01 (d, J=7.35 Hz, 1H), 7.12 (m, 6H), 7.23 (d, J=8.09 Hz, 2H), 7.32 (m, 2H), 7.64 (t, J=7.72 Hz, 1H), 7.88 (m, 5H), 8.64 (d, J=4.78 Hz, 1H).

### EXAMPLE 99

### methyl(1S)-1-[({(1S,3S,4S)-4-[((2S)-3,3-dimethyl-2-{3-[(1-methyl-1H-benzimidazol-2-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 2C (0.025 g, 0.047 mmol) in THF (0.5 mL) was treated with the product from Example 59F (0.020 g, 0.061 mmol), DEPBT (0.021 g, 0.071 mmol), and N,N-diisopropylethylamine (0.041 mL, 0.235 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The reaction was purified by reversed phase chromatography on a C18 column eluting with 5-100% acetonitrile in water (0.1% TFA). The residue was partitioned between ethyl acetate and saturated NaHCO₃, and the organic phase was washed with brine and dried over MgSO₄, filtered and concentrated to give the title compound (0.006 g, 14% yield). ¹H NMR (300 MHz, DMSO-d₆), δ ppm 0.83 (s, 9H), 0.89 (s, 9H), 1.26 (m, 1H), 1.52 (m, 2H), 2.32 (m, 1H), 2.70 (m, 4H), 2.98 (m, 1H), 3.09 (m, 2H), 3.46 (s, 1H), 3.50 (s, 3H), 3.81 (s, 3H), 4.15 (m, 3H), 4.53 (dd, J=11.40, 3.68 Hz, 2H), 4.70 (d, J=15.44 Hz, 1H), 6.63 (d, J=9.56 Hz, 1H), 6.93 (m, 3H), 7.07 (d, J=6.62 Hz, 2H), 7.24 (m, 6H), 7.59 (m, 3H), 7.88 (m, 4H), 8.63 (d, J=3.31 Hz, 1H).

### EXAMPLE 100

### methyl(1S)-1-[({(1S,2S,4S)-4-[((2S)-3,3-dimethyl-2-{3-[(1-methyl-1H-benzimidazol-2-yl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate

A solution containing the product from Example 23S (0.011 g, 0.021 mmol) in THF (0.3 mL) was treated with the product from Example 59F (0.007 g, 0.020 mmol), DEPBT (0.009 g, 0.030 mmol), and N,N-diisopropylethylamine (0.018 mL, 0.103 mmol), stirred at 25° C. for 3 hours, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18-column eluting with 5-100% acetonitrile in water (0.1% TFA). The product was partitioned between ethyl acetate and saturated NaHCO₃, and the organic phase was washed with brine and dried over MgSO₄, filtered and concentrated to give the title compound (0.009 g, 51% yield). ¹H NMR (300 MHz, DMSO-d₆), δ ppm 0.82 (s, 9H), 0.86 (s, 9H), 1.27 (m, 1H), 1.53 (m, 2H), 2.36 (m, 2H), 2.72 (m, 3H), 2.98 (m, 1H), 3.10 (m, 1H), 3.48 (d, J=13.97 Hz, 3H), 3.61 (m, 1H), 3.80 (s, 3H), 3.93 (m, 2H), 4.16 (m, 2H), 4.60 (m, 2H), 4.83 (d, J=5.52 Hz, 1H), 6.91 (m, 4H), 7.02 (m, 2H), 7.20 (m, 2H), 7.29 (m, 3H), 7.58 (m, 3H), 7.87 (m, 5H), 8.63 (d, J=4.78 Hz, 1H).

### EXAMPLE 101A

### tert-butyl(2S)-2-{3-[(2-isopropyl-1,3-thiazol-4-yl)methyl]-2-oxo-1-imidazolidinyl}-3,3-dimethylbutanoate

A solution containing the product from Example 6F (0.060 g, 0.253 mmol) in a mixture of benzene (0.7 mL) and methanol (0.7 mL) was treated with the product from Example 97A (0.043 g, 0.278 mmol), heated at 50° C. for 3 hours, cooled to 0° C., treated with sodium borohydride (0.019 g, 0.506 mmol), stirred at 25° C. for 16 hours, quenched with sodium bicarbonate solution and partitioned between ethyl acetate and water. The organic phase was washed with brine and dried over MgSO₄, filtered and concentrated. A solution of the concentrate (0.253 mmol) in toluene (1.5 mL) was treated with bis(4nitrophenyl)carbonate (0.092 g, 0.304 mmol), heated at 1001C for 16 hours, cooled and partitioned between ethyl acetate and saturated NaHCO₃. The organic phase was washed with brine and dried over MgSO₄, filtered and concentrated. The residue was chromatographed on silica gel eluting with 0-25% ethyl acetate in dichloromethane to give the title compound (0.075 g, 75% yield).

### EXAMPLE 101B

### (2S)-2-{3-[(2-isopropyl-1,3-thiazol-4-yl)methyl]-2-oxo-1-imidazolidinyl}-3,3-dimethylbutanoic Acid

A solution containing the product from Example 97B (0.075 g, 0.190 mmol) in dichloromethane (0.5 mL) was treated with trifluoracetic acid (0.5 mL), was stirred at 25° C. for 2 hours, and concentrated to give the title compound as the trifluoroacetic acid salt, which was used without further purification.

### EXAMPLE 101C

### methyl(1S)-1-[({(1S,3S,4S)-3-hydroxy-4-[((2S)-2-{3-[(2-isopropyl-1,3-thiazol-4-yl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate

A solution containing the product from Example 2C (0.020 g, 0.038 mmol) in THF (0.5 mL) was treated with the product from Example 101B (0.015 g, 0.045 mmol), DEPBT (0.017 g, 0.057 mmol), and N,N-diisopropylethylamine (0.033 mL, 0.189 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.019 g, 59% yield). ¹H NMR (300 MHz, DMSO-d), δ ppm 0.83 (s, 9H), 0.88 (s, 9H), 1.31 (m, 6H), 1.53 (m, 2H), 2.30 (m, 1H), 2.62 (m, 3H), 2.79 (m, 1H), 3.02 (m, 2H), 3.22 (m, 2H), 3.50 (s, 3H), 3.66 (m, 1H), 3.85 (d, J=9.56 Hz, 1H), 4.20 (m, 4H), 4.45 (m, 1H), 4.53 (d, J=7.35 Hz, 1H), 6.63 (d, J=9.93 Hz, 1H), 7.03 (m, 5H), 7.28 (m, 4H), 7.45 (d, J=9.56 Hz, 1H), 7.86 (m, 5H), 8.64 (d, J=4.41 Hz, 1H).

### EXAMPLE 102

### methyl(1S)-1-[({(1S,2S,4S)-2-hydroxy-4-[((2S)-2-{3-[(2-isopropyl-1,3-thiazol-4-yl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate

A solution containing the product from Example 23S (0.018 g, 0.033 mmol) in THF (0.5 mL) was treated with the product from Example 101B (0.014 g, 0.041 mmol), DEPBT (0.015 g, 0.051 mmol), and N,N-diisopropylethylamine (0.030 mL, 0.171 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.018 g, 62% yield). ¹H NMR (300 MHz, DMSO-d₆), δ ppm 0.81 (s, 9H), 0.86 (s, 9H), 132 (d, J=6.99 Hz, 6H), 1.52 (m, 2H), 2.38 (m, 2H), 2.64 (d, J=9.93 Hz, 1H), 2.77 (d, J=6.99 Hz, 2H), 3.01 (n, 2H), 3.23 (m, 2H), 3.51 (s, 3H), 3.61 (m, 1H), 3.95 (m, 2H), 4.30 (m, 4H), 4.82 (d, J=5.52 Hz, 1H), 6.79 (d, J=9.19 Hz, 1H), 7.00 (m, 5H), 7.24 (s, 1H), 7.30 (m, 3H), 7.58 (d, J=9.56 Hz, 1H), 7.87 (m, 5H), 8.63 (d, J=4.41 Hz, 1H).

### EXAMPLE 103A

### benzyl(1S,2S,4S)-4-amino-2-hydroxy-5-phenyl-1-[4-(3-pyridinyl)benzyl]pentylcarbamate

A solution containing the product from Example 67A (0.059 g, 0.093 mmol) in a mixture of methanol (3 mL) and aqueous HCl (1 mL, 1 N), stirred at 50° C. for 2 hours, and concentrated to give the title compound as the hydrochloride salt, which was used without further purification.

### EXAMPLE 103B

### (2S,3S,5S)-2,5-diamino-6-phenyl-1-[4-(3-pyridinyl)phenyl]-3-hexanol

A solution containing the product from Example 103A (0.093 mmol) in methanol (2 mL) was treated with Pd(OH) on carbon (0.050 g, 20% Pd by wt.) and HCl solution (0.040 mL, 4N in dioxane), stirred under a hydrogen atmosphere (balloon pressure) at 25° C. for 2 hours, filtered through a bed of celite®, rinsed with methanol, and concentrated to give the title compound as the hydrochloride salt, which was used without further purification.

### EXAMPLE 103C

### methyl(1S,4S,5S,7S,10S)-7-benzyl-1,10-ditert-butyl-5-hydroxy-2,9,12-trioxo-4-[4-(3-pyridinyl)benzyl]-13-oxa-3,8,11-triazatetradec-1-ylcarbamate

A solution containing the product from Example 103B (0.093 mmol) in THF (1 mL) was treated with the product from Example 1F (0.040 g, 0.211 mmol), DEPBT (0.085 g, 0.284 mmol), and N,N-diisopropylethylamine (0.175 mL, 1.00 mmol), stirred at 25° C. for 1 hour, and partitioned between ethyl acetate and 10% Na₂CO₃ solution. The organic phase was washed with additional 10% Na₂CO₃ solution and brine, dried over MgSO₄, filtered and concentrated. The residue was purified by chromatography on silica gel eluting with 0-100% ethyl acetate/dichloromethane, followed by 0-5% methanol in ethyl acetate, to give the title compound (0.035 g, 55% yield). ¹H NMR (300 MHz, DMSO-d₆), δ ppm 0.77 (s, 9H), 0.83 (s, 9H), 1.50 (m, 2H), 2.73 (m, 4H), 3.48 (s, 3H), 3.54 (s, 3H), 3.64 (m, 1H), 3.80 (d, J=10.30 Hz, 1H), 3.93 (d, J=9.19 Hz, 1H), 4.12 (m, 2H), 4.84 (d, J=5.52 Hz, 1H), 6.61 (d, J=9.56 Hz, 1H), 6.78 (d, J=8.82 Hz, 1H), 7.11 (m, 5H), 7.32 (d, J=8.09 Hz, 2H), 7.47 (dd, J=7.72, 5.15 Hz, 1H), 7.56 (m, 3H), 7.73 (d, J=8.46 Hz, 1H), 8.01 (m, 1H), 8.54 (dd, J=4.60, 1.65 Hz, 1H), 8.84 (d, J=1.84 Hz, 1H).

### EXAMPLE 104A

### benzyl(1S,2S,4S)-2-hydroxy-4-({(2S)-2-[(methoxycarbonyl)amino]-3,3-dimethylbutanoyl}amino)-5-phenyl-1-[4-(4-pyridinyl)benzyl]pentylcarbamate

A solution containing the product from Example 73B (0.045 mmol) in THF (0.45 mL) was treated with the product from Example 1F (0.010 g, 0.053 mmol), DEPBT (0.020 g, 0.067 mmol), and N,N-diisopropylethylamine (0.080 mL, 0.459 mmol), stirred at 25° C. for 0.5 hours, and partitioned between ethyl acetate and 10% Na₂CO₃ solution. The organic phase was washed with additional 10% Na₂CO₃ solution and brine, dried over MgSO₄, filtered and concentrated to give the title compound, which was used without further purification.

### EXAMPLE 104B

### methyl(1S)-1-[({(1S,3S,4S)-4-amino-1-benzyl-3-hydroxy-5-[4-(4-pyridinyl)phenyl]pentyl}amino)carbonyl]-2,2-dimethylpropylcarbamate

A solution containing the product from Example 104A (0.045 mmol) in methanol (0.5 mL) was treated with Pd(OH)₂ on carbon (0.010 g, 20% Pd by wt.) and HCl solution (0.035 mL, 4N in dioxane), stirred under a hydrogen atmosphere (balloon pressure) for 4 hours at 25° C., filtered through a bed of celite®, rinsed with methanol, and concentrated to give the title compound as the hydrochloride salt, which was used without further purification.

### EXAMPLE 104C

### methyl(1S,4S,5S,7S,10S)-7-benzyl-1,10-ditert-butyl-5-hydroxy-2,9,12-trioxo-4-[4-(4-pyridinyl)benzyl]-13-oxa-3,8,11-triazatetradec-1-ylcarbamate

A solution containing the product from Example 104B (0.045 mmol) in THF (0.45 mL) was treated with the product from Example 1F (0.010 g, 0.053 mmol), DEPBT (0.020 g, 0.067 mmol), and N,N-diisopropylethylamine (0.080 mL, 0.459 mmol), stirred at 25° C. for 1 hour, and partitioned between ethyl acetate and 10% Na₂CO₃ solution. The organic phase was washed with additional 10% Na₂CO₃ solution and brine, dried over MgSO₄, filtered and concentrated. The residue was purified by reversed phase chromatography on a C18 column eluting with 5-100% acetonitrile in water (0.1% TFA). The product was partitioned between ethyl acetate and saturated NaHCO₃, and the organic phase was washed with brine and dried over MgSO₄, filtered and concentrated to give the title compound (0.008 g, 25% yield). ¹H NMR (300 MHz, DMSO-d₆), δ ppm 0.77 (s, 9H), 0.82 (s, 9H), 1.48 (m, 2H), 2.75 (m, 4H), 3.49 (s, 3H), 3.54 (s, 3H), 3.63 (m, 1H), 3.80 (d, J=9.93 Hz, 1H), 3.93 (d, J=9.56 Hz, 1H), 4.10 (m, 2H), 4.85 (d, J=5.52 Hz, 1H), 6.60 (d, J=9.19 Hz, 1H), 6.76 (d, J=10.30 Hz, 1H), 7.10 (m, 5H), 7.33 (d, J=8.09 Hz, 2H), 7.64 (m, 6H), 8.61 (m, 2H).

### EXAMPLE 105

### methyl(1S)-1-[({(1S,3S,4S)-4-[((2S)-3,3-dimethyl-2-{3-[(2-methyl-1,3-thiazol-4-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 2C (0.044 g, 0.082 mmol) in THF (0.7 mL) was treated with the product from Example 14B (0.033 g, 0.107 mmol), DEPBT (0.037 g, 0.124 mmol), and N,N-diisopropylethylamine (0.072 mL, 0.412 mmol), stirred at 25 C for 2 hours, and partitioned between ethyl acetate and 10% Na₂CO₃ solution. The organic phase was washed with additional 10% Na₂CO₃ solution and brine, dried over MgSO₄, filtered and concentrated. The residue was purified by chromatography on silica gel eluting with 0-100% ethyl acetate dichloromethane, followed by 0-5% methanol in ethyl acetate, to give the title compound (0.056 g, 83% yield). ¹H NMR (300 MHz, DMSO-d₆), δ ppm 0.83 (s, 9H), 0.88 (s, 9H), 1.54 (m, 2H), 2.36 (q, J=9.31 Hz, 1H), 2.61 (m, 5H), 2.78 (m, 1H), 3.01 (m, 2H), 3.22 (m, 2H), 3.50 (s, 3H), 3.66 (m, 1H), 3.85 (d, J=9.56 Hz, 1H), 4.17 (m, 4H), 4.41 (m, 1H), 4.54 (d, J=7.35 Hz, 1H), 6.63 (d, J=9.56 Hz, 1H), 7.06 (m, 5H), 7.21 (s, 1H), 7.24 (s, 2H), 7.31 (m, 1H), 7.45 (d, J=9.56 Hz, 1H), 7.87 (m, 5H), 8.63 (d, J=4.41 Hz, 1H).

### EXAMPLE 106

### methyl(1S)-1-[({(1S,2S,4S)-4-[((2S)-3,3-dimethyl-2-{3-[(2-methyl-1,3-thiazol-4-yl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate

A solution containing the product from Example 23S (0.038 g, 0.071 mmol) in THF (0.7 mL) was treated with the product from Example 14B (0.029 g, 0.092 mmol), DEPBT (0.032 g, 0.107 mmol), and N,N-diisopropylethylamine (0.062 mL, 0.355 mmol) stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was purified by chromatography on silica gel eluting with 0-100% ethyl acetate/dichloromethane, followed by 0-5% methanol in ethyl acetate, to give the title compound (0.041 g, 70% yield). ¹H NMR (300 MHz, DMSO-d₆), δ ppm 0.81 (s, 9H), 0.86 (s, 9H), 1.53 (m, 2H), 2.39 (m, 2H), 2.64 (m, 4H), 2.77 (d, J=6.62 Hz, 2H), 3.00 (m, 2H), 3.19 (m, 1H), 3.51 (s, 3H), 3.61 (m, 1H), 3.96 (m, 2H), 4.32 (m, 4H), 4.82 (d, J=5.52 Hz, 1H), 6.79 (d, J=9.56 Hz, 1H), 7.04 (m, 5H), 7.21 (s, 1H), 7.30 (m, 3H), 7.58 (d, J=8.82 Hz, 1H), 7.87 (m, 5H), 8.63 (d, J=4.78 Hz, 1H).

### EXAMPLE 107A

### ethyl(5R)-5-{(1S)-1-[(tert-butoxycarbonyl)amino]-2-phenylethyl}-2-oxotetrahydro-3-furancarboxylate

A solution of tert-Butyl(1S)-1-[(2S)-oxiran-2-yl]-2-phenylethylcarbamate (10.0 g, 38.0 mmol) and diethyl malonate (9.0 ml, 59.3 mmol) in ethanol (27 mL) at 0° C. was treated with a solution of NaOEt (16 mL, 21% in ethanol) over 10 minutes, stirred at 70° C. for 2 hours, cooled to 0° C. and quenched with 10% citric acid solution, and partitioned between ethyl acetate and water. The organic phase was washed with saturated NaHCO₃ and brine, dried over MgSO₄, filtered and concentrated. The residue was chromatographed on silica gel eluting with 0-35% ethyl acetate in hexanes to give the title compound (13.3 g, 93% yield).

### EXAMPLE 107B

### tert-butyl(18)-1-{(2R)-5-oxo-4-[4-(2-pyridinyl)benzyl]tetrahydro-2-furanyl}-2-phenylethylcarbamate

A solution of the product from Example 107A (13.3 g, 35.27 mmol) in ethanol (140 mL) at 0° C. was treated with a solution of NaOEt (14.9 mL, 21% in ethanol) and solid 2-[4-(bromomethyl)phenyl]pyridine (12.05 g, 48.59 mmol), stirred at 25° C. for 16 hours, treated with a solution of LiOH monohydrate (8.9 g, 212.11 mmol) in water (35 mL), stirred at 25° C. for 5 hours, cooled to 0° C., adjusted to pH 5 by addition of 10% citric acid and then partitioned between dichloromethane and water. The organic phase was washed with brine and dried over MgSO₄, filtered and concentrated. A solution of the concentrate in toluene (1 L) was heated at reflux for 16 hours, cooled and concentrated to give the title compound (10.55 g, 63% yield), which was used without further purification.

### EXAMPLE 107C

### (4R,5S)-5-[(tert-butoxycarbonyl)amino]-4-{[tert-butyl(dimethyl)silyl]oxy}-6-phenyl-2-[4-(2-pyridinyl)benzyl]hexanoic Acid

A solution containing the product from Example 107B (10.55 g, 22.35 mmol) in a mixture of dioxane (130 mL) and water (65 mL) was treated with sodium hydroxide solution (33.5 mL, 1N), stirred for 30 minutes at 25° C., concentrated, cooled to 0° C., acidified to pH 5 using 10% citric acid, and partitioned between dichloromethane and water. The organic phase layer was washed with brine, dried over Na₂SO₄, filtered and concentrated. A solution of the concentrate in dimethylformamide (130 mL) was treated with imidazole (18.3 g, 268.80 mmol) and t-butyldimethylsilyl chloride (20.2 g, 134.01 mmol), stirred at 25° C. for 16 hours, and concentrated. The concentrate was combined with ice and extracted with ethyl acetate. The organic phase was washed with 10% citric acid and brine, dried over MgSO₄, filtered, and concentrated. A solution of the residue in a mixture of THF (100 mL), acetic acid (100 mL) and water (33 mL) was stirred at 25° C. for 2 hours, and concentrated under reduced pressure. The residue was dissolved in toluene and concentrated several time, followed by drying under high vacuum to give the title compound, which was used without further purification.

### EXAMPLE 107D

### benzyl(3R,4S)-4-[(tert-butoxycarbonyl)amino]-3-hydroxy-5-phenyl-1-[4-(2-pyridinyl)benzyl]pentylcarbamate

A solution of the product from Example 107C (22.35 mmol) in toluene (500 mL) was treated with DPPA (5.3 mL, 24.59 mmol) and triethylamine (3.75 mL, 26.90 mmol) was heated at reflux for 2 hours, cooled to 25° C., treated with benzyl alcohol (6.9 mL, 66.68 mmol), heated at reflux for an additional 16 hours, cooled and concentrated. A solution of the concentrate in THF (100 mL) was treated with TBAF solution in THF (67 mL, 1N), stirred at 25° C. for 40 hours, concentrated, and partitioned between ethyl acetate and water. The organic phase was washed with brined, dried over MgSO₄, filtered and concentrated, to give the title compound (4.98 g, 37% yield), which was used without further purification.

### EXAMPLE 107E

### tert-butyl(1S,2R)-4-amino-1-benzyl-2-hydroxy-5-[4-(2-pyridinyl)phenyl]pentylcarbamate

A solution containing the product from Example 107D (0.5 g, 0.840 mmol) in a mixture of methanol (4 mL) and ethyl acetate (4 mL) was treated with Pd(OH)₂ on carbon (0.175 g, 20% Pd by wt.) and HCl solution (0.40 mL, 4N in dioxane), stirred under a hydrogen atmosphere (balloon pressure) at 25° C. for 2 hours, filtered through a bed of celite®, rinsed with methanol, and concentrated to give the title compound as the hydrochloride salt.

### EXAMPLE 107F

### tert-butyl(1S,2R,4S)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate

A solution containing the product from Example 1E (0.840 mmol) in THF (8 mL) was treated with the product from Example 1F (0.175 g, 0.926 mmol), DEPBT (0.375 g, 1.194 mmol), and N,N-diisopropylethylamine (0.75 mL, 4.31 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give a mixture of products (0.254 g, 48% yield). A portion of the mixture (0.112 g) was chromatographed on silica gel eluting with 0-100% tert-butyl methyl ether/dichloromethane, to give the lower Rf compound (0.033 g).

### EXAMPLE 107G

### tert-butyl(1S,2R,4R)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate

A solution containing the product from Example 1E (0.840 mmol) in THF (8 mL) was treated with the product from Example 1F (0.175 g, 0.926 mmol), DEPBT (0.375 g, 1.194 mmol), and N,N-diisopropylethylamine (0.75 mL, 4.31 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane to give a mixture of products (0.254 g, 48% yield). A portion of the mixture (0.112 g) was chromatographed on silica gel eluting with 0-100% tert-butyl methyl ether/dichloromethane, to give the higher Rf compound (0.042 g).

### EXAMPLE 107H

### methyl(1S)-1-[({(1S,3R,4S)-4-amino-3-hydroxy-5-phenyl-1-[4-(2-pyridinyl)benzyl]pentyl}amino)carbonyl]-2,2-dimethylpropylcarbamate

A solution containing the product from Example 107F (0.033 g, 0.052 mmol) in dichloromethane (1 mL) was treated with trifluoroacetic acid (1-mL), stirred at 25° C. for 1 hour, concentrated, and partitioned between ethyl acetate and saturated NaHCO₃ solution. The organic phase was washed with brine, dried over MgSO₄, filtered and concentrated.

### EXAMPLE 107I

### methyl(1S)-1-[({(1S,3R,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 107H (0.052 mmol) in THF (0.6 mL) was treated with the product from Example 10D (0.021 g, 0.063 mmol), DEPBT (0.024 g, 0.078 mmol), and N,N-diisopropylethylamine (0.045 mL, 0.261 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.024 g, 56% yield). ¹H NMR (300 MHz, DMSO-d₆), δ ppm 0.71 (s, 6H), 0.89 (m, 12H), 1.25 (s, 1H), 1.48 (m, 1H), 1.71 (m, 1H), 2.46 (m, 3H), 2.63 (m, 1H), 2.74 (m, 1H), 2.98 (m, 3H), 3.22 (m, 1H), 3.50 (m, 5H), 3.82 (m, 2H), 4.02 (s, 1H), 4.20 (m, 1H), 4.38 (m, 2H), 4.91 (d, J=6.62 Hz, 1H), 6.77 (d, J=9.93 Hz, 1H), 7.06 (m, 5H), 7.16 (d, J=7.72 Hz, 1H), 7.26 (d, J=8.46 Hz, 1H), 7.32 (m, 3H), 7.69 (m; 1H), 7.87 (m, 6H), 8.64 (d, J=4.78 Hz, 1H).

### EXAMPLE 108A

### methyl(1S)-1-[({(1R,3R,4S)-4-amino-3-hydroxy-5-phenyl-1-[4-(2-pyridinyl)benzyl]pentyl}amino)carbonyl]-2,2-dimethylpropylcarbamate

A solution containing the product from Example 107G (0.042 g, 0.066 mmol) in dichloromethane (1 mL) was treated with trifluoroacetic acid (1 mL), stirred at 25° C. for 1 hour, concentrated, and partitioned between ethyl acetate and saturated NaHCO₃ solution. The organic phase was washed with brine, dried over MgSO₄, filtered and concentrated.

### EXAMPLE 108B

### methyl(1S)-1-[({(1R,3R,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

A solution containing the product from Example 108A (0.066 mmol) in THF (0.7 mL) was treated with the product from Example 10D (0.027 g, 0.080 mmol), DEPBT (0.030 g, 0.100 mmol), and N,N-diisopropylethylamine (0.057 mL, 0.332 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate, to give the title compound (0.027 g, 49% yield). ¹H NMR (300 MHz, DMSO-d), δ ppm 0.88 (d, J=1.10 Hz, 18H), 1.25 (s, 1H), 1.51 (m, 2H), 2.46 (s, 3H), 2.75 (d, J=6.25 Hz, 2H), 2.87 (m, 1H), 3.05(m, 2H), 3.23 (m, 1H), 3.48 (m, 5H), 3.84 (m, 2H), 3.99 (s, 1H), 4.14 (m, 1H), 4.36 (m, 2H), 4.67 (d, J=5.52 Hz, 1H), 6.81 (d, J=9.56 Hz, 1H), 7.02 (m, 5H), 7.15 (d, J=7.72 Hz, 1H), 7.30 (m, 4H), 7.67 (m, 2H), 7.87 (m, 5H), 8.64 (d, J=4.78 Hz, 1H).

### EXAMPLE 109A

### 4-(chloromethyl)-2-methyl-1,3-thiazole

A solution of thioacetamide (2.45 g, 32.6 mmol) in 2-propanol (130 mL) was treated with dichloroacetone (4.14 g, 32.6 mmol) was heated at 60° C. for 2 hours, cooled and concentrated under reduced pressure. The solid product was added cautiously to a saturated NaHCO₃solution (gas evolution) and the mixture was partitioned between chloroform and saturated NaHCO₃. The organic phase was washed with water, dried over Na₂SO₄, filtered and concentrated. The residue was purified by chromatography on silica gel eluting with chloroform to give the title compound.

### EXAMPLE 109B

### N-methyl(2-methyl-1,3-thiazol-4-yl)methanamine

An aqueous solution of methylamine (18 mL, 40%) was treated with the product from Example 109A (2.0 g, 13.5 mmol) in portions over 0.5 hours, stirred at 25° C. for 16 hours, and concentrated. The residue was chromatographed on silica gel eluting with 5% methanol in chloroform to give the title compound (1.23 g, 64% yield).

### EXAMPLE 109C

### methyl(2S,3S)-3-methyl-2-{[(4-nitrophenoxy)carbonyl]amino}pentanoate

A solution of L-iso-leucine methyl ester hydrochloride (2.5 g, 13.75 mmol) in dichloromethane (35 mL) at 0° C. was treated with 4-nitrophenyl chloroformate (3.05, 15.13 mmol) and 4-methylmorpholine (3.2 mL, 29.11 mmol), stirred at 25° C. for 64 hours, and partitioned between dichloromethane and saturated NaHCO₃. The organic phase was washed with brine and dried over MgSO₄, filtered and concentrated to give the title compound (4.19 g, 98% yield).

### EXAMPLE 109D

### methyl(2S,3S)-3-methyl-2-[({methyl[(2-methyl-1,3-thiazol-4-yl)methyl]amino}carbonyl)amino]pentanoate

A solution containing the product from Example 109B (0.200 g, 1.4 mmol) in THF (6 mL) was treated with the product from Example 109C (0.415 g, 1.4 mmol), triethylamine (0.196 mL, 1.4 mmol), and DMAP (0.020 g, 0.16 mmol) at 25° C., stirred at reflux for 1 hour, cooled and concentrated. The residue was partitioned between ethyl acetate and 5% K₂CO₃. The organic phase was washed with brine and dried over Na₂SO₄, filtered and concentrated to give the title compound (0.38 mg, 86% yield).

### EXAMPLE 109E

### (2S,3S)-3-methyl-2-[({methyl[(2-methyl-1,3-thiazol-4-yl)methyl]amino}carbonyl)amino]pentanoic Acid

A solution of the product from Example 109D (0.38 g, 1.2 mmol) in dioxane (5 mL) was treated with an aqueous solution of lithium hydroxide (5.0 mL, 0.5 M), stirred for 0.5 hours at 25° C., treated with aqueous HCl (2.5 mL, 1 N), and partitioned between ethyl acetate and water. The organic phase was washed with brine and dried over Na₂SO₄, filtered and concentrated to give the title compound.

### EXAMPLE 109F

### methyl(1S,4S,6S,7S,10S)-7-benzyl-10-sec-butyl-1-tert-butyl-6-hydroxy-13-methyl-14-(2-methyl-1,3-thiazol-4-yl)-2,9,12-trioxo-4-[4-(2-pyridinyl)benzyl]-3,8,11,13-tetraazatetradec-1-ylcarbamate

A solution containing the product from Example 2C (0.025 g, 0.047 mmol) in THF (0.5 mL) was treated with the product from Example 109E (0.018 g, 0.061 mmol), DEPBT (0.021 g, 0.071 mmol), and N,N-diisopropylethylamine (0.041 mL, 0.235 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-10% methanol in ethyl acetate, to give the title compound (0.030 g, 78% yield). ¹H NMR (300 MHz, DMSO-d₆), δ ppm 0.78 (m, 16H), 1.01 (m, 1H), 1.36 (m, 1H), 1.50 (m, 2H), 1.70 (m, 1H), 2.61 (s, 3H), 2.73 (m, 3H), 2.86 (s, 3H), 3.49 (s, 3H), 3.62 (m, 1H), 3.83 (d, J=9.93 Hz, 1H), 3.98 (t, J=7.91 Hz, 1H), 4.11 (m, 2H), 4.43 (m, 2H), 4.86 (d, J=5.88 Hz, 1H), 6.19 (d, J=8.09 Hz, 1H), 6.62 (d, J=9.56 Hz, 1H), 7.16 (m, 8H), 7.31 (m, 1H), 7.42 (d, J=9.19 Hz, 1H), 7.77 (d, J=8.09 Hz, 1H), 7.85 (m, 4H), 8.63 (d, J=4.78 Hz, 1H).

### EXAMPLE 110

### methyl(1S,4S,5S,7S,10S)-7-benzyl-10-sec-butyl-1-tert-butyl-5-hydroxy-13-methyl-14-(2-methyl-1,3-thiazol-4-yl)-2,9,12-trioxo-4-[4-(2-pyridinyl)benzyl]-3,8,11,13-tetraazatetradec-1-ylcarbamate

A solution containing the product from Example 23S (0.025 g, 0.047 mmol) in THF (0.5 mL) was treated with the product from Example 109E (0.018 g, 0.061 mmol), DEPBT (0.021 g, 0.071 mmol), and N,N-diisopropylethylamine (0.041 mL, 0.235 mmol), stirred at 25° C. for 2 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-10% methanol in ethyl acetate. The product was then purified by reversed phase chromatography on a C18 column eluting with 5-100% acetonitrile in water (0.1% TFA). The product was partitioned between ethyl acetate and saturated NaHCO₃, and the organic phase was washed with brine and dried over MgSO₄, filtered and concentrated to give the title compound (0.011 g, 29% yield). ¹H NMR (300 MHz, DMSO-d₆), δ ppm 0.64 (d, J=6.62 Hz, 3H), 0.72 (t, J=7.35 Hz, 3H), 0.83 (s, 9H), 0.93 (m, 1H), 1.28 (m, 2H), 1.49 (m, 2H), 1.60 (m, 1H), 2.61 (s, 3H), 2.73 (m, 3H), 2.84 (s, 3H), 3.50 (s, 3H), 3.62 (m, 1H), 3.91 (m, 2H), 4.10 (m, 2H), 4.41 (m, 2H), 4.80 (d, J=5.52 Hz, 1H), 6.01 (d, J=8.46 Hz, 1H), 6.76 (d, J=9.93 Hz, 1H), 7.12 (m, 6H), 7.31 (m, 3H), 7.60 (m, 2H), 7.86 (m, 4H), 8.63 (d, J=4.41 Hz, 1H).

### EXAMPLE 111

### methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate

A solution containing the product from Example 2C (1.09 g, 2.05 mmol) in THF (20 mL) was treated with the product from Example 70A (0.71 g, 2.45 mmol), DEPBT (1.0 g, 3.34 mmol), and N,N-diisopropylethylamine (2.0 mL, 11.5 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 1% methanol in ethyl acetate to give the title compound (1.197 g, 73% yield). ¹H NMR (300 MHz, DMSO-d₆), δ ppm 0.83 (s, 9H), 0.89 (s, 9H), 1.56 (m, 2H), 2.33 (q, J=9.2 Hz, 1H), 2.58 (dd, J=13.6, 8.8 Hz, 1H), 2.67 (m, 2H), 2.78 (dd, J=13.6, 3.3 Hz, 1H), 2.84 (m, 1H), 2.94 (q, J=9.2 Hz, 1H), 3.19 (m, 1H), 3.50 (s, 3H), 3.67 (m, 1H), 3.85 (d, J=9.93 Hz, 1H), 4.09 (s, 1H), 4.19 (m, 2H), 4.31 (s, 2H), 4.55 (d, J=7.72 Hz, 1H), 6.63 (d, J=9.56 Hz, 1H), 7.07 (m, 5H), 7.22 (d, J=8.5 Hz, 2H), 7.29 (m, 4H), 7.36 (m, 2H), 7.47 (d, J=9.56 Hz, 1H), 7.85 (m, 3H), 7.89 (d, J=8.5 Hz, 2H), 8.63 (d, J=4.78 Hz, 1H).

### EXAMPLE 112A

### methyl(2S)-3-(4-bromophenyl)-2-[(tert-butoxycarbonyl)amino]propanoate

A mixture of (L)-4-bromophenylalanine (1.0 g, 4.1 mmol), NaHCO₃ (0.9 g, 10.7 mmol), and di-tert-butyldicarbonate (1.34 g, 6.1 mmol) in 4:1 1,4-dioxane:water (25 mL) was stirred at 25° C. for 18 hours, diluted with water (20 mL) and extracted with dichloromethane (50 mL). The aqueous phase was adjusted to pH-2 using 1N HCl, and extracted with ethyl acetate (2×50 mL). The combined organic phase was dried over Na₂SO₄, filtered and concentrated. A solution of the concentrate in methanol (20 mL) was cooled to 0° C., treated with a solution of trimethylsilyl diazomethane (2.0 M in Et₂O), stirred at 25° C. for 18 hours, then concentrated. The residue was chromatographed on silica ge, eluting with dichloromethane to afford the title compounds (1.15 g, 78%).

### EXAMPLE 112B

### tert-butyl(1S)-2-hydroxy-1-[4-(2-pyridinyl)benzyl]ethylcarbamate

(i) A solution containing the product from Example 112A (1.15 g, 3.2 mmol) in anhydrous THF (20 mL) at 0° C. was treated dropwise with a solution of lithium aluminumhydride (3.2 mL, 1N in THF), stirred at (PC for 1 h, treated with ethyl acetate (2 mL), washed with water (10 mL), 15% aq. NaOH, and water (10 mL), dried over Na₂SO₄, filtered and concentrated. The residue was purified by column chromatography on silica gel, eluting with 30-50% ethyl acetate in hexanes.

(ii) A solution of the product from step (i) (0.20 g, 0.61 mmol), 3-tri-n-butylstannyl)pyridine (0.9 g, 2.44 mmol), and dichlorobis(triphenylphosphine)palladium (0.13 g, 0.19 mmol) in dry acetonitrile (4 mL) was stirred at 80° C. for 18 hours, filtered and concentrated. The concentrate was chromatographed on silica gel, eluting with 30-60% ethyl acetate in hexanes to give the title compound (0.18 g, 90%).

### EXAMPLE 112C

### 2,5-bis[(tert-butoxycarbonyl)amino]-1,2,5,6-tetradeoxy-1,6-bis[4-(2-pyridinyl)phenyl]-L-iditol

(i) A solution of oxalyl chloride (0.42 mL, 2.0 M in CH₂Cl₂, 0.84 mmol) in anhydrous dichloromethane (2 mL) at −63° C. (CHCl₃-dry ice bath) was treated dropwise with a solution of DMSO (80 [ ]L, 88 mg, 1.13 mmol) in dichloromethane (2 mL). To this solution was treated with, dropwise, a solution of the product from Example 112B (0.18 g, 0.55 mmol) in anhydrous dichloromethane (1 mL). The resulting mixture was stirred for 20 min at −63° C., treated with triethylamine (0.31 mL, 0.23 g, 2.22 mmol), stirred for 30 min at −63° C., warmed to 25° C., treated with 10% citric acid (5 mL) and hexanes (5 mL), and the layers were separated. The aqueous layer was washed with diethyl ether (2×5 mL). The combined organic layers were dried over Na₂SO₄, filtered and concentrated. The crude product was purified by column chromatography on silica gel, eluting with 1:1 ethyl acetate:hexanes to give the aldehyde (0.12 g, 67%).

(ii) A solution of vanadium (III) chloride-THF complex (1:3) in anhydrous CH₂Cl₂ (0.5 M, 0.4 mL, 0.2 mmol) under N₂ was treated with Zn (7 mg, 0.11 mmol), stirred at 25° C. for 30 min. To this mixture was added a solution of the aldehyde from step (i) (60 mg, 0.20 mmol) in anhydrous dichloromethane (0.5 mL), and the resulting mixture was stirred at 25° C. for 18 hours. The mixture was treated with 0.2 M HCl (2 mL), stirred at 25° C. for 1 h, extracted with dichloromethane (3×2 mL), dried over Na₂SO₄, filtered and concentrated. The crude product was chromatographed on silica gel, eluting 60-100% ethyl acetate in hexanes to give the title compound (11 mg, 9%).

### EXAMPLE 112D

### 1,2,5,6-tetradeoxy-2,5-bis({(2S)-2-[(methoxycarbonyl)amino]-3,3-dimethylbutanoyl}amino)-1,6-bis[4-(2-pyridinyl)phenyl]-L-iditol

A solution of the product from Example 112C (9 mg, 14 mmol) in a 1:1 mixture of methanol and 4N HCl (0.2 mL) was stirred at 25° C. for 4 hours, and concentrated in vacuo. A solution of the residue in dimethylformamide (0.2 mL) was treated with the product from Example 1F (7 mg, 40 μmol), DEPBT (18 mg, 60 μmol), and triethylamine (12 μL, 9 mg, 86 μmol), stirred at 25° C. for 18 hours, and partitioned between saturated NaHCO₃ (0.5 mL) and ethyl acetate (3×1 mL). The organic phase was dried over Na₂SO₄ filtered and concentrated. The residue was chromatographed on silica gel, eluting with 50-100% ethyl acetate in hexanes to afford the title compound (6 mg, 55%).

1H NMR (300 MHz, CDCl₃) δ ppm 0.81(s, 18H), 3.02(m, 4H), 3.49 (m, 2H), 3.59(s, 6H), 3.74(m, 2H), 4.14(m, 4H), 5.19(m, 2H), 6.33(m, 2H), 7.34 (d, J=8.09 Hz, 4H), 7.74(m, 6H), 7.88(d, J=8.46 Hz, 4H), 8.67(m, 2H).

### EXAMPLE 113A

### Methyl 6-(tributylstannyl)-2-pyridinyl Ether

A solution containing 2-bromo-6-methoxypyridine (0.65 mL, 5.3 mmol) in ether (11 mL) at −78° C. was treated with n-butyllithium (4.0 mL, 1.6 M in hexanes) dropwise, warmed to 0° C. for 10 minutes, cooled to −78° C., treated with tributyltin chloride (2.25 mL, 8.30 mmol), stirred at −78° C. for 0.5 hours, and then at 0° C. for 0.5 hours. The reaction was quenched with saturated ammonium chloride solution and partitioned between ether and water. The organic phase was washed with brine and dried over MgSO₄, filtered and concentrated to give the title compound.

### EXAMPLE 113B

### benzyl(1S,3S,4S)-4-[(tert-butoxycarbonyl)amino]-3-{[tert-butyl(dimethyl)silyl]oxy}-1-[4-(6-methoxy-2-pyridinyl)benzyl]-5-phenylpentylcarbamate

A solution containing the product from Example 92D (0.20 g, 0.28 mmol) in DMF (3 mL) was treated with LiCl (0.119 g, 2.8 mmol), dichlorobis(triphenylphosphine)palladium(II) (0.060 g, 0.085 mmol), and the product from Example 113A (0.336 g, 0.84 mmol), heated at 100° C. for 16 hours, cooled, filtered through celite®, and partitioned between ethyl acetate and water. The organic phase was washed with brine and dried over MgSO₄, filtered and concentrated to give the title compound.

### EXAMPLE 113C

### benzyl(1S 3S,4S)-4-[(tert-butoxymethyl)amino]-3-hydroxy-1-[4-(6-methoxy-2-pyridinyl)benzyl]-5-phenylpentylcarbamate

The product from Example 113B (0.28 mmol) was treated with TBAF solution in THF (1.4 mL, 1N), stirred at 25° C. for 16 hours, concentrated and partitioned between ethyl acetate and water. The organic phase was washed with brined, dried over MgSO₄, filtered and concentrated. The residue was chromatographed on silica gel eluting with 20% ethyl acetate in dichloromethane, to give the title compound (0.055 g, 31% yield).

### EXAMPLE 113D

### benzyl(1S,3S,4S)-4-amino-3-hydroxy-1-[4-(6-methoxy-2-pyridinyl)benzyl]-5-phenylpentylcarbamate

A solution of the product from Example 113C (0.093 g, 0.15 mmol) in THF (1 mL) was treated with an HCl solution (0.26 mL, 4 N in dioxane), stirred at 25° C. for 64 hours, and concentrated. The concentrate was treated with ethanol and concentrated several times to give the title compound as the hydrochloride salt.

### EXAMPLE 113E

### benzyl(1S,3S,4S)-4[((2S)-3,3-dimethyl-2-{3-[(6-methyl-2-pyridinyl)methyl]-2-oxo 1-imidazolidinyl}butanoyl)amino]-3-hydroxy-1-[4-(6-methoxy-2-pyridinyl)benzyl]-5-phenylpentylcarbamate

A solution containing the product from Example 113D (0.049 mmol) in THF (0.5 mL) was treated with the product from Example 10D (0.017 g, 0.049 mmol), DEPBT (0.030 g, 0.099 mmol), and N,N-diisopropylethylamine (0.043 mL, 0.246 mmol), stirred at 25° C. for 5 hours, and partitioned between chloroform and 10% Na₂CO₃ solution. The organic phase was washed with additional 10% Na₂CO₃ solution and brine, dried over MgSO₄, filtered and concentrated.

### EXAMPLE 113F

### (2S)-N-{(1S,2S,4S)-4-amino-1-benzyl-2-hydroxy-5-[4-(6-methoxy-2-pyridinyl)phenyl]pentyl}-3,3-dimethyl-2-{3-[(6-methyl-2-pyridinyl)methyl]-2-oxo-1-imidazolidinyl}butanamide

A solution containing the product from Example 113E (0.049 mmol) in methanol (1 mL) was treated with Pd on carbon (0.005 g, 10% Pd by wt.) and HCl solution (0.050 mL, 4N in dioxane), stirred under a hydrogen atmosphere (balloon pressure) at 25° C. for 16 hours, filtered through a bed of celite®, rinsed with methanol, and concentrated to give the title compound as the hydrochloride salt.

### EXAMPLE 113G

### methyl(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-1-[4-(6-methoxy-2-pyridinyl)benzyl]-5-phenylpentyl}amino)carbonyl]-2,2-dimethylpropylcarbamate

A solution containing the product from Example 113F (0.049 mmol) in THF (0.5 mL) was treated with the product from Example 1F (0.010 g, 0.054 mmol), DEPBT (0.029 g, 0.098 mmol), and N,N-diisopropylethylamine (0.043 mL, 0.245 mmol), stirred at 25° C. for 16 hours, and partitioned between chloroform and 10% Na₂CO₃ solution. The organic phase was washed with additional 10% Na₂CO₃ solution and brine, dried over MgSO₄, filtered and concentrated. The residue was chromatographed on silica gel eluting with 2% methanol in chloroform. The product was purified by reversed phase chromatography on a C18 column eluting with 20-100% acetonitrile in water (0.1% TFA). The product was partitioned between ethyl acetate and saturated NaHCO₃, and the organic phase was washed with brine and dried over MgSO₄, filtered and concentrated to give the title compound (0.0065 g, 16% yield). ¹H NMR (300 MHz, CDCl₃) δ ppm 0.94(s, 9H), 0.98(s, 9H), 1.77-1.63(m, 2H), 2.69-2.59(m, 1H), 2.59(bs, 3H), 2.86-2.80(m, 4H), 3.21-3.04(m, 2H), 3.41-3.34(m, 1H), 3.62(s, 3H), 3.77-3.72(m, 2H), 4.02(s, 1H), 4.03(s, 3H), 4.34-4.16(m, 2H), 4.64-4.46(m, 2H), 5.36-5.34(d, J=7.72 Hz, 1H), 6.04-6.01(d, J=7.35 Hz, 1H), 6.43-6.40(d, J=8.82 Hz, 1H), 6.68-6.66(d, J=7.72 Hz, 1H), 7.33-7.09(m, 10H), 7.64-7.59(m, 2H), 7.95-7.92(d, J=8.09 Hz, 2H).

### EXAMPLE 114A

### benzyl(1S,3S,4S)-4-{[(2S)-2-(3-benzyl-2-oxo-1-imidazolidinyl)-3,3-dimethylbutanoyl]amino}-3-hydroxy-1-[4-(6-methoxy-2-pyridinyl)benzyl]-5-phenylpentylcarbamate

A solution containing the product from Example 113D (0.049 mmol) in THF (0.5 mL) was treated with the product from Example 70A (0.016 g, 0.055 mmol), DEPBT (0.030 g, 0.099 mmol), and N,N-diisopropylethylamine (0.043 mL, 0.246 mmol), stirred at 25° C. for 5 hours, and partitioned between chloroform and 10% Na₂CO₃ solution. The organic phase was washed with additional 10% Na₂CO₃ solution and brine, dried over MgSO₄, filtered and concentrated.

### EXAMPLE 114B

### (2S)-N-{(1S,2S,4S)-4-amino-1-benzyl-2-hydroxy-5-[4-(6-methoxy-2-pyridinyl)phenyl]pentyl}-2-(3-benzyl-2-oxo-1-imidazolidinyl)-3,3-dimethylbutanamide

A solution containing the product from Example 114A (0.049 mmol) in methanol (1 mL) was treated with Pd on carbon (0.005 g, 10% Pd by wt.) and HCl solution (0.050 mL, 4N in dioxane), stirred under a hydrogen atmosphere (balloon pressure) at 25° C. for 3 hours, filtered through a bed of celite®, rinsed with methanol, and concentrated to give the title compound as the hydrochloride salt.

### EXAMPLE 114C

### methyl(1S)-1-[({(1S,3S,4S)-4-{[(2S)-2-(3-benzyl-2-oxo-1-imidazolidinyl)-3,3-dimethylbutanoyl]amino}-3-hydroxy-1-[4-(6-methoxy-2-pyridinyl)benzyl]-5-phenylpentyl}amino)carbonyl]-2,2-dimethylpropylcarbamate

A solution containing the product from Example 114B (0.049 mmol) in THF (0.5 mL) was treated with the product from Example 1F (0.010 g, 0.054 mmol), DEPBT (0.029 g, 0.098 mmol), and N,N-diisopropylethylamine (0.043 mL, 0.245 mmol), stirred at 25 C for 16 hours, and partitioned between chloroform and 10% Na₂CO₃ solution. The organic phase was washed with additional 10% Na₂CO₃ solution and brine, dried over MgSO₄, filtered and concentrated. The residue was chromatographed on silica gel eluting with 2% methanol in chloroform. The product was purified by reversed phase chromatography on a C18 column eluting with a gradient starting with 40-100% acetonitrile in water (0.1% TFA) and ending with acetonitrile. The product was partitioned between ethyl acetate and saturated NaHCO₃, and the organic phase was washed with brine and dried over MgSO₄, filtered and concentrated to give the title compound (0.0065 g, 16% yield). ¹H NMR (300 MHz, CDCl₃) δ ppm 0.94(s, 9H), 0.98(s, 9H), 1.77-1.63(m, 2H), 2.64-2.55(q, J=9.19 Hz, 1H), 2.95-2.81(m, 5H), 3.06-2.97(m, 1H), 3.37-3.30(m, 1H), 3.62(s, 3H), 3.78-3.71(m, 2H), 4.03(s, 4H), 4.31-4.16(m, 2H), 4.43-4.32(m, 2H), 5.38-5.35(d, J=7.35 Hz, 1H), 6.07-6.04(d, J=7.72 Hz, 1H), 6.43-6.40(d, J=9.19 Hz, 1H), 6.69-6.66(d, J=8.46 Hz, 1H), 7.18-7.06(m, 5H), 7.23-7.20(d, J=8.46 Hz, 2H), 7.37-7.26(m, 6H), 7.65-7.60(m, 1H), 7.95-7.92(d, J=8.46 Hz, 2H).

### EXAMPLE 115A

### 2,2-dimethyl-5-hexen-3-ol

A solution of trimethylacetaldehyde (10.2 mL, 90.9 mmol) in diethyl ether (200 mL) at 0° C. was treated with allylmagnesium bromide (100 mL, 1 M in ether), stirred at 0° C. for 1 hour, quenched with saturated ammonium chloride and extracted with diethyl ether. The organic phase was washed with brine and dried over Na₂SO₄, filtered and concentrated to give the title compound (11.6 g).

### EXAMPLE 115B

### Ethyl 5-(2-hydroxy-3,3-dimethylbutyl)-4,5-dihydro-3-isoxazolecarboxylate

A solution containing the product from Example 115A (7.83 g, 61.1 mmol) and ethyl chloroimidoacetate (20.4 g, 134.4 mmol) at 0° C. in diethyl ether (180 mL) was treated with a solution of triethylamine (24.7 mL, 177.1 mmol) in diethyl ether (200 mL) over 2 hours, stirred at 0° C. for 1 hour, filtered and concentrated. The concentrate was purified by chromatography on silica gel eluting with 10% ethyl acetate in dichloromethane to give the title compound (6.76 g).

### EXAMPLE 115C

### Ethyl 5-(3,3-dimethyl-2-oxobutyl)-4,5-dihydro-3-isoxazolecarboxylate

A solution of DMSO (3.94 mL, 55.6 mmol) in dichloromethane (90 mL) at −78° C. was treated dropwise with oxalyl chloride (20.8 mL, 2 M in dichloromethane), stirred for at −78° C. for 15 minutes, treated with a solution of the product from Example 115B (6.76 g, 27.8 mmol) in dichloromethane (230 mL) over 10 minutes, stirred at −78° C. for 20 minutes, treated opwise with triethylamine (16.7 mL, 119.5 mmol) dr at −78° C., and after 10 minutes the reaction was warmed to 0° C., stirred for an additional 10 minutes. The reaction mixture was quenched with water and partitioned between dichloromethane and water. The organic phase was washed with brine and dried over Na₂SO₄, filtered and concentrated. The residue was chromatographed on silica gel eluting with 5% ethyl acetate in chloroform to give the title compound (4.9 g, 73% yield).

### EXAMPLE 115D

### Ethyl 6-tert-butyl-2-pyridinecarboxylate

A solution of the product from Example 115C (4.95 g, 20.5 mmol) in ethanol (400 mL) was treated with Raney nickel (20.10 g) and 48% HBF₄ solution (4.13 mL), and the reaction was shaken under a hydrogen atmosphere (50 psi) at 25° C. for 1 hour. The reaction mixture was filtered, diluted with water, basified with dilute NaOH solution, and partitioned between dichloromethane and water. The organic phase was washed with brine and dried over MgSO₄, filtered and concentrated. The residue was chromatographed on silica gel eluting with 10% ethyl acetate in hexane to give the title compound (1.1 g, 26% yield).

### EXAMPLE 115E

### (6-tert-butyl-2-pyridinyl)methanol

A solution containing the product from Example 115D (1.1 g, 5.3 mmol) in THF (20 mL) at −30° C. was treated with a solution of lithium aluminum hydride (5.3 mL, 1 M in THF), stirred at −30° C. for 5 minutes, treated with water (0.20 mL), 15% NaOH (0.20 mL), and water (0.40 mL) sequentially, stirred for 15 minutes at 25° C., filtered, rinsed with ethyl acetate, and concentrated to give the title compound (0.88 g, quantitative).

### EXAMPLE 115F

### 6-tert-butyl-2-pyridinecarbaldehyde

A solution of DMSO (0.90 mL, 12.7 mmol) in dichloromethane (10 mL) at −78° C. was treated with oxalyl chloride (3.1 mL, 2 M in dichloromethane) dropwise, stirred for an additional 15 minutes at −78° C., treated with a solution of the product from Example 115E (0.88 g, 5.3 mmol) in dichloromethane (14 mL) over 10 minutes, stirred for 20 minutes, treated dropwise with triethylamine (3.6 mL, 26.1 mmol) at −78° C., stirred for 10 minutes, warmed to 0° C., stirred for an additional 10 minutes, quenched with water and partitioned between dichloromethane and water. The organic phase was washed with brine and dried over Na₂SO₄, filtered and concentrated. The residue was chromatographed on silica gel eluting with 5% ethyl acetate in chloroform to give the title compound (0.77 g, 88% yield).

### EXAMPLE 115G

### tert-butyl(2S)-2-{3-[(6-tert-butyl-2-pyridinyl)methyl]-2-oxo-1-imidazolidinyl}-3,3-dimethylbutanoate

A solution containing the product from Example 6F (1.14 g, 5.0 mmol) in dichloromethane (12 mL) was treated with the product from Example 115F (0.77 mL, 4.7 mmol) and MgSO₄ (2.27 g, 18.9 mmol), stirred at 25° C. for 16 hours, filtered and concentrated. A solution of the residue in methanol (18 mL) was treated with sodium borohydride (0.27 g, 7.1 mmol), stirred at 25° C. for 1 hour, quenched with acetone (6 mL) and concentrated. The concentrate was partitioned between ethyl acetate and saturated NaHCO₃, and the organic phase was washed with brine and dried over Na₂SO₄, filtered and concentrated. A solution of the residue (4.7 mmol) in 1,2-dichloroethane (18 mL) was treated with N,N-disuccinimidyl carbonate (1.45 g, 5.70 mmol) and triethylamine (0.66 mL, 4.70 mmol), stirred at 25° C. for 16 hours, and partitioned with 10% NaHCO₃. The aqueous phase was extracted with additional dichloromethane. The combined organic phase was washed with brine, dried over Na₂SO₄, filtered and concentrated. The residue was chromatographed on silica gel eluting with 2% methanol in chloroform to give the title compound (1.42 g, 75% yield).

### EXAMPLE 115H

### (2S)-2-{3-[(6-tert-butyl-2-pyridinyl)methyl]-2-oxo-1-imidazolidinyl}-3,3-dimethylbutanoic Acid

A solution containing the product from Example 115G (1.27 g, 3.15 mmol) in dichloromethane (6 mL) was treated with trifluoracetic acid (3 mL), stirred at 25° C. for 3 hours, and concentrated. The residue was dissolved in ethyl acetate and concentrated several times to give the crude product as the trifluoroacetic acid salt.

### EXAMPLE 115I

### methyl(1S)-1-[({(1S,3S,4S)-4-[((2S)-2-{3-[(6-tert-butyl-2-pyridinyl)methyl]-2-oxo-1-imidazolidinyl}-3,3-dimethylbutanoyl)amino]-3-hydroxy-5-phenyl-1-[4-(2-pyridinyl)benzyl]pentyl}amino)carbonyl]-2,2-dimethylpropylcarbamate

A solution containing the product from Example 2C (0.035 g, 0.066 mmol) in THF (0.66 mL) was treated with the product from Example 115H (0.035 g, 0.079 mmol), DEPBT (0.029 g, 0.098 mmol), and N,N-diisopropylethylamine (0.012 mL, 0.069 mmol), stirred at 25° C. for 16 hours, and partitioned between chloroform and 10% Na₂CO₃ solution. The organic phase was washed with additional 10% Na₂CO₃ solution and brine, dried over MgSO₄, filtered and concentrated. The residue was purified by chromatography on silica gel eluting with 0-100% ethyl acetate/dichloromethane, followed by 0-5% methanol in ethyl acetate to give the title compound (0.023 g, 40% yield). ¹H NMR (300 MHz, DMSO-d₆) δ ppm 0.83(s, 9H), 0.90(s, 9H), 1.31(s, 9H), 1.60-1.47(m, 2H), 2.37-2.34(m, J=8.82 Hz, 1H), 2.61-2.53(m, 1H), 2.68-2.65(d, J=7.35 Hz, 2H), 2.80-2.76(m, 1H), 3.03-2.98(m, 1H), 3.24-3.16(m, 1H), 3.50(s, 3H), 3.70-3.60(m, 1H), 3.86-3.83(d, J=9.56 Hz, 1H), 4.08(s, 1H), 4.26-4.10(m, 2H), 4.47-4.33(m, 2H), 4.55-4.52(d, J=7.72 Hz, 1H), 6.67-6.63(d, J=9.93 Hz, 1H), 7.11-7.03(m, 6H), 7.23-7.21(d, J=8.09 Hz, 2H), 7.33-7.30(m, 2H), 7.49-7.45(d, J=9.56 Hz, 1H), 7.75-7.69(t, J=7.72 Hz, 1H), 7.91-7.82(m, 5H), 8.64-8.63(d, J=4.78 Hz, 1H).

### EXAMPLE 116

### methyl(1S,4S,5S,7S,10S)-1,10-ditert-butyl-5-hydroxy-2,9,12-trioxo-4,7-bis[4-(2-pyridinyl)benzyl]-13-oxa-3,8,11-triazatetradec-1-ylcarbamate

(i) A solution of the product from Example 112D (14 mg, 18 μmol) and thiocarbonyldiimidazole (10 mg, 56 μmol) in anhydrous THF (0.3 mL) was stirred at 60° C. 3 days. The solvent was concentrated, and the crude product was purified on by column chromatography on silica gel, eluting with 50-80% ethyl acetate in hexanes (7.7 mg, 52%).

(ii) A solution of the product from step (i) in anhydrous toluene (0.2 mL) was treated with tributyltin hydride (5 μL, 17 μmol) and 2,2′-azobisisobutyronitrile (2 mg, 12 μmol). The resulting mixture was heated at reflux for 90 min, cooled to 25° C., and the crude product was purified by column chromatography on silica gel, eluting with 50-100% ethyl acetate in hexanes (2.6 mg, 36%). ¹H NMR (300 MHz, CDCl₃) δ ppm 0.89(m, 18H), 2.34(m, 2H), 2.88 (m, 4H), 3.62(s, 6H), 3.80(m, 2H), 4.00(m, 1H), 4.18(m, 1H), 5.33(m, 2H), 6.08(m, 1H), 6.21(m, 1H), 7.31(m, 2H), 7.72(m, 6H), 7.89(m, 4H), 8.67(m, 2H).

### EXAMPLE 117A

### tert-butyl(2S)-2-3-[(6-acetyl-2-pyridinyl)methyl]-2-oxo-1-imidazolidinyl}-3,3-dimethylbutanoate

A solution of the product from Example 17D (1.95 g, 4.815 mmol) in tetrahydrofuran (50 mL) at −78° C. was treated methylmagnesium bromide in butyl ether (5.7 mL, 1 M). The mixture was stirred 0.5 hours at −78° C., quenched with acetone (3 mL) and 10% citric acid. The reaction mixture was partitioned between ethyl acetate and 1 N NaHCO₃, and the organic phase layer was decanted and concentrated. The residue was purified by flash chromatography on silica gel eluting with 25%-50% ethyl acetate in hexane give the title compound (1.6 g, 85% yield).

### EXAMPLE 117B

### tert-butyl(2S)-2-{3-[(6-isopropenyl-2-pyridinyl)methyl]-2-oxo-1-imidazolidinyl}-3,3-dimethylbutanoate

A solution of methyltriphenylphosphonium bromide (0.33 g, 0.923 mmol) in THF (2.5 mL) was treated with a solution of potassium tert-butoxide in THF (0.89 mL, 1 M) dropwise, stirred for 1 hour at 25° C., treated with a solution of the product from Example 117A (0.116 g, 0.298 mmol) in THF (2 mL), stirred at 25° C. for 16 hours, quenched with saturated ammonium chloride solution and partitioned between ethyl acetate and water. The organic phase was washed with brine and dried over MgSO₄, filtered and concentrated. The residue was chromatographed on silica gel eluting with 15%-25% ethyl acetate in hexane to give the title compound (0.040 g, 35% yield).

### EXAMPLE 117C

### tert-butyl(2S)-2-{3-[(6-isopropyl-2-pyridinyl)methyl]-2-oxo-1-imidazolidinyl}-3,3-dimethylbutanoate

A solution containing the product from Example 117B (0.038 g, 0.098 mmol) in methanol (1 mL) was treated with 10% Pd on carbon (0.005 g) and the reaction was stirred under an atmosphere of hydrogen (balloon pressure) for 2 hours. The reaction was filtered and the solvent was concentrated to give the title compound, which was used without further purification.

### EXAMPLE 117D

### (2S)-2-{3-[(6-isopropyl-2-pyridinyl)methyl]-2-oxo-1-imidazolidinyl}-3,3-dimethylbutanoic Acid

A solution of the product from Example 117C (0.098 mmol) in dichloromethane (0.5 mL) was treated with trifluoroacetic acid (0.5 mL) and the mixture was stirred for 1 hour at 25° C. The solvent was removed under reduced pressure and the residue was purified by reversed phase chromatography on a C18 column eluting with 5-100% acetonitrile in water (0.1% TFA) to give the title compound as the trifluroacetic acid salt (0.022 g, 52% yield).

### EXAMPLE 117E

### methyl(1S)-1-[({(1R,3S,4S)-3-hydroxy-4-[((2S)-2-{3-[(6-isopropyl-2-pyridinyl)methyl]-2-oxo-1-imidazolidinyl 1-3,3-dimethylbutanoyl)amino]-5-phenyl-1-[4-(2-pyridinyl)benzyl]pentyl}amino)carbonyl]-2,2-dimethylpropylcarbamate

A solution containing the product from Example 1H (0.025 g, 0.046 mmol) in THF (0.5 mL) was treated with the product from Example 117D (0.022 g, 0.051 mmol), DEPBT (0.021 g, 0.070 mmol), and N,N-diisopropylethylamine (0.057 mL, 0.325 mmol), stirred at 25° C. for 16 hours, and partitioned between chloroform and 10% Na₂CO₃ solution. The organic phase was washed with additional 10% Na₂CO₃ solution and brine, dried over MgSO₄, filtered and concentrated. The product was purified by chromatography on silica gel eluting with 0-100% ethyl acetate/dichloromethane, followed by 0-5% methanol in ethyl acetate to give the title compound (0.024 g, 62% yield). ¹H NMR (300 MHz, DMSO-d6) δ ppm 0.80(s, 9H), 0.88(s, 9H), 1.27-1.25(d, J=6.99 Hz, 6H), 1.42-1.23(m, 1H), 1.58-1.47(m, 1H), 2.70-2.53(m, 3H), 2.87-2.78(m, 1H), 3.31-2.98(m, 4H), 3.57-3.50(m, 1H), 3.57(s, 3H), 3.85-3.82(d, J=9.56 Hz, 1H), 3.99-3.87(m, 1H), 4.03(s, 1H), 4.20-4.10, 4.23-4.13(m, 2H), 4.45(s, 2H), 6.91-6.87(d, J=9.93 Hz, 1H), 7.10-7.06(m, 5H), 7.28-7.21(m, 4H), 7.44-7.37(m, 2H), 7.58-7.55(d, J=9.19 Hz, 1H), 7.97-7.86(m, 5H), 8.69-8.67(m, 1H).

### EXAMPLE 118

### methyl(1S)-1-[({(1R,3S,4S)-4-[((2S)-2-{3-[(6-tert-butyl-2-pyridinyl)methyl]-2-oxo-1-imidazolidinyl}-3,3-dimethylbutanoyl)amino]-3-hydroxy-5-phenyl-1-[4-(2-pyridinyl)benzyl]pentyl}amino)carbonyl]-2,2-dimethylpropylcarbamate

A solution containing the product from Example 1H (0.019 g, 0.036 mmol) in THF (0.43 mL) was treated with the product from Example 115H (0.019 g, 0.043 mmol), DEPBT (0.016 g, 0.054 mmol), and N,N-diisopropylethylamine (0.063 mL, 0.360 mmol), stirred at 25° C. for 16 hours, and partitioned between chloroform and 10% Na₂CO₃ solution. The organic phase was washed with additional 10% Na₂CO₃ solution and brine, dried over MgSO₄, filtered and concentrated. The residue was purified by chromatography on silica gel eluting with 0-100% ethyl acetate/dichloromethane, followed by 0-5% methanol in ethyl acetate to give the title compound (0.012 g, 39% yield). ¹H NMR (300 MHz, DMSO-d₆) δ ppm 0.80(s, 9H), 0.88(s, 9H), 1.30(s, 9H), 1.42-1.23(m, 1H), 1.58-1.47(m, 1H), 2.47-2.42(m, 1H), 2.73-2.55(m, 2H), 2.87-2.78(m, 1H), 3.05-2.99(m, 1H), 3.31-3.18(m, 1H), 3.57-3.50(m, 1H), 3.57(s, 3H), 3.85-3.82(d, J=9.56 Hz, 1H), 3.99-3.87(m, 1H), 4.02(s, 1H), 4.23-4.13(m, 1H), 4.44-4.32(m, 2H), 4.44-4.42(d, J=7.35 Hz, 1H), 6.90-6.87(d, J=9.19 Hz, 1H), 7.09-7.04(m, 6H), 7.25-7.22(d, J=8.46 Hz, 2H), 7.34-7.29(m, 2H), 7.54-7.51(d, J=9.91 Hz, 1H), 7.73-7.68(t, J=7.72 Hz, 1H), 7.90-7.83(m, 3H), 7.97-7.94(d, J=8.09 Hz, 2H), 8.65-8.64(m, 1H).

### EXAMPLE 119A

### benzyl(4S,5S)-5-{(2S)-2-[(tert-butoxycarbonyl)amino]-3-phenylpropyl}-4-[4-(6-methoxy-2-pyridinyl)benzyl]-2,2-dimethyl-1,3-oxazolidine-3-carboxylate

A solution containing the product from Example 23I (0.20 g, 0.28 mmol) in DMF (3 mL) was treated with LiCl (0.119 g, 2.8 mmol), dichlorobis(triphenylphosphine)palladium(II) (0.060 g, 0.085 mmol), and the product from Example 113A (0.338 g, 0.85 mmol), heated at 85C for 64 hours, cooled and partitioned between ethyl acetate and water. The organic phase was washed with brine and dried over MgSO₄, filtered and concentrated. The residue was chromatographed on silica gel eluting with 20% ethyl acetate in hexanes to give the title compound (0.097 g, 51% yield).

### EXAMPLE 119B

### benzyl(1S,2S,4S)-4-amino-2-hydroxy-1-[4-(6-methoxy-2-pyridinyl)benzyl]-5-phenylpentylcarbamate

A solution containing the product from Example 119A (0.095 g, 0.14 mmol) in THF (1 mL) was treated with a solution of HCl in dioxane (0.25 mL, 4 N), stirred at 50° C. for 16 hours, cooled and concentrated under reduced pressure. The residue was dissolved in ethanol and concentrated several times to give the title compound as hydrochloride salt, which was used without further purification.

### EXAMPLE 119C

### benzyl(1S,2S,4S)-4-{[(2S)-2-(3-benzyl-2-oxo-1-imidazolidinyl)-3,3-dimethylbutanoyl]amino}-2-hydroxy-1-[4-(6-methoxy-2-pyridinyl)benzyl]-5-phenylpentylcarbamate

A solution containing the product from Example 119B (0.048 mmol) in THF (2 mL) was treated with the product from Example 70A (0.014 g, 0.048 mmol), DEPBT (0.029 g, 0.095 mmol), and N,N-diisopropylethylamine (0.042 mL, 0.235 mmol), stirred at 25° C. for 5 hours. The mixture was partitioned between chloroform and 10% Na₂CO₃ solution. The organic phase was washed with additional 10% Na₂CO₃ solution and brine, dried over MgSO₄, filtered and concentrated. The residue was chromatographed on silica gel eluting with 2% methanol in chloroform to give the title compound (0.024 g, 62% yield).

### EXAMPLE 119D

### (2S)-N-{(1S,3S,4S)-4-amino-1-benzyl-3-hydroxy-5-[4-(6-methoxy-2-pyridinyl)phenyl]pentyl}-2-(3-benzyl-2-oxo-1-imidazolidinyl)-3,3-dimethylbutanamide

A solution containing the product from Example 119C (0.024 g, 0.030 mmol) in methanol (1 mL) was treated with 10% Pd on carbon (0.003 g) and HCl solution (0.030 mL, 4 N in dioxane), stirred under a hydrogen atmosphere (balloon pressure) at 25° C. for 16 hours, filtered through a bed of celite and rinsed with methanol. The solvent was concentrated to give the crude product as a hydrochloride salt, which was used without further purification.

### EXAMPLE 119E

### methyl(1S)-1-[({(1S,2S,4S)-4-{[(2S)-2-(3-benzyl-2-oxo-1-imidazolidinyl)-3,3-dimethylbutanoyl]amino}-2-hydroxy-1-[4-(6-methoxy-2-pyridinyl)benzyl]-5-phenylpentyl}amino)carbonyl]-2,2-dimethylpropylcarbamate

A solution containing the product from Example 119D (0.030 mmol) in THF (1 mL) was treated with the product from Example 1F (0.006 g, 0.033 mmol), DEPBT (0.018 g, 0.059 mmol), and N,N-diisopropylethylamine (0.026 mL, 0.148 mmol), stirred at 25° C. for 16 hours, and partitioned between chloroform and 10% Na₂CO₃ solution. The organic phase was washed with additional 10% Na₂CO₃ solution and brine, dried over MgSO₄, filtered and concentrated. The residue was purified by chromatography on silica gel eluting with 2% methanol in chloroform, to give the title compound. ¹H NMR (300 MHz, CDCl₃) δ ppm 0.96(s, 9H), 1.00(s, 9H), 1.67-1.55(m, 2H), 2.67-2.60(m, 1H), 2.96-2.73m, 5H), 3.08-2.99q, J=8.46 Hz, 1H), 3.43-3.36(m, 1H), 3.62(bs, 4H), 3.82-3.79(d, J=8.82 Hz, 1H), 4.00(s, 1H), 4.04(s, 3H), 4.16-4.0Sm, 2H), 4.45-4.25(m, 2H), 5.34-5.27(m, 1H), 6.12-6.09(m, 2H), 6.69-6.66(d, J=8.09 Hz, 1H), 7.15-7.06(m, 6H), 7.36-7.23(m, 7H), 7.65-7.59(m, 1H), 7.95-7.92(d, J=8.09 Hz, 2H).

### EXAMPLE 120A

### benzyl(1S,2S,4S)-2-hydroxy-4-({(2S)-2-[(methoxycarbonyl)amino]-3,3-dimethylbutanoyl}amino)-1-[4-(6-methoxy-2-pyridinyl)benzyl]-5-phenylpentylcarbamate

A solution containing the product from Example 119B (0.048 mmol) in THF (2 mL) was treated with the product from Example 1F (0.009 g, 0.048 mmol), DEPBT (0.029 g, 0.095 mmol), and N,N-diisopropylethylamine (0.042 mL, 0.235 mmol), stirred at 25° C. for 5 hours, and partitioned between chloroform and 10% Na₂CO₃ solution. The organic phase was washed with additional 10% Na₂CO₃ solution and brine, dried over MgSO₄, filtered and concentrated. The residue was chromatographed on silica gel eluting with 2% methanol in chloroform to give the title compound (0.028 g, 85% yield).

### EXAMPLE 120B

### methyl(1S)-1-[({(1S,3S,4S)-4-amino-1-benzyl-3-hydroxy-5-[4-(6-methoxy-2-pyridinyl)phenyl]pentyl}amino)carbonyl]-2,2-dimethylpropylcarbamate

A solution containing the product from Example 120A (0.028 g, 0.041 mmol) in methanol (1 mL) was treated with 10% Pd on carbon (0.003 g) and HCl solution (0.030 mL, 4 N in dioxane), and the reaction was stirred under a hydrogen atmosphere (balloon pressure) at 25° C. for 16 hours. The reaction was filtered through a bed of celite and rinsed with methanol. The solvent was concentrated to give the title compound as the hydrochloride salt, which was used without further purification.

### EXAMPLE 120C

### methyl(1S,4S,5S,7S,10S)-7-benzyl-1,10-ditert-butyl-5-hydroxy-4-[4-(6-methoxy-2-pyridinyl)benzyl]-2,9,12-trioxo-13-oxa-3,8,11-triazatetradec-1-ylcarbamate

A solution containing the product from Example 120B (0.041 mmol) in THF (1 mL) was treated with the product from Example 1F (0.009 g, 0.045 mmol), DEPBT (0.024 g, 0.082 mmol), and N,N-diisopropylethylamine (0.036 mL, 0.204 mmol) and the mixture was stirred at 25° C. for 16 hours. The mixture was partitioned between chloroform and 10% Na₂CO₃ solution. The organic phase was washed with additional 10% Na₂CO₃ solution and brine, dried over MgSO₄, filtered and concentrated. The residue was purified by chromatography on silica gel eluting with 2% methanol in chloroform, to give the title compound. ¹H NMR (300 MHz, CDCl₃) δ ppm 0.91(s, 9H), 0.94(s, 9H), 1.67-1.54(m, 2H), 2.80-2.74(m, 2H), 2.89-2.87(d, J=7.35 Hz, 2H), 3.62(s, 3H), 3.67(s, 3H), 3.74-3.61(m, 2H), 3.82-3.79(d, J=9.19 Hz, 1H), 4.00-3.93(m, 1H), 4.04(s, 3H), 4.13-4.04(m, 1H), 5.32-5.28(m, 2H), 5.96-5.94(d, J=6.99 Hz, 1H), 6.14-6.11(d, J=8.82 Hz, 1H), 6.69-6.67(d, J=7.72 Hz, 1H), 7.08-7.06(d, J=6.62 Hz, 2H), 7.33-7.15(m, 6H), 7.66-7.60(m, 1H), 7.95-7.92(d, J=8.09 Hz, 2H).

### EXAMPLE 121

### methyl(1S,4S,5S,7S,10S)-4-benzyl-1,10-ditert-butyl-5-hydroxy-7-[4-(6-methoxy-2-pyridinyl)benzyl]-2,9,12-trioxo-13-oxa-3,8,11-triazatetradec-1-ylcarbamate

A solution of the product from Example 113C (0.074 g, 0.12 mmol) in THF (2 mL) was treated with an HCl solution (0.21 mL, 4 N in dioxane), and the reaction was stirred at 50° C. for 16 hours. The solvent was removed under reduced pressure and ethanol added and concentrated several times. A solution of the concentrate (0.12 mmol) in methanol (2 mL) was treated with Pd on carbon (0.007 g, 10% Pd by wt.), and the reaction was stirred under a hydrogen atmosphere (balloon pressure) at 25° C. for 16 hours. The reaction mixture was filtered through a bed of celite®, rinsed with methanol, and concentrated. A solution of the concentrate (0.12 mmol) in THF (0.5 mL) was treated with the product from Example 1F (0.047 g, 0.25 mmol), DEPBT (0.142 g, 0.47 mmol), and N,N-diisopropylethylamine (0.207 mL, 1.19 mmol), stirred at 25° C. for 16 hours, and partitioned between chloroform and 10% Na₂CO₃ solution. The organic phase was washed with additional 10% Na₂CO₃ solution and brine, dried over MgSO₄, filtered and concentrated. The residue was chromatographed on silica gel eluting with 1.5% methanol in chloroform, to give the title compound. ¹H NMR (300 MHz, CDCl₃) δ ppm 0.93(s, 18H), 1.65-1.58(m, 2H), 2.90-2.74(m, 4H), 3.63(s, 3H), 3.68(s, 3H), 3.80-3.63(m, 3H), 3.98-3.92(m, 1H), 4.04(s, 3H), 4.20-4.11(m, 1H), 5.32-5.35(m, 2H), 6.02-6.00(d, J=8.09 Hz, 1H), 6.11-6.08(d, J=8.82 Hz, 1H), 6.69-6.67(d, J=7.72 Hz, 1H), 7.33-7.14(m, 8H), 7.65-7.60(m, 1H), 7.94-7.91(d, J=8.09 Hz, 2H).

### EXAMPLE 122A

### benzyl(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methoxy-2-pyridinyl)benzyl]-5-phenylpentylcarbamate

A solution containing the product from Example 119B (0.048 mmol) in THF (2 mL) was treated with the product from Example 10D (0.016 g, 0.048 mmol), DEPBT (0.029 g, 0.095 mmol), and N,N-diisopropylethylamine (0.042 mL, 0.235 mmol), stirred at 25° C. for 5 hours, and partitioned between chloroform and 10% Na₂CO₃ solution. The organic phase was washed with additional 10% Na₂CO₃ solution and brine, dried over MgSO₄, filtered and concentrated. The residue was chromatographed on silica gel eluting with 2% methanol in chloroform to give the title compound (0.018 g, 47% yield).

### EXAMPLE 122B

### (2S)-N-{(1S,3S,4S)-4-amino-1-benzyl-3-hydroxy-5-[4-(6-methoxy-2-pyridinyl)phenyl]pentyl}-3,3-dimethyl-2-{3-[(6-methyl-2-pyridinyl)methyl]-2-oxo-1-imidazolidinyl}butanamide

A solution containing the product from Example 122A (0.018 g, 0.022 mmol) in methanol (1 mL) was treated with 10% Pd on carbon (0.002 g) and HCl solution (0.030 mL, 4 N in dioxane), stirred under a hydrogen atmosphere (balloon pressure) at 25° C. for 16 hours, filtered through a bed of celite and rinsed with methanol. The solvent was concentrated to give the crude product as the hydrochloride salt, which was used without further purification.

### EXAMPLE 122C

### methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methoxy-2-pyridinyl)benzyl]-5-phenylpentyl}amino)carbonyl]-2,2-dimethylpropylcarbamate

A solution containing the product from Example 122B (0.022 mmol) in THF (1 mL) was treated with the product from Example 1F (0.005 g, 0.024 mmol), DEPBT (0.013 g, 0.044 mmol), and N,N-diisopropylethylamine (0.020 mL, 0.11 mmol), stirred at 25° C. for 16 hours, and partitioned between chloroform and 10% Na₂CO₃ solution. The organic phase was washed with additional 10% Na₂CO₃ solution and brine, dried over MgSO₄, filtered and concentrated. The residue was purified by chromatography on silica gel eluting with 2% methanol in chloroform, to give the title compound. ¹H NMR (300 MHz, CDCl₃) δ ppm 0.96(s, 9H), 1.01(s, 9H), 1.67-1.59(m, 2H), 2.55(s, 3H), 2.67-2.59(m, 1H), 2.84-2.73(m, 2H), 2.91-2.89(d, J=7.72 Hz, 2H), 3.10-3.02(m, 1H), 3.23-3.14(q, J=8.95 Hz, 1H), 3.46-3.39(m, 1H), 3.68-3.62(m, 1H), 3.62(s, 3H), 3.82-3.79(d, J=9.19 Hz, 1H), 3.99(s, 1H), 4.04(s, 3H), 4.17-4.07(m, 2H), 4.59-4.34(m, 2H), 5.32-5.29(d, J=8.46 Hz, 1H), 6.12-6.09(d, J=9.19 Hz, 1H), 6.21-6.11(m, 1H), 6.68-6.66(d, J=7.72 Hz, 1H), 7.14-7.01(m, 7H), 7.33-7.27(m, 3H), 7.58-7.53(t, J=7.72 Hz, 1H), 7.64-7.61(m, 1H), 7.95-7.93(d, J=8.46 Hz, 2H).

### EXAMPLE 123A

### (2S)-3,3-dimethyl-2-[3-(2-nitrobenzyl)-2-oxo-1-imidazolidinyl]butanoic Acid

A solution containing the product from Example 6F (0.162 g, 0.702 mmol) in a mixture of benzene (3.5 mL) and methanol (3.5 mL) was treated with 2-nitrobenzaldehyde (0.112 mL, 0.737 mmol), stirred at 50° C. for 16 hours, cooled to 25° C., treated with sodium borohydride (0.053 g, 1.4 mmol), stirred at 25° C. for 2 hours, quenched with saturated NaHCO₃, and partitioned between ethyl acetate and saturated NaHCO₃. The organic phase was washed with brine and dried over MgSO₄, filtered and concentrated. A solution of the concentrate (0.702 mmol) in 1,2-dichloroethane (7 mL) was treated with N,N-disuccinimidyl carbonate (0.216 g, 0.842 mmol) and triethylamine (0.117 mL, 0.842 mmol), stirred at 25° C. for 16 hours, diluted with dichloromethane and partitioned with 10% Na₂CO₃. The organic phase was washed with brine, dried over MgSO₄, filtered and concentrated. A solution of the concentrate (0.702 mmol) in dichloromethane (3.5 mL) was treated with trifluoracetic acid (3.5 mL), stirred at 25° C. for 2 hours and concentrated. The residue was purified by reversed phase chromatography on a C18 column eluting with a gradient starting with 5-100% acetonitrile in water (0.1% TFA), to give the title compound (0.12 g, 50% yield).

### EXAMPLE 123B

### methyl(1S)-1-[({(1S,3S,4S)-4-({(2S)-3,3-dimethyl-2-[3-(2-nitrobenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 2C (0.082 g, 0.154 mmol) in THF (1.5 mL) was treated with the product from Example 123A (0.057 g, 0.17 mmol), DEPBT (0.069 g, 0.231 mmol), and N,N-diisopropylethylamine (0.135 mL, 0.77 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% yl acetate/dichloromethane, followed by 0-5% methanol in ethyl acetate to give the title compound (0.080 g, 61% yield).

### EXAMPLE 123C

### methyl(1S)-1-[({(1S,3S,4S)-4-({(2)-2-[3-(2-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate

A solution containing the product from Example 123B (0.027 g, 0.031 mmol) in ethanol (1 mL) was treated with 10% Pd on carbon (0.010 g), stirred under an atmosphere of hydrogen (balloon pressure) at 25° C. for 2.5 hours, filtered and concentrated under reduced pressure. The residue was purified by reversed phase chromatography on a C18 column eluting with 5-100% acetonitrile in water (0.1% TFA). The product was partitioned between ethyl acetate and saturated NaHCO₃, and the organic phase was washed with brine and dried over MgSO₄, filtered and concentrated to give the title compound (0.014 g, 55% yield). ¹H NMR (300 MHz, DMSO-d₆) δ ppm 0.84(s, 9H), 0.86(s, 9H), 1.63-1.46(m, 2H), 2.16-2.07(m, 1H), 2.65-2.54(m, 3H), 3.00-2.74(m, 3H), 3.18-3.08(m, 1H), 3.50(s, 3H), 3.71-3.62(m, 1H), 3.87-3.83(d, J=9.56 Hz, 1H), 4.05(s, 1H), 4.28-4.10(m, 4H), 4.53-4.51(d, J=7.72 Hz, 1H), 5.20(s, 2H), 6.56-6.51(t, J=7.35 Hz, 1H), 6.68-6.64(m, 2H), 7.08-6.92(m, 7H), 7.24-7.21(d, J=8.09 Hz, 2H), 7.33-7.29 (m, 1H), 7.43-7.40(d, J=9.93 Hz, 1H), 7.91-7.82(m, 5H), 8.64-8.63(d, J=4.41 Hz, 1H).

### EXAMPLE 124A

### (2S)-3,3-dimethyl-2-[3-(4-nitrobenzyl)-2-oxo-1-imidazolidinyl]butanoic Acid

A solution containing the product from Example 6F (0.161 g, 0.700 mmol) in a mixture of benzene (3.5 mL) and methanol (3.5 mL) was treated with 4-nitrobenzaldehyde (0.111 mL, 0.735 mmol), stirred at 50° C. for 16 hours, cooled to 25° C., treated with sodium borohydride (0.053 g, 1.4 mmol), stirred at 25° C. for 2 hours, quenched with saturated NaHCO₃, and partitioned between ethyl acetate and saturated NaHCO₃. The organic phase was washed with brine and dried over MgSO₄, filtered and concentrated. A solution of the concentrate (0.700 mmol) in 1,2-dichloroethane (7 mL) was treated with N,N-disuccinimidyl carbonate (0.215 g, 0.839 mmol) and triethylamine (0.117 mL, 0.842 mmol), stirred at 25° C. for 16 hours, diluted with dichloromethane and partitioned with 10% Na₂CO₃. The organic phase was washed with brine, dried over MgSO₄, filtered and concentrated. A solution containing of the concentrate (0.700 mmol) in dichloromethane (3 mL) was treated with trifluoracetic acid (3 mL), and the mixture was stirred at 25° C. for 2 hours. The solvent was concentrated, and the residue was purified by reversed phase chromatography on a C18 column eluting with a gradient starting with 5-100% acetonitrile in water (0.1% TFA), to give the title compound (0.17 g, 62% yield).

### EXAMPLE 124B

### methyl(1S)-1-[({(1S,3S,4S)-4-({(2S)-3,3-dimethyl-2-[3-(4-nitrobenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 2C (0.074 g, 0.138 mmol) in THF (1.5 mL) was treated with the product from Example 124A (0.051 g, 0.152 mmol), DEPBT (0.062 g, 0.207 mmol), and N,N-diisopropylethylamine (0.120 mL, 0.691 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate to give the title compound (0.066 g, 56% yield).

### EXAMPLE 124C

### methyl(1S)-1-[({(1S,3S,4S)-4-({(2S)-2-[3-(4-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate

A solution containing the product from Example 124B (0.065 g, 0.076 mmol) in ethanol (1.5 mL) was treated with 10% Pd on carbon (0.024 g), stirred under an atmosphere of hydrogen (balloon pressure) at 25° C. for 2.5 hours, filtered concentrated under reduced pressure. The residue was purified by reversed phase chromatography on a C18 column eluting with a gradient starting with 5-100% acetonitrile in water (0.1% TFA). The product was partitioned between ethyl acetate and saturated NaHCO₃, and the organic phase was washed with brine and dried over MgSO₄, filtered and concentrated to give the title compound (0.024 g, 39% yield). ¹H NMR (300 MHz, DMSO-d₆) δ ppm 0.83(s, 9H), 0.87(s, 9H), 1.60-1.48(m, 2H), 2.29-2.20(m, 1H), 2.67-2.53(m, 3H), 2.92-2.75(m, 3H), 3.16-3.08(m, 1H), 3.50(s, 3H), 3.71-3.61(m, 1H), 3.86-3.83(d, J=9.93 Hz, 1H), 4.07(s, 1H), 4.11(s, 2H), 4.23-4.09(m, 2H), 4.56-4.53(d, J=7.72 Hz, 1H), 5.00(s, 2H), 6.55-6.52(d, J=8.46 Hz, 2H), 6.67-6.64(d, J=9.93 Hz, 1H), 6.94-6.91(d, J=8.46 Hz, 2H), 7.10-7.02(m, 5H), 7.23-7.21(d, J=8.46 Hz, 2H), 7.33-7.29(m, 1H), 7.45-7.42(d, J=9.56 Hz, 1H), 7.91-7.82(m, 5H), 8.64-8.62(m, 1H).

### EXAMPLE 125A

### tert-butyl(2S)-3,3-dimethyl-2-[3-(3-nitrobenzyl)-2-oxo-1-imidazolidinyl]butanoate

A solution containing the product from Example 6F (0.215 g, 0.933 mmol) in a mixture of benzene (3 mL) and methanol (3 mL) was treated with 3-nitrobenzaldehyde (0.148 mL, 0.98 mmol), and the mixture was stirred at 50° C. for 16 hours, cooled to 25° C., treated with sodium borohydride (0.071 g, 1.88 mmol), stirred at 25° C. for 2 hours, quenched with saturated NaHCO₃, and partitioned between ethyl acetate and saturated NaHCO₃. The organic phase was washed with brine and dried over MgSO₄, filtered and concentrated. A solution of the concentrate (0.933 mmol) in 1,2-dichloroethane (9 mL) was treated with N,N-disuccinimidyl carbonate (0.287 g, 1.12 mmol) and triethylamine (0.156 mL, 1.12 mmol), stirred at 25° C. for 16 hours, diluted with dichloromethane and partitioned with 10% Na₂CO₃. The organic phase was washed with brine, dried over MgSO₄, filtered and concentrated. The residue was chromatographed on silica gel eluting with 0-25% ethyl acetate in hexane to give the title compound (0.209 g, 56% yield).

### EXAMPLE 125B

### (2S)-3,3-dimethyl-2-[3-(3-nitrobenzyl)-2-oxo-1-imidazolidinyl]butanoic Acid

A solution containing the product from Example 125B (0.209 g, 0.53 mmol) in dichloromethane (2.5 mL) was treated with trifluoracetic acid (2.5 mL), and the mixture was stirred at 25° C. for 1 hour. The solvent was concentrated and the residue was dissolved in ethyl acetate and concentrated several times to give the crude product, which was used without further purification.

### EXAMPLE 125C

### methyl(1S)-1-[({(1S,3S,4S)-4-({(2S)-3,3-dimethyl-2-[3-(3-nitrobenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate

A solution containing the product from Example 2C (0.065 g, 0.123 mmol) in THF (1 mL) was treated with the product from Example 125B (0.049 g, 0.147 mmol), DEPBT (0.055 g, 0.185 mmol), and N,N-diisopropylethylamine (0.102 mL, 0.615 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The residue was chromatographed on silica gel eluting with 0-100% ethyl acetate/dichloromethane, followed by 0-5% methanol in ethyl acetate to give the title compound (0.077 g, 74% yield).

### EXAMPLE 125D

### methyl(1S)-1-[({(1S,3S,4S)-4-({(2S)-2-[3-(3-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate

A solution containing the product from Example 125C (0.077 g, 0.091 mmol) in ethanol (2 mL) was treated with 10% Pd on carbon (0.029 g), stirred under an atmosphere of hydrogen (balloon pressure) at 25° C. for 3 hours, filtered and concentrated under reduced pressure. The residue was purified by reversed phase chromatography on a C18 column eluting with 5-100% acetonitrile in water (0.1% TFA. The product was partitioned between ethyl acetate and saturated NaHCO₃, and the organic phase was washed with brine and dried over MgSO₄, filtered and concentrated to give the title compound (0.035 g, 47% yield). ¹H NMR (300 MHz, DMSO-d₆) δ ppm 0.83(s, 9H), 0.89(s, 9H), 1.60-1.49(m, 2H), 2.38-2.28(m, 1H), 2.62-2.53(m, 1H), 2.68-2.66(m, 2H), 2.86-2.76(m, 2H), 2.98-2.89(q, J=9.19 Hz, 1H), 3.20-3.14(m, 1H), 3.50(s, 3H), 3.70-3.62(m, 1H), 3.86-3.83(d, J=9.93 Hz, 1H), 4.07(s, 1H), 4.14(s, 2H), 4.25-4.10(m, 2H), 4.55-4.53(d, J=7.35 Hz, 1H), 5.03(s, 2H), 6.42-6.39(d, J=7.35 Hz, 1H), 6.48-6.66(m, 2H), 6.62-6.59(d, J=9.93 Hz, 1H), 7.01-6.96(t, J=7.91 Hz, 1H), 7.11-7.04(m, 5H), 7.24-7.21(d, J=8.09 Hz, 2H), 7.33-7.28(m, 1H), 7.40-7.37(d, J=9.19 Hz, 1H), 7.91-7.80(m, 5H), 8.64-8.63(d, J=4.41 Hz, 1H).

### EXAMPLE 126

### tert-butyl(1S,3S,4S)-4-amino-1-benzyl-3-hydroxy-5-phenylpentylcarbamate, Succinate Salt

The title compound was prepared from L-phenylalanine using the procedures as described in U.S. Pat. No. 5,914,332, Examples 1A to 1F-2.

### EXAMPLE 127

### (2S,3S,5S)-2,5-diamino-1,6-diphenyl-3-hexanol

The title compound was prepared from Cbz-L-phenylalaninol using the procedures as described in Kempf, D. J.; Marsh K. C.; Codacovi Fino, L.; Bryant, P.; Craig-Kennard, A.; Sham, H. L.; Zhao, C.; Vasavanonda, S.; Kohlbrenner, W. E.; Wideburg, N. E.; Saldivar, A.; Green, B. E.; Herrin, T.; Norbeck, D. W. Biiorganic and Medicinal Chemistry 1994, 2, 847-858, and in Kempf, D. J.; Sowin, T. J.; Doherty, E. M.; Hannick, S. M.; Codavoci, L.; Henry, R. F.; Green, B. E.; Spanton, S. G.; Norbeck, D. W. Journal of Organic Chemistry 1992, 57, 5692-5700.

### EXAMPLE 128A

### benzyl(3S,4S)-1-(4-bromobenzyl)-4-[(tert-butoxycarbonyl)amino]-3-hydroxy-5-phenylpentylcarbamate

A solution of a mixture of the products from Examples 92D and Example 92E (prior to separation by chromatography) (2.4 g, 3.37 mmol) was treated with TBAF solution in THF (19 mL, 1N), stirred at 25° C. for 16 hours, concentrated, and partitioned between ethyl acetate and water. The organic was washed with brine, dried over MgSO₄, filtered and concentrated to give the product, which was used without further purification.

### EXAMPLE 128B

### tert-butyl(4S,5S)-4-benzyl-5-[2-{[(benzyloxy)carbonyl]amino}-3-(4-bromophenyl)propyl]-2,2-dimethyl-1,3-oxazolidine-3-carboxylate

A solution containing the product from Example 128A (3.37 mmol) in 2,2-dimethoxypropane (35 mL) was treated with p-toluenesulfonic acid monohydrate (0.032 g, 0.17 mmol), stirred at 25° C. for 1 hour, treated with triethylamine (0.14 mL, 1.0 mmol), and the reaction was partitioned between ethyl acetate and water. The organic phase was washed with brine, dried over MgSO₄, filtered and concentrated to give the product (1.16 g, 54% yield), which was used without further purification.

### EXAMPLE 128C

### tert-butyl(4S,5S)-4-benzyl-5-{2-{[(benzyloxy)carbonyl]amino)-3-[4-(5-methyl-2-pyridinyl)phenyl]propyl}-2,2-dimethyl-1,3-oxazolidine-3-carboxylate

A solution containing the product from Example 128B (0.50 g, 0.78 mmol) in DMF (8 mL) was treated with dichlorobis(triphenylphosphine)palladium(II) (0.165 g, 0.235 mmol), and the product from Example 74A (0.60 g, 1.63 mmol), stirred at 100° C. for 6 hours, cooled to 25° C., filtered through celite®, and partitioned between ethyl acetate and water. The organic was washed with brine, dried over MgSO₄, filtered and concentrated. The residue was chromatographed on silica gel eluting with 0-15% ethyl acetate in chloroform to give the product (0.322 g, 63% yield).

### EXAMPLE 128D

### benzyl(3S,4S)-4-amino-3-hydroxy-1-[4-(5-methyl-2-pyridinyl)benzyl]-5-phenylpentylcarbamate

A solution containing the product from Example 128C (0.322 g, 0.496 mmol) in a mixture of THF (5 mL), methanol (5 mL), and aqueous HCl (5 mL, 1 N) was stirred at 69C for 16 hours. The solvent was removed under reduced pressure to give the title compound as the hydrochloride salt.

### EXAMPLE 128E

### benzyl(3S,4S)-4-[((2S)-3,3-dimethyl-2-{3-[(6-methyl-2-pyridinyl)methyl]-2-oxo-1-imidazolidinyl}butanoyl)amino]-3-hydroxy-1-[4-(5-methyl-2-pyridinyl)benzyl]-5-phenylpentylcarbamate

A solution containing the product from Example 128D (0.496 mmol) in THF (5 mL) was treated with the product from Example 10D (0.205 g, 0.600 mmol), DEPBT (0.225 g, 0.753 mmol), and N,N-diisopropylethylamine (0.875 mL, 5.02 mmol), stirred at 25° C. for 3 hours, and partitioned between ethyl acetate and 10% Na₂CO₃ solution. The organic phase was washed with additional 10% Na₂CO₃ solution and then brine, dried over MgSO₄, filtered and concentrated. The residue was chromatographed on silica gel eluting with 0-100% ethyl acetate/dichloromethane, followed by 50% methanol in ethyl acetate to give the product (0.127 g, 32% yield).

### EXAMPLE 128F

### (2S)-N-{(1S,2S)-4-amino-1-benzyl-2-hydroxy-5-[4-(5-methyl-2-pyridinyl)phenyl]pentyl}-3,3-dimethyl-2-{3-[(6-methyl-2-pyridinyl)methyl]-2-oxo-1-imidazolidinyl}butanamide

A solution containing the product from Example 128E (0.127 g, 0.159 mmol) in methanol (2 mL) was treated with Pd(OH)₂ on carbon (0.035 g, 20% Pd by wt.) and HCl solution (0.12 mL, 4N in dioxane), stirred under a hydrogen atmosphere (balloon pressure) at 25° C. for 16 hours, filtered through a bed of celite® and rinsed with methanol. The solvent was concentrated to give the title compound as the hydrochloride salt, which was used without further purification.

### EXAMPLE 128G

### methyl(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-1-[4-(5-methyl-2-pyridinyl)benzyl]-5-phenylpentyl}amino)carbonyl]-2,2-dimethylpropylcarbamate

A solution containing the product from Example 128F (0.159 mmol) in THF (1.6 mL) was treated with the product from Example 1F (0.036 g, 0.190 mmol), DEPBT (0.075 g, 0.251 mmol), and N,N-diisopropylethylamine (0.30 mL, 1.72 mmol), stirred at 25° C. for 16 hours, and partitioned between ethyl acetate and 10% Na₂CO₃ solution. The organic phase was washed with additional 10% Na₂CO₃ solution and brine, dried over MgSO₄, filtered and concentrated. The product was purified by chromatography on silica gel eluting with 0-50% acetone in dichloromethane, to give the lower Rf (50% acetone in dichloromethane) product of the mixture (0.01 g). ¹H NMR (300 MHz, DMSO-d₆), δ ppm 0.83 (s, 9H), 0.90 (s, 9H), 1.55 (m, 2H), 2.32 (s, 3H), 2.38 (q, J=9.2 Hz, 1H), 2.46 (s, 3H), 2.57 (m, 1H), 2.67 (d, J=7.0 Hz, 2H), 2.79 (m, 1H), 2.97 (m, 1H), 3.09 (q, J=8.95 Hz, 1H), 3.21 (m, 1H), 3.51 (s, 3H), 3.67 (m, 1H), 3.85 (d, J=9.6 Hz, 1H), 4.08 (s, 1H), 4.12 (m, 3H), 4.35 (m, 2H), 4.54 (d, 37.35 Hz, 1H), 6.63 (d, J=9.56 Hz, 1H), 7.09 (m, 5H), 7.14 (d, J=7.7 Hz, 1H), 7.18 (d, J=8.6 Hz, 2H), 7.48 (d, J=9.56 Hz, 1H), 7.66 (m, 1H), 7.68 (t, J=7.7 Hz, 1H), 7.86 (m, 4H), 8.46 (br s, 1H).

### EXAMPLE 129

### methyl(1S)-1-[({(1R,3S,4S)-4-[((2S)-3,3-dimethyl-2-{3-[(6-methyl-2-pyridinyl)methyl]-2-oxo-1-imidazolidinyl}butanoyl)amino]-3-hydroxy-1-[4-(5-methyl-2-pyridinyl)benzyl]-5-phenylpentyl}amino)carbonyl]-2,2-dimethylpropylcarbamate

A solution containing the product from Example 128F (0.159 mmol) in THF (1.6 mL) was treated with the product from Example 1F (0.036 g, 0.190 mmol), DEPBT (0.075 g, 0.251 mmol), and N,N-diisopropylethylamine (0.30 mL, 1.72 mmol), stirred at 25° C. for 16 hours, and partitioned between ethyl acetate and 10% Na₂CO₃ solution. The organic was washed with additional 10% Na₂CO₃ solution and then brine, dried over MgSO₄, filtered and concentrated. The residue was purified by chromatography on silica gel eluting with 0-50% acetone in dichloromethane, to give the higher Rf (50% acetone in dichloromethane) product of the mixture (0.01 g). ¹H NMR (300 MHz, DMSO-d₆), δ ppm 0.80 (s, 9H), 0.88 (s, 9H), 1.37 (m, 1H), 1.52 (m, 1H), 2.32 (s, 3H), 2.45 (s, 3H), 2.66 (m, 3H), 2.83 (dd, J=13.79, 6.07 Hz, 1H), 3.03 (m, 2H), 3.23 (m, 1H), 3.53 (m, 4H), 3.83 (d, J=9.56 Hz, 1H), 4.01 (m, 2H), 4.03 (s, 1H), 4.16 (m, 1H), 4.33 (m, 2H), 4.43 (d, J=6.99 Hz, 1H), 6.88 (d, J=9.56 Hz, 1H), 7.09 (m, 5H), 7.14 (d, J=7.35 Hz, 1H), 7.21 (d, J=8.09 Hz, 2H), 7.54 (d, J=9.56 Hz, 1H), 7.67 (m, 2H), 7.81 (m, 2H), 7.93 (d, J=8.45 Hz, 2H), 8.48 (br s, 1H).

### EXAMPLE 130A

### benzyl(1S,3S,4S)-4-amino-3-hydroxy-5-phenyl-1-[4-(2-pyridinyl)benzyl]pentylcarbamate

A solution containing the product from Example 1C (0.088 g, 0.15 mmol) in a mixture CC THF (2 mL) and aqueous HCl (0.26 mL, 4 N) was stirred at 25° C. for 16 hours, then heated at 60° C. for 2 hours, cooled and concentrated. The residue was treated with ethanol and concentrated several times to give the title compound as the hydrochloride salt, which was used without further purification.

### EXAMPLE 130B

### benzyl(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentylcarbamate

A solution containing the product from Example 130A (0.15 mmol) in DMF (1 mL) was treated with the product from Example 10D (0.045 g, 0.15 mmol), EDAC (0.072 g, 0.375 mmol), HOBT (0.051 g, 0.375 mmol), and NMM (0.222 mL, 2.02 mmol), stirred at 25° C. for 40 hours, and partitioned between ethyl acetate and water. The organic was washed with brine, dried over Na₂SO₄, filtered and concentrated. The residue was purified by chromatography on silica gel eluting with 1-5% methanol in chloroform, to give the product (0.067 g, 58% yield).

### EXAMPLE 130C

### (2S)-N-{(1S,2S,4S)-4-amino-1-benzyl-2-hydroxy-5-[4-(2-pyridinyl)phenyl]pentyl}-3,3-dimethyl-2-{3-[(6-methyl-2-pyridinyl)methyl]-2-oxo-1-imidazolidinyl}butanamide

A solution containing the product from Example 130B (0.067 g, 0.086 mmol) in methanol (3 mL) was treated with a solution of HCl in dioxane (0.025 mL, 4 M) and Pd on carbon (0.007 g, 10% Pd by wt.), stirred under a hydrogen atmosphere (balloon pressure) at 25° C. for 16 hours, filtered through a bed of celite® and rinsed with methanol. The solvent was concentrated to give the title compound as the hydrochloride salt (0.053 g), which was used without further purification.

### EXAMPLE 130D

### tert-butyl(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate

To a solution containing the product from Example 130C (0.053 g, 0.081 mmol) in DMF (1 mL) was treated with Boc-L-tert-leucine (0.019 g, 0.081 mmol), EDAC (0.023 g, 0.122 mmol), HOBT (0.016 g, 0.122 mmol), and NMM (0.018 mL, 0.162 mmol), stirred at 25° C. for 40 hours, and partitioned between ethyl acetate and water. The organic phase was washed with brine, and then dried over MgSO₄, filtered and concentrated. The residue was purified by chromatography on silica gel eluting with 20% ethyl acetate in chloroform, to give the product (0.024 g, 34% yield).

### EXAMPLE 130E

### (2S)-2-amino-N-{(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}-3,3-dimethylbutanamide

A solution containing the product from Example 130D (0.024 g, 0.028 mmol) in a mixture of THF (1 mL) and aqueous HCl (0.050 mL, 4 N) was stirred at 25° C. for 16 hours, and concentrated under reduced pressure. The residue was treated with ethanol and concentrated several times to give the title compound as the hydrochloride salt, which was used without further purification.

### EXAMPLE 130F

### Tert-Butyl 2-({(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropyl}amino)-2-oxoethylcarbamate

A solution containing the product from Example 130E (0.028 mmol) in DMF (1 mL) were added Boc-glycine (0.005 g, 0.028 mmol), EDAC (0.008 g, 0.042 mmol), HOBT (0.0056 g, 0.042 mmol), and NMM (0.018 mL, 0.162 mmol), stirred at 25° C. for 40 hours, and partitioned between ethyl acetate and water. The organic phase was washed with brine, dried over MgSO₄, filtered and concentrated. The residue was purified by chromatography on silica gel eluting with 2% methanol in chloroform, to give the product. ES-MS: m/z 920 [M+H]⁺.

### EXAMPLE 131

### methyl(1S)-1-[({(1S,3S,4S)-3-hydroxy-4-[((2S)-2-{3-[(6-isopropyl-2-pyridinyl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate

A solution containing the product from Example 2C (0.025 g, 0.066 mmol) in THF (0.66 mL) was treated with the product from Example 117D (0.034 g, 0.079 mmol), DEPBT (0.030 g, 0.099 mmol), and N,N-diisopropylethylamine (0.115 mL, 0.658 mmol), stirred at 25° C. for 16 hours, and partitioned between chloroform and 10% Na₂CO₃ solution. The organic phase was washed with additional 10% Na₂CO₃ solution and brine, dried over MgSO₄, filtered and concentrated. The residue was purified by chromatography on silica gel eluting with 0-100% ethyl acetate/dichloromethane, followed by 0-5% methanol in ethyl acetate to give the product (0.030 g, 54% yield). ¹H NMR (300 MHz, DMSO-d), δ ppm 0.82 (s, 9H), 0.89 (s, 9H), 1.21 (d, J=2.2 Hz, 3H), 1.23 (d, J=2.2 Hz, 3H), 1.41-1.57 (m, 2H), 2.33 (q, J=8.5 Hz, 1H), 2.57 (m, 1H), 2.66 (d, J=7.0 Hz, 2H), 2.79 (m, 1H), 2.98 (m, 2H), 3.19 (m, 1H), 3.50 (s, 3H), 3.66 (m, 1H), 3.85 (d, J=9.56 Hz, 1H), 4.08 (s, 1H), 4.13-4.25 (m, 2H), 4.38 (m, 2H), 4.53 (d, J=7.7 Hz, 1H), 6.65 (d, J=9.55 Hz, 1H), 7.03-7.13 (m, 7H), 7.15-7.22 (m, 3H), 7.31 (m, 1H), 7.47 (d, J=9.56 Hz, 1H), 7.71 (t, J=7.7 Hz, 1H), 7.81-7.90 (m, 3H), 8.62 (m, 1H).

### EXAMPLE 132A

### tert-butyl(1S,3S,4S)-1-benzyl-4-[((2S)-3,3-dimethyl-2-{3-[(6-methyl-2-pyridinyl)methyl]-2-oxo-1-imidazolidinyl}butanoyl)amino]-3-hydroxy-5-[4-(2-pyridinyl)phenyl]pentylcarbamate

A solution containing the product from Example 23Q (0.074 mmol) in THF (0.8 mL) was treated with the product from Example 10D (0.032 g, 0.094 mmol), DEPBT (0.035 g, 0.117-mmol), and N,N-diisopropylethylamine (0.10 mL, 0.574 mmol), stirred at 25° C. for 1 hour, and partitioned between chloroform and 10% Na₂CO₃ solution. The organic phase was washed with additional 10% Na₂CO₃ solution and then brine, dried over MgSO₄, filtered and concentrated. The product was purified by chromatography on silica gel eluting with 0-100% ethyl acetate/dichloromethane, followed by 0-0.5% methanol in ethyl acetate to give the product (0.017 g, 31% yield).

### EXAMPLE 132B

### (2S)-N-{(1S,2S,4S)-4-amino-2-hydroxy-5-phenyl-1-[4-(2-pyridinyl)benzyl]pentyl}-3,3-dimethyl-2-{3-[(6-methyl-2-pyridinyl)methyl]-2-oxo-1-imidazolidinyl}butanamide

A solution containing the product from Example 132A (0.017 g, 0.023 mmol) in dichloromethane (1 mL) was added trifluoroacetic acid (1 mL), and the mixture was stirred at 25° C. for 1 hour, and concentrated. The residue was partitioned between ethyl acetate and saturated NaHCO₃, and the organic phase was washed with brine and dried over MgSO₄, filtered and concentrated to give the product, which was used without further purification.

### EXAMPLE 132C

### methyl(1S)-1-[({(1S,3S,4S)-1-benzyl-4-[((2S)-3,3-dimethyl-2-{3-[(6-methyl-2-pyridinyl)methyl]-2-oxo-1-imidazolidinyl}butanoyl)amino]-3-hydroxy-5-[4-(2-pyridinyl)phenyl]pentyl}amino)carbonyl]-2,2-dimethylpropylcarbamate

A solution containing the product from Example 132B (0.023 mmol) in THF (0.25 mL) were added the product from Example 1F (0.005 g, 0.026 mmol), DEPBT (0.010 g, 0.033 mmol), and N,N-diisopropylethylamine (0.020 mL, 0.115 mmol), stirred at 25° C. for 16 hours, and partitioned between chloroform and 10% Na₂CO₃ solution. The organic phase was washed with additional 10% Na₂CO₃ solution and then brine, dried over MgSO₄, filtered and concentrated. The product was purified by chromatography on silica gel eluting with 0-100% ethyl acetate/dichloromethane, followed by 0-5% methanol in ethyl acetate to give the product. The product was re-purified by preparative TLC eluting with 5% methanol in ethyl acetate to give the title compound (0.002 g, 11% yield). ¹H NMR (300 MHz, DMSO-d6), δ ppm 0.82 (s, 9H), 0.91 (s, 9H), 1.25 (m, 1H), 1.52 (m, 2H), 2.41 (m, 1H), 2.43 (s, 3H), 2.74 (m, 3H), 2.97 (q, J=9.2 Hz, 1H), 3.24 (m, 1H), 3.54 (s, 3H), 3.66 (m, 1H), 3.82 (d, J=9.9 Hz, 2H), 4.09 (s, 1H), 4.17 (m, 1H), 4.25 (d, J=16 Hz, 1H), 4.35 (d, J=16 Hz, 1H), 4.53 (d, J=7.4 Hz, 1H), 6.63 (d, J=9.9 Hz, 1H), 7.11 (m, 8H), 7.21 (d, J=8.09 Hz, 2H), 7.51 (d, J=8.56 Hz, 1H), 7.62 (t, J=7.7 Hz, 1H), 7.75 (m, 2H), 7.83 (d, J=8.09 Hz, 1H), 8.60 (d, J=4.1 Hz, 1H).

### EXAMPLE 133A

### tert-butyl(1S,2S,4S)-1-benzyl-4-[(tert-butoxycarbonyl)amino]-2-hydroxy-5-[4-(2-pyridinyl)phenyl]pentylcarbamate

To a solution of the product from Example 1B (7.32 g, 12.1 mmol) in toluene (400 mL) were treated with DPPA (5.2 mL, 24.2 mmol) and triethylamine (3.4 mL, 24.4 mmol), heated at reflux for 2 hours, cooled, treated with tert-Butyl alcohol (41.6 mL), triethylamine (4 mL), and DMAP (0.30 g), heated at reflux for an additional 64 hours, cooled and concentrated. A solution of the residue in THF (60 mL) was treated with TBAF solution in THF (36 mL, 1N), stirred at 25° C. for 40 hours, concentrated and partitioned between ethyl acetate and water. The organic phase was washed with brined, dried over MgSO₄, filtered and concentrated. The residue was chromatographed on silica gel eluting with 0-50% ethyl acetate in dichloromethane to give 0.614 g (9% yield) of the lower Rf product by TLC (25% ethyl acetate in dichloromethane).

### EXAMPLE 133B

### (2S,3S,5S)-2,5-diamino-1-phenyl-6-[4-(2-pyridinyl)phenyl]-3-hexanol

A solution containing the product from Example 133A (0.60 g, 1.07 mmol) in dichloromethane (10 mL) was treated with trifluoroacetic acid (10 mL), stirred at 25° C. for 1 hour, concentrated and partitioned between chloroform and saturated NaHCO₃. The organic was dried over Na₂SO₄, filtered and concentrated to give the title compound (0.386 g, 98% yield).

### EXAMPLE 133C

### methyl(1S,4S,5S,7S,10S)-4-benzyl-1,10-disec-butyl-5-hydroxy-2,9,12-trioxo-7-[4-(2-pyridinyl)benzyl]-13-oxa-3,8,11-triazatetradec-1-ylcarbamate

A solution containing the product from Example 133B (0.045 g, 0.125 mmol) in DMF (1.2 mL) was treated with the product from Example 5A (0.060 g, 0.317 mmol), EDAC (0.075 g, 0.391 mmol), HOBT (0.050 g, 0.370 mmol), and NMM (0.030 mL, 0.272 mmol), stirred at 25° C. for 16 hours, and partitioned between ethyl acetate and water. The organic phase was washed with brine, dried over MgSO₄, filtered and concentrated. The residue was purified by reversed phase chromatography on a C18 column, eluting with 5-p100% acetonitrile in water (0.1% TFA). The product was partitioned between ethyl acetate and saturated NaHCO₃, and the organic phase was washed with brine and dried over MgSO₄, filtered and concentrated to give the product (0.0072 g, 8% yield). ¹H NMR (300 MHz, DMSO-d), δ ppm 0.56 (d, J=7.0 Hz, 3H), 0.64 (t, J=7.0 Hz, 3H), 0.72 (t, J=8.5 Hz, 3H), 0.90-1.07 (m, 2H), 1.20-1.34 (m, 3H), 1.43-1.67 (m, 4H), 2.57 (m, 1H), 2.69-2.77 (m, 3H), 3.50 (s, 3H), 3.53 (s, 3H), 3.60 (m, 1H), 3.71-3.83 (m, 2H), 4.03-4.21 (m, 2H), 6.87 (d, J=9.2 Hz, 1H), 6.70 (d, J=8.5 Hz, 1H), 7.10-7.21 (m, 7H), 7.30 (m, 1H), 7.70 (d, J=8.8 Hz, 1H), 7.81-7.96 (m, 5H), 8.63 (m, 1H).

### EXAMPLE 134A

### 9H-fluoren-9-ylmethyl(1S,2S,4S)-1-benzyl-2-hydroxy-4-({(2S,3S)-3-methyl-2-[2-oxo-3-(4-quinolinylmethyl)-1-imidazolidinyl]pentanoyl}amino)-5-phenylpentylcarbamate

A solution containing the product from Example 3B (0.150 g, 0.276 mmol) in DMF (3 mL) was treated with the product from Example 4A (0.110 g, 0.332 mmol), EDAC (0.080 g, 0.417 mmol), HOBT (0.055, 0.407 mmol), and NMM (0.090 mL, 0.819 mmol) at 0° C., stirred at 25° C. for 16 hours, and partitioned between ethyl acetate and water. The organic phase was washed with 10% citric acid, dilute sodium bicarbonate, and brine, dried over MgSO₄ filtered and concentrated. The residue was purified by reversed phase chromatography on a C18 column, eluting with 5-100% acetonitrile in water (0.1% TFA) to give the product (0.122 g, 53% yield).

### EXAMPLE 134B

### (2S,3S)-N-[(1S,3S,4S)-4-amino-1-benzyl-3-hydroxy-5-phenylpentyl]-3-methyl-2-[2-oxo-3-(4-quinolinylmethyl)-1-imidazolidinyl]pentanamide

A solution containing the product from Example 134A (0.122 g, 0.147 mmol) in DMF (6 mL) was treated with diethylamine (1.5 mL), stirred at 25° C. for 1 hour, and partitioned between ethyl acetate and water. The organic phase was washed with brine and dried over MgSO₄, filtered and concentrated to give the title compounds.

### EXAMPLE 134C

### methyl(1S)-1-({[(1S,2S,4S)-1-benzyl-2-hydroxy-4-({(2S)-3-methyl-2-[2-oxo-3-(4-quinolinylmethyl)-1-imidazolidinyl]pentanoyl}amino)-5-phenylpentyl]amino}carbonyl)-2,2-dimethylpropylcarbamate

A solution containing the product from Example 134B (0.147 mmol) in DMF (2 mL) was treated with the product from Example 1F (0.035 g, 0.185 mmol), EDAC (0.045 g, 0.235 mmol), HOBT (0.030 g, 0.222 mmol), and NMM (0.050 mL, 0.455 mmol) at 0° C., stirred at 25° C. for 16 hours, and partitioned between ethyl acetate and water. The organic phase was washed with 10% citric acid, dilute sodium bicarbonate, and brine, dried over MgSO₄, filtered and concentrated. The residue was purified by reversed phase chromatography on a C18 column, eluting with 5-100% acetonitrile in water (0.1% TFA) to give the title compound (0.019 g, 17% yield). ¹H NMR (300 MHz, DMSO-d), δ ppm 0.68 (d, J=6.6 Hz, 3H), 0.93 (m, 12H), 0.88-0.97 (m, 1-H), 1.22-1.33 (m, 1H), 1.47-1.56 (m, 2H), 1.72-1.84 (m, 1H), 2.38-2.45 (m, 1H), 2.62-2.73 (m, 3H), 2.79-2.86 (m, 1H), 2.98-3.10 (m, 3H), 3.55-3.63 (m, 4H), 3.89-3.94 (m, 2H), 4.07-4.21 (m, 2H), 4.80 (s, 2H), 6.79 (d, J=9.56 Hz, 1H), 6.93-7.20 (m, 10H), 7.41 (d, J=4.41 Hz, 1H), 7.48-7.53 (m, 1H), 7.57-7.62 (m, 1H), 7.75-7.80 (m, 1H), 7.87 (d, J=9.19 Hz, 1H), 8.06 (d, J=8.45 Hz, 1H), 8.30 (d, J=8.08 Hz, 1H), 8.88 (d, J=4.41 Hz, 1H).

The following additional compounds of the present invention can be prepared by one skilled in the art using known synthetic methodology or by using synthetic methodology described in the Schemes and Examples contained herein. The additional compounds encompassed by the following tables can be described by taking one core from Table 1, one R¹ substituent from Table 2, one R² substituent from Table 3, one R³ substituent from Table 4, one R⁶ substituent from Table 5, one R⁷ substituent from Table 6, and one R⁸ substituent from Table 7, one R⁹ substituent from Table 8, or one R¹¹ substituent from Table 9; wherein X₁ in the tables of substituents represents the Core Ring Structure.

TABLE 1
Examples of Core Ring Structures

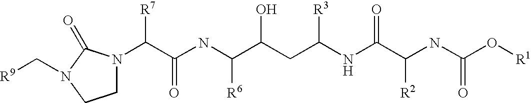

1

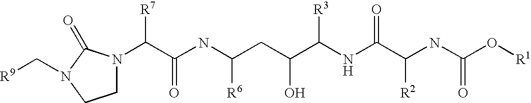

2

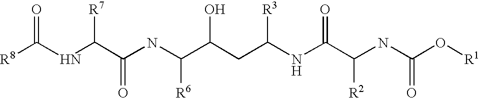

2

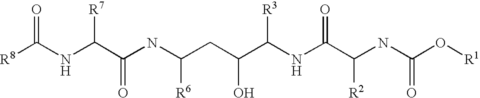

4

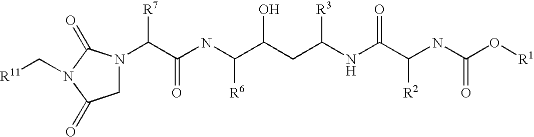

5

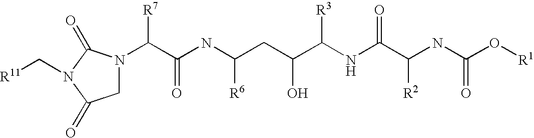

6

TABLE 2
Examples of R¹ Substituents

| X₁—CH₃ | 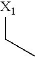 | 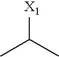 | 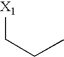 |
|---|---|---|---|
| 1 | 2 | 3 | 4 |
| 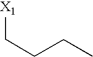 | 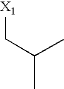 | 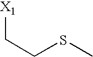 | 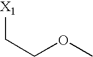 |
| 5 | 6 | 7 | 8 |

TABLE 3
Examples of R² Substituents

| X₁—H | X₁—CH₃ | 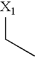 | 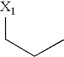 |
|---|---|---|---|
| 1 | 2 | 3 | 4 |
| 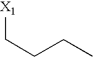 | 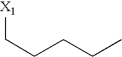 | 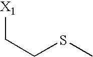 | 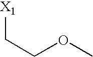 |
| 5 | 6 | 7 | 8 |
| 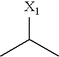 | 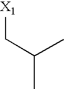 | 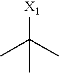 | 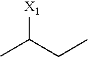 |
| 9 | 10 | 11 | 12 |
| 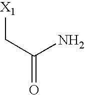 | 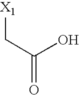 | 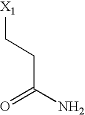 | 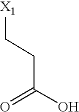 |
| 13 | 14 | 15 | 16 |
| 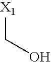 | 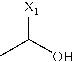 | 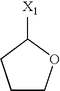 | 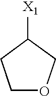 |
| 17 | 18 | 19 | 20 |

TABLE 4
Examples of R³ Substituents

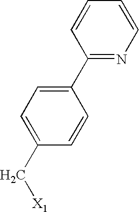

1

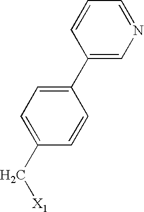

2

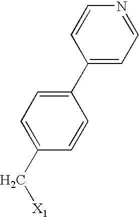

3

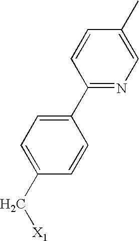

4

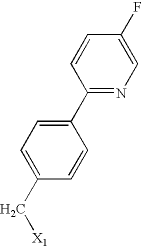

5

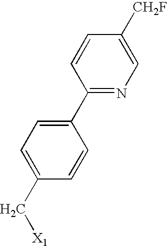

6

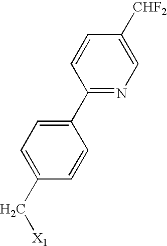

7

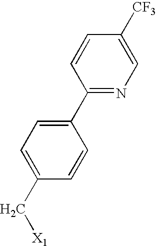

8

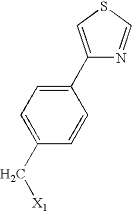

9

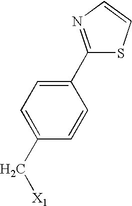

10

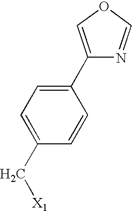

11

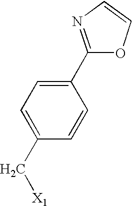

12

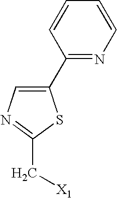

13

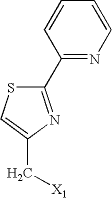

14

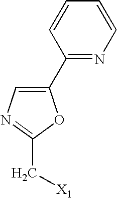

15

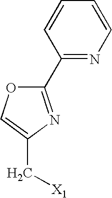

16

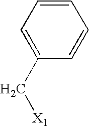

17

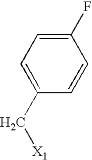

18

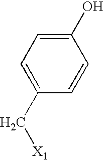

19

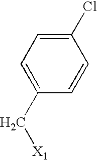

20

TABLE 5
Examples of R⁶ Substituents

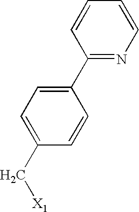

1

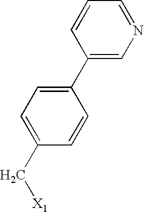

2

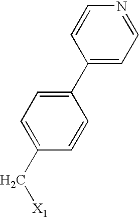

3

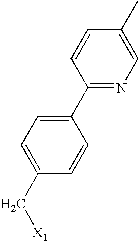

4

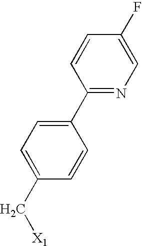

5

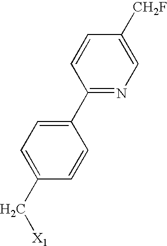

6

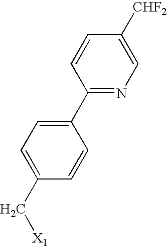

7

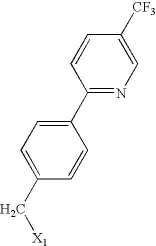

8

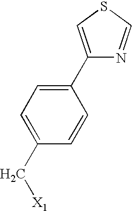

9

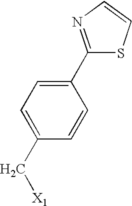

10

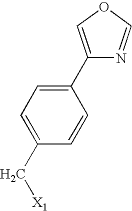

11

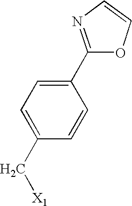

12

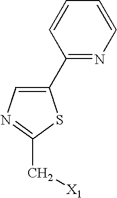

13

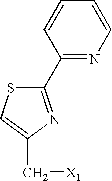

14

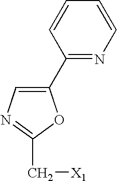

15

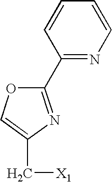

16

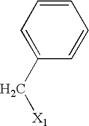

17

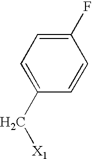

18

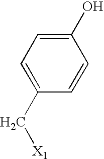

19

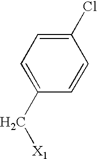

20

TABLE 6
Examples of R⁷ Substituents

| X₁—H | X₁—CH₃ | 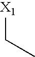 | 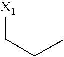 |
|---|---|---|---|
| 1 | 2 | 3 | 4 |
| 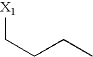 | 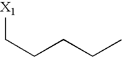 | 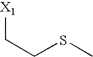 | 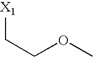 |
| 5 | 6 | 7 | 8 |
| 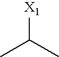 | 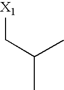 | 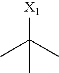 | 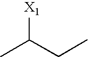 |
| 9 | 10 | 11 | 12 |
| 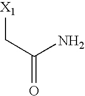 | 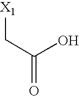 | 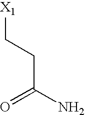 | 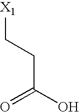 |
| 13 | 14 | 15 | 16 |
| 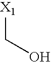 | 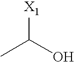 | 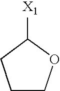 | 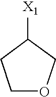 |
| 17 | 18 | 19 | 20 |

TABLE 7
Examples of R⁸ Substituents
X₁—OCH₃
1

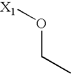

2

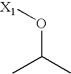

3

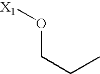

4

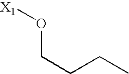

5

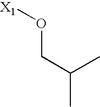

6

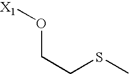

7

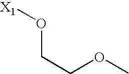

8

TABLE 8
Examples of R⁹ Substituents

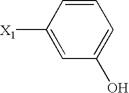

1

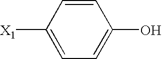

2

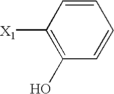

3

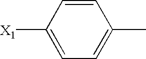

4

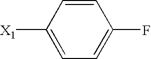

5

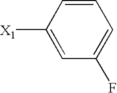

6

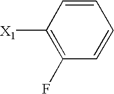

7

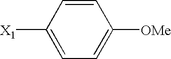

8

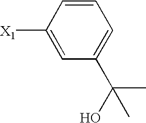

9

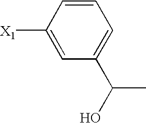

10

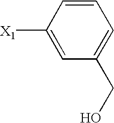

11

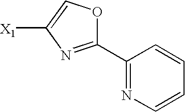

12

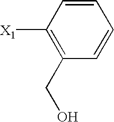

13

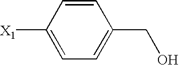

14

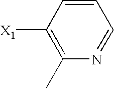

15

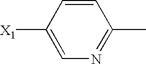

16

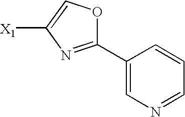

17

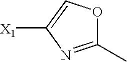

18

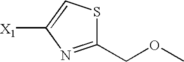

19

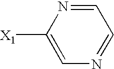

20

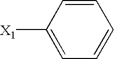

21

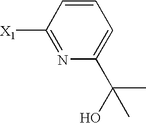

22

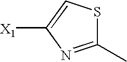

23

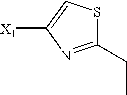

24

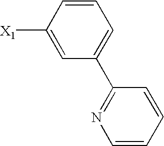

25

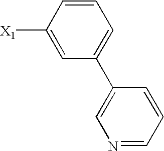

26

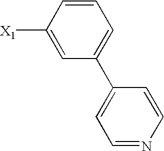

27

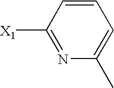

28

TABLE 9
Examples of R¹¹ Substituents

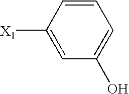

1

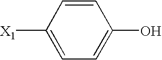

2

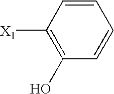

3

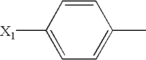

4

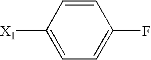

5

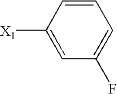

6

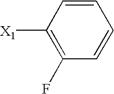

7

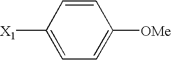

8

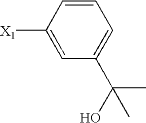

9

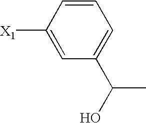

10

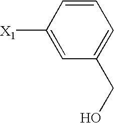

11

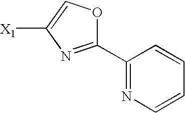

12

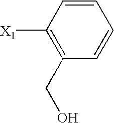

13

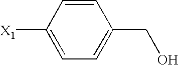

14

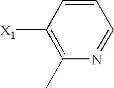

15

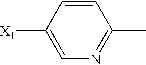

16

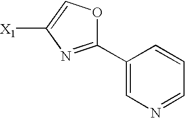

17

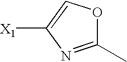

18

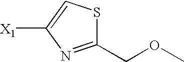

19

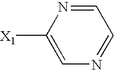

20

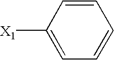

21

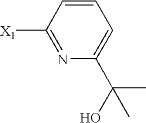

22

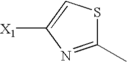

23

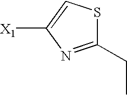

24

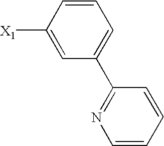

25

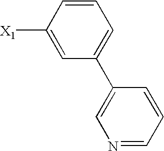

26

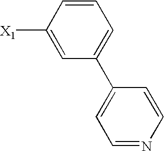

27

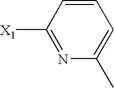

28

The foregoing is merely illustrative of the invention and is not intended to limit the invention to the disclosed compounds. Variations and changes which are obvious to one skilled in the art are intended to be within the scope and nature of the invention which are defined in the appended claims.

1. A compound of formula (I), 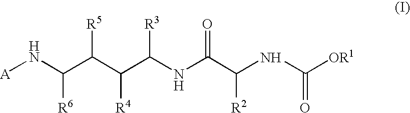
or a pharmaceutically acceptable salt form, stereoisomer, ester, salt of an ester, prodrug, salt of a prodrug, or combination thereof, wherein:
A is 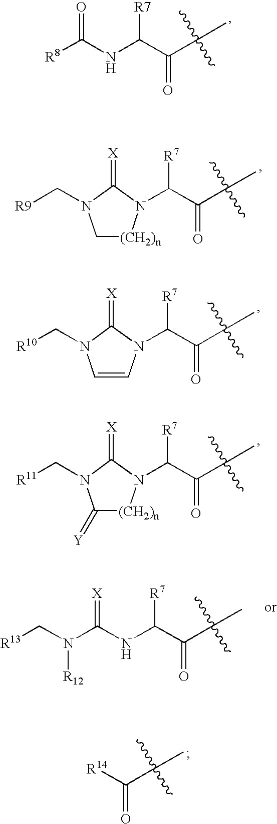
X is O, S or NH;
Y is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)OR¹, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylN-H₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, alkylN(R_{b})C(O)Rₐ, alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), 7alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁸ is —ORₐ or -alkylORₐ;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁴ is —ORₐ or -alkylORₐ;
R¹⁶ is hydrogen or R¹⁵;
R¹⁵ is 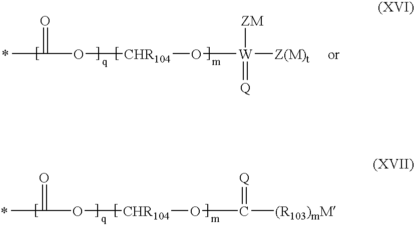
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring systems is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

2. The compound of claim 1 wherein R⁴ is H and R⁵ is OR¹⁶.

3. The compound of claim 1 wherein R⁴ is OR¹⁶ and R⁵ is H.

4. The compound of claim 1, or a pharmaceutically acceptable salt form, stereoisomer, ester, salt of an ester, prodrug, salt of a prodrug, or combination thereof, selected from the group consisting of:
methyl 7-benzyl-1,10-ditert-butyl-6-hydroxy-2,9,12-trioxo-4-[4-(2-pyridinyl)benzyl]-13-oxa-3,8,11-triazatetradec-1-ylcarbamate;
methyl 4-benzyl-1,10-ditert-butyl-5-hydroxy-2,9,12-trioxo-7-[4-(2-pyridinyl)benzyl]-13-oxa-3,8,11-triazatetradec-1-ylcarbamate;
methyl 1-{[(1-benzyl-3-hydroxy-4-{[3-methyl-2-(2-oxo-3-{[2-(2-pyridinyl)-1,3-thiazol-4-yl]methyl}-1-imidazolidinyl)pentanoyl]amino}-5-phenylpentyl)amino]carbonyl}-2,2-dimethylpropylcarbamate;
methyl 1-({{[1-benzyl-3-hydroxy-4-({3-methyl-2-[2-oxo-3-(4-quinolinylmethyl)-1-imidazolidinyl]pentanoyl}amino)-5-phenylpentyl]amino}carbonyl)-2,2-dimethylpropylcarbamate;
methyl 1-({[1-benzyl-3-hydroxy-4-({3-methyl-2-[2-oxo-3-(4-quinolinylmethyl)-1-imidazolidinyl]pentanoyl}amino)-5-phenylpentyl]amino}carbonyl)-2-methylbutylcarbamate;
methyl 1-{[(1-benzyl-3-hydroxy-4-{[2-(3-{[2-(methoxymethyl)-1,3-thiazol-4-yl]methyl}-2-oxo-1-imidazolidinyl)-3,3-dimethylbutanoyl]amino}-5-phenylpentyl)amino]carbonyl}-2,2-dimethylpropylcarbamate;
methyl 1-[({1-benzyl-3-hydroxy-4-[(3-methyl-2-{3-[(6-methyl-2-pyridinyl)methyl]-2-oxo-1-imidazolidinyl}pentanoyl)amino]-5-phenylpentyl}amino)carbonyl]-2,2-dimethylpropylcarbamate;
methyl 1-[({1-benzyl-2-hydroxy-4-[(3-methyl-2-{3-[(6-methyl-2-pyridinyl)methyl]-2-oxo-1-imidazolidinyl}pentanoyl)amino]-5-phenylpentyl}amino)carbonyl]-2,2-dimethylpropylcarbamate;
methyl 1-[({1-benzyl-2-hydroxy-4-[(3-methyl-2-{3-[(6-methyl-2-pyridinyl)methyl]-2-oxo-1-imidazolidinyl}pentanoyl)amino]-5-phenylpentyl}amino)carbonyl]-2-methylbutylcarbamate;
methyl 1-[({I-benzyl-4-[(3,3-dimethyl-2-{3-[(6-methyl-2-pyridinyl)methyl]-2-oxo-1-imidazolidinyl}butanoyl)amino]-2-hydroxy-5-phenylpentyl}amino)carbonyl]-2,2-dimethylpropylcarbamate;
methyl 1-[({1-benzyl-2-hydroxy-4-[(3-methyl-2-{3-[(2-methyl-1,3-thiazol-5-yl)methyl]-2-oxo-1-imidazolidinyl}pentanoyl)amino]-5-phenylpentyl}amino)carbonyl]-2,2-dimethylpropylcarbamate;
methyl 1-{[(1-benzyl-2-hydroxy-4-{[3-methyl-2-(2-oxo-3-{[2-(3-pyridinyl)-1,3-thiazol-4-yl]methyl}-1-imidazolidinyl)pentanoyl]amino}-5-phenylpentyl)amino]carbonyl}-2,2-dimethylpropylcarbamate;
methyl 1-[({1-benzyl-2-hydroxy-4-[(3-methyl-2-{3-[(6-methyl-3-pyridinyl)methyl]-2-oxo-1-imidazolidinyl}pentanoyl)amino]-5-phenylpentyl}amino)carbonyl]-2,2-dimethylpropylcarbamate;
methyl 1-[({1-benzyl-4-[(3,3-dimethyl-2-{3-[(2-methyl-1,3-thiazol-4-yl)methyl]-2-oxo-1-imidazolidinyl}butanoyl)amino]-2-hydroxy-5-phenylpentyl}amino)carbonyl]-2,2-dimethylpropylcarbamate;
methyl 1-[({1-benzyl-2-hydroxy-4-[(3-methyl-2-{3-[(2-methyl-3-pyridinyl)methyl]-2-oxo-1-imidazolidinyl}pentanoyl)amino]-5-phenylpentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-{[(1-benzyl-2-hydroxy-4-{[2-(3-{[6-(1-hydroxy-1-methylethyl)-2-pyridinyl]methyl}-2-oxo-1-imidazolidinyl)-3,3-dimethylbutanoyl]amino}-5-phenylpentyl)amino]carbonyl}-2,2-dimethylpropylcarbamate;
methyl 1-[({3-hydroxy-4-[(3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-{[(1-benzyl-4-{[3,3-dimethyl-2-(2-oxo-3-{[2-(3-pyridinyl)-1,3-thiazol-4-yl]methyl}-1-imidazolidinyl)butanoyl]amino}-2-hydroxy-5-phenylpentyl)amino]carbonyl}-2,2-dimethylpropylcarbamate;
methyl 1-({[1-benzyl-4-({3,3-dimethyl-2-[2-oxo-3-(3-pyridinylmethyl)-1-imidazolidinyl]butanoyl}amino)-2-hydroxy-5-phenylpentyl]amino}carbonyl)-2,2-dimethylpropylcarbamate;
methyl 1-[({3-hydroxy-4-[(3-methyl-2-{3-[(2-methyl-3-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({1-benzyl-4-[(3,3-dimethyl-2-{3-[(6-methyl-2-pyridinyl)methyl]-2-oxo-1-imidazolidinyl}butanoyl)amino]-3-hydroxy-5-phenylpentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl 1-[({1-benzyl-4-[(3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentyl}amino)carbonyl]-2,2-dimethylpropylcarbamate;
methyl 7-benzyl-1,10-ditert-butyl-5-hydroxy-2,9,12-trioxo-4-[4-(2-pyridinyl)benzyl]-13-oxa-3,8,11-triazatetradec-1-ylcarbamate;
1:1 mixture of (3R,3aS,6aR)-hexahydrofuro[2,3-b]furan-3-yl 1-benzyl-3-hydroxy-4-({2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl 1-benzyl-3-hydroxy-4-({2-[(methoxycarbonyl)amino]-3,3-dimethylbutanoyl}amino)-5-[4-(2-pyridinyl)phenyl]pentylcarbamate;
1:1 mixture of (3R,3aS,6aR)-hexahydrofuro[2,3-b]furan-3-yl 1-benzyl-2-hydroxy-4-({2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl 1-benzyl-2-hydroxy-4-({2-[(methoxycarbonyl)amino]-3,3-dimethylbutanoyl}amino)-5-[4-(2-pyridinyl)phenyl]pentylcarbamate;
methyl 1-[({2-hydroxy-4-[(3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 4-benzyl-10-tert-butyl-5-hydroxy-1-[1-methyl-1-(methylsulfanyl)ethyl]-2,9,12-trioxo-7-[4-(2-pyridinyl)benzyl]-13-oxa-3,8,11-triazatetradec-1-ylcarbamate;
methyl 4-benzyl-10-tert-butyl-5-hydroxy-1-[1-methyl-1-(methylsulfonyl)ethyl]-2,9,12-trioxo-7-[4-(2-pyridinyl)benzyl]-13-oxa-3,8,11-triazatetradec-1-ylcarbamate;
methyl 4-benzyl-10-tert-butyl-6-hydroxy-1-[1-methyl-1-(methylsulfanyl)ethyl]-2,9,12-trioxo-7-[4-(2-pyridinyl)benzyl]-13-oxa-3,8,11-triazatetradec-1-ylcarbamate;
methyl 4-benzyl-10-tert-butyl-6-hydroxy-1-[1-methyl-1-(methylsulfonyl)ethyl]-2,9,12-trioxo-7-[4-(2-pyridinyl)benzyl]-13-oxa-3,8,11-triazatetradec-1-ylcarbamate;
methyl 1-[({4-{[(2S)-3,3-dimethyl-2-(2-oxo-3-{[2-(3-pyridinyl)-1,3-thiazol-4-yl]methyl}-1-imidazolidinyl)butanoyl]amino}-3-hydroxy-5-phenyl-1-[4-(2-pyridinyl)benzyl]pentyl}amino)carbonyl]-2,2-dimethylpropylcarbamate;
methyl 1-[({4-{[3,3-dimethyl-2-(2-oxo-3-{[2-(3-pyridinyl)-1,3-thiazol-4-yl]methyl}-1-imidazolidinyl)butanoyl]amino}-2-hydroxy-5-phenyl-1-[4-(2-pyridinyl)benzyl]pentyl}amino)carbonyl]-2,2-dimethylpropylcarbamate;
methyl 1-[({3-hydroxy-4-[(3-methyl-2-{3-[(6-methyl-2-pyridinyl)methyl]-2,4-dioxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({2-hydroxy-4-[(3-methyl-2-{3-[(6-methyl-2-pyridinyl)methyl]-2,4-dioxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({4-{[(2,6-dimethylphenoxy)acetyl]amino}-3-hydroxy-5-phenyl-1-[4-(2-pyridinyl)benzyl]pentyl}amino)carbonyl]-2,2-dimethylpropylcarbamate;
methyl 1-[({4-{[(2,6-dimethylphenoxy)acetyl]amino}-2-hydroxy-5-phenyl-1-[4-(2-pyridinyl)benzyl]pentyl}amino)carbonyl]-2,2-dimethylpropylcarbamate;
methyl 1-[({3-hydroxy-4-({(2S)-2-[3-(imidazo[1,5-a]pyridin-3-ylmethyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({2-hydroxy-4-({2-[3-(imidazo[1,5-a]pyridin-3-ylmethyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({4-({3,3-dimethyl-2-[2-oxo-3-(4-quinolinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({(2S)-3,3-dimethyl-2-[2-oxo-3-(4-quinolinylmethyl)-1-imidazolidinyl]butanoyl}amino)-2-hydroxy-5-phenyl-1-[4-(2-pyridinyl)benzyl]pentyl}amino)carbonyl]-2,2-dimethylpropylcarbamate;
methyl 1-[({2-hydroxy-4-{[2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl 1-[({3-hydroxy-4-{[2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2,4-dioxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
1:1 mixture of (3R,3aS,6aR)-hexahydrofuro[2,3-b]furan-3-yl 1-benzyl-2-hydroxy-4-({2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl 1-benzyl-2-hydroxy-4-({2-[(methoxycarbonyl)amino]-3,3-dimethylbutanoyl}amino)-5-[4-(2-pyridinyl)phenyl]pentylcarbamate;
methyl 1-[({4-{[3,3-dimethyl-2-(2-oxo-3-{[2-(3-pyridinyl)-1,3-thiazol-4-yl]methyl}-1-imidazolidinyl)butanoyl]amino}-3-hydroxy-5-phenyl-1-[4-(2-pyridinyl)benzyl]pentyl}amino)carbonyl]-2,2-dimethylpropylcarbamate;
methyl 1-[({4-{[(2,6-dimethylphenoxy)acetyl]amino}-3-hydroxy-5-phenyl-1-[4-(2-pyridinyl)benzyl]pentyl}amino)carbonyl]-2,2-dimethylpropylcarbamate;
methyl 1-[({3-hydroxy-4-{[2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2,4-dioxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2,4-dioxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[2-oxo-3-(4-quinolinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[(phenoxyacetyl)amino]butanoyl}amino)-3-hydroxy-5-phenyl-1-[4-(2-pyridinyl)benzyl]pentyl}amino)carbonyl]-2,2-dimethylpropylcarbamate;
methyl 7-benzyl-1,10-ditert-butyl-6-hydroxy-2,9,12-trioxo-4-[4-(2-pyridinyl)benzyl]-14-oxa-3,8,11-triazapentadec-1-ylcarbamate;
methyl 1-[({3-hydroxy-4-[(2-{3-[(2-isopropyl-1,3-thiazol-4-yl)methyl]-2,4-dioxo-1-imidazolidinyl}-3-methylpentanoyl)amino]-5-phenyl-1-[4-(2-pyridinyl)benzyl]pentyl}amino)carbonyl]-2,2-dimethylpropylcarbamate;
methyl 1-[({4-{[2-(2,4-dioxo-3-{[2-(3-pyridinyl)-1,3-thiazol-4-yl]methyl}-1-imidazolidinyl)-3-methylpentanoyl]amino}-3-hydroxy-5-phenyl-1-[4-(2-pyridinyl)benzyl]pentyl}amino)carbonyl]-2,2-dimethylpropylcarbamate;
methyl 1-[({4-{[3,3-dimethyl-2-({[(6-methyl-3-pyridinyl)oxy]acetyl}amino)butanoyl]amino}-3-hydroxy-5-phenyl-1-[4(2-pyridinyl)benzyl]pentyl}amino)carbonyl]-2,2-dimethylpropylcarbamate;
methyl 1-[({4-[(3,3-dimethyl-2-{3-[(1-methyl-1H-benzimidazol-2-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(2-methyl-1,3-thiazol-4-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
3-pyridinylmethyl 4-benzyl-1,10-ditert-butyl-5-hydroxy-2,9,12-trioxo-7-[4-(2-pyridinyl)benzyl]-13-oxa-3,8,11-triazatetradec-1-ylcarbamate;
benzyl 4-benzyl-1,10-ditert-butyl-5-hydroxy-2,9,12-trioxo-7-[4-(2-pyridinyl)benzyl]-13-oxa-3,8,11-triazatetradec-1-ylcarbamate;
methyl 7-benzyl-1,10-ditert-butyl-6-hydroxy-13-methyl-2,9,12-trioxo-14-phenyl-4-[4-(2-pyridinyl)benzyl]-3,8,11,13-tetraazatetradec-1-ylcarbamate;
methyl 7-benzyl-1,10-ditert-butyl-6-hydroxy-13-methyl-2,9,12-trioxo-14-phenyl-4-[4-(2-pyridinyl)benzyl]-3,8,11,13-tetraazatetradec-1-ylcarbamate;
methyl 1-[({4-({3,3-dimethyl-2-[3-(2-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[3-(3-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(2-methyl-1,3-thiazol-4-yl)methyl]-2-oxo-1-imidazolidinyl}butanoyl)amino]-2-hydroxy-5-phenyl-1-[4-(3-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-1-[4-(6-methyl-2-pyridinyl)benzyl]-5-phenylpentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-5-phenyl-1-[4-(3-pyridinyl)benzyl]pentyl}amino)carbonyl]-2,2-dimethylpropylcarbamate;
methyl 1-[({4-{[2-(3-benzyl-2-oxo-1-imidazolidinyl)-3,3-dimethylbutanoyl])amino}-2-hydroxy-5-phenyl-1-[4-(3-pyridinyl)benzyl]pentyl}amino)carbonyl]-2,2-dimethylpropylcarbamate;
methyl 1-[({3-hydroxy-4-({2-[3-(3-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({4-{[2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-5-phenyl-1-[4-(4-pyridinyl)benzyl]pentyl}amino)carbonyl]-2,2-dimethylpropylcarbamate;
methyl 1-[({4-[((2S)-3,3-dimethyl-2-{3-[(6-methyl-2-pyridinyl)methyl]-2-oxo-1-imidazolidinyl}butanoyl}amino]-2-hydroxy-1-[4-(5-methyl-2-pyridinyl)benzyl]-5-phenylpentyl}amino)carbonyl]-2,2-dimethylpropylcarbamate;
methyl 1-[({3-hydroxy-4-({2-[3-(2-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({4-({3,3-dimethyl-2-[3-(2-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[3-(3-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({3-hydroxy-4-({2-[3-(2-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({3-hydroxy-4-({2-[3-(3-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-1-[4-(4-methyl-2-pyridinyl)benzyl]-5-phenylpentyl}amino)carbonyl]-2,2-dimethylpropylcarbamate;
methyl 1-[({4-{[2-(3-benzyl-2-oxo-1-imidazolidinyl)-3,3-dimethylbutanoyl]amino}-2-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(2-methyl-3-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-3-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[2-oxo-3-(3-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[2-oxo-3-(4-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 1-[({4-({3,3-dimethyl-2-[2-oxo-3-(2-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl 7-benzyl-1,10-ditert-butyl-5-hydroxy-4-[4-(6-methyl-3-pyridinyl)benzyl]-2,9,12-trioxo-13-oxa-3,8,11-triazatetradec-1-ylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-1-[4-(6-methyl-3-pyridinyl)benzyl]-5-phenylpentyl}amino)carbonyl]-2,2-dimethylpropylcarbamate;
methyl 1-[({4-[(3,3-dimethyl-2-{3-[(2-methyl-1,3-thiazol-4-yl)methyl]-2-oxo-1-imidazolidinyl}butanoyl)amino]-2-hydroxy-1-[4-(5-methyl-2-pyridinyl)benzyl]-5-phenylpentyl}amino)carbonyl]-2,2-dimethylpropylcarbamate;
methyl 7-benzyl-1,10-ditert-butyl-5-hydroxy-4-[4-(5-methyl-2-pyridinyl)benzyl]-2,9,12-trioxo-13-oxa-3,8,11-triazatetradec-1-ylcarbamate;
methyl 1-[({4[(3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2-methylbutylcarbamate;
methyl 4-benzyl-1,10-ditert-butyl-5-hydroxy-7-[4-(5-methyl-2-pyridinyl)benzyl]-2,9,12-trioxo-13-oxa-3,8,11-triazatetradec-1-ylcarbamate;
methyl 1-[({4-{[2-(3-benzyl-2-oxo-1-imidazolidinyl)-3,3-dimethylbutanoyl]amino}-3-hydroxy-1-[4-(5-methyl-2-pyridinyl)benzyl]-5-phenylpentyl}amino)carbonyl]-2,2-dimethylpropylcarbamate;
methyl 1-[({1-benzyl-4-[(3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentyl}amino)carbonyl]-2,2-dimethylpropylcarbamate;
methyl 4-benzyl-10-tert-butyl-5-hydroxy-1-[1-methyl-1-((R)-methylsulfinyl)ethyl]-2,9,12-trioxo-7-[4-(2-pyridinyl)benzyl]-13-oxa-3,8,11-triazatetradec-1-ylcarbamate;
methyl 1-[({2-hydroxy-4-[(3-methyl-2-{3-[(1-methyl-1H-benzimidazol-2-yl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({2-hydroxy-4-[(2-{3-[(2-isopropyl-1,3-thiazol-4-yl)methyl]-2-oxo-1-imidazolidinyl}-3-methylpentanoyl)amino]-5-phenyl-1-[4-(2-pyridinyl)benzyl]pentyl}amino)carbonyl]-2,2-dimethylpropylcarbamate;
methyl 1-[({3-hydroxy-4-[(3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl 1-[({-[(3,3-dimethyl-2-{3-[(1-methyl-1H-benzimidazol-2-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4[(3,3-dimethyl-2-{3-[(1-methyl-1H-benzimidazol-2-yl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl 1-[({3-hydroxy-4-[(2-{3-[(2-isopropyl-1,3-thiazol-4-yl)methyl]-2-oxo-1-imidazolidinyl}-3,3dimethylbutanoyl)amino]-5-phenyl-1-[4-(2-pyridinyl)benzyl]pentyl}amino)carbonyl]-2,2-dimethylpropylcarbamate;
methyl 1-[({2-hydroxy-4-[(2-{3-[(2-isopropyl-1,3-thiazol-4-yl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl 7-benzyl-1,10-ditert-butyl-5-hydroxy-2,9,12-trioxo-4-[4-(3-pyridinyl)benzyl]-13-oxa-3,8,11-triazatetradec-1-ylcarbamate;
methyl 7-benzyl-1,10-ditert-butyl-5-hydroxy-2,9,12-trioxo-4-[4-(4-pyridinyl)benzyl]-13-oxa-3,8,11-triazatetradec-1-ylcarbamate;
methyl 1-[({4-[(3,3-dimethyl-2-{3-[(2-methyl-1,3-thiazol-4-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(2-methyl-1,3-thiazol-4-yl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl 1-[({4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl 7-benzyl-10-sec-butyl-1-tert-butyl-6-hydroxy-13-methyl-14-(2-methyl-1,3-thiazol-4-yl)-2,9,12-trioxo-4-[4-(2-pyridinyl)benzyl]-3,8,11,13-tetraazatetradec-1-ylcarbamate;
methyl 7-benzyl-10-sec-butyl-1-tert-butyl-5-hydroxy-13-methyl-14-(2-methyl-1,3-thiazol-4-yl)-2,9,12-trioxo-4-[4-(2-pyridinyl)benzyl]-3,8,11,13-tetraazatetradec-1-ylcarbamate;
methyl 1-[({4-{[2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate;
1,2,5,6-tetradeoxy-2,5-bis({2-[(methoxycarbonyl)amino]-3,3-dimethylbutanoyl}amino)-1,6-bis[4-(2-pyridinyl)phenyl]-D-iditol;
methyl 1-[({4-[(3,3-dimethyl-2-{3-[(6-methyl-2-pyridinyl)methyl]-2-oxo-1-imidazolidinyl}butanoyl)amino]-3-hydroxy-1 [4-(6-methoxy-2-pyridinyl)benzyl]-5-phenylpentyl}amino)carbonyl]-2,2-dimethylpropylcarbamate;
methyl 1-[({4-{[2-(3-benzyl-2-oxo-1-imidazolidinyl)-3,3-dimethylbutanoyl]amino}-3-hydroxy-1-[4-(6-methoxy-2-pyridinyl)benzyl]-5-phenylpentyl}amino)carbonyl]-2,2-dimethylpropylcarbamate;
methyl 1-[({4-[(2-{3-[(6-tert-butyl-2-pyridinyl)methyl]-2-oxo-1-imidazolidinyl}-3,3-dimethylbutanoyl)amino]-3-hydroxy-5-phenyl-1-[4-(2-pyridinyl)benzyl]pentyl}amino)carbonyl]-2,2-dimethylpropylcarbamate;
methyl 1,10-ditert-butyl-5-hydroxy-2,9,12-trioxo-4,7-bis[4-(2-pyridinyl)benzyl]-13-oxa-3,8,11-triazatetradec-1-ylcarbamate;
methyl 1-[({3-hydroxy-4-[(2-{3-[(6-isopropyl-2-pyridinyl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl 1-[({4-[(2-{3-[(6-tert-butyl-2-pyridinyl)methyl]-2-oxo-1-imidazolidinyl}-3,3-dimethylbutanoyl)amino]-3-hydroxy-5-phenyl-1-[4-(2-pyridinyl)benzyl]pentyl}amino)carbonyl]-2,2-dimethylpropylcarbamate;
methyl 1-[({-4-{[2-(3-benzyl-2-oxo-1-imidazolidinyl)-3,3-dimethylbutanoyl]amino}-2-hydroxy-1-[4-(6-methoxy-2-pyridinyl)benzyl]-5-phenylpentyl}amino)carbonyl]-2,2-dimethylpropylcarbamate;
methyl 7-benzyl-1,10-ditert-butyl-5-hydroxy-4-[4-(6-methoxy-2-pyridinyl)benzyl]-2,9,12-trioxo-13-oxa-3,8,11-triazatetradec-1-ylcarbamate;
methyl 4-benzyl-1,10-ditert-butyl-5-hydroxy-7-[4-(6-methoxy-2-pyridinyl)benzyl]-2,9,12-trioxo-13-oxa-3,8,11-triazatetradec-1-ylcarbamate;
methyl 1-[({4-[(3,3-dimethyl-2-{3-[(6-methyl-2-pyridinyl)methyl]-2-oxo-1-imidazolidinyl}butanoyl)amino]-2-hydroxy-1-[4-(6-methoxy-2-pyridinyl)benzyl]-5-phenylpentyl}amino)carbonyl]-2,2-dimethylpropylcarbamate;
methyl 1-[({4-({2-[3-(2-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl 1-[({4-({2-[3-(4-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl 1-[({4-({2-[3-(3-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-3-hydroxy-1-[4-(5-methyl-2-pyridinyl)benzyl]-5-phenylpentyl}amino)carbonyl]-2,2-dimethylpropylcarbamate;
methyl 1-[({4-[(3,3-dimethyl-2-{3-[(6-methyl-2-pyridinyl)methyl]-2-oxo-1-imidazolidinyl}butanoyl)amino]-3-hydroxy-1-[4-(5-methyl-2-pyridinyl)benzyl]-5-phenylpentyl}amino)carbonyl]-2,2-dimethylpropylcarbamate;
methyl 1-[({3-hydroxy-4-[(2-{3-[(6-isopropyl-2-pyridinyl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl 1-[({1-benzyl-4-[(3,33-dimethyl-2-{3-[(6-methyl-2-pyridinyl)methyl]-2-oxo-1-imidazolidinyl}butanoyl)amino]-3-hydroxy-5-[4-(2-pyridinyl)phenyl]pentyl}amino)carbonyl]-2,2-dimethylpropylcarbamate;
methyl 4-benzyl-1,10-disec-butyl-5-hydroxy-2,9,12-trioxo-7-[4-(2-pyridinyl)benzyl]-13-oxa-3,8,11-triazatetradec-1-ylcarbamate; and
methyl 1-({[1-benzyl-2-hydroxy-4-({3-methyl-2-[2-oxo-3-(4-quinolinylmethyl)-1-imidazolidinyl]pentanoyl}amino)-5-phenylpentyl]amino}carbonyl)-2,2-dimethylpropylcarbamate.

5. The compound of claim 1 having formula (II) 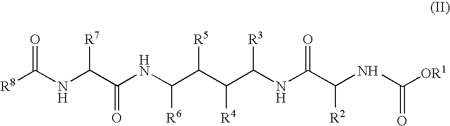
or a pharmaceutically acceptable salt form, stereoisomer, ester, salt of an ester, prodrug, salt of a prodrug, or combination thereof, wherein:
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(R_{b})C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R¹)C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SOₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁸ is —ORₐ or -alkylORₐ;
R¹⁶ is hydrogen or R¹⁵;
R¹⁵ is 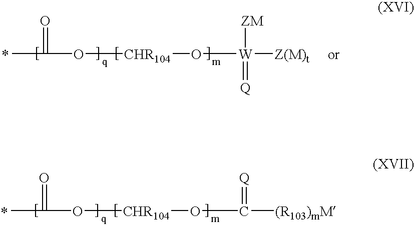
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅; —S(O)R₁₀₅, —SO₂R₁₀₅, —OCF₃, —SR₁₀₆-SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring systems is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c}; and
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂.

6. The compound of claim 5 wherein R⁴ is H and R⁵ is OR¹⁶.

7. The compound of claim 5 wherein R⁴ is OR¹⁶ and R⁵ is H.

8. The compound of claim 5 wherein R⁴ is H, R⁵ is OR¹⁶ and R² is alkyl.

9. The compound of claim 5 wherein R⁴ is OR¹⁶, R⁵ is H, and R² is alkyl.

10. The compound of claim 5 wherein R⁴ is H, R⁵ is OR¹⁶, R² is alkyl, R¹ is arylalkyl substituted with R^{3a}, and R^{3a} is aryl or heteroaryl.

11. The compound of claim 5 wherein R⁴ is OR¹⁶, R⁵ is H, R² is alkyl, R³ is arylalkyl substituted with R^{3a}, and R^{3a} is aryl or heteroaryl.

12. The compound of claim 5, or a pharmaceutically acceptable salt form, stereoisomer, ester, salt of an ester, prodrug, salt of a prodrug, or combination thereof, selected from the group consisting of:
methyl(1S,4R,6S,7S,10S)-7-benzyl-1,10-ditert-butyl-6-hydroxy-2,9,12-trioxo-4-[4-(2-pyridinyl)benzyl]-13-oxa-3,8,11-triazatetradec-1-ylcarbamate;
methyl(1S,4S,5S,7S,10S)-4-benzyl-1,10-ditert-butyl-5-hydroxy-2,9,12-trioxo-7-[4-(2-pyridinyl)benzyl]-13-oxa-3,8,11-triazatetradec-1-ylcarbamate;
methyl(1S,4S,5S,7S,10S)-7-benzyl-1,10-ditert-butyl-5-hydroxy-2,9,12-trioxo-4-[4-(2-pyridinyl)benzyl]-13-oxa-3,8,11-triazatetradec-1-ylcarbamate;
methyl(1R,4S,5S,7S,10S)-4-benzyl-10-tert-butyl-5-hydroxy-1-[1-methyl-1-(methylsulfanyl)ethyl]-2,9,12-trioxo-7-[4-(2-pyridinyl)benzyl]-13-oxa-3,8,11-triazatetradec-1-ylcarbamate;
methyl(1R,4S,5S,7S,10S)-4-benzyl-10-tert-butyl-5-hydroxy-1-[1-methyl-1-(methylsulfonyl)ethyl]-2,9,12-trioxo-7-[4-(2-pyridinyl)benzyl]-13-oxa-3,8,11-triazatetradec-1-ylcarbamate;
methyl(1R,4S,6S,7S,10S)-4-benzyl-10-tert-butyl-6-hydroxy-1-[1-methyl-1-(methylsulfanyl)ethyl]-2,9,12-trioxo-7-[4-(2-pyridinyl)benzyl]-13-oxa-3,8,11-triazatetradec-1-ylcarbamate;
methyl(1R,4S,6S,7S,10S)-4-benzyl-10-tert-butyl-6-hydroxy-1-[1-methyl-1-(methylsulfonyl)ethyl]-2,9,12-trioxo-7-[4-(2-pyridinyl)benzyl]-13-oxa-3,8,11-triazatetradec-1-ylcarbamate;
methyl(1S)-1-[({(1S,3S,4S)-4-({(2S)-3,3-dimethyl-2-[(phenoxyacetyl)amino]butanoyl}amino)-3-hydroxy-5-phenyl-1-[4-(2-pyridinyl)benzyl]pentyl}amino)carbonyl]-2,2-dimethylpropylcarbamate;
methyl(1S,4S,6S,7S,10S)-7-benzyl-1,10-ditert-butyl-6-hydroxy-2,9,12-trioxo-4-[4-(2-pyridinyl)benzyl]-14-oxa-3,8,11-triazapentadec-1-ylcarbamate;
methyl(1S)-1-[({(1S,3S,4S)-4-{[(2S)-3,3-dimethyl-2-({[(6-methyl-3-pyridinyl)oxy]acetyl}amino)butanoyl]amino}-3-hydroxy-5-phenyl-1-[4-(2-pyridinyl)benzyl]pentyl}amino)carbonyl]-2,2-dimethylpropylcarbamate;
3-pyridinylmethyl(1S,4S,5S,7S,10S)-4-benzyl-1,10-ditert-butyl-5-hydroxy-2,9,12-trioxo-7-[4-(2-pyridinyl)benzyl]-13-oxa-3,8,11-triazatetradec-1-ylcarbamate;
benzyl(1S,4S,5S,7S,10S)-4-benzyl-1,10-ditert-butyl-5-hydroxy-2,9,12-trioxo-7-[4-(2-pyridinyl)benzyl]-13-oxa-3,8,11-triazatetradec-1-ylcarbamate;
methyl(1S,4S,5S,7S,10S)-7-benzyl-1,10-ditert-butyl-5-hydroxy-4-[4-(6-methyl-3-pyridinyl)benzyl]-2,9,12-trioxo-13-oxa-3,8,11-triazatetradec-1-ylcarbamate;
methyl(1S,4S,5S,7S,10S)-7-benzyl-1,10-ditert-butyl-5-hydroxy-4-[4-(5-methyl-2-pyridinyl)benzyl]-2,9,12-trioxo-13-oxa-3,8,11-triazatetradec-1-ylcarbamate;
methyl(1S,4S,5S,7S,10S)-4-benzyl-1,10-ditert-butyl-5-hydroxy-7-[4-(5-methyl-2-pyridinyl)benzyl]-2,9,12-trioxo-13-oxa-3,8,11-triazatetradec-1-ylcarbamate;
1:1 mixture of methyl(1R,4S,5S,7S,10S)-4-benzyl-10-tert-butyl-5-hydroxy-1-[1-methyl-1-((R)-methylsulfinyl)ethyl]-2,9,12-trioxo-7-[4-(2-pyridinyl)benzyl]-13-oxa-3,8,11-triazatetradec-1-ylcarbamate and methyl(1R,4S,5S,7S,10S)-4-benzyl-10-tert-butyl-5-hydroxy-1-[1-methyl-1-((S)-methylsulfinyl)ethyl]-2,9,12-trioxo-7-[4-(2-pyridinyl)benzyl]-13-oxa-3,8,11-triazatetradec-1-ylcarbamate;
methyl(1S,4S,5S,7S,10S)-7-benzyl-1,10-ditert-butyl-5-hydroxy-2,9,12-trioxo-4-[4-(3-pyridinyl)benzyl]-13-oxa-3,8,11-triazatetradec-1-ylcarbamate;
methyl(1S,4S,5S,7S,10S)-7-benzyl-1,10-ditert-butyl-5-hydroxy-2,9,12-trioxo-4-[4-(4-pyridinyl)benzyl]-13-oxa-3,8,11-triazatetradec-1-ylcarbamate;
1,2,5,6-tetradeoxy-2,5-bis({(2S)-2-[(methoxycarbonyl)amino]-3,3-dimethylbutanoyl}amino)-1,6-bis[4-(2-pyridinyl)phenyl]-D-iditol;
methyl(1S,4R,5R,7R,10S)-1,10-ditert-butyl-5-hydroxy-2,9,12-trioxo-4,7-bis[4-(2-pyridinyl)benzyl]-13-oxa-3,8,11-triazatetradec-1-ylcarbamate;
methyl(1S,4S,5S,7S,10S)-7-benzyl-1,10-ditert-butyl-5-hydroxy-4-[4-(6-methoxy-2-pyridinyl)benzyl]-2,9,12-trioxo-13-oxa-3,8,11-triazatetradec-1-ylcarbamate;
methyl(1S,4S,5S,7S,10S)-4-benzyl-1,10-ditert-butyl-5-hydroxy-7-[4-(6-methoxy-2-pyridinyl)benzyl]-2,9,12-trioxo-13-oxa-3,8,11-triazatetradec-1-ylcarbamate; and
methyl(1S,4S,5S,7S,10S)-4-benzyl-1,10-disec-butyl-5-hydroxy-2,9,12-trioxo-7-[4-(2-pyridinyl)benzyl]-13-oxa-3,8,11-triazatetradec-1-ylcarbamate.

13. The compound of claim 1 having formula (III) 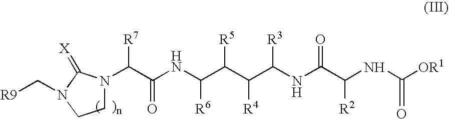
or a pharmaceutically acceptable salt form, stereoisomer, ester, salt of an ester, prodrug, salt of a prodrug, or combination thereof, wherein:
X is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(O)C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R⁹ is alkyl, alkenyl, alkynyl, cycloalkyl, cycloalkenyl, aryl, heteroaryl or heterocycle; wherein each R⁹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{9a};
R^{9a} is cycloalkyl, cycloalkenyl, heterocycle, aryl or heteroaryl; wherein each R^{9a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁶ is hydrogen or R¹⁵;
R¹⁵ is 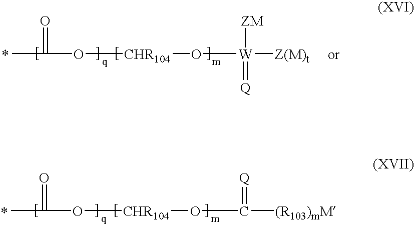
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring systems is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each P, and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

14. The compound of claim 13 wherein R⁴ is H and R⁵ is OR¹⁶.

15. The compound of claim 13 wherein R⁴ is OR¹⁶ and R⁵ is H.

16. The compound of claim 13 wherein R⁴ is H, R⁵ is OR¹⁶, X is O and R² is alkyl.

17. The compound of claim 13 wherein R⁴ is OR¹⁶, R⁵ is H, X is O and R² is alkyl.

18. The compound of claim 13 wherein R⁴ is H, R⁵ is OR¹⁶, X is O R² is alkyl, R³ is arylalkyl substituted with R^{3a}, and R^{3a} is aryl or heteroaryl.

19. The compound of claim 13 wherein R⁴ is OR¹⁶, R⁵ is H, X is O, R² is alkyl, R³ is arylalkyl substituted with R^{3a}, and R^{3a} is aryl or heteroaryl.

20. The compound of claim 13 wherein R⁴ is H R⁵ is OR¹⁶, X is O, R² is alkyl, R³ is arylalkyl substituted with R^{3a}, R⁹ is aryl or heteroaryl, and R^{3a} is aryl or heteroaryl.

21. The compound of claim 13 wherein R⁴ is OR¹⁶, R⁵ is H, X is O, R² is alkyl, R³ is arylalkyl substituted with R^{3a}, R⁹ is aryl or heteroaryl, and R^{3a} is aryl or heteroaryl.

22. The compound of claim 13, or a pharmaceutically acceptable salt form, stereoisomer, ester, salt of an ester, prodrug, salt of a prodrug, or combination thereof, selected from the group consisting of:
methyl(1S)-1-{[((1S,3S,4S)-1-benzyl-3-hydroxy-4-{[(2S)-3-methyl-2-(2-oxo-3-{[2-(2-pyridinyl)-1,3-thiazol-4-yl]methyl}-1-imidazolidinyl)pentanoyl]amino}-5-phenylpentyl)amino]carbonyl}-2,2-dimethylpropylcarbamate;
methyl(1S)-1-({[(1S,3S,4S)-1-benzyl-3-hydroxy-4-({(2S)-3-methyl-2-[2-oxo-3-(4-quinolinylmethyl)-1-imidazolidinyl]pentanoyl}amino)-5-phenylpentyl]amino}carbonyl)-2,2-dimethylpropylcarbamate;
methyl(1S)-1-({[(1S,3S,4S)-1-benzyl-3-hydroxy-4-({(2S)-3-methyl-2-[2-oxo-3-(4-quinolinylmethyl)-1-imidazolidinyl]pentanoyl}amino)-5-phenylpentyl]amino}carbonyl)-2-methylbutylcarbamate;
methyl(18)-1-{[((1S,3S,4S)-1-benzyl-3-hydroxy-4-{[(2S)-2-(3-{[2-(methoxymethyl)-1,3-thiazol-4-yl]methyl}-2-oxo-1-imidazolidinyl)-3,3-dimethylbutanoyl]amino}-5-phenylpentyl)amino]carbonyl}-2,2-dimethylpropylcarbamate;
methyl(1S)-1-[({(1S,3S,4S)-1-benzyl-3-hydroxy-4-[((2S)-3-methyl-2-{3-[(6-methyl-2-pyridinyl)methyl]-2-oxo-1-imidazolidinyl}pentanoyl)amino]-5-phenylpentyl}amino)carbonyl]-2,2-dimethylpropylcarbamate;
methyl(1S)-1-[({(1S,2S,4S)-1-benzyl-2-hydroxy-4-[((2S)-3-methyl-2-{3-[(6-methyl-2-pyridinyl)methyl]-2-oxo-1-imidazolidinyl}pentanoyl)amino]-5-phenylpentyl}amino)carbonyl]-2,2-dimethylpropylcarbamate;
methyl(1S)-1-[({(1S,2S,4S)-1-benzyl-2-hydroxy-4-[((2S)-3-methyl-2-{3-[(6-methyl-2-pyridinyl)methyl]-2-oxo-1-imidazolidinyl}pentanoyl)amino]-5-phenylpentyl}amino)carbonyl]-2-methylbutylcarbamate;
methyl(1S)-1-[({(1S,2S,4S)-1-benzyl-4-[((2S)-3,3-dimethyl-2-{3-[(6-methyl-2-pyridinyl)methyl]-2-oxo-1-imidazolidinyl}butanoyl)amino]-2-hydroxy-5-phenylpentyl}amino)carbonyl]-2,2-dimethylpropylcarbamate;
methyl(1S)-1-[({(1S,2S,4S)-1-benzyl-2-hydroxy-4-[((2S)-3-methyl-2-{3-[(2-methyl-1,3-thiazol-5-yl)methyl]-2-oxo-1-imidazolidinyl}pentanoyl)amino]-5-phenylpentyl}amino)carbonyl]-2,2-dimethylpropylcarbamate;
methyl(1S)-1-{[((1S,2S,4S)-1-benzyl-2-hydroxy-4-{[(2S)-3-methyl-2-(2-oxo-3-{[2-(3-pyridinyl)-1,3-thiazol-4-yl]methyl}-1-imidazolidinyl)pentanoyl]amino}-5-phenylpentyl)amino]carbonyl}-2,2-dimethylpropylcarbamate;
methyl(1S)-1-[({(1S,2S,4S)-1-benzyl-2-hydroxy-4-[((2S)-3-methyl-2-{3-[(6-methyl-3-pyridinyl)methyl]-2-oxo-1-imidazolidinyl}pentanoyl)amino]-5-phenylpentyl}amino)carbonyl]-2,2-dimethylpropylcarbamate;
methyl(1S)-1-[({(1S,2S,4S)-1-benzyl-4-[((2S)-3,3-dimethyl-2-{3-[(2-methyl-1,3-thiazol-4-yl)methyl]-2-oxo-1-imidazolidinyl}butanoyl)amino]-2-hydroxy-5-phenylpentyl}amino)carbonyl]-2,2-dimethylpropylcarbamate;
methyl(1S)-1-[({(1S,2S,4S)-1-benzyl-2-hydroxy-4-[((2S)-3-methyl-2-{3-[(2-methyl-3-pyridinyl)methyl]-2-oxo-1-imidazolidinyl}pentanoyl)amino]-5-phenylpentyl}amino)carbonyl]-2,2-dimethylpropylcarbamate;
methyl(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-{[((1S,2S,4S)-1-benzyl-2-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenylpentyl)amino]carbonyl}-2,2-dimethylpropylcarbamate;
methyl(1S)-1-[({(1S,3S,4S)-3-hydroxy-4-[((2S)-3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-{[((1S,2S,4S)-1-benzyl-4-{[(2S)-3,3-dimethyl-2-(2-oxo-3-{[2-(3-pyridinyl)-1,3-thiazol-4-yl]methyl}-1-imidazolidinyl)butanoyl]amino}-2-hydroxy-5-phenylpentyl)amino]carbonyl}-2,2-dimethylpropylcarbamate;
methyl(1S)-1-({[(1S,2S,4S)-1-benzyl-4-({(2S)-3,3-dimethyl-2-[2-oxo-3-(3-pyridinylmethyl)-1-imidazolidinyl]butanoyl}amino)-2-hydroxy-5-phenylpentyl]amino}carbonyl)-2,2-dimethylpropylcarbamate;
methyl(1S)-1-[({(1S,3S,4S)-3-hydroxy-4-[((2S)-3-methyl-2-{3-[(2-methyl-3-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1S,3S,4S)-1-benzyl-4-[((2S)-3,3-dimethyl-2-{3-[(6-methyl-2-pyridinyl)methyl]-2-oxo-1-imidazolidinyl}butanoyl)amino]-3-hydroxy-5-phenylpentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-1-benzyl-4-[((2S)-3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentyl}amino)carbonyl]-2,2-dimethylpropylcarbamate;
methyl(1S)-1-[({(1S,2S,4S)-2-hydroxy-4-[((2S)-3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1R,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-{[(2S)-3,3-dimethyl-2-(2-oxo-3-{[2-(3-pyridinyl)-1,3-thiazol-4-yl]methyl}-1-imidazolidinyl)butanoyl]amino}-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-{[(2S)-3,3-dimethyl-2-(2-oxo-3-{[2-(3-pyridinyl)-1,3-thiazol-4-yl]methyl}-1-imidazolidinyl)butanoyl]amino}-2-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-3-hydroxy-4-({(2S)-2-[3-(imidazo[1,5-a]pyridin-3-ylmethyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1S,2S,4S)-2-hydroxy-4-({(2S)-2-[3-(imidazo[1,5-a]pyridin-3-ylmethyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[2-oxo-3-(4-quinolinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-({(2S)-3,3-dimethyl-2-[2-oxo-3-(4-quinolinylmethyl)-1-imidazolidinyl]butanoyl}amino)-2-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-2-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl(1S)-1-[({(1S,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl(1S)-1-[({(1R,3S,4S)-4-{[(2S)-3,3-dimethyl-2-(2-oxo-3-{[2-(3-pyridinyl)-1,3-thiazol-4-yl]methyl}-1-imidazolidinyl)butanoyl]amino}-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate;
methyl(1S)-1-[({(1R,3S,4S)-4-({(2S)-3,3-dimethyl-2-[2-oxo-3-(4-quinolinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-4-[((2S)-3,3-dimethyl-2-{3-[(1-methyl-1H-benzimidazol-2-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-4-[((2S)-3,3-dimethyl-2-{3-[(2-methyl-1,3-thiazol-4-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[3-(2-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[3-(3-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(2-methyl-1,3-thiazol-4-yl)methyl]-2-oxo-1-imidazolidinyl}butanoyl)amino]-2-hydroxy-5-phenyl-1-[4-(3-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methyl-2-pyridinyl)benzyl]-5-phenylpentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-5-phenyl-1-[4-(3-pyridinyl)benzyl]pentyl}amino)carbonyl]-2,2-dimethylpropylcarbamate;
methyl(1S)-1-[({(1S,2S,4S)-4-{[(2S)-2-(3-benzyl-2-oxo-1-imidazolidinyl)-3,3-dimethylbutanoyl]amino}-2-hydroxy-5-phenyl-1-[4-(3-pyridinyl)benzyl]pentyl}amino)carbonyl]-2,2-dimethylpropylcarbamate;
methyl(1S)-1-[({(1S,3S,4S)-3-hydroxy-4-({(2S)-2-[3-(3-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1R,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-5-phenyl-1-[4-(4-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(5-methyl-2-pyridinyl)benzyl]-5-phenylpentyl}amino)carbonyl]-2,2-dimethylpropylcarbamate;
methyl(1S)-1-[({(1S,3S,4S)-3-hydroxy-4-({(2S)-2-[3-(2-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1R,3S,4S)-4-({(2S)-3,3-dimethyl-2-[3-(2-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-4-({(2S)-3,3-dimethyl-2-[3-(3-methylbenzyl)-2-oxo-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-3-hydroxy-4-({(2S)-2-[3-(2-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1R,3S,4S)-3-hydroxy-4-({(2S)-2-[3-(3-methoxybenzyl)-2-oxo-1-imidazolidinyl]-3,3-dimethylbutanoyl}amino)-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(4-methyl-2-pyridinyl)benzyl]-5-phenylpentyl}amino)carbonyl]-2,2-dimethylpropylcarbamate;
methyl(1S)-1-[({(1S,2S,4S)-4-{[(2S)-2-(3-benzyl-2-oxo-1-imidazolidinyl)-3,3-dimethylbutanoyl]amino}-2-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(2-methyl-3-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(6-methyl-3-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[2-oxo-3-(3-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[2-oxo-3-(4-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-3,3-dimethyl-2-[2-oxo-3-(2-pyridinylmethyl)-1-imidazolidinyl]butanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methyl-3-pyridinyl)benzyl]-5-phenylpentyl}amino)carbonyl]-2,2-dimethylpropylcarbamate;
methyl(1S)-1-[({(1S,2S,4S)-4-[((2S)-3,3-dimethyl-2-{3-[(2-methyl-1,3-thiazol-4-yl)methyl]-2-oxo-1-imidazolidinyl}butanoyl)amino]-2-hydroxy-1-[4-(5-methyl-2-pyridinyl)benzyl]-5-phenylpentyl}amino)carbonyl]-2,2-dimethylpropylcarbamate;
methyl(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2-methylbutylcarbamate;
methyl(1S)-1-[({(1S,3S,4S)-4-{[(2S)-2-(3-benzyl-2-oxo-1-imidazolidinyl)-3,3-dimethylbutanoyl]amino}-3-hydroxy-1-[4-(5-methyl-2-pyridinyl)benzyl]-5-phenylpentyl}amino)carbonyl]-2,2-dimethylpropylcarbamate;
methyl(1S)-1-[({(1S,2S,4R)-1-benzyl-4-[((2S)-3,3-dimethyl-2-{3-[(6-methyl-2-pyridinyl)methyl]-2-oxo-1-imidazolidinyl}butanoyl)amino]-2-hydroxy-5-[4-(2-pyridinyl)phenyl]pentyl}amino)carbonyl]-2,2-dimethylpropylcarbamate;
methyl(1S)-1-[({(1S,2S,4S)-2-hydroxy-4-[((2S)-3-methyl-2-{3-[(1-methyl-1H-benzimidazol-2-yl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1S,2S,4S)-2-hydroxy-4-[((2S)-2-{3-[(2-isopropyl-1,3-thiazol-4-yl)methyl]-2-oxo-1-imidazolidinyl}-3-methylpentanoyl)amino]-5-phenyl-1-[4-(2-pyridinyl)benzyl]pentyl}amino)carbonyl]-2,2-dimethylpropylcarbamate;
methyl(1S)-1-[({(1R,3S,4S)-3-hydroxy-4-[((2S)-3-methyl-2-{3-[(6-methyl-2-pyridinyl)methyl]-2-oxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(1-methyl-1H-benzimidazol-2-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(1-methyl-1H-benzimidazol-2-yl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-3-hydroxy-4-[((2S)-2-{3-[(2-isopropyl-1,3-thiazol-4-yl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl(1S)-1-[({(1S,2S,4S)-2-hydroxy-4-[((2S)-2-{3-[(2-isopropyl-1,3-thiazol-4-yl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(2-methyl-1,3-thiazol-4-yl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(2-methyl-1,3-thiazol-4-yl)methyl]-2-oxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl(1S)-1-[({(1S,3R,4S)-4-[((2S)-3,3-dimethyl-2-{3-[(6-methyl-2-pyridinyl)methyl]-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1R,3R,4S)-4-[((2S)-3,3-dimethyl-2-{3-[(6-methyl-2-pyridinyl)methyl}-2-oxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate;
pyridinyl)methyl]-2-oxo-1-imidazolidinyl}butanoyl)amino]-3-hydroxy-1-[4-(6-methoxy-2-pyridinyl)benzyl]-5-phenylpentyl}amino)carbonyl]-2,2-dimethylpropylcarbamate;
methyl(1S)-1-[({(1S,3S,4S)-4-{[(2S)-2-(3-benzyl-2-oxo-1-imidazolidinyl)-3,3-dimethylbutanoyl]amino}-3-hydroxy-1-[4-(6-methoxy-2-pyridinyl)benzyl]-5-phenylpentyl}amino)carbonyl]-2,2-dimethylpropylcarbamate;
methyl(1S)-1-[({(1S,3S,4S)-4-[((2S)-2-{3-[(6-tert-butyl-2-pyridinyl)methyl]-2-oxo-1-imidazolidinyl}-3,3-dimethylbutanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1R,3S,4S)-3-hydroxy-4-[((2S)-2-{3-[(6-isopropyl-2-pyridinyl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl(1S)-1-[({(1R,3S,4S)-4-[((2S)-2-{3-[(6-tert-butyl-2-pyridinyl)methyl]-2-oxo-1-imidazolidinyl}-3,3-dimethylbutanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-{[(2S)-2-(3-benzyl-2-oxo-1-imidazolidinyl)-3,3-dimethylbutanoyl]amino}-2-hydroxy-1-[4-(6-methoxy-2-pyridinyl)benzyl]-5-phenylpentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2-oxo-1-imidazolidinyl}butanoyl)amino]-2-hydroxy-1-[4-(6-methoxy-2-pyridinyl)benzyl]-5-phenylpentyl}amino)carbonyl]-0,2,2-dimethylpropylcarbamate;
methyl(1S)-1-[({(1S,3S,4S)-4-({(2S)-2-[3-(2-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-2-[3-(4-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-({(2S)-2-[3-(3-aminobenzyl)-2-oxo-1-imidazolidinyl]-3,3-dimethylbutanoyl}amino)-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(6-methyl-2-pyridinyl)methyl]-2-oxo-1-imidazolidinyl}butanoyl)amino]-3-hydroxy-1-[4-(5-methyl-2-pyridinyl)benzyl]-5-phenylpentyl}amino)carbonyl]-2,2-dimethylpropylcarbamate;
methyl(1S)-1-[({(1R,3S,4S)-4-[((2S)-3,3-dimethyl-2-{3-[(6-methyl-2-pyridinyl)methyl]-2-oxo-1-imidazolidinyl}butanoyl)amino]-3-hydroxy-1-[4-(5-methyl-2-pyridinyl)benzyl]-5-phenylpentyl}amino)carbonyl]-2,2-dimethylpropylcarbamate;
methyl(1S)-1-[({(1S,3S,4S)-3-hydroxy-4-[((2S)-2-{3-[(6-isopropyl-2-pyridinyl)methyl]-2-oxo-1-imidazolidinyl}-3,3-dimethylbutanoyl)amino]-5-phenyl-1-[4-(2-pyridinyl)benzyl]pentyl}amino)carbonyl]-2,2-dimethylpropylcarbamate;
methyl(1S)-1-[({(1S,3S,4S)-1-benzyl-4-[((2S)-3,3-dimethyl-2-{3-[(6-methyl-2-pyridinyl)methyl]-2-oxo-1-imidazolidinyl}butanoyl)amino]-3-hydroxy-5-[4-(2-pyridinyl)phenyl]pentyl}amino)carbonyl]-2,2-dimethylpropylcarbamate; and
methyl(8S)-1-({[(1S,2S,4S)-1-benzyl-2-hydroxy-4-({(2S)-3-methyl-2-[2-oxo-3-(4-quinolinylmethyl)-1-imidazolidinyl]pentanoyl}amino)-5-phenylpentyl]amino}carbonyl)-2,2-dimethylpropylcarbamate.

23. The compound of claim 1 having formula (IV) 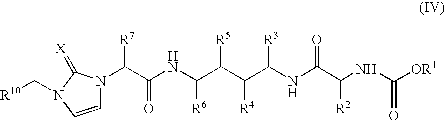
or a pharmaceutically acceptable salt form, stereoisomer, ester, salt of an ester, prodrug, salt of a prodrug, or combination thereof, wherein:
X is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐ, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), 7alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹⁰ is alkyl, alkenyl, alkynyl, cycloalkyl, cycloalkenyl, aryl, heteroaryl or heterocycle; wherein each R¹⁰ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{10a};
R^{10a} is cycloalkyl, cycloalkenyl, heterocycle, aryl or heteroaryl; wherein each R^{10a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁶ is hydrogen or R¹⁵;
R¹⁵ is 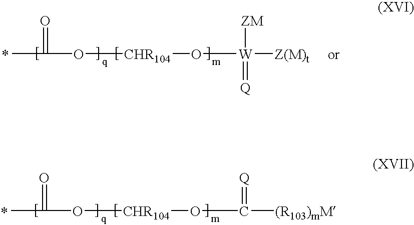
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring systems is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c}; and
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂.

24. The compound of claim 1 having formula (V) 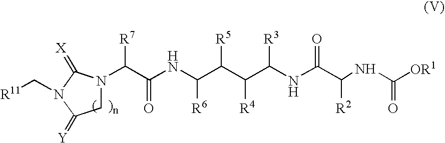
or a pharmaceutically acceptable salt form, stereoisomer, ester, salt of an ester, prodrug, salt of a prodrug, or combination thereof, wherein:
X is O, S or NH;
Y is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)N—H₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹¹ is alkyl, alkenyl, alkynyl, cycloalkyl, cycloalkenyl, aryl, heteroaryl or heterocycle; wherein each R¹¹ is susbstituted with 0, 1, 2 or 3 susbstituents independently selected from the group consisting of alkyl, alkenyl, alkynyl, cyano, formyl, halo, nitro, oxo, —ORₐ, —SRₐ, —SORₐ, —SO₂Rₐ, —SO₂NRₐ, —SO₂ORₐ, —NRₐR_{b}, —N(R_{b})C(O)Rₐ, —N(R_{b})SO₂Rₐ, —N(R_{b})SO₂NRₐR_{b}, —N(R_{b})C(O)NRₐR_{b}, —N(R_{b})C(O)ORₐ, —C(O)Rₐ, —C(O)NRₐR_{b}, —C(O)ORₐ, haloalkyl, nitroalkyl, cynaoalkyl, formylalkyl, -alkylORₐ, -alkylNRₐR_{b}, -alkylN(R_{b})C(O)ORₐ, -alkylN(R_{b})SO₂NRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkylC(O)Rₐ, -alkylC(O)NRₐR_{b} and R^{11a};
R^{11a} is cycloalkyl, cycloalkenyl, heterocycle, aryl or heteroaryl; wherein each R^{11a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹⁶ is hydrogen or R¹⁵;
R¹⁵ is 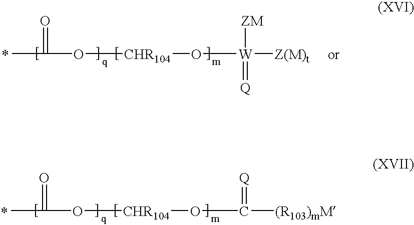
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring systems is substituted with 0, 1, 2, 3 or 4 substituents selected from the group consisting of oxo, —OR₄₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each F, and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂; and
n is 1 or 2.

25. The compound of claim 24 wherein X is O and Y is O.

26. The compound of claim 24 wherein X is O, Y is O, R⁴ is H and R⁵ is OR¹⁶.

27. The compound of claim 24 wherein X is O, Y is O, R⁴ is OR¹⁶ and R⁵ is H.

28. The compound of claim 24 wherein X is O, Y is O, R⁴ is H, R⁵ is OR¹⁶ and R² is alkyl.

29. The compound of claim 24 wherein X is O, Y is O, R⁴ is OR¹⁶, R⁵ is H, and R² is alkyl.

30. The compound of claim 24 wherein X is O, Y is O, R⁴ is H, R⁵ is OR¹⁶, R² is alkyl, R³ is arylalkyl substituted with R^{3a}, and R^{3a} is aryl or heteroaryl.

31. The compound of claim 24 wherein X is O, Y is O, R⁴ is OR¹⁶, R⁵ is H, R² is alkyl, R³ is arylalkyl substituted with R^{3a}, and R^{3a} is aryl or heteroaryl.

32. The compound of claim 24, or a pharmaceutically acceptable salt form, stereoisomer, ester, salt of an ester, prodrug, salt of a prodrug, or combination thereof, selected from the group consisting of:
methyl(1S)-1-[({(1S,3S,4S)-3-hydroxy-4-[((2S)-3-methyl-2-{3-[(6-methyl-2-pyridinyl)methyl]-2,4-dioxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1S,2S,4S)-2-hydroxy-4-[((2S)-3-methyl-2-{3-[(6-methyl-2-pyridinyl)methyl]-2,4-dioxo-1-imidazolidinyl}pentanoyl)amino]-5-phenyl-1-[4-(2-pyridinyl)benzyl]pentyl}amino)carbonyl]-2,2-dimethylpropylcarbamate;
methyl(1S)-1-[({(1R,3S,4S)-4-[((2S)-3,3-dimethyl-2-{3-[(6-methyl-2-pyridinyl)methyl]-2,4-dioxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-4-[((2S)-3,3-dimethyl-2-{3-[(6-methyl-2-pyridinyl)methyl]-2,4-dioxo-1-imidazolidinyl}butanoyl)amino]-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-[((2S)-3,3-dimethyl-2-{3-[(6-methyl-2-pyridinyl)methyl]-2,4-dioxo-1-imidazolidinyl}butanoyl)amino]-2-hydroxy-5-phenyl-1-[4-(2-pyridinyl)benzyl]pentyl}amino)carbonyl]-2,2-dimethylpropylcarbamate;
methyl(1S)-1-[({(1S,3S,4S)-3-hydroxy-4-[((2S)-2-{3-[(2-isopropyl-1,3-thiazol-4-yl)methyl]-2,4-dioxo-1-imidazolidinyl-}3-methylpentanoyl)amino]-5-phenyl-1-[4-(2-pyridinyl)benzyl]pentyl}amino)carbonyl]-2,2-dimethylpropylcarbamate; and
methyl(1S)-1-[({(1S,3S,4S)-4-{[(2S)-2-(2,4-dioxo-3-{[2-(3-pyridinyl)-1,3-thiazol-4-yl]methyl}-1-imidazolidinyl)-3-methylpentanoyl]amino}-3-hydroxy-5-phenyl-1-[4-(2-pyridinyl)benzyl]pentyl}amino)carbonyl]-2,2-dimethylpropylcarbamate.

33. The compound of claim 1 having formula (VI) 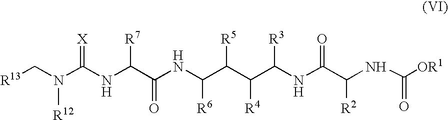
or a pharmaceutically acceptable salt form, stereoisomer, ester, salt of an ester, prodrug, salt of a prodrug, or combination thereof, wherein:
X is O, S or NH;
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R⁷ is alkyl, alkenyl, alkynyl, cycloalkyl, cycloalkenyl, heterocycle, aryl or heteroaryl; wherein each R⁷ is substituted with 0, 1 or 2 substituents independently selected from the group consisting of halo, —ORₐ, —SRₐ, —SORₐ, —SO₂Rₐ, —NRₐR_{b}, —N(R_{b})C(O)Rₐ, —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ, and R^{7a};
R^{7a} is cycloalkyl, cycloalkenyl, heterocycle, aryl or heteroaryl; wherein each R^{7a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(14)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R¹² is alkyl, alkenyl, cycloalkyl, cycloalkenyl, cycloalkylalkyl or cycloalkenylalkyl; wherein each R¹² is substituted with 0, 1 or 2 substituents independently selected from the group consisting of hydroxy, alkoxy and halo;
R¹³ is alkyl, alkenyl, alkynyl, cycloalkyl, cycloalkenyl, aryl, heteroaryl or heterocycle; wherein each R¹³ is susbstituted with 0, 1, 2 or 3 susbstituents independently selected from the group consisting of alkyl, alkenyl, alkynyl, cyano, halo, nitro, oxo, —ORₐ, —SRₐ, —SORₐ, —SO₂Rₐ, —SO₂NRₐ, —SO₂₀Rₐ, —NRₐR_{b}, —N(R_{b})C(O)Rₐ, —N(R_{b})C(O)ORₐ, —C(O)NRₐR_{b}, —C(O)ORₐ, haloalkyl, nitroalkyl, cynaoalkyl, -alkylORₐ, -alkylNRₐR_{b}, -alkylN(R_{b})C(O)Rₐ, -alkylN(R_{b})C(O)NRₐR_{b}, -alkylN(R_{b})SO₂Rₐ, -alkylC(O)ORₐ, -alkyl-C(O)NRₐR_{b} and R^{13a};
R^{13a} is cycloalkyl, cycloalkenyl, heterocycle, aryl or heteroaryl; wherein each R^{13a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R¹⁶ is hydrogen or R¹⁵;
R¹⁵ is 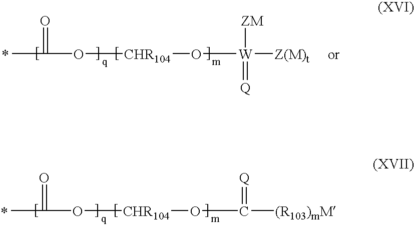
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₁₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆; —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring systems is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c}; and
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂.

34. The compound of claim 33 wherein R⁴ is H and R⁵ is OR¹⁶.

35. The compound of claim 33 wherein R⁴ is OR¹⁶ and R⁵ is H.

36. The compound of claim 33 wherein R⁴ is H, R⁵ is OR¹⁶, R³ is arylalkyl substituted with R^{3a}, and R^{3a} is aryl or heteroaryl.

37. The compound of claim 33 wherein R⁴ is OR¹⁶, R⁵ is H, R³ is arylalkyl substituted with R^{3a}, and R^{3a} is aryl or heteroaryl.

38. The compound of claim 33, or a pharmaceutically acceptable salt form, stereoisomer, ester, salt of an ester, prodrug, salt of a prodrug, or combination thereof, selected from the group consisting of:
methyl(1S,4S,6S,7S,10S)-7-benzyl-1,10-ditert-butyl-6-hydroxy-13-methyl-2,9,12-trioxo-14-phenyl-4-[4-(2-pyridinyl)benzyl]-3,8,11,13-tetraazatetradec-1-ylcarbamate;
methyl(1S,4R,6S,7S,10S)-7-benzyl-1,10-ditert-butyl-6-hydroxy-13-methyl-2,9,12-trioxo-14-phenyl-4-[4-(2-pyridinyl)benzyl]-3,8,11,13-tetraazatetradec-1-ylcarbamate;
methyl(1S,4S,6S,7S,10S)-7-benzyl-10-sec-butyl-1-tert-butyl-6-hydroxy-13-methyl-14-(2-methyl-1,3-thiazol-4-yl)-2,9,12-trioxo-4-[4-(2-pyridinyl)benzyl]-3,8,11,13-tetraazatetradec-1-ylcarbamate; and
methyl(1S,4S,5S,7S,10S)-7-benzyl-10-sec-butyl-1-tert-butyl-5-hydroxy-13-methyl-14-(2-methyl-1,3-thiazolyl)-2,9,12-trioxo-4-[4-(2-pyridinyl)benzyl]-3,8,11,13-tetraazatetradec-1-ylcarbamate.

39. The compound of claim 1 having formula (VII) 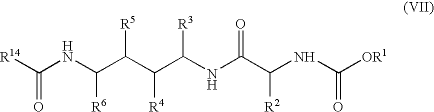
or a pharmaceutically acceptable salt form, stereoisomer, ester, salt of an ester, prodrug, salt of a prodrug, or combination thereof, wherein
R¹ is alkyl, alkenyl, alkynyl, cycloalkyl, cycloalkenyl, heterocycle, aryl or heteroaryl; wherein each R¹ is substituted with 0, 1 or 2 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂, and R^{1a};
R^{1a} is cycloalkyl, cycloalkenyl, heterocycle, aryl or heteroaryl; wherein each R^{1a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkylC(O)N(alkyl)₂;
R² is alkyl, alkenyl, alkynyl, cycloalkyl, cycloalkenyl, heterocycle, aryl or heteroaryl; wherein each R² is substituted with 0, 1 or 2 substituents independently selected from the group consisting of halo, —ORₐ, —SRₐ, —SORₐ, —SO₂Rₐ, —NRₐR_{b}, —NR_{b}C(O)Rₐ —N(R_{b})C(O)ORₐ, —N(Rₐ)C(═N)NRₐR_{b}, —N(Rₐ)C(O)NRₐR_{b}, —C(O)NRₐR_{b}, —C(O)ORₐ and R^{2a};
R^{2a} is cycloalkyl, cycloalkenyl, heterocycle, aryl or heteroaryl; wherein each R^{2a} is substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl) and -alkyl-C(O)N(alkyl)₂;
R³ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)NRₐR_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{3a};
R^{3a} is cycloalkyl, cycloalkenyl, heterocycle, aryl or heteroaryl; wherein each R^{3a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, —alkylC(O)NH₂, -alkylC(O)N(H)(alkyl), and -alkylC(O)N(alkyl)₂;
R⁴ is H and R⁵ is OR¹⁶; or
R⁵ is H and R⁴ is OR¹⁶; or
R⁴ and R⁵ are —OR¹⁶;
R⁶ is alkyl, alkenyl, alkynyl, haloalkyl, haloalkenyl, -alkylORₐ, -alkylSRₐ, -alkylSORₐ, -alkylSO₂Rₐ, -alkylNRₐR_{b}, -alkylN(R_{b})C(O)Rₐ, -alkylN(R_{b})C(O)ORₐ, -alkylN(Rₐ)C(═N)NRₐR_{b}, -alkylN(Rₐ)C(O)NRₐR_{b}, -alkylC(O)N % R_{b}, -alkylC(O)ORₐ, cycloalkyl, cycloalkylalkyl, cycloalkenyl, cycloalkenylalkyl, heterocycle, heterocyclealkyl, aryl, arylalkyl, heteroaryl or heteroarylalkyl; wherein the cycloalkyl, cycloalkenyl, heterocycle, aryl, heteroaryl, cycloalkyl moiety of the cycloalkylalkyl, cycloalkenyl moiety of the cycloalkenylalkyl, heterocycle moiety of the heterocyclealkyl, heteroaryl moiety of the heteroarylalkyl and the aryl moiety of the arylalkyl are independently substituted with 0, 1, 2, 3 or 4 substituents independently selected from the group consisting of cyano, halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R^{6a};
R^{6a} is cycloalkyl, cycloalkenyl, heterocycle, aryl or heteroaryl; wherein each R^{6a} is substituted with 0, 1, 2, 3 or 4 substituents independently selected from the group consisting of cyano, halo, oxo, alkyl, alkenyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), and -alkylC(O)N(alkyl)₂;
R¹⁴ is —ORₐ or -alkylORₐ;
R¹⁶ is hydrogen or R¹⁵;
R¹⁵ is 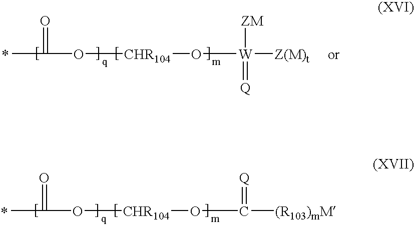
R₁₀₃ is C(R₁₀₅)₂, O or —N(R₁₀₅);
R₁₀₄ is hydrogen, alkyl, haloalkyl, alkoxycarbonyl, aminocarbonyl, alkylaminocarbonyl or dialkylaminocarbonyl,
each M is independently selected from the group consisting of H, Li, Na, K, Mg, Ca, Ba, —N(R₁₀₅)₂, alkyl, alkenyl, and R₄₀₆; wherein 1 to 4 —CH₂ radicals of the alkyl or alkenyl, other than the —CH₂ radical that is bound to Z, is optionally replaced by a heteroatom group selected from the group consisting of O, S, S(O), SO₂ and N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
Z is CH₂, O, S, —N(R₁₀₅), or, when M is absent, H;
Q is O or S;
W is P or S; wherein when W is S, Z is not S;
M′ is H, alkyl, alkenyl or R₁₀₆; wherein 1 to 4 —CH₂ radicals of the alkyl or alkenyl is optionally replaced by a heteroatom group selected from O, S, S(O), SO₂, or N(R₁₀₅); and wherein any hydrogen in said alkyl, alkenyl or R₁₀₆ is optionally replaced with a substituent selected from the group consisting of oxo, —OR₁₀₅, —R₁₀₅, —N(R₁₀₅)₂, —CN, —C(O)OR₁₀₅, —C(O)N(R₁₀₅)₂, —SO₂N(R₁₀₅), —N(R₁₀₅)C(O)R₁₀₅, —C(O)R₁₀₅, —SR₁₀₅, —S(O)R₁₀₅, —SO₂R₁₀₅, —OCF₃, —SR₁₀₆, —SOR₁₀₆, —SO₂R₁₀₆, —N(R₁₀₅)SO₂R₁₀₅, halo, —CF₃ and NO₂;
R₁₀₆ is a monocyclic or bicyclic ring system selected from the group consisting of aryl, cycloalkyl, cycloalkenyl heteroaryl and heterocycle; wherein any of said heteroaryl and heterocycle ring systems contains one or more heteroatom selected from the group consisting of O, N, S, SO, SO₂ and N(R₁₀₅); and wherein any of said ring system is substituted with 0, 1, 2, 3, 4, 5 or 6 substituents selected from the group consisting of hydroxy, alkyl, alkoxy, and —OC(O)alkyl;
each R₁₀₅ is independently selected from the group consisting of H or alkyl; wherein said alkyl is optionally substituted with a ring system selected from the group consisting of aryl, cycloalkyl, cycloalkenyl, heteroaryl and heterocycle; wherein any of said heteroaryl and heterocycle ring systems contains one or more heteroatoms selected from the group consisting of O, N, S, SO, SO₂, and N(R₁₀₅); and wherein any one of said ring systems is substituted with 0, 1, 2, 3 or 4 substituents selected from the group consisting of oxo, —OR₁₀₅, —R₁₀₅, —N(R₁₀₅)₂, —N(R₁₀₅)C(O)R₁₀₅, —CN, —C(O)OR₁₀₅, —C(O)N(R₁₀₅)₂, halo and —CF₃;
q is 0 or 1;
m is 0 or 1;
t is 0 or 1;
Rₐ and R_{b} at each occurrence are independently selected from the group consisting of hydrogen, alkyl, alkenyl, alkynyl, cycloalkyl, aryl, heteroaryl and heterocycle; wherein each Rₐ and R_{b}, at each occurrence, is independently substituted with 0, 1, 2 or 3 substituents independently selected from the group consisting of cyano, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c};
alternatively, Rₐ and R_{b}, together with the nitrogen atom to which they are attached, form a ring selected from the group consisting of heteroaryl and heterocycle; wherein each of the heteroaryl and heteroacycle is independently substituted with 0, 1, 2 or 3 substituents independently selected from the group consisting of alkyl, alkenyl, alkynyl, cyano, formyl, nitro, halo, oxo, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, cyanoalkyl, formylalkyl, nitroalkyl, haloalkyl, hydroxyalkyl, alkoxyalkyl, -alkylNH₂, -alkylN(H)(alkyl), -alkylN(alkyl)₂, -alkylN(H)C(O)NH₂, -alkylN(H)C(O)N(H)(alkyl), -alkylN(H)C(O)N(alkyl)₂, -alkylC(O)OH, -alkylC(O)Oalkyl, -alkylC(O)NH₂, -alkylC(O)N(H)(alkyl), -alkylC(O)N(alkyl)₂ and R_{c}; and
R_{c} is aryl, heteroaryl or heterocycle; wherein each R_{c} is independently substituted with 0, 1, 2, 3 or 4 substituents independently selected from the group consisting of halo, nitro, oxo, alkyl, alkenyl, alkynyl, hydroxy, alkoxy, —NH₂, —N(H)(alkyl), —N(alkyl)₂, —SH, —S(alkyl), —SO₂(alkyl), —N(H)C(O)alkyl, —N(alkyl)C(O)alkyl, —N(H)C(O)NH₂, —N(H)C(O)N(H)(alkyl), —N(H)C(O)N(alkyl)₂, —C(O)OH, —C(O)Oalkyl, —C(O)NH₂, —C(O)N(H)(alkyl), —C(O)N(alkyl)₂, haloalkyl, hydroxyalkyl, alkoxyalkyl, -alkyl-NH₂, -alkyl-N(H)(alkyl), -alkyl-N(alkyl)₂, -alkyl-N(H)C(O)NH₂, -alkyl-N(H)C(O)N(H)(alkyl), -alkyl-N(H)C(O)N(alkyl)₂, -alkyl-C(O)OH, -alkyl-C(O)Oalkyl, -alkyl-C(O)NH₂, -alkyl-C(O)N(H)(alkyl) and -alkyl-C(O)N(alkyl)₂.

40. The compound of claim 39 wherein R⁴ is H, R⁵ is OR¹⁶ and R² is alkyl.

41. The compound of claim 39 wherein R⁴ is OR¹⁶, R⁵ is H and R² is alkyl.

42. The compound of claim 39 wherein R⁴ is H, R⁵ is OR¹⁶, R² is alkyl, R³ is arylalkyl substituted with R^{3a}, and R^{3a} is aryl or heteroaryl.

43. The compound of claim 39 wherein R⁴ is OR¹⁶, R⁵ is H, R² is alkyl, R³ is arylalkyl substituted with R^{3a}, and R^{3a} is aryl or heteroaryl.

44. The compound of claim 39, or a pharmaceutically acceptable salt form, stereoisomer, ester, salt of an ester, prodrug, salt of a prodrug, or combination thereof, selected from the group consisting of:
1:1 mixture of (3R,3aS,6aR)-hexahydrofuro[2,3-b]furan-3-yl(1S,3S,4S)-1-benzyl-3-hydroxy-4-({(2S)-2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl(1S,3S,4S)-1-benzyl-3-hydroxy-4-({(2S)-2-[(methoxycarbonyl)amino]-3,3-dimethylbutanoyl}amino)-5-[4-(2-pyridinyl)phenyl]pentylcarbamate;
1:1 mixture of (3R,3aS,6aR)-hexahydrofuro[2,3-b]furan-3-yl(1S,2S,4S)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl(1S,2S,4S)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate;
methyl(1S)-1-[({(1S,3S,4S)-4-{[(2,6-dimethylphenoxy)acetyl]amino}-3-hydroxy-5-phenyl-1-[4-(2-pyridinyl)benzyl]pentyl}amino)carbonyl]-2,2-dimethylpropylcarbamate;
methyl(1S)-1-[({(1S,2S,4S)-4-{[(2,6-dimethylphenoxy)acetyl]amino}-2-hydroxy-5-phenyl-1-[4-(2-pyridinyl)benzyl]pentyl}amino)carbonyl]-2,2-dimethylpropylcarbamate;
1:1 mixture of (3R,3aS,6aR)-hexahydrofuro[2,3-b]furan-3-yl(1S,2S,4R)-1-benzyl-2-hydroxy-4-({(2S)-2-[(methoxycarbonyl)amino]-3,3-dimethylbutanoyl}amino)-5-[4-(2-pyridinyl)phenyl]pentylcarbamate and (3R,3aR,6aS)-hexahydrofuro[2,3-b]furan-3-yl(1S,2S,4R)-1-benzyl-2-hydroxy-4({(2S)-2-[(methoxycarbonyl)amino]-3,3-dimethylbutanoyl}amino)-5-[4-(2-pyridinyl)phenyl]pentylcarbamate; and
methyl(1S)-1-[({(1R,3S,4S)-4-{[(2,6-dimethylphenoxy)acetyl]amino}-3-hydroxy-5-phenyl-1-[4-(2-pyridinyl)benzyl]pentyl}amino)carbonyl]-2,2-dimethylpropylcarbamate.

45. Methyl(1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof.

46. Methyl(1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of an ester, prodrug, salt of a prodrug, or combination thereof.

47. The compound of claim 13 wherein R¹ is alkyl.

48. The compound of claim 13 wherein R¹ is methyl.

49. The compound of claim 13 wherein R² is alkyl.

50. The compound of claim 13 wherein R² is tert-butyl.

51. The compound of claim 13 wherein R³ is arylalkyl.

52. The compound of claim 13 wherein R³ is phenylmethyl.

53. The compound of claim 13 wherein R⁶ is arylalkyl.

54. The compound of claim 13 wherein R⁶ is phenylmethyl.

55. The compound of claim 13 wherein R⁷ is alkyl.

56. The compound of claim 13 wherein R⁷ is tert-butyl.

57. The compound of claim 13 wherein R⁹ is aryl.

58. The compound of claim 13 wherein R⁹ is phenyl.

59. The compound of claim 13 wherein R⁹ is heteroaryl.

60. The compound of claim 13 wherein R⁹ is pyridyl.

61. The compound of claim 13 wherein R⁴ is H, R⁵ is OR¹⁶, X is O and R^{11s} alkyl.

62. The compound of claim 13 wherein R⁴ is OR¹⁶, R⁵ is H, X is O and R⁶ is alkyl.

63. The compound of claim 13 wherein R⁴ is H, R⁵ is OR¹⁶, X is O and R³ is arylalkyl.

64. The compound of claim 13 wherein R⁴ is OR¹⁶, R⁵ is H, X is O and R³ is arylalkyl.

65. The compound of claim 13 wherein R⁴ is H R⁵ is OR¹⁶, X is O and R⁶ is arylalkyl.

66. The compound of claim 13 wherein R⁴ is OR¹⁶, R⁵ is H, X is O and R⁶ is arylalkyl.

67. The compound of claim 13 wherein R⁴ is H, R⁵ is OR¹⁶, X is O and R⁷ is alkyl.

68. The compound of claim 13 wherein R⁴ is OR¹⁶, R⁵ is H, X is O and R⁷ is alkyl.

69. The compound of claim 13 wherein R⁴ is H, R⁵ is OR¹⁶, X is O and R⁹ is aryl.

70. The compound of claim 13 wherein R⁴ is OR¹⁶, R⁵ is H, X is O and R⁹ is aryl.

71. The compound of claim 13 wherein R⁴ is H, R⁵ is OR¹⁶, X is O and R⁹¹ is heteroaryl.

72. The compound of claim 13 wherein R⁴ is OR¹⁶, R⁵ is H, X is O and R⁹ is heteroaryl.

73. The compound of claim 13 wherein R¹ is alkyl, R² is alkyl, R³ is arylalkyl, R⁴ is H, R⁵ is OR¹⁶, R⁶ is arylalkyl, R⁷ is alkyl and R⁹ is aryl.

74. The compound of claim 13 wherein R¹ is methyl, R² is tert-butyl, R³ is phenylmethyl, R⁴ is H, R⁵ is OH, R⁶ is phenylmethyl, R⁷ is tert-butyl and R⁹ is phenyl.

75. The compound of claim 13 wherein R¹ is alkyl, R² is alkyl, R³ is arylalkyl, R⁴ is H, R⁵ is OR¹⁶, R⁶ is arylalkyl, R⁷ is alkyl and R⁹ is heteroaryl.

76. The compound of claim 13 wherein R¹ is methyl, R² is tert-butyl, R³ is phenylmethyl, R⁴ is H, R⁵ is OH, R⁶ is phenylmethyl, R⁷ is tert-butyl and R⁹ is pyridyl.

77. The compound of claim 13 wherein R¹ is alkyl, R² is alkyl, R³ is arylalkyl, R⁴ is OR¹⁶, R⁵ is H, R⁶ is arylalkyl, R⁷ is alkyl and R⁹ is aryl.

78. The compound of claim 13 wherein R¹ is methyl, R² is tert-butyl, R³ is phenylmethyl, R⁴ is OH, R⁵ is H, R⁶ is phenylmethyl, R⁷ is tert-butyl and R⁹ is phenyl.

79. The compound of claim 13 wherein R¹ is alkyl, R² is alkyl, R³ is arylalkyl, R⁴ is OR¹⁶, R⁵ is H, R⁶ is arylalkyl, R⁷ is alkyl and R⁹ is heteroaryl.

80. The compound of claim 13 wherein R¹ is methyl, R² is tert-butyl, R³ is phenylmethyl, R⁴ is OH, R⁵ is H, R⁶ is phenylmethyl, R⁷ is tert-butyl and R⁹ is pyridyl.

81. A pharmaceutical composition comprising a therapeutically effective amount of a compound or combination of compounds of claim 1, and a pharmaceutically acceptable carrier.

82. A pharmaceutical composition comprising a therapeutically effective amount of methyl (1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate, or a pharmaceutically acceptable salt form, stereoisomer, ester, salt of a ester, prodrug, salt of a prodrug, or combination thereof, and a pharmaceutically acceptable carrier.

83. A pharmaceutical composition comprising a therapeutically effective amount of methyl (15)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate, or a pharmaceutically acceptable salt, stereoisomer, ester, salt of a ester, prodrug, salt of a prodrug, or combination thereof, and a pharmaceutically acceptable carrier.

84. A pharmaceutical composition comprising a therapeutically effective amount of a compound or combination of compounds of claim 1, and one, two, three, four, five or six agents selected from the group consisting of a second HIV protease inhibitor, a HIV reverse transcriptase inhibitor, an HIV entry/fusion inhibitor, an HIV integrase inhibitor and an HIV budding/maturation inhibitor, and a pharmaceutically acceptable carrier.

85. The pharmaceutical composition of claim 84 wherein the second HIV protease inhibitor is selected from the group consisting of ritonavir, lopinavir, saquinavir, amprenavir, fosamprenavir, nelfinavir, tipranavir, indinavir, atazanavir, TMC-126, TMC-114, mozenavir (DMP-450), JE-2147 (AG1776), L-756423, RO0334649, KNI-272, DPC-681, DPC-684 and GW640385X.

86. The pharmaceutical composition of claim 84 wherein the HIV reverse transcriptase inhibitor is selected from the group consisting of lamivudine, stavudine, zidovudine, abacavir, zalcitabine, didanosine, tenofovir, emtricitabine, amdoxovir, elvucitabine, alovudine, MIV-210, Racivir (t-FTC), D-D4FC (Reverset, DPC-817), SPD754, nevirapine, delavirdine, efavirenz, capravirine, emivirine, calanolide A, GW5634, BMS-56190 (DPC-083), DPC-961, MIV-150, TMC-120 and TMC-125.

87. The pharmaceutical composition of claim 84 wherein the HIV entry/fusion inhibitor is selected from the group consisting of enfuvirtide (T-20), T-1249, PRO 2000, PRO 542, PRO 140, AMD-3100, BMS-806, FP21399, GW873140, Schering C (SCH-C), Schering D (SCH-D), TNX-355 and UK-427857.

88. The pharmaceutical composition of claim 84 wherein the HIV integrase inhibitor is selected from the group consisting of S-1360, zintevir (AR-177), L-870812 and L-870810.

89. The pharmaceutical composition of claim 84 wherein the HIV budding/maturation inhibitor is PA-457.

90. The pharmaceutical composition of claim 84 wherein the compound of claim 1 is methyl (1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate.

91. The pharmaceutical composition of claim 84 wherein the compound of claim 1 is methyl (1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate.

92. A pharmaceutical composition comprising a therapeutically effective amount of a compound or combination of compounds of claim 1, or a pharmaceutically acceptable salt form, stereoisomer, ester, salt of an ester, prodrug, salt of a prodrug, or combination thereof, ritonavir and a pharmaceutically acceptable carrier.

93. The pharmaceutical composition of claim 92 wherein the compound of claim 1 is methyl (1S)-1-[({(1R,3S,4S)-3-hydroxy-4-{[(2S)-2-(3-{[6-(1-hydroxy-1-methylethyl)-2-pyridinyl]methyl}-2-oxo-1-imidazolidinyl)-3,3-dimethylbutanoyl]amino}-5-phenyl-1-[4-(2-pyridinyl)benzyl]pentyl}amino)carbonyl]-2,2-dimethylpropylcarbamate.

94. The pharmaceutical composition of claim 92 wherein the compound of claim 1 is methyl (1S)-1-[({(1S,3S,4S)-4-{[(2S)-2-(3-benzyl-2-oxo-1-imidazolidinyl)-3,3-dimethylbutanoyl]amino}-3-hydroxy-5-phenyl-1-[4-(2-pyridinyl)benzyl]pentyl}amino)carbonyl]-2,2-dimethylpropylcarbamate.

95. A method of inhibiting the replication of an HIV virus comprising contacting said virus with a therapeutically effective amount of a compound or combination of compounds of any one of claims 1, 45 and 46.

96. A method of inhibiting HIV protease comprising contacting said HIV protease with a therapeutically effective amount of a compound or combination of compounds of any one of claims 1, 45 and 46.

97. A method for treating or preventing an HIV infection comprising administering to a patient in need of such treatment a therapeutically effective amount of a compound or combination of compounds of any one of claims 1, 45 and 46.

98. A method for treating or preventing an HIV infection comprising administering to a patient in need of such treatment a pharmaceutical composition of any one of claims 81, 82, 83, 84, 85, 86, 87, 88, 89, 90, 91, 92, 93 and 94.
